(19) United States
(12) Patent Application Publication
(10) Pub. No.: US 2026/0158175 A1
(43) Pub. Date: Jun. 11, 2026

(54) RADIOLABELED AMINO ACIDS FOR CANCER IMAGING, THERAPY AND CORRESPONDING LABELING METHODS THEREOF

(71) Applicant: THE UNIVERSITY OF NORTH CAROLINA AT CHAPEL HILL, Chapel Hill, NC (US)

(72) Inventors: Anthony Casarez, San Diego, CA (US); David A. Nicewicz, Durham, NC (US); Zibo Li, Chapel Hill, NC (US)

(21) Appl. No.: 19/229,858

(22) Filed: Jun. 5, 2025

Related U.S. Application Data
(63) Continuation-in-part of application No. PCT/US24/58920, filed on Dec. 6, 2024.

(51) Int. Cl. A61K 51/04; A61K 49/10; C07C 229/36; C07D 205/04; C07D 295/185

(52) U.S. Cl. CPC A61K 51/0446; A61K 49/10; A61K 51/0402; A61K 51/0444; C07C 229/36; C07D 205/04; C07D 295/185; A61K 2121/00

(57) ABSTRACT

The disclosure relates to double halogenated phenylalanine derivatives, which can be radiolabeled with PET or SPECT radioisotopes, and their use in treating and/or imaging cancer.

### RELATED APPLICATION DATA

The present application claims priority pursuant to 35 U.S.C. § 120 to Patent Cooperation Treaty Application Number PCT/US2024/58920 filed Dec. 6, 2024, which is incorporated herein by reference in its entirety.

### GOVERNMENTAL SUPPORT

This invention was made with government support under Grant No. EB029451 awarded by National Institutes of Health. The government has certain rights in the invention.

### FIELD OF INVENTION

The disclosure relates to poly halogenated phenylalanine derivatives, which can be radiolabeled with PET or SPECT radioisotopes and therapeutic radioisotopes, and their use in treating and/or imaging cancer.

### BACKGROUND OF THE INVENTION

Pancreatic ductal adenocarcinoma (PDAC) is one of the deadliest human malignancies characterized by a fast and aggressive progression. Due to a lack of methods to detect pancreatic carcinoma at an early stage and its aggressive progression, the disease is often far advanced in patients by the time a definite diagnosis is established, resulting in only 20% of patients having the surgically resectable disease at the time of diagnosis. For patients with locally advanced PDAC, a combination of chemotherapeutic agents has become a standard of care aiming to improve overall survival compared to single agents but at the cost of increased toxicity. Unfortunately, only 40% of patients remain disease-free at 3 years after surgical resection plus the use of potent but toxic chemotherapies.

Just like in many other cancer types, energy metabolism reprogramming has also been implicated in the tumorigenesis and development of pancreatic cancer. Accumulating evidence suggests that amino acids metabolism orchestrated by genetic alterations contributes to pancreatic cancer malignant characteristics including cell proliferation, invasion, metastasis, angiogenesis and redox balance. In the nutrient-deficient tumor microenvironment (TME), the interactions between cancer cells and stromal components and salvaging processes play critical roles in fulfilling the metabolic requirements and supporting growth of PDAC. Advanced pancreatic tumors, in particular, have been shown to be notoriously difficult to treat because of their highly concentrated fibrous network of stroma that prevents resection and acts as a roadblock to xenobiotic delivery.

Hence, there is an urgent need to develop effective therapeutic and prognostic methods for PDAC management.

### SUMMARY OF THE INVENTION

The current application discloses poly halogenated phenylalanine derivates exhibiting efficient tumor uptake with low background accumulation to be used as imaging agents and/or radionuclide-based therapy agents for various cancers, including but not limited to PDAC.

As such, one aspect of the current application is a compound of Formula (I):

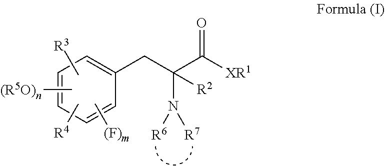

wherein X is —O—, —NH—, —N[(C₃-C₆) cycloalkyl]-, or —N[(C₁-C₆) alkyl]-;
R¹ and R² are independently selected from the group consisting of —H, -PG, —CH₂Ar, —(C₁-C₄) alkyl and —(C₃-C₇) cycloalkyl, wherein in some instances X and R¹ together form a (C₃-C₅) heterocycle,
R³ is —I, —Br or a radioisotope R* selected from the group consisting of [123]I, [124]I, [125]I, [131]I, [210]At, [211]At, [76]Br, [77]Br and [82]Br;
R⁴ is —H, —F or radioisotope [18]F;
R⁵ is -PG, —(C₁-C₆) alkyl or —(C₃-C₆) cycloalkyl;
R⁶ and R⁷ are independently selected from the group consisting of —H, -PG, —CH₂Ar-(C₁-C₄) alkyl, and —(C₃-C₆) cycloalkyl, or R⁶ and R⁷ together form a (C₃-C₅) heterocycle;
PG, in each instance, is a protecting group;
m is 0, 1, 2, or 3; and
n is 0, 1 or 2; and
any stereoisomer and/or pharmaceutically acceptable salt thereof.

Another aspect disclosed herein is directed toward methods for the preparation of compounds of Formula (I), wherein R³ is a radioisotope R* selected from the group consisting of [123]I, [124]I, [125]I, [131]I, [210]At, [211]At, [76]Br, [77]Br and [82]Br, the method comprising
(a) obtaining a starting material of Formula (IV):

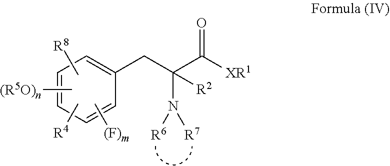

wherein X is —O—, —NH—, —N[(C₃-C₆) cycloalkyl]-, or —N[(C₁-C₆) alkyl]-;
R¹ is selected from the group consisting of —H, -PG, —CH₂Ar, —(C₁-C₄) alkyl and —(C₃-C₇) cycloalkyl, wherein in some instances X and R¹ together form a (C₃-C₅) heterocycle;
R² is selected from the group consisting of —H, —(C₁-C₄) alkyl and —(C₃-C₇) cycloalkyl,
R⁴ is —H or —F;
R⁵ is -PG, —(C₁-C₆) alkyl or —(C₃-C₆) cycloalkyl;
R⁶ and R⁷ are independently selected from the group consisting of —H, -PG, —CH₂Ar, —(C₁-C₄) alkyl, and —(C₃-C₆) cycloalkyl, or R⁶ and R⁷ together form a (C₃-C₅) heterocycle;
R⁸ is selected from the group consisting of —B[O(C₁-C₆) alkyl]₂, —B[—O((C₁-C₆) alkyl)O—], —B(OH)₂, —BF₃K, N-methyliminodiacetic acid boronate, —Sn(C₁-C₄ alkyl)₃, —Ge[(C₁-C₆) alkyl]₃ and —Si[(C₁-C₆) alkyl]₃;
PG, in each instance, is a protecting group;
m is 0, 1, 2, or 3; and
n is 0, 1 or 2; and
any stereoisomer and/or pharmaceutically acceptable salt thereof; and
(b) contacting the starting material with a radioisotope source in the presence of a suitable oxidant to render a compound of Formula (I), wherein R³ is a radioisotope R* selected from the group consisting of [123]I, [124]I, [125]I, [131]I, [210]At, [211]At, [76]Br, [77]Br and [82]Br.

Another aspect disclosed herein is a method of imaging a subject for diagnosing a disease or assessing efficacy of a treatment, the method comprising:
(a) administering to the subject in need thereof an effective amount of a compound of Formula (I), wherein the compound contains a radioisotope; and
(b) acquiring at least one image of at least one portion of the subject.

Another aspect disclosed herein is a method of treating a subject in need thereof, the method comprising administering to the patient in need thereof a compound of Formula (I).

Another aspect of the disclosure is directed to a pharmaceutical composition comprising a compound as disclosed herein or a pharmaceutically acceptable salt thereof, and one or more pharmaceutically acceptable carrier(s).

Another aspect disclosed herein is a method for treating a disease or condition that is treatable by modulation of amino acid metabolism or by utilization of an amino acid transport mechanism, the method comprising administering to a subject in need thereof a therapeutically effective amount of a compound, prodrug, or a pharmaceutical composition as disclosed herein. In some embodiments, the disease is cancer. In some embodiments, the cancer is pancreatic cancer or brain cancer.

### BRIEF DESCRIPTION OF THE DRAWINGS

FIG. 1 shows reaction schemes of the three different disclosed radiofluorination methods A-C employed for the preparation of [18]F radiolabeled compounds disclosed herein.

FIG. 2 shows UV-Vis HPLC (212 nm/254 nm) and RadioHPLC analyses of (R)-2-((Diphenylmethylene)amino)-3-(3-fluoro-5-(fluoro-¹⁸F)-2-iodo-6-methoxyphenyl)propanoic acid and (R)-2-((Diphenylmethylene)amino)-3-(3-fluoro-5-(fluoro-¹⁸F)-6-iodo-2-methoxyphenyl)propanoic acid by using HPLC condition 1-1.

FIG. 3 shows UV-Vis HPLC (212 nm/254 nm) and RadioHPLC analyses of (R)-2-Amino-3-(3-fluoro-5-(fluoro-¹⁸F)-2-iodo-6-methoxyphenyl)propanoic acid and (R)-2-Amino-3-(3-fluoro-5-(fluoro-¹⁸F)-6-iodo-2-methoxyphenyl)propanoic acid by using HPLC condition 2.

FIG. 4 shows quality control, according to UV-Vis HPLC (212 nm/254 nm) and RadioHPLC analyses, of (R)-2-Amino-3-(3-fluoro-5-(fluoro-¹⁸F)-2-iodo-6-methoxyphenyl)propanoic acid and (R)-2-Amino-3-(3-fluoro-5-(fluoro-¹⁸F)-6-iodo-2-methoxyphenyl)propanoic acid using HPLC condition 2.

FIG. 5 shows F-18 imaging, biodistribution for (R)-2-amino-3-(3-fluoro-5-(fluoro-¹⁸F)-2-iodo-6-methoxyphenyl)propanoic acid and (R)-2-amino-3-(3-fluoro-5-(fluoro-¹⁸F)-6-iodo-2-methoxyphenyl)propanoic acid. MCF7 tumor volume ˜609.65 mm³. Average injected dose: 27.1 uCi; Scan time: 28 min, 77 min, and 154 min p.i.

FIG. 6 shows UV-Vis HPLC (212 nm/254 nm) and RadioHPLC analyses of (S)-2-((Diphenylmethylene)amino)-3-(3-fluoro-5-(fluoro-¹⁸F)-2-iodo-6-methoxyphenyl)propanoic acid and (S)-2-((Diphenylmethylene)amino)-3-(3-fluoro-5-(fluoro-¹⁸F)-6-iodo-2-methoxyphenyl)propanoic acid by using HPLC condition 1-1.

FIG. 7 shows UV-Vis HPLC (212 nm/254 nm) and RadioHPLC analyses of (S)-2-Amino-3-(3-fluoro-5-(fluoro-¹⁸F)-2-iodo-6-methoxyphenyl)propanoic acid and (S)-2-Amino-3-(3-fluoro-5-(fluoro-¹⁸F)-6-iodo-2-methoxyphenyl)propanoic acid by using HPLC condition 2.

FIG. 8 shows quality control, according to UV-Vis HPLC (212 nm/254 nm) and RadioHPLC analyses, of (S)-2-Amino-3-(3-fluoro-5-(fluoro-¹⁸F)-2-iodo-6-methoxyphenyl)propanoic acid and (S)-2-Amino-3-(3-fluoro-5-(fluoro-¹⁸F)-6-iodo-2-methoxyphenyl)propanoic acid using HPLC condition 2.

FIG. 9 shows F-18 imaging, biodistribution for (S)-2-amino-3-(3-fluoro-5-(fluoro-¹⁸F)-2-iodo-6-methoxyphenyl)propanoic acid and (S)-2-amino-3-(3-fluoro-5-(fluoro-¹⁸F)-6-iodo-2-methoxyphenyl)propanoic acid. Average injected dose: 77.6±2.1 uCi; Scan time: 47 min, 150 min, and 244 min p.i.

FIG. 10 shows UV-Vis HPLC (212 nm/254 nm) and RadioHPLC analyses of (S)-2-((Diphenylmethylene)amino)-3-(2-fluoro-5-(fluoro-¹⁸F)-3-iodo-6-methoxyphenyl)propanoic acid by using HPLC condition 1-2.

FIG. 11 shows UV-Vis HPLC (212 nm/254 nm) and RadioHPLC analyses of (R)-2-((Diphenylmethylene)amino)-3-(2-fluoro-5-(fluoro-¹⁸F)-3-iodo-6-methoxyphenyl)propanoic acid by using HPLC condition 1-1.

FIG. 12 shows UV-Vis HPLC (212 nm/254 nm) and RadioHPLC analyses of (R)-2-Amino-3-(2-fluoro-5-(fluoro-¹⁸F)-3-iodo-6-methoxyphenyl)propanoic acid by using HPLC condition 2.

FIG. 13 shows UV-Vis HPLC (212 nm/254 nm) and RadioHPLC analyses of (S)-2-amino-3-(5-fluoro-4-(fluoro-¹⁸F)-2-iodophenyl)propanoic acid under procedure B1.

FIG. 14 shows UV-Vis HPLC (212 nm/254 nm) and RadioHPLC analyses of (S)-2-amino-3-(5-fluoro-4-(fluoro-¹⁸F)-2-iodophenyl)propanoic acid under procedure B2; HPLC condition 3.

FIG. 15 shows quality control, according to UV-Vis HPLC (254 nm) and RadioHPLC, of S)-2-amino-3-(5-fluoro-4-(fluoro-¹⁸F)-2-iodophenyl)propanoic acid by using HPLC Condition 3.

FIG. 16 shows UV-Vis HPLC (212 nm/254 nm) analysis of ¹⁹F-standard of S)-2-amino-3-(5-fluoro-4-(fluoro-¹⁸F)-2-iodophenyl)propanoic acid.

FIG. 17 shows F-18 imaging, biodistribution for (S)-2-amino-3-(5-fluoro-4-(fluoro-¹⁸F)-2-iodophenyl)propanoic acid. Average injected dose: 88.2±9.8 uCi; Scan time: 43 min, 87 min, and 199 min p.i.

FIG. 18 shows UV-Vis HPLC (212 nm/254 nm) and RadioHPLC analyses of (R)-2-amino-3-(5-fluoro-4-(fluoro-¹⁸F)-2-iodophenyl)propanoic acid using HPLC condition 4.

FIG. 19 shows co-injection analysis by spiking with the ¹⁹F-standard for (R)-2-amino-3-(5-fluoro-4-(fluoro-¹⁸F)-2-iodophenyl)propanoic acid; HPLC condition 4 [UV-Vis HPLC (212 nm/254 nm) and RadioHPLC analyses].

FIG. 20 shows UV-Vis HPLC (212 nm/254 nm) analysis ¹⁹F-standard for (R)-2-amino-3-(5-fluoro-4-(fluoro-¹⁸F)-2-iodophenyl)propanoic acid; HPLC condition 4.

FIG. 21 shows F-18 imaging, biodistribution for (R)-2-amino-3-(5-fluoro-4-(fluoro-¹⁸F)-2-iodophenyl)propanoic acid. Average injected dose: 110.9±11.1 uCi; Scan time: 33 min, 101 min, and 169 min p.i.

FIG. 22 shows purification of (S)-2-amino-3-(4,5-difluoro-2-(iodo-¹²⁴I)phenyl)propanoic acid by HPLC condition 6 [UV-Vis HPLC (212 nm/254 nm)].

FIG. 23 shows quality control of (S)-2-amino-3-(4,5-difluoro-2-(iodo-¹²⁴I)phenyl)propanoic acid by HPLC condition 6.

FIG. 24 shows purification of (R)-2-amino-3-(4,5-difluoro-2-(iodo-¹²⁴I)phenyl)propanoic acid by HPLC condition 5 [UV-Vis HPLC (254 nm) and RadioHPLC analyses].

FIG. 25 shows the ¹²⁷I-standard for (R)-2-amino-3-(4,5-difluoro-2-(iodo-¹²⁴I)phenyl)propanoic acid.

FIG. 26 shows quality control for (R)-2-amino-3-(4,5-difluoro-2-(iodo-¹²⁴I)phenyl)propanoic acid by HPLC condition 6 [UV-Vis HPLC (212 nm) and RadioHPLC analyses].

FIG. 27 shows in-vitro stability testing for (R)-2-amino-3-(4,5-difluoro-2-(iodo-¹²⁴I)phenyl)propanoic acid with 7-hour post in vivo formulation [UV-Vis HPLC (212 nm) and RadioHPLC analyses].

FIG. 28 shows in-vitro stability testing for (R)-2-amino-3-(4,5-difluoro-2-(iodo-¹²⁴I)phenyl)propanoic acid with 24-hour post in vivo formulation [UV-Vis HPLC (212 nm) and RadioHPLC analyses].

FIG. 29 shows I-124 imaging, biodistribution for (R)-2-amino-3-(4,5-difluoro-2-(iodo-¹²⁴I)phenyl)propanoic acid. Average injected dose: 152.2±2.3 uCi; Scan time: 68 min and 222 min p.i.

FIG. 30 shows purification of (R)-2-amino-3-(4,5-difluoro-2-(iodo-¹³¹I)phenyl)propanoic acid by HPLC condition 5 [UV-Vis HPLC (212 nm/254 nm) and RadioHPLC analyses].

FIG. 31 shows quality control for R)-2-amino-3-(4,5-difluoro-2-(iodo-¹³¹I)phenyl)propanoic acid; HPLC condition 6 [UV-Vis HPLC (212 nm) and RadioHPLC analyses].

FIG. 32 illustrates inhibited tumor growth in MCF7 xenografts subsequent to injection of a radiolabled compound described herein according to some embodiments.

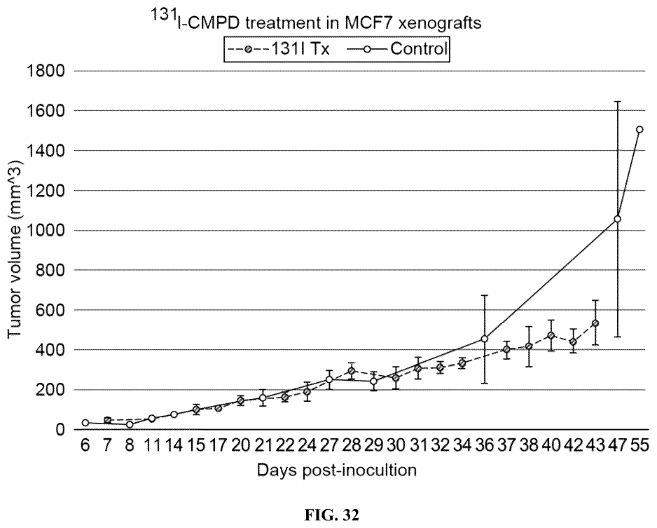

FIG. 33 shows quality control, according to UV-Vis HPLC (212 nm/254 nm) and RadioHPLC analyses, of (R)-2-amino-3-(2-(astato-²¹¹At)-4,5-difluorophenyl)propanoic acid using HPLC condition 1.

FIG. 34 shows quality control, according to UV-Vis HPLC (212 nm/254 nm) and RadioHPLC analyses, of (R)-2-amino-3-(2-(astato-²¹¹At)-4,5-difluorophenyl)propanoic acid using HPLC condition 2.

FIG. 35 shows quality control, according to UV-Vis HPLC (212 nm/254 nm) and RadioHPLC analyses, of (R)-2-amino-3-(2-(astato-²¹¹At)-4,5-difluorophenyl)propanoic acid using HPLC condition 3.

FIG. 36 illustrates stability of ²¹¹At-radiolabeled product after 2 hours of incubation of the in-vitro formulation as assessed by HPLC condition 3.

FIG. 37 illustrates averaged tumor volumes for the efficacy study in MCF-7 tumorized mice employing ²¹¹At-radiolabeled product.

FIG. 38 illustrates averaged body weights for the efficacy study in MCF-7 tumorized mice ²¹¹At-radiolabeled product.

FIG. 39 illustrates individual tumor volumes for the efficacy study in MCF-7 tumorized mice employing ²¹¹At-radiolabeled product.

FIG. 40 illustrates individual body weights for the efficacy study in MCF-7 tumorized mice employing ²¹¹At-radiolabeled product.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

Associated with extremely poor prognosis, pancreatic ductal adenocarcinoma (PDAC) is typically diagnosed at late stages in which cancer has already metastasized. Although various treatments have been developed, PDAC remains one of the most fatal malignancies, with a 5-year survival rate of <9%. Key features that make PDAC so extraordinarily difficult to treat include pronounced alterations in stromal responses and immune surveillance programs that prevent successful local therapy. PDAC tumors are characterized by isolated nests of tumor cells surrounded by extensive networks of fibrous growth. As a result, 60-90% of the PDAC tumor volume is made of dense stroma containing extracellular matrix, cancer-associated fibroblasts, and a variety of immunosuppressive cells. This creates a pro-tumorigenic and immunosuppressive microenvironment that downregulates the body's ability to detect and kill cancer cells. Furthermore, the stromal bulk creates elevated interstitial fluid pressure within the tumor, which prevents efficient drug diffusion into the tumor parenchyma. Therapies targeting tumor cells alone are therefore unlikely to be successful due to physical barriers such as dense desmoplasia and the unique hypoperfused tumor microenvironment (TME) of PDAC. Therefore, there is a clear need to develop innovative therapeutic agents for PDAC management.

The current disclosure is directed towards developing innovative imaging and therapeutic agents for the management of PDAC and other cancers by employing the radiolabeled phenylalanine derivatives disclosed herein. Unlike most current imaging agents and anti-cancer agents which rely on glucose metabolism for screening patients, monitoring treatment, or providing a prognosis, the compounds disclosed herein rely on the amino acid metabolism of cancer cells. Amino acid metabolism is usually enhanced in tumor progression and therefore represents another important pathway for developing imaging agents and/or therapeutic compounds. The capability to monitor amino acid metabolism can not only be used for tumor detection, but also provides important information on tumor characteristics (which could guide therapy and lead to personalized medicine). In PDAC and other cancers, amino acid metabolism plays a vital role in the initiation and progression of the cancer, which provides a unique opportunity to image and treat cancers, such as pancreatic cancer once imaging/therapy radionuclides are introduced to the amino acid backbone.

Thus, the disclosure is directed to 1) highly innovative photoredox methods (used to generate radiofluorinated amino acids), which not only allow easy conversion of traditional drug molecules to PET/SPECT agents, but also could be used to produce established agents on a large scale that were previously complicated to synthesize. The recent development of S_{N}Ar radiofluorination allows precise control of the labeling position. Unlike traditional labeling reactions, the disclosed photoredox system features mild labeling conditions, and is a metal-free catalyst system (eliminating the need to analyze residual metal contaminants). Indeed, this method provides easy access to a unique library of phenylalanine derivatives for fast screening based on PET imaging; 2) developing theranostic agents. As already mentioned above, the unique role of amino acids in PDAC and other cancers, allows for the development of poly halogenated agents that can be used for cancer prognosis (based on [18]F) and radionuclide-based therapy (based primarily on [131]I and [211]At), all of which are anticipated to impact the care of PDAC patients. Importantly, [131]I based agents and the corresponding [18]F imaging agents possess the same atom connectivity as their non-radioactive ([127]I/[19]F) isotope-bearing analogs, making them a true theranostic pair.

Not to be bound theory, but it is believed that poly halogenated phenylalanine derivatives exhibit prominent tumor uptake with low background accumulation, which can be used as imaging/therapy agents for PDAC and other cancer management. For example, some poly halogenated phenylalanine derivatives contain one fluorine atom and one iodine atom. The role of the F atom could be: (1) The fluorination position allows for tuning the tumor uptake and tumor to background ratio; and (2) The [19]F atom could be converted to a [18]F atom, which will lead to a companion diagnostic PET agent of the radionuclide-based therapy agent. The role of the I atom could be: (1) The iodine in phenylalanine derivatives could be converted to [131]I to deliver beta-particles to the tumor site (beta particle therapy); and (2) The iodine atom could be converted to [211]At to be used as an alpha-particle emitting therapeutic agent. Given the important role of amino acids in crosstalk between cancer cells and stromal components, these agents can be developed as TME-targeted therapeutics against PDAC or other cancers.

### I. Definitions

As used in the specification and the appended claims, the singular forms “a,” “an” and “the” include plural referents unless the context clearly dictates otherwise. Thus, for example, reference to “an alkyl group” or “a phenyl” includes mixtures of two or more such alkyl groups or phenyls.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed. Further, unless specified by the term “integer,” the number specified includes fractions or numbers with decimals. For example, the range of “from about 1 to about 5” includes numbers such as 1, 1.1, 1.5, 2.0, 2.2, and so on. As used herein, the term “integer” refers to a number that is a whole number, and not a fraction.

References in the specification and concluding claims to parts by weight of a particular element or component in a composition denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5 and are present in such ratio regardless of whether additional components are contained in the compositions.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the term “alkyl group” refers to a saturated hydrocarbon radical containing 1 to 8, 1 to 6, 1 to 4, or 5 to 8 carbons. In some embodiments, the saturated radical contains more than 8 carbons. An alkyl group is structurally similar to a noncyclic alkane compound modified by the removal of one hydrogen from the noncyclic alkane and the substitution therefore of a non-hydrogen group or radical. Alkyl group radicals can be branched or unbranched. Lower alkyl group radicals have 1 to 4 carbon atoms. Higher alkyl group radicals have 5 to 8 carbon atoms. Examples of alkyl, lower alkyl, and higher alkyl group radicals include, but are not limited to, methyl, ethyl, n-propyl, isopropyl, n-butyl, sec-butyl, isobutyl, t-butyl, amyl, t-amyl, n-pentyl, n-hexyl, i-octyl and like radicals.

As used therein, the term “PG” stands for protecting group, which is a functionality that is designed to protect amines, hydroxyl, phenoxyl and/or acids from certain reaction conditions. A skilled artisan would generally be familiar with this methodology and would consider “Greene's Protective Groups in Organic Synthesis” by Peter G. M. Wuts, Theodora W. Greene, First published:10 Apr. 2006, John Wiley & Sons, Inc.

As used herein, the term “Ar” stands for an aromatic ring such as aryls and heteroaryls.

The term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “heteroaryl” refers to an aromatic 5-10 membered ring system where the heteroatoms are selected from O, N, or S, and the remainder ring atoms being carbon (with appropriate hydrogen atoms unless otherwise indicated). Heteroaryl groups may be optionally substituted with one or more substituents. In one embodiment, 0, 1, 2, 3, or 4 atoms of each ring of a heteroaryl group may be substituted by a substituent. Examples of heteroaryl groups include pyridyl, furanyl, thienyl, pyrrolyl, oxazolyl, oxadiazolyl, imidazolyl, thiazolyl, isoxazolyl, quinolinyl, pyrazolyl, isothiazolyl, pyridazinyl, pyrimidinyl, pyrazinyl, triazinyl, isoquinolinyl, indazolyl, and the like.

As used herein, the designations “C(═O),” “CO” and “C(O)” are used to indicate a carbonyl moiety. Examples of suitable carbonyl moieties include, but are not limited to, those found in ketones and aldehydes.

The term “cycloalkyl” refers to a hydrocarbon with 3-8 members or 3-7 members or 3-6 members or 3-5 members or 3-4 members and can be monocyclic or bicyclic. The ring may be saturated or may have some degree of unsaturation. In some cases, the degree of saturation encompasses aromatic compounds. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, phenyl, and the like.

As used herein, the term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Included herein are also 7-12 membered bicyclic, or 10-14 membered tricyclic ring system comprising 1-3 heteroatoms, wherein at least one of the rings is non-aromatic but the other ring systems can be aromatic. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include azetidinyl, pyrrolidinyl, piperidinyl, piperazinyl, tetrahydropyranyl, morpholinyl, thiomorpholinyl, 1,3-dioxolanyl, tetrahydrofuryl, tetrahydrothienyl, thienyl, and the like.

As used herein, the terms “halo,” “halogen,” and “halide” refer to any suitable halogen, including —F, —Cl, —Br, —I and —At.

As used herein, the term “ester”, used alone or as part of another group, refers to a —C(O)OR radical, where R is any suitable substituent such as alkyl, cycloalkyl, alkenyl, alkynyl or aryl.

As used herein, the term “amide”, used alone or as part of another group, refers to a —C(O)NR^{a}R^{b} radical, where R^{a} and R^{b} are any suitable substituent such as alkyl, cycloalkyl, alkenyl, alkynyl or aryl.

As used herein, the terms “increase,” “increases,” “increased,” “increasing”, “improve,” “enhance,” and similar terms indicate an elevation in the specified parameter of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more.

As used herein, the terms “reduce,” “reduces,” “reduced,” “reduction,” “inhibit,” and similar terms refer to a decrease in the specified parameter of at least about 5%, 10%, 15%, 20%, 25%, 30%, 35%, 40%, 45%, 50%, 55%, 60%, 65%, 70%, 75%, 80%, 85%, 90%, 95%, 97%, or 100%.

As used herein, the “contacting” refers to reagents in close proximity so that a reaction may occur.

As used herein, the term “stereoisomer” refers to compounds which have identical chemical constitution, but differ with regards to the arrangement of the atoms or groups in space. These “stereoisomers” have a “stereogenic center” which may be a chiral center.

As used herein, the term “chiral” refers to molecules which have the property of non-superimposability of the mirror image partner, while the term “achiral” refers to molecules which are superimposable on their mirror image partner.

As used herein, the term “diastereomers” refers to a stereoisomer with two or more centers of chirality and whose molecules are not mirror images of one another. Diastereomers have different physical properties, e.g., melting points, boiling points, spectral properties, and reactivity. Mixtures of diastereomers may separate under high-resolution analytical procedures such as electrophoresis and chromatography.

As used herein, the term “enantiomers” refers to two stereoisomers of a compound which are non-superimposable mirror images of one another. Stereochemical definitions and conventions used herein generally follow S. P. Parker, Ed., McGraw-Hill Dictionary of Chemical Terms (1984) McGraw-Hill Book Company, New York; and Eliel, E. and Wiley, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including, but not limited to, diastereomers, enantiomers and atropisomers, as well as mixtures thereof such as racemic mixtures, form part of the present invention. Mixtures of stereoisomers may separate under high-resolution analytical procedures such as electrophoresis, chiral salt formation and chromatography.

As used herein, the term “theranostic agent” refers to compounds that are able to detect as well as treat a disease or condition. For example, the compounds disclosed herein can contain two different halogens, such as a fluorine atom, an iodine atom and an astatine atom. A theranostic agent may be one compound labeled with [18]F used for imaging and another theranostic agent may be labeled with [131]I or [211]At for treatment. Both theranostic agents maintain the same atom connectivity regardless of the radioisotope and are referred to as a “theranostic pair”.

As used herein, the term “subject” broadly refers to any animal, including but not limited to, human and non-human animals (e.g., mice, rats, dogs, pigs, cats, cows, horses, sheep, poultry, fish, crustaceans, etc.). As used herein, the term “patient” typically refers to a subject that is being treated for a disease or condition.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., cell cycle checkpoint inhibitor and one or more additional therapeutics) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition or formulation” refers to the combination of an active agent with a carrier, inert or active, making the composition or formulation especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject. “Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Non-limiting examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, dextrin or cyclodextrins; chelating agents such as EDTA; sugar alcohols such as mannitol or sorbitol; salt-forming counterions such as sodium, potassium, calcium, and magnesium; and/or nonionic surfactants such as TWEEN™, polyethylene glycol (PEG), and poloxamers (PLURONICS™). In certain embodiments, the pharmaceutically acceptable carrier is a non-naturally occurring pharmaceutically acceptable carrier.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter of biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder.

As used herein, a subject is “in need of a treatment” if such a subject would benefit biologically, medically or in quality of life from such treatment.

As used herein, the term “a therapeutically effective amount” of a compound of the present invention refers to an amount of the compound of the present invention that will elicit the biological or medical response of a subject, for example, reduction or inhibition of an enzyme or a protein activity, reduction in tumor volume, or ameliorate symptoms, alleviate condition, slow or delay disease progression or prevent a disease, etc.

As used herein, the term “anticancer agent” or antineoplastic agent, refers to a therapeutic agent that is useful for treating or controlling the growth of cancerous cells.

### II. Compounds

Provided herein are compounds that can be employed for PET/SPECT imaging, radionuclide-based therapy, and/or as therapeutics for treating cancer and/or hyperproliferative disorders or conditions. In some embodiments, such compounds can modulate or utilize the amino acid metabolism and/or transport mechanisms of cells (e.g., cancer cells).

In one aspect, the compounds disclosed herein comprise a compound of Formula (I):

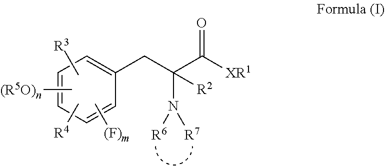

wherein X is —O—, —NH—, —N[(C₃-C₆) cycloalkyl]-, or —N[(C₁-C₆) alkyl]-;
R¹ and R² are independently selected from the group consisting of —H, -PG, —CH₂Ar, —(C₁-C₄) alkyl and —(C₃-C₇) cycloalkyl, wherein in some instances X and R¹ together form a (C₃-C₅) heterocycle,
R³ is —I, —Br or a radioisotope R* selected from the group consisting of [123]I, [124]I, [125]I, [131]I, [210]At, [211]At, [76]Br, [77]Br and [82]Br;
R⁴ is —H, —F or radioisotope [18]F;
R⁵ is -PG, —(C₁-C₆) alkyl or —(C₃-C₆) cycloalkyl;
R⁶ and R⁷ are independently selected from the group consisting of —H, -PG, —CH₂Ar, —(C₁-C₄) alkyl, and —(C₃-C₆) cycloalkyl, or R⁶ and R⁷ together form a (C₃-C₅) heterocycle;
PG, in each instance, is a protecting group;
m is 0, 1, 2, or 3; and
n is 0, 1 or 2; and
any stereoisomer and/or pharmaceutically acceptable salt thereof.

In some embodiments, n is 2. In some embodiments, n is 1. In some embodiments, n is 0.

In some embodiments, R⁵ is —(C₁-C₆) alkyl. In some embodiments, R⁵ is selected from the group consisting of —CH₃, —CH₂CH₃, —CH(CH₃)₂, —CH₂CH₂CH₃, —CH₂CH₂CH₂CH₃, —CH₂CH(CH₃)₂, —C(CH₃)₃, —CH₂CH₂CH₂CH₂CH₃, —CH₂C(CH₃)₃, —CH₂CH₂CH(CH₃)₂, —CH₂CH₂CH₂CH₂CH₂CH₃, —CH₂CH₂C(CH₃)₃ and —CH₂CH₂CH₂CH(CH₃)₂. In some embodiments, R⁵ is selected from the group consisting of CH₃, —CH₂CH₃, —CH(CH₃)₂, and —CH₂CH₂CH₃. In some embodiments, R⁵ is —CH₃.

In some embodiments, R⁵ is —(C₃-C₆) cycloalkyl. In some embodiments R⁵ is selected from the group consisting of cyclopropyl, cyclobutyl, cyclopentyl, cyclohexyl, and phenyl.

In some embodiments, n is 1 and R⁵ is —(C₁-C₆) alkyl. In some embodiments, n is 1 and R⁵ is —CH₃.

In some embodiments, n is 2 and R⁵ is —(C₁-C₆) alkyl. In some embodiments, n is 2 and R⁵ is —CH₃.

In some embodiments, R¹ and R² are independently selected from the group consisting of —(C₁-C₄) alkyl and —(C₃-C₆) cycloalkyl. In some embodiments, R¹ and R² are independently selected from the group consisting of —CH₃, —CH₂CH₃, —CH(CH₃)₂, —CH₂CH₂CH₃, —CH₂CH₂CH₂CH₃, —CH₂CH(CH₃)₂, —C(CH₃)₃. In some embodiments, R² is —CH₃. In some embodiments, R¹ is —CH₃. In some embodiments, R¹ is —H.

In some embodiments, R¹ is (C₃-C₇) cycloalkyl. In some embodiments R¹ is selected from the group consisting of cyclopropyl, cyclobutyl, cyclopentyl, and cyclohexyl.

In some embodiments, R⁶ and R⁷ together form a (C₃-C₅) heterocycle. Exemplary (C₃-C₅) heterocycles include, but are not limited to the following:

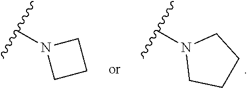 [SMILES: *N1CCC1 ; *N1CCCC1]

In some embodiments, PG is a protecting group for amines, hydroxyl and/or acid functionality. In some embodiments, PG is a protecting group for hydroxyl moieties (e.g., —OH).

In some embodiments, PG is a protecting group for amine moieties (e.g., primary amines or secondary amines). In some embodiments, PG is a protecting group for carboxylic acids (e.g. —COOH). In some embodiments, PG is a protecting group for carboxamides (e.g., —CONH₂ or —CONHR).

In some embodiments, PG is an amine protecting group such as, but not limited to, -BOC, -benzyl, -Cbz, FMoc, Teoc, Troc, SEM, MOM, TPDPS, TIPS, benzhydryl, Trityl and the like.

In some embodiments, when X is —O—, m is 1, n is 0, R⁴ is —F, R³ is [123]I, [131]I, or [211]At and R¹, R², R⁶, R⁷ are —H, then R⁴ and F cannot both be ortho to R³.

In some embodiments, when X is —O—, n is 0, R¹, R², R⁶, R⁷ are —H, R⁴ is —H or —F, then R³ cannot be a radioisotope R* selected from the group consisting of [123]I, [124]I, [125]I, [131]I, [210]At, and [211]At. Particularly, when m is 0, R⁴ is F and R⁴ and R³ are meta to each other; or when m is 1, R⁴ is H and R⁴ and R³ are meta to each other such as this:

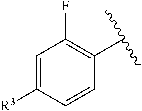

In some embodiments, when X is —O—, n is 0, R¹, R², R⁶, R⁷ are —H, and the compound of Formula (I) contains at least one halogen or more selected from —Br, —Cl, —F, or —I, then R³ cannot be a radioisotope R* selected from the group consisting of [123]I, [124]I, [125]I, [131]I, [210]At, and [211]At. Particularly, when the compound of Formula (I) only contains only one-F atom located meta to R³, such as this:

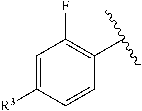

In some embodiments, X and R¹ together form a (C₃-C₅) heterocycle. In some embodiments, X and R¹ together form a (C₃-C₅) heterocycle as is demonstrated below:

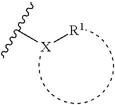

In some embodiments, X and R¹ together form a C₃-heterocyle. In some embodiments, X and R¹ together form a C₄-heterocyle. In some embodiments, X and R¹ together form a C₅-heterocyle. Exemplary (C₃-C₅) heterocycles include, but are not limited to the following:

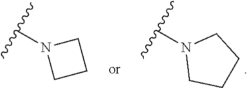 [SMILES: *N1CCC1 ; *N1CCCC1]

In some embodiments, X is —O—. In some embodiments, X is —N[(C₃-C₆) cycloalkyl]- or —N[(C₁-C₆) alkyl]-. In some embodiments, X is —N[(C₁-C₆) alkyl]-. In some embodiments, X is —N[(C₁-C₅) alkyl]-, wherein —N[(C₁-C₆) alkyl]- is selected from the group consisting of —N(CH₃)—, —N(CH₂CH₃)—, —N[CH(CH₃)₂]—, —N(CH₂CH₂CH₃)—, —N(CH₂CH₂CH₂CH₃)—, —N[CH₂CH(CH₃)₂]—, —N[C(CH₃)₃]—, —N(CH₂CH₂CH₂CH₂CH₃)—, —N[CH₂C(CH₃)₃]—, —N[CH₂CH₂CH(CH₃)₂]—, —N(CH₂CH₂CH₂CH₂CH₂CH₃)—, —N[CH₂CH₂C(CH₃)₃]— and —N[CH₂CH₂CH₂CH(CH₃)₂]—. In some embodiments, X is —N(CH₃)— or —N(CH₂CH₃)—.

In some embodiments, X is —N[(C₃-C₆) cycloalkyl]-. In some embodiments, X is —N[(C₃-C₆) cycloalkyl]- and R¹ is —H. In some embodiments, X is selected from the group consisting of —N(cyclopropyl)-, —N(cyclobutyl)-, —N(cyclopentyl)-, —N(cyclohexyl)- and N(phenyl).

In some embodiments, X is —NH—. In some embodiments, X is —NH— and R¹ is (C₁-C₆) alkyl.

In some embodiments, X is —NH— and R¹ is —CH₃, —CH₂CH₃, —CH(CH₃)₂, —CH₂CH₂CH₃, —CH₂CH₂CH₂CH₃, —CH₂CH(CH₃)₂, —C(CH₃)₃,

In some embodiments, X is —O—. In some embodiments, X is —O— or —NH—.

In some embodiments, the compounds disclosed herein comprises a compound of Formula

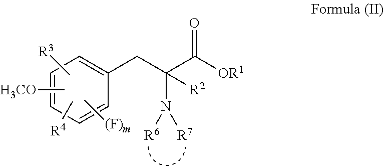

wherein R¹ and R² are independently selected from the group consisting of —H, -PG, —CH₂Ar, —(C₁-C₄) alkyl and —(C₃-C₇) cycloalkyl;
   R³ is —I, —Br or a radioisotope R* selected from the group consisting of [123]I, [124]I, [125]I, [131]I, [210]At, [211]At, [76]Br, [77]Br and [82]Br;
   R⁴ is —H, —F or radioisotope [18]F;
   R⁶ and R⁷ are independently selected from the group consisting of —H, -PG, —CH₂Ar, —(C₁-C₄) alkyl, and —(C₃-C₆) cycloalkyl, or R⁶ and R⁷ together form a (C₃-C₅) heterocycle;
   PG is a protecting group; and
   m is 0, 1 or 2; and
   any stereoisomer and/or pharmaceutically acceptable salt thereof.

In some embodiments, R¹ is (C₁-C₄) alkyl. In some embodiments, R¹ is —CH₃, —CH₂CH₃, —CH(CH₃)₂, —CH₂CH₂CH₃, —C(CH₃)₃, or —CH₂CH(CH₃)₂. In some embodiments, R¹ is —H.

In some embodiments, R² is —(C₁-C₃) alkyl. In some embodiments, R² is —CH₃ or —CH₂CH₃.

In some embodiments, R¹ and/or R² are (C₃-C₇) cycloalkyl. Exemplary (C₃-C₇) cycloalkyl groups include, but are not limited to, cyclopropyl, cyclobutyl, cyclopentyl, cyclohexyl, and cycloheptyl groups. In some embodiments, R¹ and/or R² are (C₃-C₇) cycloalkyl, wherein the (C₃-C₇) cycloalkyl can optionally be substituted with one or more alkyl groups.

In some embodiments, R³ is I and R⁴ is radioisotope [18]F. In some embodiments, R² is —H, —CH₃ or —CH₂CH₃, R³ is I, and R⁴ is radioisotope [18]F. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃ or —CH₂CH₃, R³ is I, and R⁴ is radioisotope [18]F. In some embodiments, m is 0, R¹ is —H, —CH₃, —CH₂CH₃, —CH(CH₃)₂, or —CH₂CH₂CH₃, R² is —H, —CH₃ or —CH₂CH₃, R³ is I, and R⁴ is radioisotope [18]F. In some embodiments, m is 1, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃ or —CH₂CH₃, R³ is I, and R⁴ is radioisotope [18]F.

In some embodiments, R³ is Br and R⁴ is radioisotope [18]F. In some embodiments, R² is —H, —CH₃ or —CH₂CH₃, R³ is Br, and R⁴ is radioisotope [18]F. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃ or —CH₂CH₃, R³ is Br, and R⁴ is radioisotope [18]F. In some embodiments, m is 0, R¹ is —H, —CH₃, —CH₂CH₃, —CH(CH₃)₂, or —CH₂CH₂CH₃, R² is —H, —CH₃ or —CH₂CH₃, R³ is Br, and R⁴ is radioisotope [18]F. In some embodiments, m is 1, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃ or —CH₂CH₃, R³ is Br, and R⁴ is radioisotope [18]F.

In some embodiments, R⁴ is F and R³ is radioisotope R* selected from the group consisting of [123]I, [124]I, [125]I, [131]I, [210]At, [211]At, [76]Br, [77]Br and [82]Br. In some embodiments, R² is —H, —CH₃, —CH₂CH₃, R⁴ is F and R³ is radioisotope R* selected from the group consisting of [123]I, [124]I, [125]I, [131]I, [210]At, [211]At, [76]Br, [77]Br and [82]Br. In some embodiments, R¹ is —H, —CH₃, —CH₂CH₃, —CH(CH₃)₂, or —CH₂CH₂CH₃, R² is —H, —CH₃ or —CH₂CH₃, R⁴ is F and R³ is radioisotope R* selected from the group consisting of [123]I, [124]I, [125]I, [131]I, [210]At, [211]At, [76]Br, [77]Br and [82]Br. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃ or —CH₂CH₃, m is 0, R⁴ is F and R³ is radioisotope R* selected from the group consisting of [123]I, [124]I, [125]I, [131]I, [210]At, and [211]At. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃ or —CH₂CH₃, m is 1, R⁴ is F and R³ is radioisotope R* selected from the group consisting of [123]I, [124]I, [125]I, [131]I, [210]At, and [211]At.

In some embodiments, R³ is —I and R⁴ is —F. In some embodiments, R¹ is —H, —CH₃, —CH₂CH₃, —CH(CH₃)₂, or —CH₂CH₂CH₃, R³ is —I and R⁴ is —F. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃, or —CH₂CH₃, R³ is —I and R⁴ is —F. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃, or —CH₂CH₃, m is 1, R³ is —I and R⁴ is —F. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃, or —CH₂CH₃, m is 0, R³ is —I and R⁴ is —F. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃, or —CH₂CH₃, m is 2, R³ is —I and R⁴ is —F.

In some embodiments, R⁶ and R⁷ are independently selected from the group consisting of —H, —(C₁-C₄) alkyl, and —(C₃-C₆) cycloalkyl. In some embodiments, R⁶ and R⁷ are independently selected from —H and (C₁-C₄) alkyl. In some embodiments, R⁶ and R⁷ are independently selected from —H, —CH₃, —CH₂CH₃, —CH(CH₃)₂, —CH₂CH₂CH₃, —C(CH₃)₃, or —CH₂CH(CH₃)₂. In some embodiments, R⁶ and R⁷ are —H. In some embodiments, R⁶ is —H and R⁷ is CH₃.

In some embodiments, R⁶ and R⁷ are independently selected from the group consisting of —H and —(C₃-C₆) cycloalkyl. In some embodiments, R⁶ and R⁷ are independently selected from the group consisting of —H, -cyclopropyl, -cyclobutyl, -cyclopentyl and -cyclohexyl.

In some embodiments, R⁶ and R⁷ together form a (C₃-C₅) heterocycle. In some embodiments, R⁶ and R⁷ together form a C₃-heterocycle. In some embodiments, R⁶ and R⁷ together form a C₄-heterocycle. In some embodiments, R⁶ and R⁷ together form a C₅-heterocycle. Exemplary (C₃-C₅) heterocycles include, but are not limited to, the following:

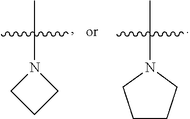 [SMILES: *N1CCC1 ; *N1CCCC1]

Exemplary compounds of Formula (I) and Formula (II) include the following structures (wherein * indicates a radionuclide), but are not limited thereto:

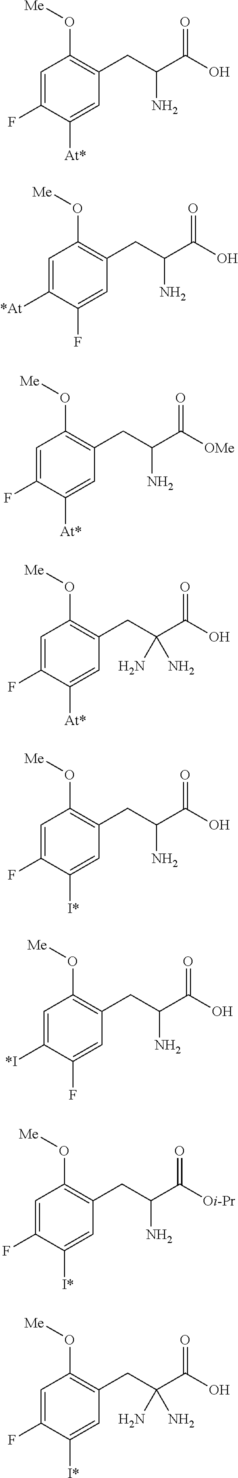

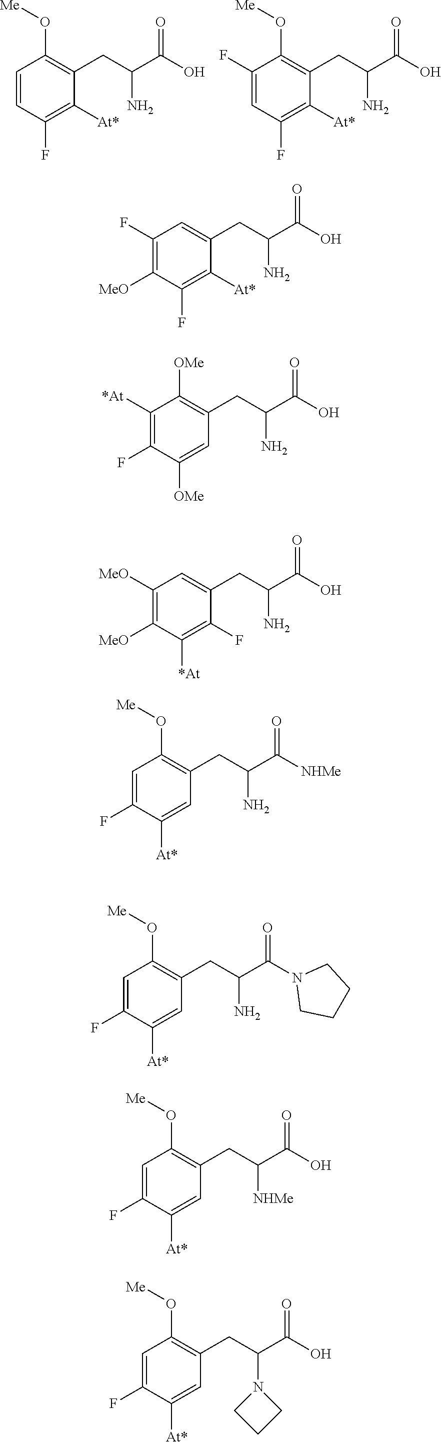

Exemplary compounds of Formula (I) and Formula (II) include the following structures (wherein * indicates a radionuclide), but are not limited thereto:

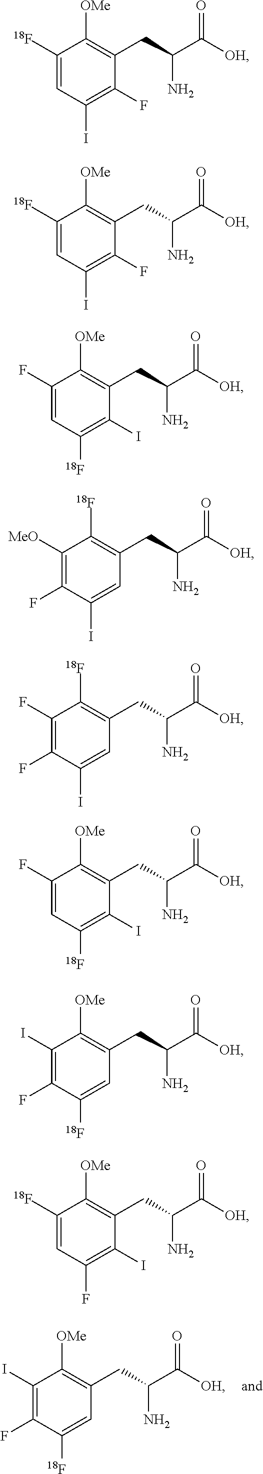

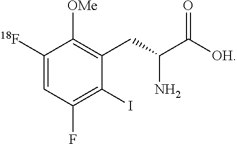 [SMILES: COc1c([18F])cc(F)c(I)c1C[C@@H](N)C(=O)O]

In some embodiments, the compounds disclosed herein comprise a compound of Formula (III):

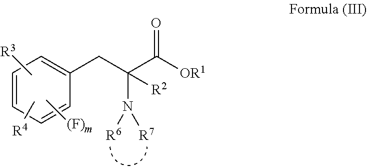

wherein R¹ and R² are independently selected from the group consisting of —H, -PG, —CH₂Ar, —(C₁-C₄) alkyl and (C₃-C₇) cycloalkyl;
R³ is —I, —Br or a radioisotope R* selected from the group consisting of [123]I, [124]I, [125]I, [131]I, [210]At, [211]At, [76]Br, [77]Br and [82]Br;
R⁴ is —H, —F or radioisotope [18]F;
R⁶ and R⁷ are independently selected from the group consisting of —H, —CH₂Ar, -PG-(C₁-C₄) alkyl, and —(C₃-C₆) cycloalkyl, or R⁶ and R⁷ together form a (C₃-C₆) heterocycle;
PG is a protecting group; and
m is 0, 1, 2, or 3; and
any stereoisomer and/or pharmaceutically acceptable salt thereof.

In some embodiments, R¹ is (C₁-C₄) alkyl. In some embodiments, R¹ is —H, —CH₃, —CH₂CH₃, —CH(CH₃)₂, —CH₂CH₂CH₃, —C(CH₃)₃, or —CH₂CH(CH₃)₂. In some embodiments, R¹ is —H.

In some embodiments, R² is —(C₁-C₃) alkyl. In some embodiments, R² is —CH₃ or —CH₂CH₃.

In some embodiments, R¹ and/or R² are —(C₃-C₇) cycloalkyl. Exemplary-(C₃-C₇) cycloalkyl groups include, but are not limited to, -cyclopropyl, -cyclobutyl, -cyclopentyl, -cyclohexyl, and -cycloheptyl groups. In some embodiments, R¹ and/or R² are —(C₃-C₇) cycloalkyl, wherein the —(C₃-C₇) cycloalkyl can optionally be substituted with one or more alkyl groups.

In some embodiments, R³ is I and R⁴ is radioisotope [18]F. In some embodiments, R² is —H, —CH₃ or —CH₂CH₃, R³ is I, and R⁴ is radioisotope [18]F. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃ or —CH₂CH₃, R³ is I, and R⁴ is radioisotope [18]F. In some embodiments, m is 0, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃ or —CH₂CH₃, R³ is I, and R⁴ is radioisotope [18]F. In some embodiments, m is 1, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃ or —CH₂CH₃, R³ is I, and R⁴ is radioisotope [18]F.

In some embodiments, R³ is Br and R⁴ is radioisotope [18]F. In some embodiments, R² is —H, —CH₃ or —CH₂CH₃, R³ is Br, and R⁴ is radioisotope [18]F. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃ or —CH₂CH₃, R³ is Br, and R⁴ is radioisotope [18]F. In some embodiments, m is 0, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃ or —CH₂CH₃, R³ is Br, and R⁴ is radioisotope [18]F. In some embodiments, m is 1, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃ or —CH₂CH₃, R³ is Br, and R⁴ is radioisotope [18]F.

In some embodiments, R⁴ is F and R³ is radioisotope R* selected from the group consisting of [123]I, [124]I, [125]I, [131]I, [210]At, [211]At, [76]Br, [77]Br and [82]Br. In some embodiments, R¹ is —H, —CH₃, —CH₂CH₃, —CH(CH₃)₂, —CH₂CH₂CH₃, —C(CH₃)₃, or —CH₂CH(CH₃)₂, R⁴ is F and R³ is radioisotope R* selected from the group consisting of [123]I, [124]I, [125]I, [131]I, [210]At, [211]At, [76]Br, [77]Br and [82]Br. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃ or —CH₂CH₃, R⁴ is F and R³ is radioisotope R* selected from the group consisting of [123]I, [124]I, [125]I, [131]I, [210]At, [211]At, [76]Br, [77]Br and [82]Br. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃ or —CH₂CH₃, m is 0, R⁴ is F and R³ is radioisotope R* selected from the group consisting of [123]I, [124]I, [125]I, [131]I, [210]At, and [211]At. In some embodiments, R¹ is —H, —CH₃, or —CH₂CH₃, R² is —H, —CH₃ or —CH₂CH₃, m is 1, R⁴ is F and R³ is radioisotope R* selected from the group consisting of [123]I, [124]I, [125]I, [131]I, [210]At, and [211]At.

In some embodiments, R³ is —I and R⁴ is —F. In some embodiments, R¹ is —H, —CH₃, —CH₂CH₃, —CH(CH₃)₂, —CH₂CH₂CH₃, —C(CH₃)₃, or —CH₂CH(CH₃)₂, R³ is —I and R⁴ is —F. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃, or —CH₂CH₃, R³ is —I and R⁴ is —F. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃, or —CH₂CH₃, m is 1, R³ is —I and R⁴ is —F. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃, or —CH₂CH₃, m is 0, R³ is —I and R⁴ is —F. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃, or —CH₂CH₃, m is 2, R³ is —I and R⁴ is —F. In some embodiments, R¹ is —H, —CH₃, —CH₂CH₃, —CH(CH₃)₂, —CH₂CH₂CH₃, —C(CH₃)₃, or —CH₂CH(CH₃)₂, R² is —H, —CH₃, or —CH₂CH₃, m is 3, R³ is —I and R⁴ is —F.

In some embodiments, R⁶ and R⁷ are independently selected from the group consisting of —H, —(C₁-C₄) alkyl, and —(C₃-C₆) cycloalkyl. In some embodiments, R⁶ and R⁷ are independently selected from —H and (C₁-C₃) alkyl. In some embodiments, R⁶ and R⁷ are independently selected from —H, —CH₃, —CH₂CH₃, —CH(CH₃)₂, —CH₂CH₂CH₃, —C(CH₃)₃, —CH₂CH(CH₃)₂. In some embodiments, R⁶ and R⁷ are —H. In some embodiments, R⁶ is —H and R⁷ is —CH₃.

In some embodiments, R⁶ and R⁷ are independently selected from the group consisting of —H and —(C₃-C₅) cycloalkyl. In some embodiments, R⁶ and R⁷ are independently selected from the group consisting of —H, -cyclocropyl, -cyclobutyl, -cyclopentyl and -cyclohexyl.

In some embodiments, R⁶ and R⁷ together form a (C₃-C₅) heterocycle. In some embodiments, R⁶ and R⁷ are independently selected from the group consisting of C₃-heterocycle, C₄-heterocycle and C₅-heterocycle. Exemplary (C₃-C₅) heterocycles include, but are not limited to, the following:

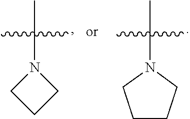 [SMILES: *N1CCC1 ; *N1CCCC1]

Exemplary compounds of Formula (III) include, but are not limited to, the following structures (wherein * indicates a radionuclide):

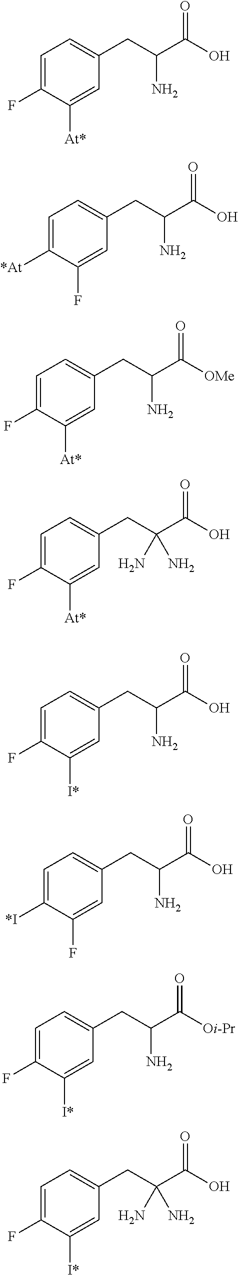

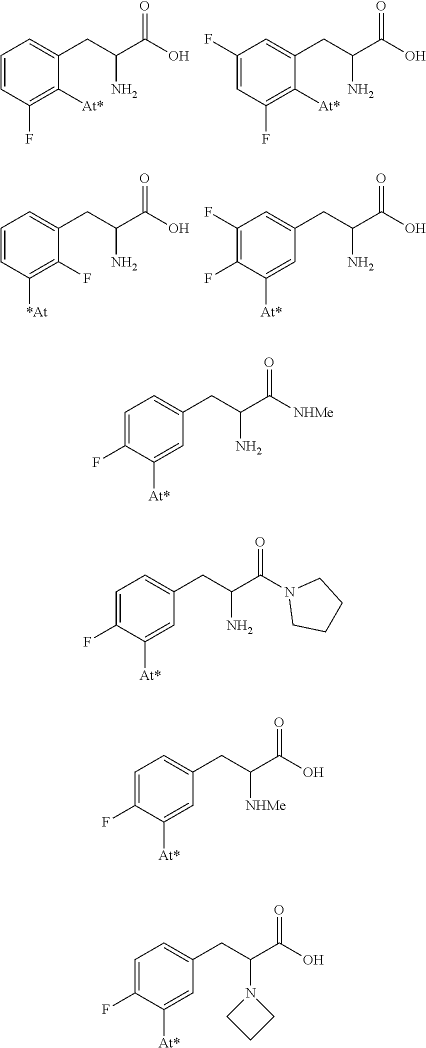

Exemplary compounds of Formula (III) include the following structures (wherein * indicates a radionuclide), but are not limited thereto:

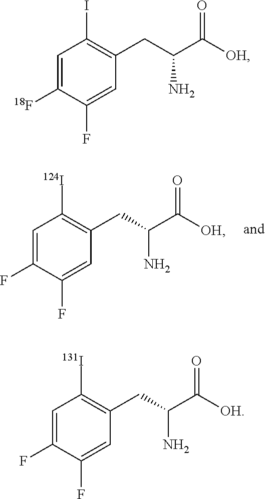 [SMILES: N[C@H](Cc1cc(F)c([18F])cc1I)C(=O)O ; N[C@H](Cc1cc(F)c(F)cc1[124I])C(=O)O ; N[C@H](Cc1cc(F)c(F)cc1[131I])C(=O)O]

In some embodiments, the present disclosure provides a compound of:

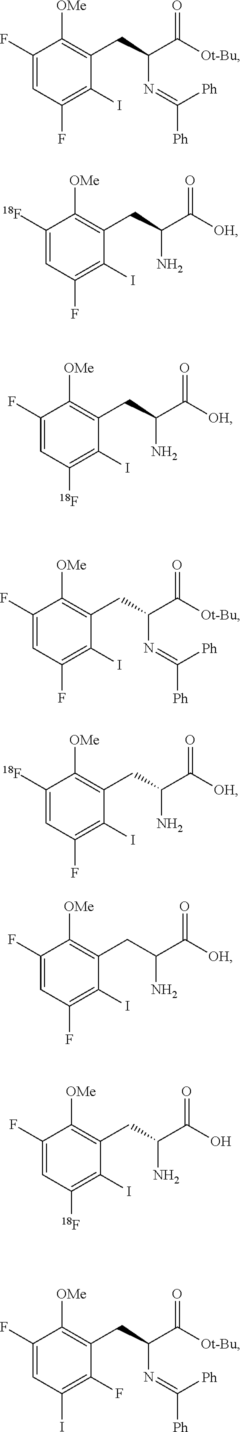

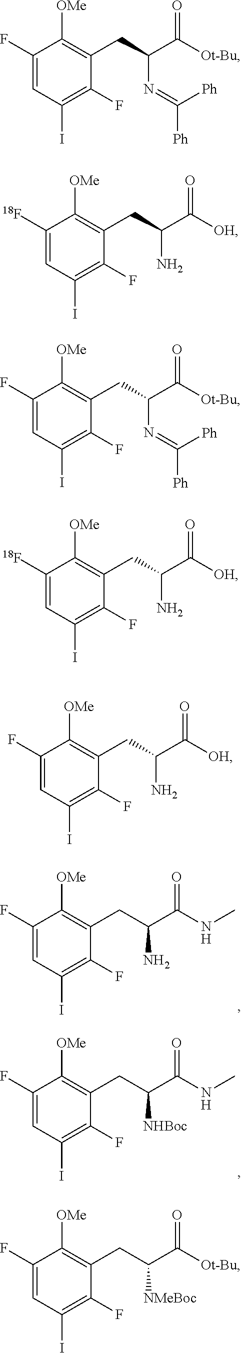

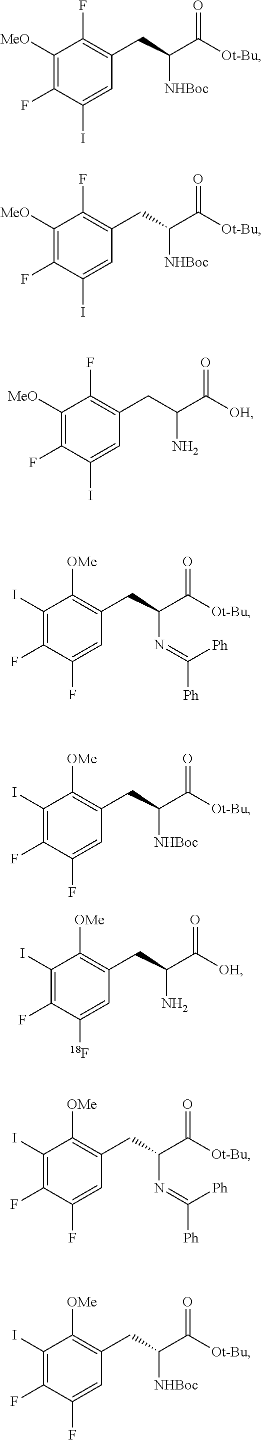

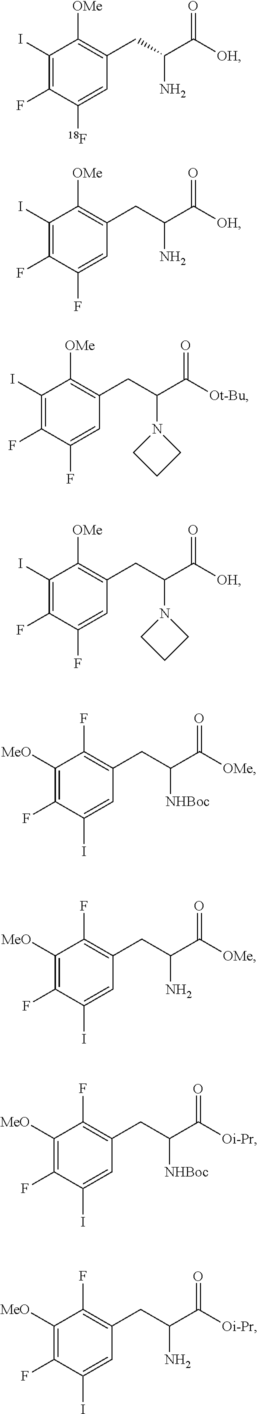

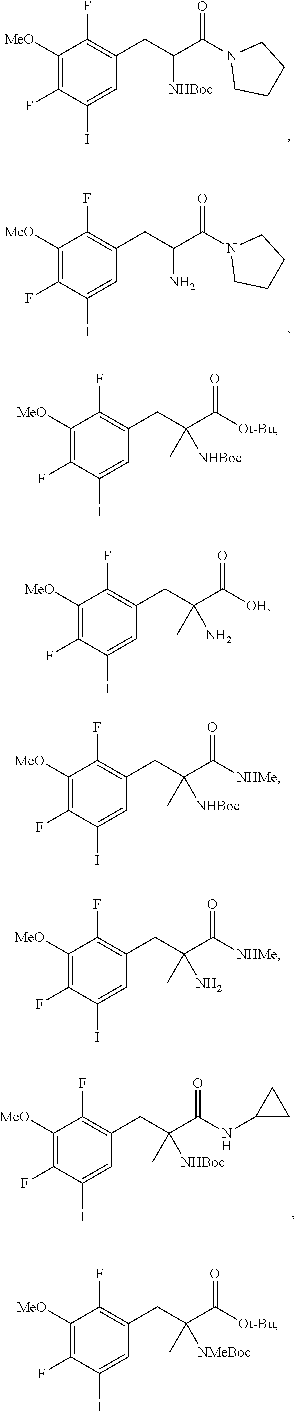

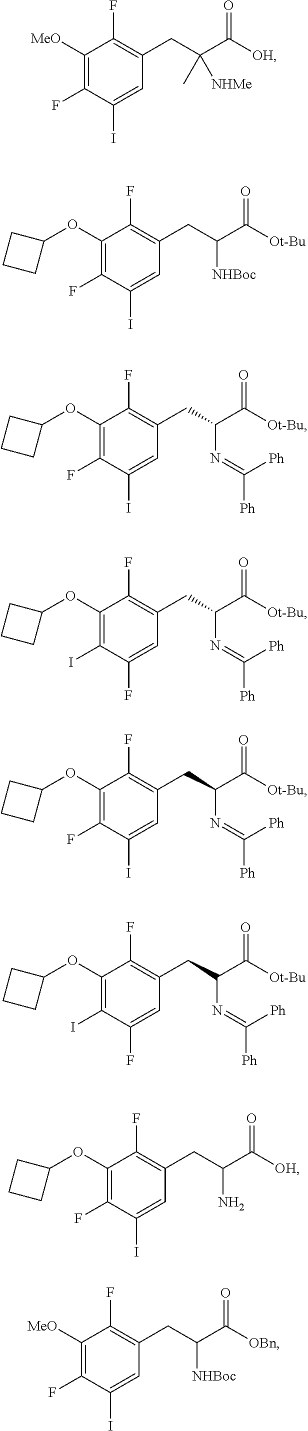

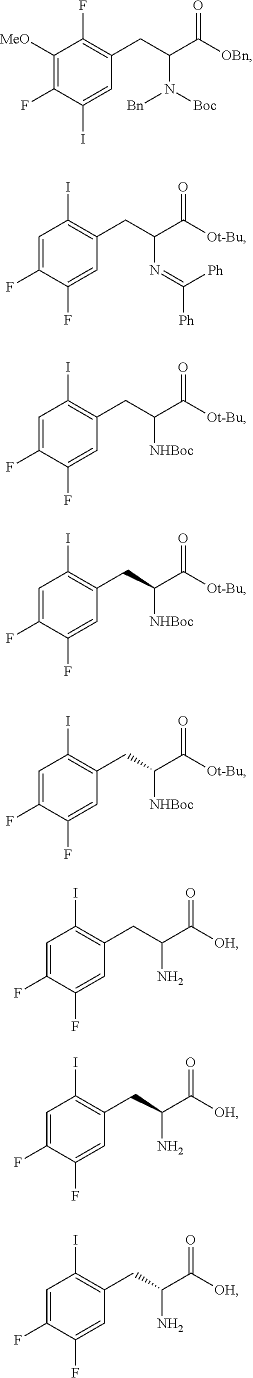

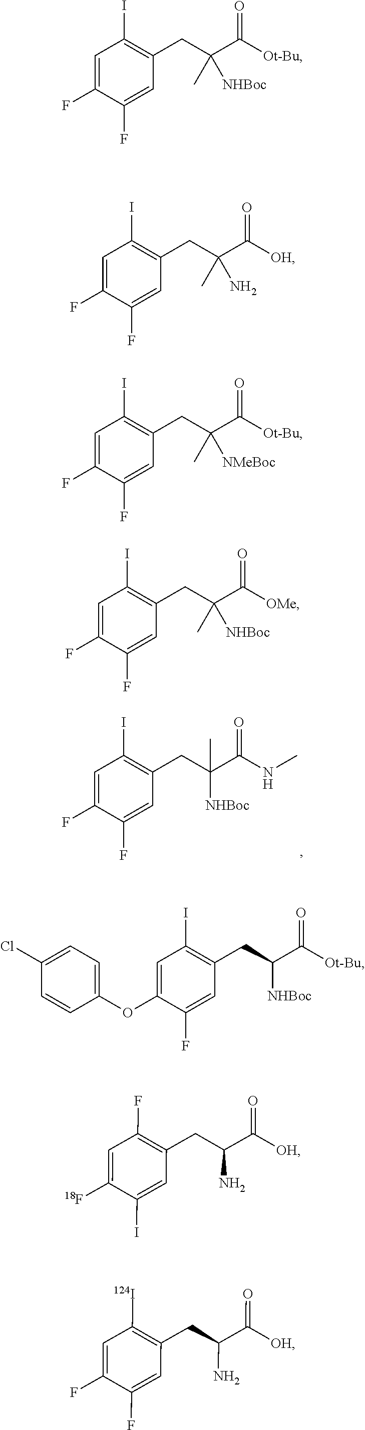

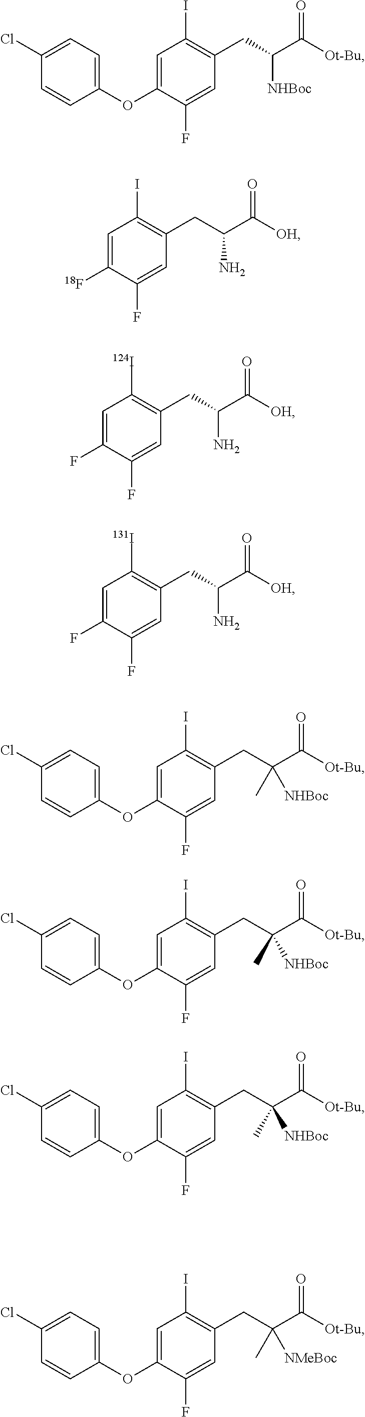

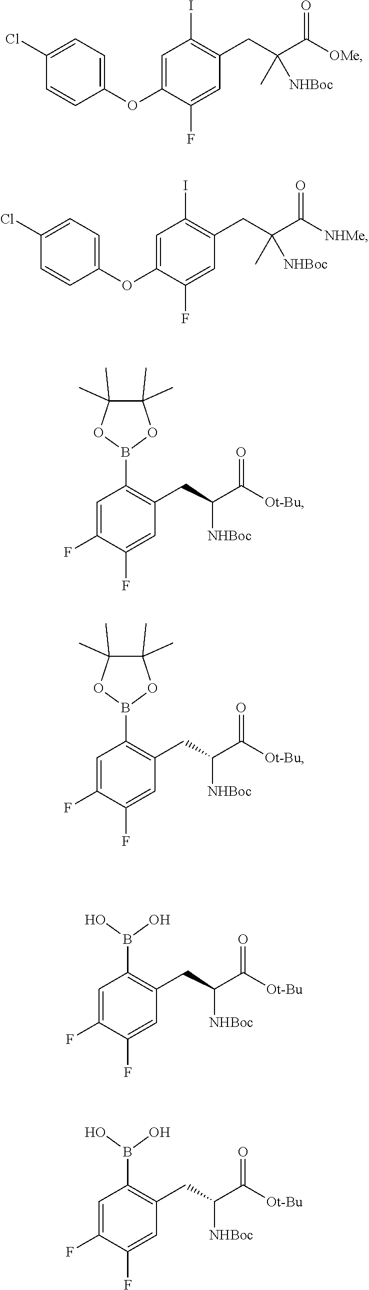

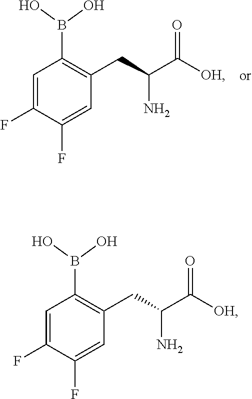 [SMILES: N[C@@H](Cc1cc(F)c(F)cc1B(O)O)C(=O)O ; N[C@H](Cc1cc(F)c(F)cc1B(O)O)C(=O)O]

or a pharmaceutically acceptable salt thereof.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography and/or recrystallization or by the forming diastereomers, including diastereomeric salts, and separation thereof (Jean Jacques, Andre Collet, Samuel H. Wilen, “Enantiomers, Racemates and Resolutions”, John Wiley And Sons, Inc., 1981). Stereoisomers may also be obtained by stereoselective synthesis using synthetic methods known in the art. In some embodiments, the compounds disclosed herein are enantiomers having an enantiomeric excess (% ee) of at least about 50%, about 60%, about 70%, about 80%, about 90%, about 95%, about 98%, or about 99.5%. In some embodiments, the compounds disclosed herein are diastereomers having a diastereomeric excess (% de) of at least about 50%, about 60%, about 70%, about 80%, about 90%, about 95%, about 98%, or about 99.5%. In some embodiments, the compounds disclosed herein are present as enantiomeric or diastereomeric mixtures. In some embodiments, the compounds disclosed herein are derivatives of phenylalanine, an amino acid. In such embodiments, the compounds disclosed herein are the L-isomer of phenylalanine and/or its derivative thereof. In some embodiments, the compound disclosed herein is the L-isomer of any given amino acid, natural or un-natural (not present in nature). In some embodiments, the compounds disclosed herein are the D-isomer of phenylalanine and/or its derivative thereof. In some embodiments, the compound disclosed herein is the D-isomer of any given amino acid, natural or un-natural (not present in nature)

The methods and compositions described herein include the use of amorphous forms as well as crystalline forms (also known as polymorphs). The compounds described herein may be in the form of pharmaceutically acceptable salts. Active metabolites of these compounds having the same type of activity are included in the scope of the present disclosure.

In some embodiments, the compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

In some embodiments, the compounds and salts described herein include isotopically-labeled compounds. In general, isotopically-labeled compounds are identical to those recited in the various formulae and structures presented herein, but for the fact that one or more atoms are replaced by an atom having an atomic mass or mass number different from the atomic mass or mass number most common in nature. Examples of isotopes that can be incorporated into the present compounds include isotopes of hydrogen, carbon, nitrogen, oxygen, fluorine and chlorine, for example, 2H, 3H, 13C, 14C, 15N, 18O, 17O, 35S, 18F, 36Cl, respectively. However, additional isotopes for iodine, bromine, and astatine are also included herein. Certain isotopically-labeled compounds described herein, for example those into which radioactive isotopes are also incorporated as they are useful in drug and/or substrate tissue distribution assays. Further, substitution with isotopes such as deuterium, i.e., 2H, can afford certain therapeutic advantages resulting from greater metabolic stability, such as, for example, increased in vivo half-life or reduced dosage requirements.

Exemplary prodrugs for the compounds disclosed herein would be compounds of Formula (I), wherein X is —O— and R¹ is selected from the group consisting of —H, —(C₁-C₃) alkyl and —(C₃-C₆) cycloalkyl, however such prodrugs should not be limited to only these structures.

As already mentioned above, the compounds disclosed herein are able to either modulate the amino acid metabolism and/or utilize amino acid transport mechanisms of cells (e.g., cancer cells). In general, energy metabolism reprogramming has been implicated in the tumorigenesis and development of PDAC and various other cancers. Specifically, changes in amino acid metabolism contribute to PDAC and various other cancers' malignant characteristics, including cell proliferation, invasion, metastasis, angiogenesis and redox balance. Various enzymes and transporters take part in the amino acid metabolism (e.g., TCA cycle and/or urea cycle). Among such transporters are amino acid transporters which are membrane proteins required for cellular uptake of neutral branched chain amino acids and aromatic amino acids including a number of essential amino acids. One such amino acid transporter is the large neutral amino acid transporter referred to as L-type amino acid transporter 1 (LAT1, known to function as an uptake port of amino acids (e.g. phenylalanine) in cells. LAT1 is over-expressed in a variety of cancer cells and has a role in supplying amino acids as nutrients to cancer tissues.

Indeed, high expression of LAT1 has been found to predict poor prognosis of various cancers, including PDAC, and resistance to therapy. Elevated expression of LAT1 in PDAC and certain other cancers is associated with tumor size and disease stages. In the nutrient-deficient tumor microenvironment, the interactions between cancer cells, stromal components and salvaging processes play critical roles in fulfilling the metabolic requirements supporting the growth of PDAC and various other cancers. Therefore, radiolabeled amino acids such as the phenylalanine derivatives disclosed herein provide a unique opportunity to image and treat PDAC and certain other cancers by targeting both tumor cells and the TME. In other words, because amino acids play key roles in PDAC and various other cancers' progression, a radiolabeled amino acid such as the disclosed phenylalanine derivatives can act as a “Trojan horse” to be actively taken up by PDAC and certain other types of cancerous cells, leading to a corresponding imaging or therapy opportunity of the disease. Because LAT1 over-expression has been observed in a variety of cancers, the present invention and description herein can be extended to imaging and treatment of additional oncological indications.

Thus, one aspect of the current disclosure is that the disclosed compounds utilize amino acid transport mechanisms to travel into cancer cells. In some embodiments, the compounds disclosed herein enter cancer cells utilizing one or more amino acid transport of uptake mechanism in amount of at least about 0.1% to about 5%, from about 0.01% to about 3%, from about 0.01% to about 2%, from about 0.1% to about 1.5%, or from about 0.1% to about 1.0% based on the total amount of compound available. In some embodiments, the disclosed compounds enter cancer cells into an amount ranging from about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or by about 98% based on the total amount of compound available. In some embodiments, the disclosed compounds enter cancer cells into an amount ranging from about 1% to about 100%, from about 2% to about 99%, from about 5% to about 98%, from about 10% to about 95%, from about 15% to about 92%, from about 20% to about 92%, from about 25% to about 90%, from about 30% to about 88%, from about 40% to about 85%, from about 50% to about 82%, from about 60% to about 80%, or from about 70% to about 80% based on the total amount of compound available.

### III. Methods of Preparation

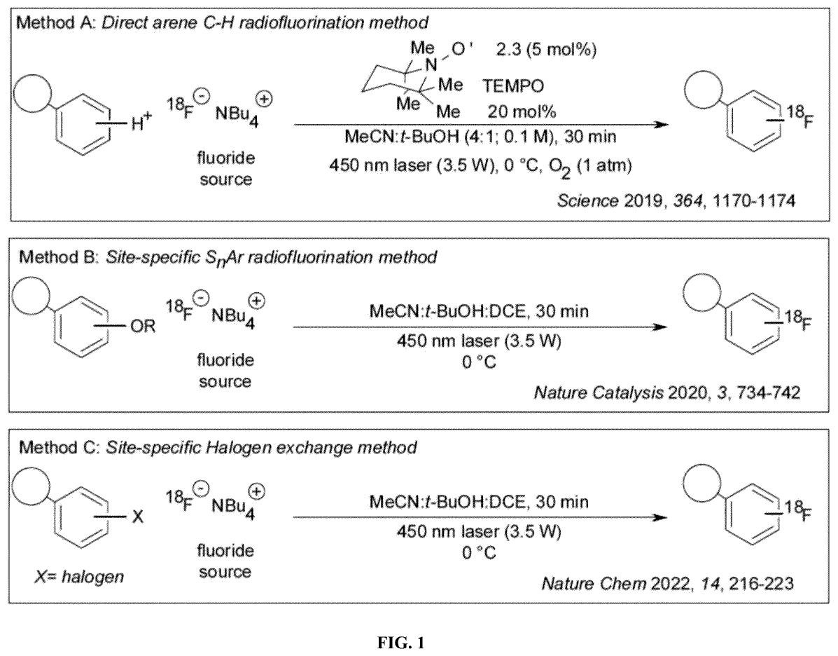

The present disclosure provides methods for preparing the compounds of Formula (I), wherein the compounds can be radioactive or non-radioactive. For example, the present disclosure provides three different methods for preparing compounds of Formula (I) containing a radioactive [18]F moiety, which is one of the most important radioisotopes in the radiopharmaceutical industry, as it possesses a diagnostically useful half-life (t_{1/2}=110 min) and decays with high efficiency by positron emission efficiency (97%). Photoredox radiochemistry and late-stage radiolabeling can be utilized to incorporate [18]F into aromatic compounds such as compounds of Formula (I). Disclosed herein are three different photoredox radiolabeling methods for the introduction of an [18]F moiety using mild reaction conditions providing rapid introduction of an [18]F moiety into aromatic compounds such as compounds of Formula (I) (FIG. 1). Method A. Direct C—H bond conversion: Unlike most of the existing methods, the developed arene C—H radiolabeling disclosed herein converts compounds of Formula (I) without harsh conditions (e.g. O2 free, moisture free, high temperature, strong acid or base etc.) or the need for complicated synthesis to achieve the desired product. Method B. Direct C—O bond conversion: Transition metal catalysis and concerted S_{N}Ar methods have been utilized for the direct fluorination of activated C—O bonds, but there is a dearth of methods for site-selective deoxyfluorinations with relatively unactivated nucleofuges. Disclosed herein is a highly efficient method—nucleophilic aromatic substitution (SNAr)—which is able to install a [18]F moiety to the target molecules in a site-specific manner using alkoxyarenes as substrates where alcohols are the leaving groups. Method C. Direct C—X (X=F, Cl, Br, I, NO₂) bond conversion. As a major substrate class for arene functionalization, aryl (pseudo)halides are commonly used intermediates en route to synthesizing organometallic or prefunctionalized arene precursors for radiofluorination. Clearly, methods that could directly radiofluorinate electron-rich aryl halides are highly desired due to their simplicity. These methods can also be used for the preparation of compounds of Formula (I) containing [19]F moieties, i.e., non-radioactive fluorine moieties.

Compounds of Formula (I) containing radioactive iodine moieties (i.e., [123]I, [124]I, [125]I and [131]I), radioactive astatine moieties (i.e., [210]At and [211]At), or radioactive bromine moieties (i.e., [76]Br, [77]Br and [82]Br), are ideally prepared from boron-, silicon-, tin- or germanium-containing starting materials. Specifically, the method for preparing a compound of Formula (I), wherein R³ is a radioisotope R* selected from the group consisting of [123]I, [124]I, [125]I, [131]I, [210]At, [211]At, [76]Br, [77]Br and [82]Br, can be prepared by, but is not limited to, the following steps:
(a) obtaining a starting material of Formula (IV):

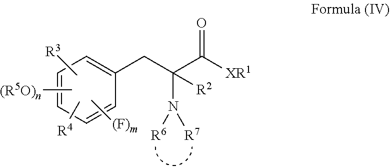

wherein X is —O—, —NH—, —N[(C₃-C₆) cycloalkyl]-, or —N[(C₁-C₆) alkyl]-;
   R¹ is selected from the group consisting of —H, —CH₂Ar, -PG, —(C₁-C₄) alkyl and —(C₃-C₇) cycloalkyl, wherein in some instances X and R¹ together form a (C₃-C₅) heterocycle;
   R² is selected from the group consisting of —H, —(C₁-C₄) alkyl and —(C₃-C₇) cycloalkyl;
   R⁴ is —H or —F;
   R⁵ is -PG, —(C₁-C₆) alkyl or —(C₃-C₆) cycloalkyl;
   R⁶ and R⁷ are independently selected from the group consisting of —H, -PG, —CH₂Ar, —(C₁-C₄) alkyl, and —(C₃-C₇) cycloalkyl, or R⁶ and R⁷ together form a (C₃-C₅) heterocycle;
   R⁸ is selected from the group consisting of —B[O(C₁-C₆) alkyl]₂, —B[—O((C₁-C₆) alkyl)O—], —B(OH)₂, —BF₃K, N-methyliminodiacetic acid boronate, —Sn(C₁-C₄ alkyl)₃, —Ge[(C₁-C₆) alkyl]₃ and —Si[(C₁-C₆) alkyl]₃;
   PG, in each instance, is a protecting group;
   m is 0, 1, 2, or 3; and
   n is 0, 1 or 2; and
   any stereoisomer and/or pharmaceutically acceptable salt thereof
(b) contacting the starting material with a radioisotope source in the presence of a suitable oxidant to render the compound of Formula (I), wherein R* is a radioisotope selected from the group consisting of [123]I, [124]I, [125]I, [131]I, [210]At, [211]At, [76]Br, [77]Br and [82]Br.

In some embodiments, PG is a protecting group for hydroxyl moieties (e.g., —OH). In some embodiments, PG is a protecting group for amine moieties (e.g., primary amines or secondary amines). In some embodiments, PG is a protecting group for carboxylic acids (e.g. —COOH). In some embodiments, PG is a protecting group for carboxamides (e.g., —CONH₂ or —CONHR).

In some embodiments, PG is an amine protecting group such as, but not limited to, -BOC, -benzyl, -Cbz, FMoc, Teoc, Troc, SEM, MOM, TPDPS, TIPS, benzhydryl, Trityl and the like.

In some embodiments, R⁸ in Formula (IV) is boronic acid pinacol ester.

In some embodiments, n is 2. In some embodiments, n is 1. In some embodiments, n is 0.

In some embodiments, R⁵ in Formula (IV) is —(C₁-C₆) alkyl. In some embodiments, R⁵ is selected from the group consisting of —CH₃, —CH₂CH₃, —CH(CH₃)₂, —CH₂CH₂CH₃, —CH₂CH₂CH₂CH₃, —CH₂CH(CH₃)₂, —C(CH₃)₃, —CH₂CH₂CH₂CH₂CH₃, —CH₂C(CH₃)₃, —CH₂CH₂CH(CH₃)₂, —CH₂CH₂CH₂CH₂CH₂CH₃, —CH₂CH₂C(CH₃)₃ and —CH₂CH₂CH₂CH(CH₃)₂. In some embodiments, R⁵ is selected from the group consisting of CH₃, —CH₂CH₃, —CH(CH₃)₂, and —CH₂CH₂CH₃. In some embodiments, R⁵ is —CH₃.

In some embodiments, R⁵ in Formula (IV) is —(C₃-C₆) cycloalkyl. In some embodiments R⁵ is selected from the group consisting of cyclopropyl, cyclobutyl, cyclopentyl, cyclohexyl and phenyl.

In some embodiments, n is 1 and R⁵ is —(C₁-C₆) alkyl. In some embodiments, n is 1 and R⁵ is —CH₃.

In some embodiments, the compound of Formula (IV) contains R¹ and R², which are independently selected from the group consisting of —H, (C₁-C₄) alkyl and (C₃-C₇) cycloalkyl. In some embodiments, R¹ and R² are independently selected from the group consisting of —CH₃, —CH₂CH₃, —CH(CH₃)₂, —CH₂CH₂CH₃, —CH₂CH₂CH₂CH₃, —CH₂CH(CH₃)₂, —C(CH₃)₃. In some embodiments, R² is —CH₃. In some embodiments, R¹ is —CH₃. In some embodiments, R¹ is —H.

In some embodiments, R¹ is (C₃-C₇) cycloalkyl. In some embodiments R¹ is selected from the group consisting of cyclopropyl, cyclobutyl, cyclopentyl, cyclohexyl and phenyl. In some embodiments, R⁶ and R⁷ together form a (C₃-C₅) heterocycle. Exemplary (C₃-C₅) heterocycles include, but are not limited to the following:

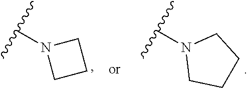 [SMILES: *N1CCC1 ; *N1CCCC1]

In some embodiments, the compound of Formula (IV) contains R⁶ and R⁷, which are independently selected from the group consisting of —H, (C₁-C₄) alkyl, (C₃-C₇) cycloalkyl, and in some instances R⁶ and R⁷ together form a (C₃-C₅) heterocycle. In some embodiments, R⁶ and R⁷ are independently selected from the group consisting of —H, (C₁-C₄) alkyl, and (C₃-C₇) cycloalkyl, or R⁶ and R⁷ together form a (C₃-C₅) heterocycle.

In some embodiments, X and R¹ in Formula (IV) together form a (C₃-C₅) heterocycle. In some embodiments, X and R¹ together form a (C₃-C₅) heterocycle as is demonstrated below:

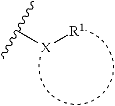

In some embodiments, X and R¹ together form a C₃-heterocyle. In some embodiments, X and R¹ together form a C₄-heterocyle. In some embodiments, X and R¹ together form a C₅-heterocyle. Exemplary (C₃-C₅) heterocycles include, but are not limited to the following:

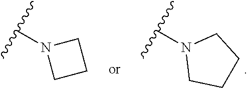 [SMILES: *N1CCC1 ; *N1CCCC1]

In some embodiments, X in Formula (IV) is —O—. In some embodiments, X is —N[(C₃-C₆) cycloalkyl]- or —N[(C₁-C₆) alkyl]-. In some embodiments, X is —N[(C₁-C₆) alkyl]-. In some embodiments, X is —N[(C₁-C₆) alkyl]-, wherein —N[(C₁-C₆) alkyl]- is selected from the group consisting of —N(CH₃)—, —N(CH₂CH₃)—, —N[CH(CH₃)₂]—, —N(CH₂CH₂CH₃)—, —N(CH₂CH₂CH₂CH₃)—, —N[CH₂CH(CH₃)₂]—, —N[C(CH₃)₃]—, —N(CH₂CH₂CH₂CH₂CH₃)—, —N[CH₂C(CH₃)₃]—, —N[CH₂CH₂CH(CH₃)₂]—, —N(CH₂CH₂CH₂CH₂CH₂CH₃)—, —N[CH₂CH₂C(CH₃)₃]— and —N[CH₂CH₂CH₂CH(CH₃)₂]—. In some embodiments, X is —N(CH₃)— or —N(CH₂CH₃)—.

In some embodiments, X in Formula (IV) is —N[(C₃-C₆) cycloalkyl]-. In some embodiments, X is —N[(C₃-C₆) cycloalkyl]- and R¹ is —H. In some embodiments, X is selected from the group consisting of —N(cyclopropyl)-, —N(cyclobutyl)-, —N(cyclopentyl)-, —N(cyclohexyl)- and N(phenyl).

In some embodiments, X in Formula (IV) is —NH—. In some embodiments, X is —NH— and R¹ is (C₁-C₄) alkyl. In some embodiments, X is —NH— and R¹ is —CH₃, —CH₂CH₃, —CH(CH₃)₂, —CH₂CH₂CH₃, —CH₂CH₂CH₂CH₃, —CH₂CH(CH₃)₂, —C(CH₃)₃.

In some embodiments, X is —O— or —NH—. In some embodiments, X is —O—.

In some embodiments, R⁴ is —H. In some embodiments, X is —O— and R⁴ is —H.

In some embodiments, m is 1 or 2. In some embodiments, R⁴ is —H and m is 1 or 2. In some embodiments, R⁴ is —H, m is 1 or 2 and X is —O—. In some embodiments, R⁴ is —H, m is 1 or 2, X is —O— and R² is —H, —CH₃, or —CH₂CH₃. In some embodiments, R⁴ is —H, m is 1 or 2, X is —O—, R² is —H, —CH₃, or —CH₂CH₃ and R⁶ is —H. In some embodiments, R⁴ is —H, m is 1 or 2, X is —O—, R² is —H, —CH₃, or —CH₂CH₃, R⁶ is —H and R⁷ is -PG or —CH₃. In some embodiments, R⁴ is —H, m is 1 or 2, X is —O—, R² is —H, —CH₃, or —CH₂CH₃, R⁶ is —H, R⁷ is -PG or —CH₃ and n is 0. In some embodiments, R⁴ is —H, m is 1 or 2, X is —O—, R² is —H, —CH₃, or —CH₂CH₃, R⁶ is —H, R⁷ is -PG or —CH₃ and n is 1. In some embodiments, R⁴ is —H, m is 1 or 2, X is —O—, R² is —H, —CH₃, or —CH₂CH₃, R⁶ is —H, R⁷ is -PG or —CH₃ and n is 2.

In some embodiments, R⁴ is —F and m is 0 or 1. In some embodiments, X is —O—, R⁴ is —F and m is 0 or 1. In some embodiments, X is —O—, R⁴ is —F, m is 0 or 1 and R² is —CH₃. In some embodiments, X is —O—, R⁴ is —F, m is 0 or 1 and R² is —CH₃ and R¹ is (C₁-C₄) alkyl.

In some embodiments, the oxidant is an N-halosuccinimide (e.g., N-bromosuccinimide, N-iodosuccinimide, N-chlorosuccinimide, and/or N-astatosuccinimide), H₂O₂, DDQ, CuO₂ or the like, although the oxidant should not be limited thereto.

In some embodiments, the N-methyliminodiacetic acid boronate is

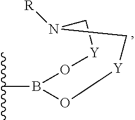

wherein R is (C₁-C₆) alkyl; and Y is —C(═O)—.

In some embodiments, the method further comprises a base activator, e.g., KOAc or KOtBu. In some embodiments, the base activator is an organic or inorganic fluoride (F⁻) source. Exemplary inorganic fluoride sources include, but are not limited to, KHF₂, MgF₂, CsF, KF, CaF₂, and NaF.

In some embodiments, the method disclosed herein is carried out around room temperature (i.e., 22-27° C.). In some embodiments, the method disclosed herein is carried out at elevated temperature (i.e. 40-150° C.).

A skilled artisan would be able to adjust amounts of each reagent, temperature and reaction time accordingly depending on what radioisotope is being used.

In summary, the current disclosure describes efficient radiolabeling methods, which includes [18]F radiolabeling to generate [18]F-labeled phenylalanine derivatives allowing for the preparation of a unique library of radiolabeled phenylalanine derivatives as will be discussed in more detail below. This particular library of compounds shows that specific substitution of arene positions of these derivatives can significantly impact both the tumor uptake/retention, and background accumulation in normal organs. Relevant PET/SPECT agents disclosed herein exhibit high and persistent tumor retention in comparison to normal pancreatic uptake. As will be discussed in more detail below, these compounds can also be converted into corresponding therapeutic agents or isotopes.

### NUMBERED EMBODIMENTS

Embodiment 1: A compound of Formula (I):

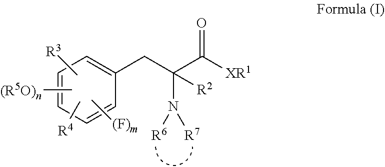

wherein X is —O—, —NH—, —N[(C₃-C₆) cycloalkyl]-, or —N[(C₁-C₆) alkyl]-;
R¹ and R² are independently selected from the group consisting of —H, -PG, —CH₂Ar, —(C₁-C₄) alkyl and —(C₃-C₇) cycloalkyl, wherein in some instances X and R¹ together form a (C₃-C₅) heterocycle,
R³ is —I, —Br or a radioisotope R* selected from the group consisting of [123]I, [124]I, [125]I, [131]I, [210]At, [211]At, [76]Br, [77]Br and [82]Br;
R⁴ is —H, —F or radioisotope [18]F;
R⁵ is -PG, —(C₁-C₆) alkyl or —(C₃-C₆) cycloalkyl;
R⁶ and R⁷ are independently selected from the group consisting of —H, -PG, —CH₂Ar, —(C₁-C₄) alkyl, and —(C₃-C₆) cycloalkyl, or R⁶ and R⁷ together form a (C₃-C₅) heterocycle;
PG, in each instance, is a protecting group;
m is 0, 1, 2, or 3; and
n is 0, 1 or 2; and
any stereoisomer and/or pharmaceutically acceptable salt thereof.
Embodiment 2: The compound of embodiment 1, wherein n is 1.
Embodiment 3: The compound of embodiment 1 or embodiment 2, wherein R⁵ is selected from the group consisting of —CH₃, —CH₂CH₃, —CH(CH₃)₂, —CH₂CH₂CH₃, —CH₂CH₂CH₂CH₃, —CH₂CH(CH₃)₂, and —C(CH₃)₃.
Embodiment 4: The compound of embodiment 1 or embodiment 2, wherein R⁵ is selected from the group consisting of cyclopropyl, cyclobutyl, cyclopentyl, cyclohexyl and phenyl.
Embodiment 5: The compound of any one of the preceding embodiments, wherein R² is —CH₃.
Embodiment 6: The compound of any one of the preceding embodiments, wherein X is —O— or —NH—.
Embodiment 7: The compound of any one of the preceding embodiments, wherein the compound is a compound of Formula (II):

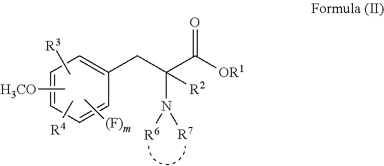

wherein R¹ and R² are independently selected from the group consisting of —H, -PG, —CH₂Ar, —(C₁-C₄) alkyl and —(C₃-C₇) cycloalkyl;
R³ is —I, —Br or a radioisotope R* selected from the group consisting of [123]I, [124]I, [125]I, [131]I, [210]At, [211]At, [76]Br, [77]Br and [82]Br;
R⁴ is —H, —F or radioisotope [18]F;
R⁶ and R⁷ are independently selected from the group consisting of —H, —CH₂Ar, -PG, —(C₁-C₄) alkyl, and —(C₃-C₆) cycloalkyl, or R⁶ and R⁷ together form a (C₃-C₆) heterocycle;
PG is a protecting group; and
m is 0, 1 or 2; and
any stereoisomer and/or pharmaceutically acceptable salt thereof.
Embodiment 8: The compound of embodiment 7, wherein R³ is I and R⁴ is radioisotope [18]F.
Embodiment 9: The compound of embodiment 7, wherein R⁴ is F and R³ is radioisotope R* selected from the group consisting of [124]I, [131]I, [210]At, and [211]At; [76]Br, [77]Br, [82]Br.
Embodiment 10: The compound of embodiment 7, wherein R³ is —I and R⁴ is —F.
Embodiment 11: The compound of any one of embodiments 7-10, wherein R¹ is —H.
Embodiment 12: The compound of any one of embodiments 7-10, wherein R¹ is selected from the group consisting of —CH₃, —CH₂CH₃, —CH(CH₃)₂ and —C(CH₃)₃.
Embodiment 13: The compound of embodiment 1, wherein n is 0.
Embodiment 14: The compound of embodiment 13, wherein R⁵ is selected from the group consisting of —CH₃, —CH₂CH₃, —CH(CH₃)₂ and —C(CH₃)₃.
Embodiment 15: The compound of embodiment 13 or embodiment 14, wherein R² is —CH₃.
Embodiment 16: The compound of any one of embodiments 13-15, wherein the compound is a compound of Formula (III):

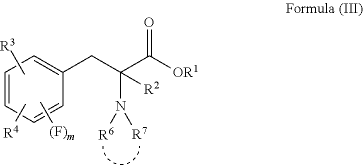

wherein R¹ and R² are independently selected from the group consisting of —H, -PG, —CH₂Ar, —(C₁-C₄) alkyl and —(C₃-C₆) cycloalkyl;
R³ is —I, —Br or a radioisotope R* selected from the group consisting of [123]I, [124]I, [125]I, [131]I, [210]At, [211]At, [76]Br, [77]Br and [82]Br;
R⁴ is —H, —F or radioisotope [18]F;
R⁶ and R⁷ are independently selected from the group consisting of —H, —CH₂Ar, -PG, —(C₁-C₄) alkyl, and —(C₃-C₆) cycloalkyl, or R⁶ and R⁷ together form a (C₃-C₅) heterocycle;
PG is a protecting group; and
m is 0, 1, 2, or 3; and
any stereoisomer and/or pharmaceutically acceptable salt thereof.
Embodiment 17: The compound of embodiment 16, wherein R³ is I and R⁴ is radioisotope [18]F.
Embodiment 18: The compound of embodiment 16, wherein R⁴ is F and R³ is radioisotope R* selected from the group consisting of [124]I, [131]I, and [211]At.
Embodiment 19: The compound of embodiment 16, wherein R³ is —I and R⁴ is —F.
Embodiment 20: The compound of any one of embodiments 16-19, wherein R¹ is —H.
Embodiment 21: The compound of any one of embodiments 16-19, wherein R¹ is selected from the group consisting of —CH₃, —CH₂CH₃, —CH(CH₃)₂ and —C(CH₃)₃.
Embodiment 22: The compound of any one of the preceding embodiments, wherein the compound is present in an enantiomeric excess of at least 95% ee.
Embodiment 23: The compound of any one of the preceding embodiments, wherein the compound is present in a diastereomeric excess of at least 95% de.
Embodiment 24: The compound of any one of the preceding embodiments, wherein the compound is in the form of an L-isomer.
Embodiment 25: The compound of any one of the preceding embodiments, wherein the compound is in the form of a D-isomer.
Embodiment 26: The compound of any one of the preceding embodiments, wherein the compound is a PET imaging agent, a SPECT imaging agent, or a radiolabeled-based therapeutic agent.
Embodiment 27: A pharmaceutical formulation comprising a compound of any one of the preceding embodiments and at least one pharmaceutically acceptable carrier.
Embodiment 28: A method of imaging a subject for diagnosing a disease or monitoring efficacy of a treatment, the method comprising:
   administering to the subject in need thereof an effective amount of a compound of Formula (I), wherein either R³ is R* selected from the group consisting of [123]I and [124]I or R⁴ is [18]F; and
   acquiring at least one image of at least one portion of the subject.
Embodiment 29: The method of embodiment 28, wherein the disease is selected from the group consisting of infection, cardiovascular disease, neurological disease, and cancer.
Embodiment 30: The method of embodiment 29, wherein the cancer is selected from the group consisting of pancreatic cancer and brain cancer (e.g., glioblastoma).
Embodiment 31: The method of any one of embodiments 28-30, wherein the subject is imaged using PET or SPECT imaging technologies.
Embodiment 32: The method of any one of embodiments 28-31, wherein the subject is a mammal.
Embodiment 33: The method of embodiments 28-32, wherein the treatment comprises administration to the subject in need thereof a therapeutically effective amount of a therapeutic agent, wherein the therapeutic agent is an anti-cancer agent.
Embodiment 34: The method of embodiment 33, wherein the anti-cancer agent is a radiolabeled-based therapeutic agent selected from
   (a) a compound of embodiment 1, wherein radioisotope R* is selected from the group consisting of [131]I and [211]At; and
   (b) a commercially available radiolabeled-based therapeutic agent.
Embodiment 35: The method of embodiment 33, wherein the anti-cancer agent is a commercially available drug.
Embodiment 36: The method of embodiment 35, wherein the anti-cancer agent is a compound of Formula (I), wherein R³ and R⁴ are independently selected from the group consisting of —F and —I;
and wherein R³ and R⁴ are not the same halogen substituents.
Embodiment 37: The method of any one of embodiments 32-35, wherein the therapeutic agent is administered prior to administration of the compound of Formula (I).
Embodiment 38: The compound of any one of the preceding embodiments, wherein the compound is a prodrug.
Embodiment 39: A method for the preparation of a compound of Formula (I),

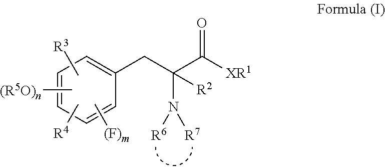

wherein X is —O—, —NH—, —N[(C₃-C₆) cycloalkyl]-, or —N[(C₁-C₆) alkyl]-;
R¹ and R² are independently selected from the group consisting of —H, -PG, —CH₂Ar, —(C₁-C₄) alkyl and (C₃-C₇) cycloalkyl, wherein in some instances X and R¹ together from a (C₃-C₅) heterocycle,
R³ is —I, —Br or a radioisotope R* selected from the group consisting of [123]I, [124]I, [125]I, [131]I, [210]At, [211]At, [76]Br, [77]Br and [82]Br;
R⁴ is —H or —F;
R⁵ is -PG, —(C₁-C₆) alkyl or —(C₃-C₆) cycloalkyl;
R⁶ and R⁷ are independently selected from the group consisting of —H, —CH₂Ar, -PG, —(C₁-C₄) alkyl, and —(C₃-C₆) cycloalkyl, or R⁶ and R⁷ together form a (C₃-C₅) heterocycle;
PG is a protecting group;
m is 0, 1, 2, or 3; and
n is 0, 1 or 2; and
any stereoisomer and/or pharmaceutically acceptable salt thereof.
comprises the following steps:
   (a) obtaining a starting material of Formula (IV):

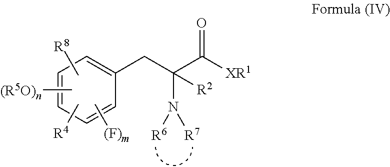

wherein X is —O—, —NH—, —N(C₃-C₆) cycloalkyl-, or —N(C₁-C₆) alkyl-;
R¹ is selected from the group consisting of —H, —CH₂Ar, -PG, —(C₁-C₄) alkyl and —(C₃-C₇) cycloalkyl;
R² is selected from the group consisting of —H, —(C₁-C₄) alkyl and —(C₃-C₇) cycloalkyl, wherein in some instances X and R¹ together form a (C₃-C₅) heterocycle,
R⁴ is —H or —F;
R⁵ is -PG, —(C₁-C₆) alkyl or —(C₁-C₆) cycloalkyl;
R⁶ and R⁷ are independently selected from the group consisting of —H, -PG, —CH₂Ar, —(C₁-C₄) alkyl, and —(C₃-C₆) cycloalkyl, or R⁶ and R⁷ together forming a (C₃-C₅) heterocycle;
R⁸ is selected from the group consisting of —B[O(C₁-C₆) alkyl]₂, —B[—O((C₁-C₆) alkyl)O—], —B(OH)₂, —BF₃K, N-methyliminodiacetic acid boronate, —Sn(C₁-C₄ alkyl)₃, —Ge[(C₁-C₆) alkyl]₃ and —Si[(C₁-C₆) alkyl]₃;
m is 0, 1, 2, or 3;
PG, in each instance, is a protecting group; and
n is 0, 1 or 2; and
any stereoisomer and/or pharmaceutically acceptable salt thereof
(b) contacting the starting material with a radioisotope source in the presence of a suitable oxidant to render the compound of Formula (I) as shown above, wherein the radioisotope is selected from the group consisting of [123]I, [124]I, [125]I, [131]I, [210]At, [211]At, [76]Br, [77]Br and [82]Br.
Embodiment 40: The method of embodiment 39, wherein the method further comprises a base activator.
Embodiment 41: The method of embodiment 40, wherein the base activator is selected from organic or inorganic fluoride sources.
Embodiment 42: The method of embodiment 41, wherein the base activator is selected from the group consisting of KOAc, KOtBu, KHF₂ and F⁻.
Embodiment 43: The method of any one of embodiments 39-42, wherein the halo N-succinimide is N-chlorosuccinimide.
Embodiment 40: A compound of Formula (I):

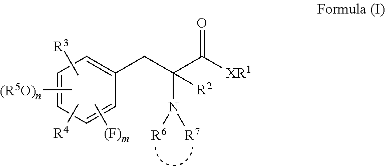

wherein:
X is —O—, —NH—, —N[(C₃-C₆) cycloalkyl]-, or —N[(C₁-C₆) alkyl]-;
R¹ and R² are independently selected from the group consisting of —H, -PG, —CH₂Ar, —(C₁-C₄) alkyl, and —(C₃-C₇) cycloalkyl;
or X and R¹ together form a (C₃-C₅) heterocycle;
R³ is a radioisotope R* selected from the group consisting of [210]At and [211]At, wherein R³ is in the 2-position on the phenyl ring;
R⁴ is —F, or radioisotope [18]F;
R⁵ is -PG, —(C₁-C₆) alkyl, or —(C₃-C₆) cycloalkyl;
R⁶ and R⁷ are independently selected from the group consisting of —H, -PG, —CH₂Ar, —(C₁-C₄) alkyl, and —(C₃-C₆) cycloalkyl;
or R⁶ and R⁷ together form a (C₃-C₅) heterocycle;
PG, in each instance, is a protecting group;
m is 1, 2, or 3; and
n is 0, 1, or 2;
or any stereoisomer and/or pharmaceutically acceptable salt thereof.
In some embodiments, the compound of Formula (I) is:

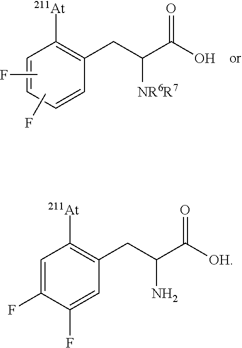

Embodiment 41: A compound of Formula (I):

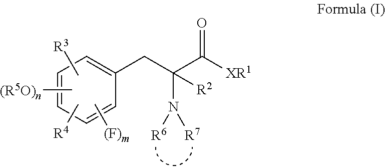

wherein:
X is —O—, —NH—, —N[(C₃-C₆) cycloalkyl]-, or —N[(C₁-C₆) alkyl]-;
R¹ and R² are independently selected from the group consisting of —H, -PG, —CH₂Ar, —(C₁-C₄) alkyl, and —(C₃-C₇) cycloalkyl;
or X and R¹ together form a (C₃-C₅) heterocycle;
R³ is a radioisotope R* selected from the group consisting of [210]At and [211]At;
R⁴ is —H, —F, or radioisotope [18]F;
R⁵ is -PG, —(C₁-C₆) alkyl, or —(C₃-C₆) cycloalkyl;
R⁶ and R⁷ are independently selected from the group consisting of —H, -PG, —CH₂Ar, —(C₁-C₄) alkyl, and —(C₃-C₆) cycloalkyl;
or R⁶ and R⁷ together form a (C₃-C₅) heterocycle;
PG, in each instance, is a protecting group;
m is 0, 1, 2, or 3; and
n is 1 or 2;
or any stereoisomer and/or pharmaceutically acceptable salt thereof.
In some embodiments, R³ is in the 2-position on the phenyl ring.

### IV. Pharmaceutical Formulation

In certain embodiments, compounds, prodrugs or salts of Formulae (I), (II), and/or (III) disclosed herein, are combined with one or more additional agents to form pharmaceutical formulations. In some embodiments, compounds, prodrugs or salts of Formulae (I), (II), and/or (III) disclosed herein are non-radioactive (meaning they contain no radioisotope) and are formulated as formulations for treating a disease or condition in a subject in need thereof. In some embodiments, compounds, prodrugs or salts of Formulae (I), (II), and/or (III) disclosed herein are radioactive (meaning they contain a radioisotope) and are formulated as radionuclide-based formulations for treating a disease or condition in a subject in need thereof. In some embodiments, compounds, prodrugs or salts of Formulae (I), (II), and/or (III) disclosed herein are formulated as imaging agents to evaluate the potential efficacy of a treatment in a subject in need thereof or to diagnose a disease or condition in a subject. A skilled artisan would be aware that the pharmaceutical formulation of compounds, prodrugs or salts of Formulae (I), (II), and/or (III) differs depending on their composition (radioactive or nonradioactive) and use (radiolabeled-based therapy agent or imaging agent).

In some embodiments, compounds of Formulae (I), (II), and/or (III) are already in the form of a prodrug. Pharmaceutical formulations may be formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure. A skilled artisan would generally be aware of the suitable excipients for pharmaceutical formulations and compositions required for any particular mode of administration.

A pharmaceutical formulation, as used herein, refers to a mixture of a compound or salt or prodrug of Formulae (I), (II) and/or (III) with any suitable substituents and functional groups disclosed herein, with other chemical components, such as carriers, stabilizers, diluents, dispersing agents, suspending agents, thickening agents, and/or excipients. The pharmaceutical formulation facilitates administration of the compound to an organism. In practicing the methods of treatment or use provided herein, therapeutically effective amounts of compounds described herein are administered in a pharmaceutical formulation to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds or salts of Formulae (I), (II) and/or (III) with any suitable substituents and functional groups disclosed herein, can be used singly or in combination with one or more therapeutic agents as components of mixtures (as in combination therapy).

The pharmaceutical formulations described herein can be administered to a subject by multiple administration routes, including but not limited to, oral, parenteral (e.g., intravenous, subcutaneous, intramuscular, intratumoral), intranasal, buccal, topical, rectal, or transdermal administration routes. Moreover, the pharmaceutical formulation described herein, which include a compound of Formula (I), (II) and/or (III) with any suitable substituents and functional groups disclosed herein, can be formulated into any suitable dosage form, including but not limited to, aqueous oral dispersions, liquids (e.g., injectables), gels, syrups, elixirs, slurries, suspensions, aerosols, fast melt formulations, effervescent formulations, lyophilized formulations, tablets, powders, pills, dragees, and capsules. In some embodiments, the pharmaceutical formulations described herein are administered to a subject by parenteral administration (e.g., intravenous, subcutaneous, intramuscular, intratumoral). In some embodiments, the pharmaceutical formulations described herein are administered to a subject intravenously. In general, a skilled artisan would be aware of the different modes of administration and as to their suitable formulations (and excipients present therein).

One may administer the compounds and/or formulations in a local rather than systemic manner, for example, via injection of the compound directly into an organ or tissue, often in a depot preparation or sustained release formulation. Such long-acting formulations may be administered by implantation (for example subcutaneously or intramuscularly) or by intramuscular injection. Furthermore, one may administer the drug in a targeted drug delivery system, for example, in a liposome coated with an organ-specific antibody. The liposomes will be targeted to and taken up selectively by the organ. In addition, the drug may be provided in the form of a rapid release formulation, in the form of an extended-release formulation, or in the form of an intermediate release formulation.

The pharmaceutical formulation will include at least one compound of Formulae (I), (II) and/or (III) disclosed herein, as an active ingredient in free-acid or free-base form, or in a pharmaceutically acceptable salt form.

In some embodiments, formulations provided herein may also include one or more preservatives to inhibit microbial activity. Suitable preservatives include, but are not limited to, quaternary ammonium compounds such as benzalkonium chloride, cetyltrimethylammonium bromide and cetylpyridinium chloride.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally recognized in the field. For other parenteral injections, appropriate formulations may include aqueous or nonaqueous solutions, preferably with physiologically compatible buffers or excipients. Such excipients are generally recognized in the field.

Parenteral injections may involve bolus injection or continuous infusion. Formulations for injection may be presented in unit dosage form, e.g., in ampoules or in multi-dose containers, with an added preservative. The pharmaceutical formulations described herein may be in a form suitable for parenteral injection as sterile suspensions, solutions, or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or various dextrans. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

In certain embodiments, delivery systems for pharmaceutical compounds may be employed, such as, for example, liposomes and emulsions. In certain embodiments, compositions provided herein also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and various dextrans.

In some embodiments, the compounds of Formulae (I), (II), and/or (III) disclosed herein are combined with other therapeutic agents, such as other anti-cancer agents, anti-allergic agents, anti-nausea agents (or anti-emetics), pain relievers, cytoprotective agents, and combinations thereof.

In another embodiment, the compounds of Formulae (I), (II) and/or (III) disclosed herein are combined with another therapeutic agent capable of inhibiting BRAF, MEK, KRAS, SOS1, CDK4/6, SHP-2, HDAC, EGFR, MET, mTOR, PI3K or AKT, or anti-PD1 drugs such as Nivolumab, Prembolamab, Cemiplimab, or anti-PDL1 drugs such as Atezolizumab, Durvalumab, Avelumab, or anti-CTL4 drugs such as Ipilimumab or Tremelinumab, or other checkpoint inhibitors including bi-specific antibodies, or PARP inhibitors such as Olaparib, Niraparib, Velaparib, Rucaparib, Talazoparib, Pamiparib, Fluzoparib, or cell therapies such as T-cell receptor therapies, tumor-infiltrating lymphocytes, CAR-T, or immunotherapies such as APC-directed and macrophage-directed antibodies, or vaccines such as mRNA neoantigen vaccines, GM-CSF producing vaccines, peptide vaccines, or a combination thereof.

Generally, an agent, such as a compound of Formula (I) disclosed herein, is administered in an amount effective for treating the disease or disorder (i.e., a therapeutically effective amount). Thus, a therapeutically effective amount can be an amount that is capable of at least partially treating, preventing or reversing a disease or disorder. The dose required to obtain an effective amount may vary depending on the agent, formulation, disease or disorder, and individual to whom the agent is administered.

Determination of effective amounts may also involve in vitro assays in which varying doses of the compound disclosed herein is administered to cells in culture and the concentration of the compound effective for ameliorating some or all symptoms is determined in order to calculate the concentration required in vivo. Effective amounts may also be based in in vivo animal studies.

A compound as disclosed herein can be administered prior to, concurrently with and subsequent to the appearance of symptoms of a disease or disorder. In some embodiments, the compound disclosed herein is administered to a subject with a family history of the disease or disorder, or who has a phenotype that may indicate a predisposition to a disease or disorder, or who has a genotype which predisposes the subject to the disease or disorder.

The dosing and administration regimes of radionuclide-based formulations containing compounds of Formula (I) to be administered is based on various factors such as the type of radionuclide present in the compound of Formula (I), the disease or disorder to be treated, and the subject (age, weight, sex, etc.). Dosing for a therapeutic is typically higher than when used as an imaging agent and can be once a day or multiple times per day for one or more consecutive days. The amount of radioactivity administered during such a treatment course may vary from dose to dose of the radioactive compound of Formula (I). The amount of radioactivity of a radioactive compound of Formula (I) and its frequency and duration of administration is determined by a skilled person in the art, e.g., a physician knowledgeable in Nuclear Medicine, as would be apparent to a skilled artisan. Specifically, a skilled artisan would be aware that for beta-particle therapy (e.g., [131]I) the radiolabeled-based therapeutic is administered over a 100-300 mCi range, whereas for alpha-particle therapy (e.g., [211]At) the radiolabeled-based therapeutic would generally be administered over a 1-10 mCi range. It would be understood by a skilled artisan that the radiolabeled-based therapeutics disclosed herein would be administered at doses encompassed by, but not limited to, the above-mentioned ranges depending on the type of therapy (alpha-particle vs. beta-particle).

### Methods of Treatment

The present disclosure provides compounds and methods for utilizing and/or modulating amino acid metabolism. In some embodiments, the present disclosure provides compounds and methods wherein the compounds disclosed herein are substrates of the L-type, also referred to as large neutral, amino acid transporter (LAT1). LAT1 is known to function as an uptake mechanism of amino acids into cells. LAT1 is often over-expressed in cancer cells and has a role in supplying amino acids as nutrients in cancer tissues.

The disclosure provides compounds and methods for treating a subject suffering from a disease, comprising administration of a compound, prodrug or salt described herein, for example, a compound, prodrug or salt of Formulae (I), (II) and/or (III) disclosed herein, to the subject. In some embodiments, the disease is selected from a disease associated with expression of cellular targets involved in amino acid metabolism (e.g., LAT1), aberrant expression, overexpression and/or activity (e.g. cancer). In certain embodiments, the disease is mediated by cellular targets involved in amino acid metabolism (e.g., LAT1) and/or expression (e.g., aberrant expression, overexpression, etc.). In some embodiments, the disease or condition is treatable by modulation of cellular targets involved in amino acid metabolism (e.g., LAT1). In some embodiments, the method comprises treating a disease or condition that is treatable by modulation of cellular targets involved in amino acid metabolism (e.g., LAT1) by administering to a subject in need thereof a therapeutically effective amount of a compound, prodrug, or a salt thereof of Formulae (I), (II), and/or (III) or a pharmaceutical composition as disclosed herein.

In some embodiments, the disclosure provides a method for treating cancer in a subject, comprising administration of a compound, prodrug or salt described herein, for example, a compound, prodrug or salt of Formulae (I), (II), and/or (III) disclosed herein, to the subject. In some embodiments, the cancer is mediated by an expression, aberrant expression, overexpression (etc.), of cellular targets (e.g. LAT1) involved in amino acid metabolism and/or activity. For example, in some embodiments, the cellular targets involved in amino acid metabolism are over-expressed in diseased cells (e.g., cancer cells) compared to healthy cells (i.e., cells free of disease).

In certain embodiments, the disclosure provides methods of treating a disease in a subject, wherein the method comprises determining if the subject has an amino acid metabolism-mediated condition (e.g., cancer, neurological diseases, cardiovascular diseases, and/or infection) and administering to the subject a therapeutically effective dose of a compound, prodrug or salt described herein, for example, a compound, prodrug or salt of Formulae (I), (II), and/or (III) as disclosed herein.

The disclosure provides methods for treating a disease by administering a compound, prodrug, or salt of Formulae (I), (II), and/or (III) disclosed herein, to a subject suffering from the disease, wherein the compound binds to or is transported by a cellular target involved in amino acid metabolism (e.g., LAT1). In some embodiments, the compound binds to or is transported by the cellular target involved in amino acid metabolism (e.g. LAT1).

The disclosure also relates to a method of treating a hyperproliferative disorder in a mammal that comprises administering to the mammal a therapeutically effective amount of a compound, prodrug, or salt of Formulae (I), (II), and/or (III) with any suitable substituents and functional groups disclosed herein. In some embodiments, the method relates to the treatment of cancer such as acute myeloid leukemia, cancer in adolescents, childhood adrenocortical carcinoma, AIDS-related cancers, e.g., lymphoma and Kaposi's Sarcoma, anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some embodiments, the method relates to the treatment of a non-cancerous hyperproliferative disorder such as benign hyperplasia of the skin, e.g., psoriasis, restenosis, or prostate, e.g., benign prostatic hypertrophy (BPH). In some cases, the method relates to the treatment of leukemia, hematologic malignancy, solid tumor cancer, prostate cancer, e.g., castration-resistant prostate cancer, breast cancer, Ewing's sarcoma, bone sarcoma, primary bone sarcoma, T-cell prolymphocyte leukemia, glioma, glioblastoma, liver cancer, e.g., hepatocellular carcinoma, or diabetes. In some embodiments, the cancer is pancreatic cancer or brain cancer. In some embodiments, brain cancer is selected from the group consisting of Meningioma, Astrocytomas, Gliomas, Glioblastoma multiforme, Medulloblastoma, Ependymoma, Oligodendroglioma, Craniopharyngioma, Pituitary adenoma, Brainstem glioma, Schwannoma, Vestibular schwannoma, Anaplastic astrocytoma, Primary central nervous system lymphoma, Germ cell tumor, Primitive neuroectodermal tumor, Pilocytic astrocytoma, Mixed glioma, Chordoma, Optic nerve glioma and diffuse Astrocytomas.

Subjects that can be treated with compounds of Formulae (I), (II), and/or (III) disclosed herein, or pharmaceutically acceptable salt, ester, prodrug, or stereoisomer of the compounds, according to the methods of this disclosure include, for example, subjects that have been diagnosed as having acute myeloid leukemia, cancer in adolescents, adrenocortical carcinoma childhood, AIDS-related cancers, e.g., lymphoma and Kaposi's Sarcoma, anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, Viral-Induced cancer, leukemia, hematologic malignancy, solid tumor cancer, prostate cancer, castration-resistant prostate cancer, breast cancer, Ewing's sarcoma, bone sarcoma, primary bone sarcoma, T-cell prolymphocyte leukemia, glioma, glioblastoma, hepatocellular carcinoma, liver cancer, or diabetes. In some embodiments subjects that are treated with the compounds of the disclosure include subjects that have been diagnosed as having a non-cancerous hyperproliferative disorder such as benign hyperplasia of the skin, e.g., psoriasis, restenosis, or prostate, e.g., benign prostatic hypertrophy (BPH).

In some embodiments, subjects that can be treated with compounds of Formulae (I), (II), and/or (III) disclosed herein, or pharmaceutically acceptable salt, ester, prodrug, or stereoisomer of the compounds, according to the methods of this disclosure include, for example, subjects that have been diagnosed as having pancreatic cancer or brain cancer. In some embodiments, brain cancer is selected from the group consisting of Meningioma, Astrocytomas, Gliomas, Glioblastoma multiforme, Medulloblastoma, Ependymoma, Oligodendroglioma, Craniopharyngioma, Pituitary adenoma, Brainstem glioma, Schwannoma, Vestibular schwannoma, Anaplastic astrocytoma, Primary central nervous system lymphoma, Germ cell tumor, Primitive neuroectodermal tumor, Pilocytic astrocytoma, Mixed glioma, Chordoma, Optic nerve glioma and diffuse Astrocytomas.

In some embodiments, subjects that can be treated with compounds of Formulae (I), (II), and/or (III) disclosed herein, or pharmaceutically acceptable salt, ester, prodrug, or stereoisomer of the compounds, according to the methods of this disclosure include, for example, subjects that have been diagnosed as having neurological diseases, cardiovascular diseases, and/or an infection.

For example, in some embodiments, the disclosure provides methods of utilizing a cellular target involved in amino acid metabolism (e.g. LAT1) in a cell by contacting the cell with an amount of a compound as disclosed herein sufficient to modulate its activity. In some embodiments, the disclosure provides methods of modulating activity of the cellular target for amino acid metabolism (e.g., LAT1) in a tissue by contacting the tissue with an amount of a compound, prodrug or salt of Formulae (I), (II) and/or (III) as disclosed herein, sufficient to modulate or utilize the activity of the cellular target for amino acid metabolism (e.g., LAT1) in the tissue. In some embodiments, compound, prodrug or salt of Formulae (I), (II) and/or (III) utilize the cellular targets for amino acid metabolism (e.g., LAT1) as a means to get transported into the diseased cell (e.g., a cancer cell).

The compositions containing the compounds or salts thereof described herein can be administered for prophylactic and/or therapeutic treatments. In therapeutic applications, the compositions are administered to a patient already suffering from a disease, in an amount sufficient to cure or at least partially arrest the symptoms of the disease. Amounts effective for this use will depend on the severity and course of the disease, previous therapy, the patient's health status, weight, and response to the drugs, and the judgment of the treating clinician.

The amount of a given agent that will correspond to such an amount will vary depending upon factors such as the particular compound, disease and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated.

### Method of Use

The disclosed compounds containing a radioisotope (also referred to as radioactive compounds) can not only be used as radiolabeled-based therapy agents but can also be used as imaging agents in imaging modalities such as PET and SPECT technologies. Typically, imaging modalities are employed to screen for and/or diagnose various disease states and/or follow treatment of various disease states in subjects. In some embodiments, the disease is a hyperproliferative disease. In some embodiments, the disease is cancer. In some embodiments, the cancer is pancreatic cancer or brain cancer. In some embodiments, the disease is cancer over-expressing LAT1.

Thus, one aspect of the current disclosure is that compounds of Formula (I) can be used as a treatment agent and as an imaging agent. The disclosure refers to such compounds as theranostic agents or as a “theranostic pair” of compounds (e.g., a first theranostic agent and a second theranostic agent). The theranostic agents disclosed herein comprise at least two halogen atoms.

In some embodiments, the first theranostic agent and the second theranostic agent are the same, comprising the same radionuclide. Such a theranostic pair would have the same agent being the radiolabeled-based therapy agent and the imaging agent (e.g., [131]I and [211]At).

In some embodiments, the first theranostic agent and the second theranostic agent are not the same. In some embodiments, the first theranostic agent is a therapeutic agent and is a compound of Formula (I) containing no radioisotopes. In such embodiments, the second theranostic agent is a compound of Formula (I) comprising a radionuclide generally known to be used in SPECT and PET imaging modalities (e.g., [18]F, [124]I, [75]Br, [76]Br, and [77]Br, [123]I, [125]I, [131]I, [210]At or [211]At).

In an alternate embodiment, the first theranostic agent is a radiolabeled-based therapy agent and is a compound of Formula (I) containing a radioisotope generally known to be used in radiolabeled-based therapy agents (e.g., [131]I and/or [211]At). In such embodiments, the second theranostic agent is a compound of Formula (I) comprising a radionuclide generally known to be used in SPECT and PET imaging modalities (e.g., [18]F, [124]I, [75]Br, [76]Br, [77]Br, [123]I, [125]I, [131]I, [210]At or [211]At).

In some embodiments, the atom connectivity (regardless of radioactivity) is the same in both theranostic agents. In other words, often a non-radioactive halogen can be replaced in one theranostic agent with the same or similar halogen but now being a radioisotope, and vice versa. Note that iodine or bromine can be swapped with radioisotopes of astatine.

Thus, one aspect of the current disclosure is to employ a radioactive compound as disclosed (i.e., a compound of Formula (I) comprising a radioisotope) herein in methods of imaging a subject for diagnosing a disease or monitoring efficacy of treatment of a disease by a) administering to a subject in need thereof a radioactive compound as disclosed herein in an effective amount; and b) acquiring at least one image of at least a portion of the subject.

The radioactive compound disclosed herein is a compound of Formula (I) containing a radioisotope suitable for use in imaging modalities such as PET and SPECT technologies. For example, a suitable radioisotope for use in PET imaging is selected from the group consisting of [18]F, [124]I, [75]Br, [76]Br, [77]Br and [210]At. A suitable radioisotope for use in SPECT imaging is [123]I, [125]I, [131]I, or [211]At. Thus, the compounds employed in the methods disclosed herein are compounds of Formula (I), (II), or (III), wherein R³ is a radioisotope R* selected from the group consisting of [123]I, [124]I, [125]I, [131]I, [76]Br, [77]Br, [82]Br, [210]At and [211]At; or R⁴ is [18]F. In some embodiments, the compounds employed in the methods disclosed herein are compounds of Formula (I), (II), or (III), wherein R³ is a radioisotope R* selected from the group consisting of [123]I, [124]I, [131]I, [210]At and [211]At; or R⁴ is [18]F.

In some embodiments, the radioactive compound disclosed herein is part of a theranostic pair as described above. In some embodiments, the radioactive compound disclosed herein is used by itself and is not part of a theranostic pair as described herein.

As already mentioned above, in some embodiments, the radioactive compound disclosed herein is formulated into a pharmaceutical composition/formulation comprising at least one pharmaceutically acceptable excipient and/or carrier. As will be apparent to those skilled in the art, that one or more pharmaceutically acceptable excipients or carriers will vary depending on the mode of administration of the radioactive compound to a subject in need thereof. In some embodiments, the pharmaceutical composition is in the form of a saline-based solution, a suspension, an emulsion, liposome-based preparation, microsphere-based preparation or any other pharmaceutical formulations in liquid form suitable for injection.

The effective amount of the radioactive compound can vary and depends on the mode of administration; the patient's age, weight, and health; as well as the area to be imaged. A skilled artisan would know how to best determine effective amounts of the disclosed radioactive compound.

In some embodiments, the imaging method disclosed herein are employed for diagnosing a disease or assessing efficacy of treatment of a disease or condition in a patient in need thereof. In some embodiments, the disease or condition is cancer. Various cancer types have already been mentioned above. In some embodiments, the cancer type is pancreatic cancer. In some embodiments, the disease or condition is a hyperproliferative disorder of the type as already mentioned above. In some embodiments, the imaging method is employed for diagnosing cancer.

In other embodiments, the imaging method disclosed herein is employed for assessing the efficacy of a treatment to treat a disease or conditions in a person in need thereof. In some embodiments, the disease or condition is cancer. In some embodiments, the treatment comprises administration to the subject in need thereof a therapeutically effective amount of at least one therapeutic agent, i.e., an anti-cancer agent. A skilled artisan would generally be familiar with current anti-cancer treatments, which include, but are not limited to, administration of one or more anti-cancer drugs, radiation, surgery, radiolabeled-based therapy, and/or any combination thereof. In some embodiments, the anti-cancer treatment comprises administration of a compound of Formula (I) as already described above. In some embodiments, the compound of Formula (I) is not radioactive. In some embodiments, the anti-cancer treatment comprises administration of a compound of Formula (I) wherein R³ and R⁴ are independently selected from the group consisting of —I, —At, and —F, and wherein R³ and R⁴ are not the same halogen substituents. In some embodiments, the compound of Formula (I) is radioactive (and thus contains a radionuclide).

In some embodiments, the anti-cancer treatment comprises administration of a commercially available anti-cancer agent. Exemplary anti-cancer agents include, but are not limited to, Altretamine, Bendamustine, Busulfan, Carmustine, Chlorambucil, Cyclophosphamide, Dacarbazine, Ifosfamide, Lomustine, Lurbinectedin, Mechlorethamine, Melphalan, Procarbazine, Streptozocin, Temozolomide, Thiotepa, Trabectedin, Carboplatin, Cisplatin, Oxaliplatin, Bleomycin, Dactinomycin, Daunorubicin, Doxorubicin, Epirubicin, Idarubicin, Mitomycin, Mitoxantrone, Plicamycin, Valrubicin, Methotrexate, Pemetrexed, Pralatrexate, Trimetrexate, Azathioprine, Cladribine, Fludarabine, Mercaptopurine, Thioguanine, Azacitidine, Capecitabine, Cytarabine, Decitabine, Floxuridine, Fluorouracil, Gemcitabine, Trifluridine/Tipracil, Aldesleukin (IL-2), Denileukin Diftitox, Interferon Gamma, Belinostat, Panobinostat, Romidepsin, Vorinostat, Antiandrogens: Abiraterone, Apalutamide, Bicalutamide, Cyproterone, Enzalutamide, Flutamide, Nilutamide, Antiestrogens (including Aromatase Inhibitors): Anastrozole, Exemestane, Fulvestrant, Letrozole, Raloxifene, Tamoxifen, Toremifene, Gonadotropin Releasing Hormone Analogues: Degarelix, Goserelin, Histrelin, Leuprolide, Relugolix, Triptorelin, Lanreotide, Octreotide, Pasireotide, Alemtuzumab, Atezolizumab, Avelumab, Bevacizumab, Blinatumomab, Brentuximab, Cemiplimab, Cetuximab, Daratumumab, Dinutuximab, Dostarlimab, Durvalumab, Elotuzumab, Gemtuzumab, Inotuzumab Ozogamicin, Ipilimumab, Mogamulizumab, Moxetumomab Pasudotox, Necitumumab, Nivolumab, Ofatumumab, Olaratumab, Panitumumab, Pembrolizumab, Pertuzumab, Ramucirumab, Rituximab, Teclistamab, Tositumomab, Trastuzumab, Tremelimumab, Abemaciclib, Acalabrutinib, Afatinib, Alectinib, Alpelisib, Axitinib, Binimetinib, Bortezomib, Bosutinib, Brigatinib, Cabozantinib, Carfilzomib, Ceritinib, Cobimetinib, Copanlisib, Crizotinib, Dabrafenib, Dacomitinib, Dasatinib, Duvelisib, Enasidenib, Encorafenib, Entrectinib, Erdafitinib, Erlotinib, Fedratinib, Futibatinib, Gefitinib, Gilteritinib, Glasdegib, Ibrutinib, Idelalisib, Imatinib, Infigratinib, Ivosidenib, Ixazomib, Lapatinib, Larotrectinib, Lenvatinib, Lorlatinib, Midostaurin, Neratinib, Nilotinib, Niraparib, Olaparib, Osimertinib, Palbociclib, Pazopanib, Pemigatinib, Pexidartinib, Ponatinib, Regorafenib, Ribociclib, Rucaparib, Ruxolitinib, Selumetinib, Sonidegib, Sorafenib, Sunitinib, Talazoparib, Trametinib, Vandetanib, Vemurafenib, Vismodegib, Zanubrutinib, Cabazitaxel, Docetaxel, Paclitaxel, Etoposide, Irinotecan, Teniposide, Topotecan, Vinblastine, Vincristine, Vinorelbine, Asparaginase (Pegaspargase), Belzutifan, Bexarotene, Cedazuridine, Eribulin, Everolimus, Hydroxyurea, Ixabepilone, Lenalidomide, Mitotane, Omacetaxine, Pomalidomide, Selinexor, Tagraxofusp, Tazemetostat, Tebentafusp, Telotristat, Temsirolimus, Thalidomide, and Venetoclax.

In some embodiments, the treatment comprises a commercially available radiolabeled-based therapeutic agent. Exemplary commercially available radiolabel-based therapeutic agent include, but are not limited to, radium-223 dichloride (Xofigo®), sodium iodide I-131 (Hicon®), lobenguane iodine-131 (Azedra®), lutetium-177 (Lutathera® and Pluvicto®) and yttrium-90 (Zevalin®).

In some embodiments, the therapeutic agent is administered prior to administration of the imaging agent disclosed herein.

Using the imaging methods disclosed herein can aid in identifying the presence or absence of tumors and/or changes in size of identified tumors.

These and other embodiments are further illustrated by the following non-limiting examples.

### EXAMPLES

### Phenylalanine Derivatives

Phenylalanine derivatives having structure and properties described herein were prepared as follows:

### Example 1: Identification of Phenylalanine Derivatives for Cancer Imaging and Therapy

Amino acid metabolism represents an important class of pathways in cancer progression. It has been shown that active transportation of amino acids plays a role during the energy metabolism reprogramming in PDAC. LAT1 is responsible for transporting amino acids such as phenylalanine. High LAT1 expression has been found to predict poor prognosis of PDAC and resistance to therapy. Elevated expressions of LAT1 in PDAC are associated with tumor size and disease stages.

To assess LAT1-mediated uptake and/or binding, a library of fluorinated amino acid agents was constructed using the methods described in FIG. 1.

### Example 2: Synthesis of Optically Pure Poly-Halogentated Compounds

Enantioenriched analogs have exhibited high tumor uptake and tumor-to-normal organ contrast

For the preparation of the disclosed compounds the well-established DuanPhos-rhodium complex can be used as the chiral catalyst to generate enantioenriched precursors, with D and L configurations, for subsequent radionuclide incorporation (Scheme 1). Both D- and L-agents will be obtained with >95% optical purity, which will be measured by chiral HPLC.

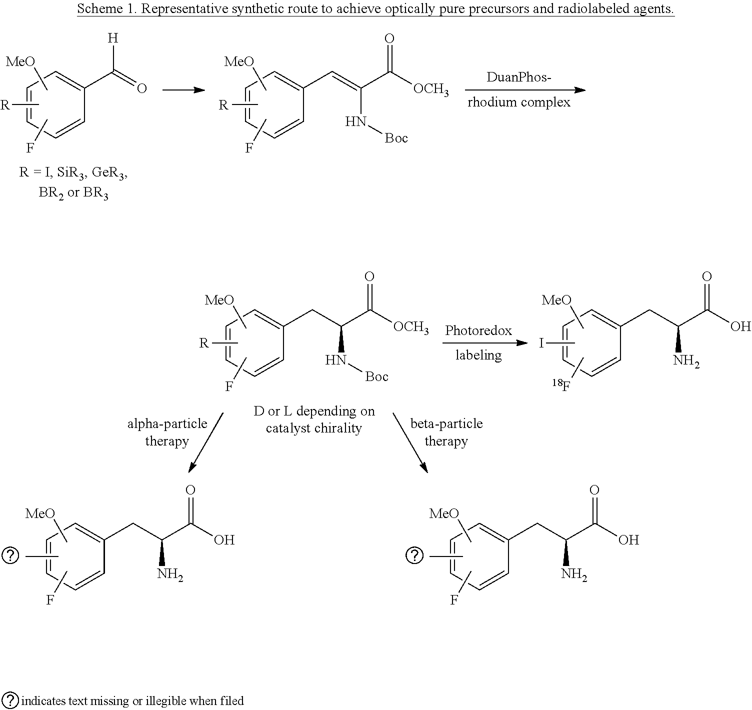

### Example 3: Radiolabeling an Initial Evaluation of Phenylalanine Derivatives

[18F] labeling: The amino acid derivatives will be labeled with [18]F based on photoredox radiofluorination methods.

Amino acid transporter assay: To investigate the involvement of amino acid transporters in the cellular uptake of the disclosed PET agents, cellular uptake assay in MIA Paca-2 PDAC cancer cell lines will be performed. In brief, cells will be plated onto 6-well tissue culture plates (2×105 cells/well) and incubated with [18]F-labeled PET agents (8 μCi/well) for 0-2 h. After incubation, cells will be washed with ice-cold PBS twice, detached, and collected. The radioactivity will be measured with a γ-counter. Data will be expressed in mean±SD percentage of cellular uptake (% Uptake). To determine the specificity of the disclosed [18]F-PET agents to L-amino acid transporter (LAT), additional assays will be performed: the cellular uptake assay will be performed in the presence of an excess amount of L-Phe or LAT1 inhibitors as a competitor to investigate whether cell uptake of [18]F-PET agents is mediated by LAT1.

Serum stability, in vivo stability, pharmacokinetics, and tissue distribution of the PET agents. Priority [18]F-labeled PET agents will be incubated with serum solution for 5, 15, 30, 60, 120, and 240 min, then analyzed by radio-HPLC to determine compound stability. In vivo metabolic study will be determined by tissue homogenization, extraction, and radioHPLC analysis. The pharmacokinetics and tissue distribution of these PET agents will be determined by biodistribution at 30 min, 1, 2, and 4 h post injection. 131I/211At labeling: The amino acid derivatives will be labeled with [131]1/[211]At from the appropriate precursor described in Formula (IV). [131]I/[211]At will be preferentially used for radionuclide-based therapy.

In summary, optically pure agents (D and L) will be synthesized and radiolabeled. Agents with low pancreatic uptake are preferred due to the low background of a normal organ.

### Example 4: PET Imaging and Biodistribution in PDAC Tumor Model

In this study, the in vivo tumor targeting efficacy and pharmacokinetics of the radiotracers prepared above will be assessed using pancreatic tumor models. In addition, the uptake in the normal pancreas will be assessed to ensure the selected agents have a high tumor to background ratio.

The constructed library of analogs will first be evaluated in MIA Paca-2 and PANC-1 orthotopic xenograft models to select promising agents with prominent tumor uptake, high contrast and relatively low pancreas uptake. In brief, 100 μCi of the PET agent will be injected and the animal will be scanned at 1, 2, 4, and 6 h post injection (p.i.). Region of interest will be drawn at the tumor and major organ area. Bio-D will be performed after the last time point is finished. The uptake and clearance profile will be compared among different [18]F agents. The selected agent should have tumor uptake >5% ID/g, labeling yield >5%, tumor retention >60% from 1 to 4 h p.i. and >50% at 6 h p.i., and tumor to muscle ratio >3. After initial screening, two selected lead agents will be tested in a syngeneic orthotopic PDAC model, which was established from genetically engineered KPC (KRasLSL.G12D/+; p53LSL.R172H/+; PdxCre on a C57Bl/6 background, using static scans at 1, 2, 4 and 6 h p.i. IHC and autoradiography would be performed to study LAT1 expression level and localization of the PET agent within the tumor region. After initial imaging screening, [131]I/[211]At-labeled analogs will be prepared for biodistribution study to further confirm in vivo distribution of the therapeutic agents at late time points. In brief, the KPC orthotopic xenograft models will be injected with ˜100 μCi of [131]I/[211]At-labeled amino acids and will be sacrificed at 1, 4, 24, 48, 72 and 96 h post-injection.

Lead agents will be evaluated in an orthotopic PDAC model established from KPC cells. Because the KPC model recapitulates many of the salient clinical and histopathological features (including poor vascularity, fibrosis, etc.) of human disease, in a future study, one would evaluate the lead agent in KPC tumors that arise spontaneously to determine uptake of lead agents in primary tumor and metastasis.

### Compound Examples

Absolute stereochemistries for compounds described herein, including the compounds provided in the following examples, have been arbitrarily assigned based on chiral HPLC elution order. Such stereochemical assignment may be subject to change when final absolute stereochemical assignment of the compounds is conducted.

### Example 5: Synthesis of tert-Butyl (S)-3-(3,5-difluoro-2-iodo-6-methoxyphenyl)-2-((diphenylmethylene)amino)propanoate and tert-Butyl (R)-3-(3,5-difluoro-2-iodo-6-methoxyphenyl)-2-((diphenylmethylene)amino)propanoate

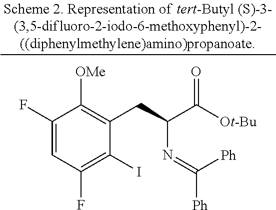 [SMILES: COc1c(F)cc(F)c(I)c1C[C@H](N=C(c1ccccc1)c1ccccc1)C(=O)OC(C)(C)C]

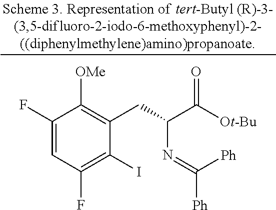 [SMILES: COc1c(F)cc(F)c(I)c1C[C@@H](N=C(c1ccccc1)c1ccccc1)C(=O)OC(C)(C)C]

The preparation of tert-Butyl (S)-3-(3,5-difluoro-2-iodo-6-methoxyphenyl)-2-((diphenylmethylene)amino)propanoate (Scheme 2) and tert-Butyl (R)-3-(3,5-difluoro-2-iodo-6-methoxyphenyl)-2-((diphenylmethylene)amino)propanoate (Scheme 3) is shown schematically in Scheme 4. To a stirred mixture of 3,5-difluoro-2-methoxybenzaldehyde (10.0 g, 58.1 mmol) and 4-chloro-2-(trifluoromethyl)aniline (5.68 g, 29.1 mmol) in trifluoroacetic acid (50 mL) and DCE (250 mL) was added Pd(OAc)₂ (3.26 g, 14.5 mmol) and NIS (15.7 g, 69.7 mmol). After heating at 60° C. 16 h under nitrogen atmosphere, the mixture was cooled to rt and concentrated under reduced pressure. The residue was purified by reverse flash chromatography (C18 silica gel, ACN-water, 30-100% with 0.1% FA). The obtained product was re-purified by silica gel column chromatography (EA-Petroleum ether, 9%) to afford the title compound (2.0 g, 11%) as a white solid. GCMS (ES, m/z): 298.05 [M].

To a stirred mixture of 3,5-difluoro-2-iodo-6-methoxybenzaldehyde (2.0 g, 6.71 mmol) in methanol (25 mL) at 0° C. was added NaBH₄ (507 mg, 13.4 mmol), portion-wise. After 1 h, the mixture was concentrated under reduced pressure and the crude residue was purified by silica gel column chromatography (EA-Petroleum ether, 20%) to afford the title compound (1.5 g, 75%) as a white solid. GCMS (ES, m/z): 300.10 [M].

To a stirred mixture of (3,5-difluoro-2-iodo-6-methoxyphenyl)methanol (1.30 g, 4.33 mmol) in DCM (20 mL) at 0° C. was added PBr₃ (2.35 g, 8.66 mmol), dropwise. After 1 h, the mixture was concentrated under reduced pressure and the crude residue was purified by silica gel column chromatography (EA-Petroleum ether, 9%) to afford the title compound (1.20 g, 76%) as a white solid. GCMS (ES, m/z): 362.20, 364.20 [M].

To a stirred mixture of 3-(bromomethyl)-1,5-difluoro-2-iodo-4-methoxybenzene (1.20 g, 3.31 mmol) and tert-butyl 2-[(diphenylmethylidene)amino]acetate (977 mg, 3.31 mmol) in MeCN (15 mL) was added tetrabutylammonium bromide (106 mg, 0.33 mmol) and K₂CO₃ (914 mg, 6.62 mmol). After 16 h the mixture was concentrated under reduced pressure and the crude residue was purified by reverse flash chromatography (C18 silica gel, ACN-water, 60-100% with 10% NH₄HCO₃). The resulting racemate was separated by prep-SFC (Column: CHIRAL ART Cellulose-SZ 5 um, 3×25 cm; Mobile Phase A: CO₂, Mobile Phase B: 12% IPA (with 1% 2M NH₃); Flow rate: 80 mL/min; Column Temperature: 35° C.; Back Pressure: 100 bar; Wavelength: 220 nm) to afford tert-butyl (S)-3-(3,5-difluoro-2-iodo-6-methoxyphenyl)-2-((diphenylmethylene)amino)propanoate (first eluting isomer, 252.0 mg, 13%) and tert-butyl (R)-3-(3,5-difluoro-2-iodo-6-methoxyphenyl)-2-((diphenylmethylene)amino)propanoate (second eluting isomer, 254.1 mg, 13%) as white semi-solids.

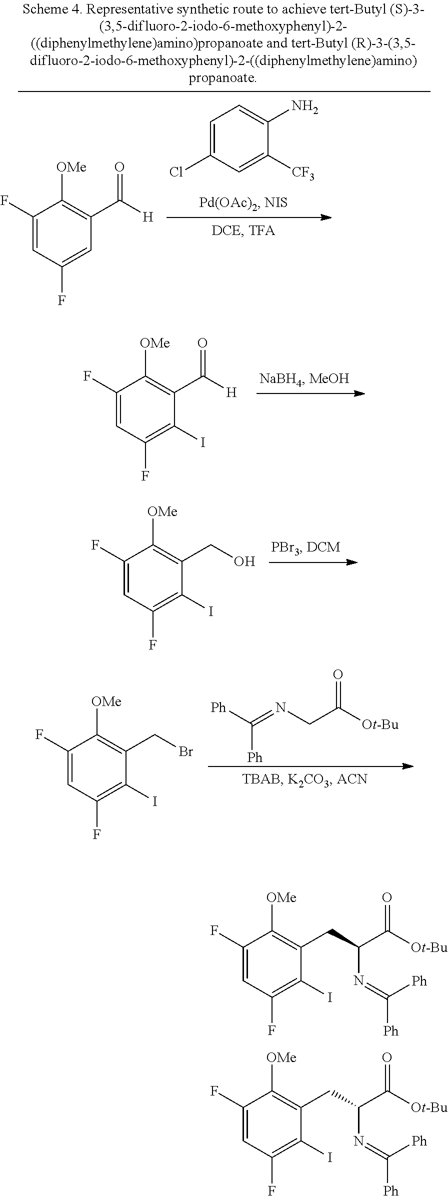

For tert-Butyl (S)-3-(3,5-difluoro-2-iodo-6-methoxyphenyl)-2-((diphenylmethylene)amino)propanoate, ¹H NMR (400 MHz, DMSO-d₆) δ 7.53-7.48 (m, 2H), 7.47-7.32 (m, 7H), 6.61 (br d, J=6.4 Hz, 2H), 4.31 (dd, J=9.6, 4.0 Hz, 1H), 3.60 (s, 3H), 3.51-3.42 (m, 1H), 3.32-3.24 (m, 1H), 1.38 (s, 9H). ¹⁹F NMR (376 MHz, DMSO-d₆) δ −92.36 (d, J=5.3 Hz), 127.15 (d, J=5.6). LCMS (ES, m/z): 578.20 [M+H]⁺; 98.2% purity (254 nm).

For tert-Butyl (R)-3-(3,5-difluoro-2-iodo-6-methoxyphenyl)-2-((diphenylmethylene)amino)propanoate, ¹H NMR (400 MHz, DMSO-d₆) δ 7.53-7.48 (m, 2H), 7.47-7.32 (m, 7H), 6.61 (br d, J=6.4 Hz, 2H), 4.31 (dd, J=9.6, 4.0 Hz, 1H), 3.60 (s, 3H), 3.51-3.42 (m, 1H), 3.32-3.24 (m, 1H), 1.38 (s, 9H). ¹⁹F NMR (376 MHz, DMSO-d₆) δ −92.36 (d, J=5.6 Hz), 127.15 (d, J=5.6 Hz). LCMS (ES, m/z): 578.15 [M+H]⁺; 98.4% purity (254 nm).

### Example 6: Synthesis of tert-Butyl (2S)-3-(2,5-difluoro-3-iodo-6-methoxyphenyl)-2-[(diphenylmethylidene)amino]propanoate and tert-Butyl (2R)-3-(2,5-difluoro-3-iodo-6-methoxyphenyl)-2-[(diphenylmethylidene)amino]propanoate

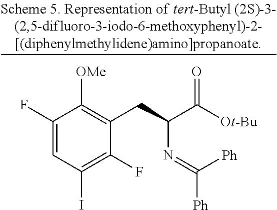 [SMILES: COc1c(F)cc(I)c(F)c1C[C@H](N=C(c1ccccc1)c1ccccc1)C(=O)OC(C)(C)C]

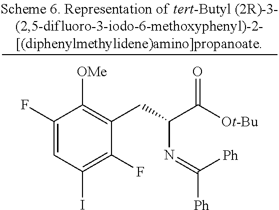 [SMILES: COc1c(F)cc(I)c(F)c1C[C@@H](N=C(c1ccccc1)c1ccccc1)C(=O)OC(C)(C)C]

The preparation of tert-Butyl (2S)-3-(2,5-difluoro-3-iodo-6-methoxyphenyl)-2-[(diphenylmethylidene)amino]propanoate (Scheme 5) and tert-Butyl (2R)-3-(2,5-difluoro-3-iodo-6-methoxyphenyl)-2-[(diphenylmethylidene)amino]propanoate (Scheme 6) is shown schematically in Scheme 7. To a stirred mixture of 3,6-difluoro-2-methoxybenzaldehyde (10.0 g, 58.1 mmol) in MeOH (100 mL) at 0° C. was added NaBH₄ (4.42 g, 116 mmol) portion-wise. After 1 h at 0° C., the mixture was concentrated under reduced pressure and the crude residue was purified by silica gel column chromatography (EA-Petroleum ether, 25%) to afford the title compound (7.00 g, 69%) as a white solid. GCMS (ES, m/z): 174.10 [M].

A mixture of (3,6-difluoro-2-methoxyphenyl)methanol (6.00 g, 34.4 mmol), iodine (8.74 g, 34.4 mmol) and PhI(OAc)₂ (11.1 g, 34.4 mmol) in HFIP (60 mL) was stirred at rt for 16 h, whereupon it was concentrated under reduced pressure. The crude residue was purified by silica gel column chromatography (EA-Petroleum ether, 25%) to afford the title compound (4.20 g, 40%) as a white solid. GCMS (ES, m/z): 299.90 [M].

To a solution of (2,5-difluoro-3-iodo-6-methoxyphenyl)methanol (2.00 g, 6.67 mmol) in DCM (20 mL) at 0° C. was added PBr₃ (3.60 g, 13.3 mmol), dropwise. After 1 h at rt, the mixture was poured into ice-water (100 mL) and extracted with DCM (2×100 mL). The combined organic layers were washed with brine (100 mL), dried over anhydrous Na₂SO₄ and concentrated under reduced pressure to afford the title compound (2.0 g, 82%) as a white solid. GCMS (ES, m/z): 362.10, 364.10 [M].

To a stirred mixture of 3-(bromomethyl)-1,4-difluoro-5-iodo-2-methoxybenzene (2.00 g, 5.51 mmol), tert-butyl 2-[(diphenylmethylidene)amino]acetate (3.26 g, 11.0 mmol), tetrabutylammonium bromide (177 mg, 0.55 mmol) in toluene (20 mL) at 0° C. was added KOH (6.18 g, 55.1 mmol, 50%) in portions. After stirring at rt for 16 h, the mixture was partially concentrated under reduced pressure and the crude residue was purified by reverse flash chromatography (C18 silica gel, ACN-water, 0-100% with 10 mM NH₄HCO₃) to afford tert-butyl 3-(2,5-difluoro-3-iodo-6-methoxyphenyl)-2-[(diphenylmethylidene)amino]propanoate (950 mg). The racemate was separated by Prep-SFC [Column: CHIRAL IG 5 μm, 3×25 cm; Mobile Phase A: CO₂, Mobile Phase B: 12% IPA (with 1% 2M NH₃ in MeOH); Flow rate: 90 mL/min; Column Temperature: 35° C.; Back Pressure: 100 bar; Wavelength: 220 nm] to afford tert-butyl (2S)-3-(2,5-difluoro-3-iodo-6-methoxyphenyl)-2-[(diphenylmethylidene)amino]propanoate (first eluting isomer, 365 mg, 11%) and tert-butyl (2R)-3-(2,5-difluoro-3-iodo-6-methoxyphenyl)-2-[(diphenylmethylidene)amino]propanoate (second eluting isomer, 383 mg, 12%) as white solids.

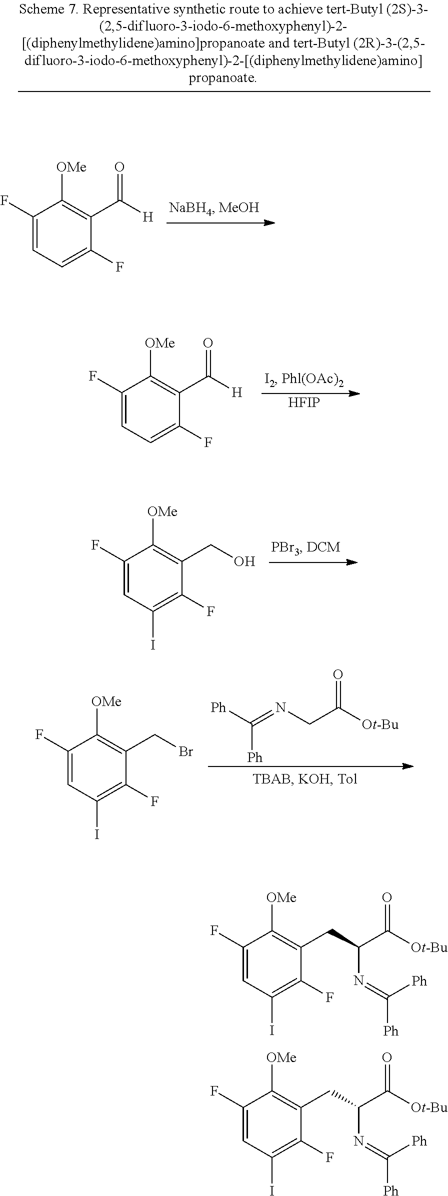

For tert-Butyl(2S)-3-(2,5-difluoro-3-iodo-6-methoxyphenyl)-2-[(diphenylmethylidene)amino]propanoate, 1H NMR (400 MHz, DMSO-d₆) δ 7.67 (dd, J=10.8, 6.0 Hz, 1H), 7.52-7.40 (m, 6H), 7.40-7.33 (m, 2H), 6.78-6.67 (m, 2H), 4.18-4.10 (m, 1H), 3.66-3.59 (m, 3H), 3.20-3.08 (m, 2H), 1.36 (s, 9H). 19F NMR (376 MHz, DMSO-d₆) δ −98.97 (d, J=14.6 Hz), 133.48 (d, J=15.0 Hz). LCMS (ES, m/z): 577.90 [M+H]⁺; 99.8% purity (254 nm).

For tert-Butyl (2R)-3-(2,5-difluoro-3-iodo-6-methoxyphenyl)-2-[(diphenylmethylidene)amino]propanoate, ¹H NMR (400 MHz, DMSO-d₆) δ 7.72-7.63 (m, 1H), 7.50-7.41 (m, 6H), 7.40-7.33 (m, 2H), 6.79-6.67 (m, 2H), 4.18-4.09 (m, 1H), 3.64-3.58 (m, 3H), 3.20-3.09 (m, 2H), 1.36 (s, 9H). ¹⁹F NMR (376 MHz, DMSO-d₆) δ −98.97 (d, J=14.7 Hz), 133.48 (d, J=15.0). LCMS (ES, m/z): 577.90 [M+H]⁺; 99.8% purity (254 nm).

### Example 7: Synthesis of (R)-2-amino-3-(2,5-difluoro-3-iodo-6-methoxyphenyl)propanoic acid

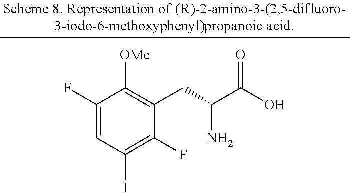 [SMILES: COc1c(F)cc(I)c(F)c1C[C@@H](N)C(=O)O]

The preparation of (R)-2-amino-3-(2,5-difluoro-3-iodo-6-methoxyphenyl)propanoic acid (Scheme 8) is shown schematically in Scheme 9. A mixture of tert-butyl (2R)-3-(2,5-difluoro-3-iodo-6-methoxyphenyl)-2-[(diphenylmethylidene)amino]propanoate (20 mg, 0.04 mmol) in HCl (6 M, 1 mL) was heated at 100° C. for 30 min, whereupon is was cooled to rt and directly purified by reverse flash chromatography (C18 silica gel, ACN-water, 0-20% with 0.1% FA) to afford the title compound (6 mg, 50%) as a white solid.

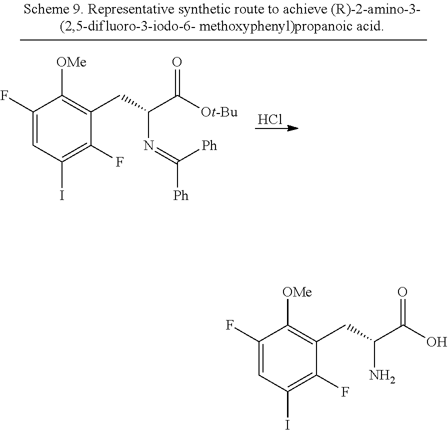 [SMILES: COc1c(F)cc(I)c(F)c1C[C@@H](N=C(c1ccccc1)c1ccccc1)C(=O)OC(C)(C)C ; Cl ; COc1c(F)cc(I)c(F)c1C[C@@H](N)C(=O)O]

For (R)-2-amino-3-(2,5-difluoro-3-iodo-6-methoxyphenyl)propanoic acid, ¹H NMR (400 MHz, DMSO-d₆) δ 7.79-7.59 (m, 1H), 3.90 (s, 3H), 3.63-3.52 (m, 1H), 3.18 (dd, J=13.8, 7.4 Hz, 1H), 3.05-2.88 (m, 1H). ¹⁹F NMR (376 MHz, DMSO-d₆) δ −100.34 (d, J=14.6 Hz), 133.51 (d, J=14.6 Hz). LCMS (ES, m/z): 358.00 [M+H]⁺; 99.6% purity (220 nm).

### Example 8: Synthesis of (2S)-2-amino-3-(2,5-difluoro-3-iodo-6-methoxyphenyl)-N-methylpropanamide

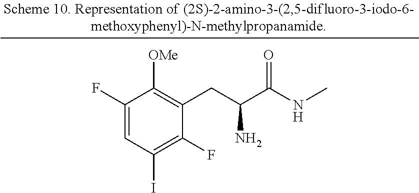 [SMILES: CNC(=O)[C@@H](N)Cc1c(F)c(I)cc(F)c1OC]

The preparation of (2S)-2-amino-3-(2,5-difluoro-3-iodo-6-methoxyphenyl)-N-methylpropanamide (Scheme 10) is shown schematically in Scheme 11. To a solution of tert-butyl (2S)-3-(2,5-difluoro-3-iodo-6-methoxyphenyl)-2-[(diphenylmethylidene)amino]propanoate (70 mg, 0.12 mmol) in EtOH/H₂O (4:1, 2 mL) was added hydroxylamine hydrochloride (84 mg, 1.21 mmol). After heating at 50° C. for 1 h, the mixture was directly purified by reversed-phase flash chromatography (C18 silica gel, ACN-water, 20-70% with 10 mM NH₄HCO₃) to afford the title compound (50 mg, 99%) as a white solid. LCMS (ES, m/z): 358.10 [M-tBu+H]⁺.

To a solution of tert-butyl (2S)-2-amino-3-(2,5-difluoro-3-iodo-6-methoxyphenyl)propanoate (45 mg, 0.11 mmol) and TEA (22 mg, 0.22 mmol) in DCM (2 mL) was added 2,5-dioxopyrrolidin-1-yl 9H-fluoren-9-ylmethyl carbonate (44 mg, 0.13 mmol). After 1 h, the mixture was concentrated under reduced pressure and purified by silica gel column chromatography (EA-petroleum ether, 20%) to afford the title compound (60 mg, 86%) as a white solid. LCMS (ES, m/z): 658.10 [M+Na]⁺.

To a solution of tert-butyl (2S)-3-(2,5-difluoro-3-iodo-6-methoxyphenyl)-2-{[(9H-fluoren-9-ylmethoxy)carbonyl]amino}propanoate (50 mg, 0.08 mmol) in DCM (2 mL) was added TFA (0.5 mL). After 30 min, the mixture was concentrated under reduced pressure. The crude residue was diluted with NaHCO₃ (satd aq, 15 mL) at 0° C. and extracted with DCM (3×15 mL). The combined organic layers were washed with brine (20 mL), dried over anhydrous Na₂SO₄ and concentrated under reduced pressure to afford the title compound (40 mg) as a yellow solid. LCMS (ES, m/z): 602.15 [M+Na]⁺.

To a solution of (2S)-3-(2,5-difluoro-3-iodo-6-methoxyphenyl)-2-{[(9H-fluoren-9-ylmethoxy)carbonyl]amino}propanoic acid (40 mg), methylamine hydrochloride (11 mg, 0.16 mmol) and HATU (60 mg, 0.16 mmol) in DMF (2 mL) was added DIEA (51 mg, 0.40 mmol). After 1 h, the mixture was directly purified by reversed phase flash chromatography (C18 silica gel, ACN-water, 10-50% with 10 mM NH₄HCO₃), affording the title compound (35 mg, 74%, 2-steps) as a white solid. LCMS (ES, m/z): 593.10 [M+H]⁺.

To a solution of 9H-fluoren-9-ylmethyl N-[(1S)-2-(2,5-difluoro-3-iodo-6-methoxyphenyl)-1-(methylcarbamoyl)ethyl]carbamate (10 mg, 0.02 mmol) in DCM (1 mL) was added piperidine (6 mg, 0.07 mmol). After 1 h, the mixture was directly purified by Prep-HPLC [Column: XBridge Prep RP OBD C18 5 μm, 30×150 mm; Mobile Phase: ACN-water, 14-44% (with 10 mM NH₄HCO₃), Flow rate: 60 mL/min; Wavelength: 254/220 nm], affording the title compound (2.6 mg, 35%) as a white solid.

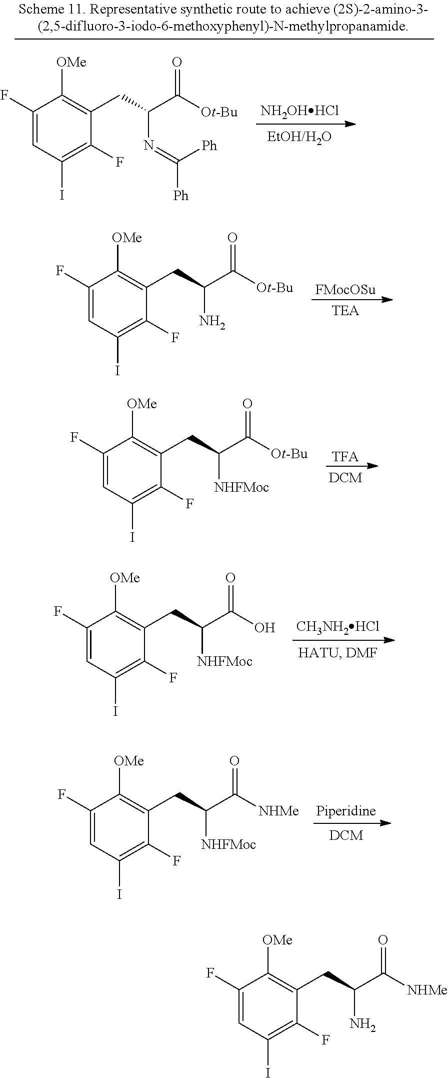

For (2S)-2-amino-3-(2,5-difluoro-3-iodo-6-methoxyphenyl)-N-methylpropanamide, ¹H NMR (400 MHz, DMSO-d₆/D₂O) δ 7.63 (dd, J=10.4, 6.0 Hz, 1H), *4.10-4.01 (m, 0.25H), 3.93-3.80 (m, 3H), *3.26 (dd, J=8.0, 6.4 Hz, 0.75H), 2.98-2.84 (m, 1H), 2.80-2.63 (m, 1H), 2.62-2.55 (m, 3H). *Partial integration due to presence of rotamers. ¹⁹F NMR (376 MHz, DMSO-d₆) δ −100.17-(−)100.23 (m), −133.32-(−)133.43 (m). LCMS (ES, m/z): 370.95 [M+H]⁺; 99.2% purity (220 nm).

### Example 9: Synthesis of tert-Butyl N-[(1S)-2-(2,5-difluoro-3-iodo-6-methoxyphenyl)-1-(methylcarbamoyl)ethyl]carbamate

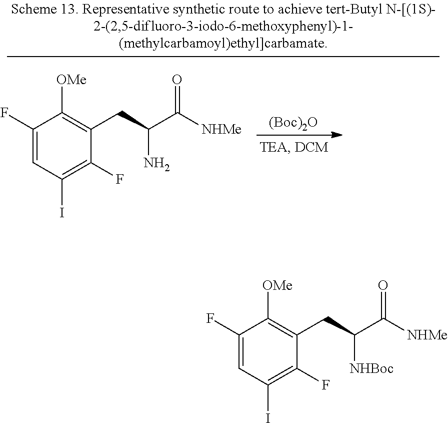 [SMILES: "(Boc)2O" ; CNC(=O)[C@@H](N)Cc1c(F)c(I)cc(F)c1OC ; CNC(=O)[C@H](Cc1c(F)c(I)cc(F)c1OC)NC(=O)OC(C)(C)C]

The preparation of tert-Butyl N-[(1S)-2-(2,5-difluoro-3-iodo-6-methoxyphenyl)-1-(methylcarbamoyl)ethyl]carbamate (Scheme 12) is shown schematically in Scheme 13. To a solution of (2S)-2-amino-3-(2,5-difluoro-3-iodo-6-methoxyphenyl)-N-methylpropanamide (8 mg, 0.02 mmol) and TEA (5 mg, 0.04 mmol) in DCM (1 mL) was added di-tert-butyl dicarbonate (6 mg, 0.03 mmol). After 1 h, the mixture was concentrated under reduced pressure and Prep-HPLC [Column: XBridge Prep OBD C18 5 μm, 30×150 mm; Mobile Phase: ACN-water, 35-65% (with 10 mM NH₄HCO₃); Flow rate: 60 mL/min; Wavelength: 254/220 nm], affording the title compound (2.0 mg, 21%) as a white solid.

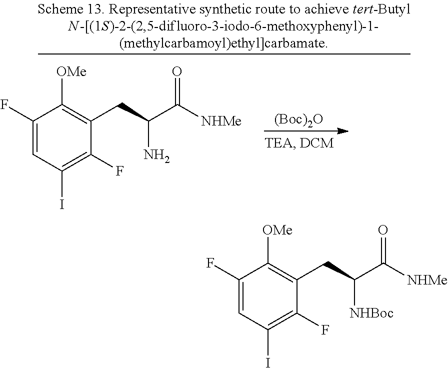 [SMILES: "(Boc)2O" ; CNC(=O)[C@@H](N)Cc1c(F)c(I)cc(F)c1OC ; CNC(=O)[C@H](Cc1c(F)c(I)cc(F)c1OC)NC(=O)OC(C)(C)C]

For tert-Butyl N-[(1S)-2-(2,5-difluoro-3-iodo-6-methoxyphenyl)-1-(methylcarbamoyl)ethyl]carbamate, ¹H NMR (400 MHz, DMSO-d₆) δ 7.90-7.72 (m, 1H), 7.71-7.63 (m, 1H), *6.68 (d, J=9.2 Hz, 0.9H), *6.39-6.29 (m, 0.1H), 4.22-4.01 (m, 1H), 3.99-3.85 (m, 3H), 3.11-2.98 (m, 1H), 2.89-2.74 (m, 1H), 2.64-2.55 (m, 3H), 1.39-1.12 (m, 9H). *Partial integration due to presence of rotamers. ¹⁹F NMR (376 MHz, DMSO-d₆) δ −100.05 (major rotamer), −100.09 (minor rotamer), −132.58 (minor rotamer), −133.62 (major rotamer). LCMS (ES, m/z): 370.90 [M-Boc+H]⁺; 99.5% purity (220 nm).

### Example 10: Synthesis of tert-Butyl (2R)-2-[(tert-butoxycarbonyl)(methyl)amino]-3-(2,5-difluoro-3-iodo-6-methoxyphenyl)propanoate

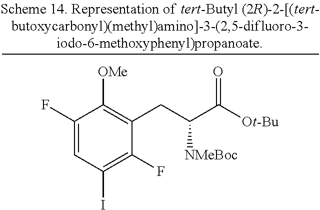

The preparation of tert-Butyl (2R)-2-[(tert-butoxycarbonyl)(methyl)amino]-3-(2,5-difluoro-3-iodo-6-methoxyphenyl)propanoate (Scheme 14) is shown schematically in Scheme 15. To a solution of tert-butyl (2R)-2-amino-3-(2,5-difluoro-3-iodo-6-methoxyphenyl)propanoate (40 mg, 0.10 mmol) and TEA (29 mg, 0.29 mmol) in DCM (2 mL) was added di-tert-butyl dicarbonate (42 mg, 0.19 mmol). After 1 h, the mixture was concentrated under reduce pressure and the crude residue was purified by silica gel chromatography (THF-petroleum ether, 20%) to afford the title compound (40 mg, 80%) as a yellow oil. LCMS (ES, m/z): 458.15 [M-tBu+H]⁺.

To a mixture of tert-butyl (2R)-2-[(tert-butoxycarbonyl)amino]-3-(2,5-difluoro-3-iodo-6-methoxyphenyl)propanoate (30 mg, 0.06 mmol) in DMF (1 mL) at 0° C. was added NaH (60% in mineral oil, 5 mg, 0.12 mmol). After 30 min, CH₃I (16 mg, 0.12 mmol) was added. The mixture was stirred for additional 2 h at 0° C., then quenched with water (0.1 mL) and purified by reverse flash chromatography (C18 silica gel, ACN-water, 20-75% with 10 mM NH₄HCO₃) to afford the title compound (20 mg, 64%) as yellow oil.

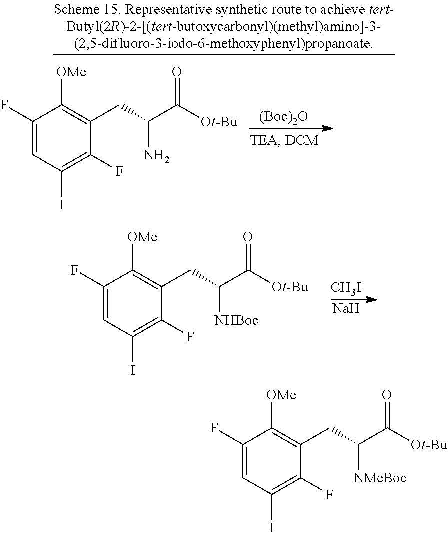

For tert-Butyl (2R)-2-[(tert-butoxycarbonyl)(methyl)amino]-3-(2,5-difluoro-3-iodo-6-methoxyphenyl)propanoate, ¹H NMR (400 MHz, Chloroform-d) δ 7.42-7.32 (m, 1H), 4.78-4.70 (m, 1H), 4.06-3.95 (m, 3H), 3.27-3.10 (m, 2H), 2.83-2.71 (m, 3H), 1.53-1.46 (m, 9H), 1.42-1.32 (m, 9H). LCMS (ES, m/z): 528.15 [M+H]⁺, 372.00 [M-Boc-tBu+H]⁺; 97% purity (254 nm).

### Example 11: Synthesis of tert-Butyl (S)-3-(4,5-difluoro-3-iodo-2-methoxyphenyl)-2-((diphenylmethylene)amino)propanoate and tert-Butyl (R)-3-(4,5-difluoro-3-iodo-2-methoxyphenyl)-2-((diphenylmethylene)amino)propanoate

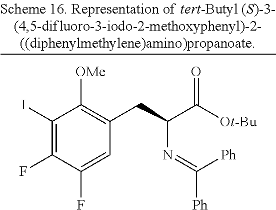 [SMILES: COc1c(C[C@H](N=C(c2ccccc2)c2ccccc2)C(=O)OC(C)(C)C)cc(F)c(F)c1I]

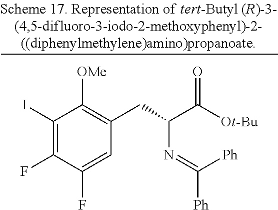 [SMILES: COc1c(C[C@@H](N=C(c2ccccc2)c2ccccc2)C(=O)OC(C)(C)C)cc(F)c(F)c1I]

The preparation of tert-Butyl (S)-3-(4,5-difluoro-3-iodo-2-methoxyphenyl)-2-((diphenylmethylene)amino)propanoate (Scheme 16) and tert-Butyl (R)-3-(4,5-difluoro-3-iodo-2-methoxyphenyl)-2-((diphenylmethylene)amino)propanoate (Scheme 17) is shown schematically in Scheme 18. A mixture of 4,5-difluoro-2-hydroxybenzaldehyde (9.00 g, 56.9 mmol) and NIS (14.1 g, 62.6 mmol) in DMF (100 mL) was stirred at rt for 16 h, whereupon it was diluted with water (200 mL) and extracted with EA (2×200 mL). The combined organic layers were washed with brine (2×200 mL), dried over anhydrous Na₂SO₄ and concentrated under reduced pressure. The crude residue was purified by reverse flash chromatography (C18 silica gel, ACN-water, 0-60% with 0.1% FA) to afford the title compound (10 g, 61%) as a white solid. GCMS (ES, m/z): 284.05 [M].

To a mixture of 4,5-difluoro-2-hydroxy-3-iodobenzaldehyde (10.0 g, 35.2 mmol) and Mel (10.0 g, 70.4 mmol) in (DMF (140 mL) was added K₂CO₃ (12.2 g, 88.0 mmol). After 16 h, the mixture was diluted with water (300 mL) and extracted with EA (3×150 mL). The combined organic layers were washed with brine (3×300 mL), dried over anhydrous Na₂SO₄ and concentrated under reduced pressure. The crude product was purified by silica gel chromatography (EA-Petroleum ether, 25%) to afford the title compound (10.0 g, 95%) as a white solid. GCMS (ES, m/z): 298.10 [M].

To a mixture of 4,5-difluoro-3-iodo-2-methoxybenzaldehyde (5.00 g, 16.8 mmol) in methanol (60 mL) at 0° C. was added NaBH₄ (1.27 g, 33.6 mmol), portion-wise. After 1 h at rt, the mixture was concentrated under reduced pressure and the crude residue was purified by silica gel chromatography (EA-Petroleum ether, 50%) to afford the title compound (3 g, 18%, 30% purity) as a white solid. GCMS (ES, m/z): 300.20 [M].

To a mixture of (4,5-difluoro-3-iodo-2-methoxyphenyl)methanol (2.70 g, 2.70 mmol, 30% purity) in DCM (50 mL) at 0° C. was added PBr₃ (4.87 g, 17.9 mmol), portion-wise. After 1 h at rt, the mixture was partially concentrated under reduced pressure and purified by silica gel chromatography (EA-Petroleum ether, 10%), affording the title compound (2.5 g, crude) as a white solid. GCMS (ES, m/z): 362.20, 364.20 [M].

To a mixture of 1-(bromomethyl)-4,5-difluoro-3-iodo-2-methoxybenzene (2.5 g, crude) and tert-butyl 2-[(diphenylmethylidene)amino]acetate (2.03 g, 6.89 mmol) in MeCN (25 mL) was added tetrabutylammonium bromide (222 mg, 0.69 mmol) and K₂CO₃ (1.90 g, 13.8 mmol). After stirring for 16 h, the mixture was concentrated under reduced pressure and the crude residue was purified by reverse flash chromatography (C18 silica gel, ACN-water, 60-100% with 10% NH₄HCO₃) to afford tert-butyl-3-(4,5-difluoro-3-iodo-2-methoxyphenyl)-2-((diphenylmethylene)amino)propanoate. The racemic mixture was separated by Prep-SFC [Column: (R, R)-WHELK-O1 5 um, 3×25 cm; Mobile Phase A: CO₂, Mobile Phase B: 14% MeOH (with 0.3% 7M NH₃-MeOH); Flow rate: 90 mL/min; Column Temperature: 35° C.; Back Pressure: 100 bar; Wavelength: 220 nm] to afford tert-butyl (S)-3-(4,5-difluoro-3-iodo-2-methoxyphenyl)-2-((diphenylmethylene)amino)propanoate (first eluting isomer, 241 mg, 15%) and tert-butyl (R)-3-(4,5-difluoro-3-iodo-2-methoxyphenyl)-2-((diphenylmethylene)amino)propanoate (second eluting isomer, 243 mg, 15%) as white semi-solids.

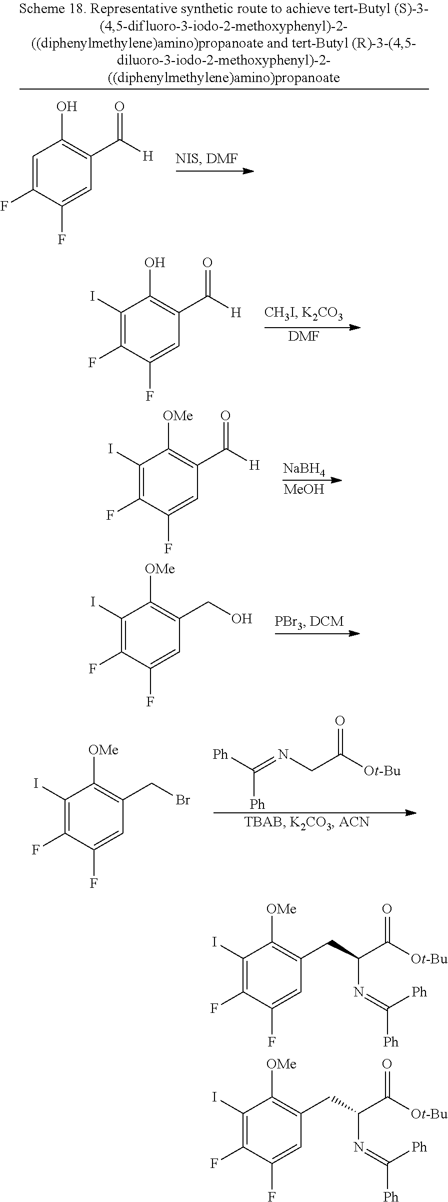

For tert-Butyl (S)-3-(4,5-difluoro-3-iodo-2-methoxyphenyl)-2-((diphenylmethylene)amino)propanoate, ¹H NMR (400 MHz, DMSO-d₆) δ 7.53-7.30 (m, 8H), 7.28-7.20 (m, 1H), 6.76-6.65 (m, 2H), 4.12 (dd, J=8.8, 4.6, 1H), 3.54 (s, 3H), 3.19 (dd, J=13.6, 4.6 Hz, 1H), 3.07 (dd, J=13.2, 8.8 Hz, 1H), 1.37 (s, 9H). ¹⁹F NMR (376 MHz, DMSO-d₆) δ −117.77 (d, J=25.9 Hz), 141.24 (dd, J=25.6, 14.3 Hz). LCMS (ES, m/z): 578.05 [M+H]⁺; 98.5% purity (220 nm).

For tert-Butyl (R)-3-(4,5-difluoro-3-iodo-2-methoxyphenyl)-2-((diphenylmethylene)amino)propanoate, ¹H NMR (400 MHz, DMSO-d₆) δ 7.53-7.31 (m, 8H), 7.28-7.20 (m, 1H), 6.76-6.65 (m, 2H), 4.12 (dd, J=8.8, 4.6, 1H), 3.54 (s, 3H), 3.19 (dd, J=13.6, 4.6 Hz, 1H), 3.07 (dd, J=13.2, 8.8 Hz, 1H), 1.37 (s, 9H). ¹⁹F NMR (376 MHz, DMSO-d₆) δ −117.77 (d, J=25.9 Hz), −141.24 (dd, J=25.6, 14.3 Hz). LCMS (ES, m/z): 578.05 [M+H]⁺; 98.5% purity (220 nm).

### Example 12: Synthesis of tert-Butyl (2S)-2-[(tert-butoxycarbonyl)amino]-3-(4,5-difluoro-3-iodo-2-methoxyphenyl)propanoate and tert-Butyl (2R)-2-[(tert-butoxycarbonyl)amino]-3-(4,5-difluoro-3-iodo-2-methoxyphenyl)propanoate

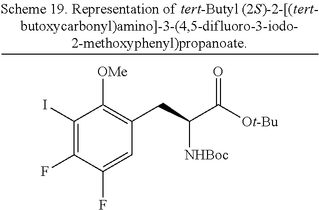 [SMILES: COc1c(C[C@H](NC(=O)OC(C)(C)C)C(=O)OC(C)(C)C)cc(F)c(F)c1I]

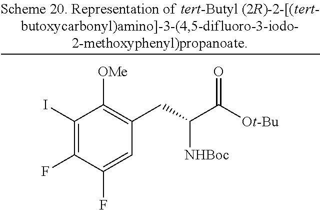 [SMILES: COc1c(C[C@@H](NC(=O)OC(C)(C)C)C(=O)OC(C)(C)C)cc(F)c(F)c1I]

The preparation of tert-Butyl (2S)-2-[(tert-butoxycarbonyl)amino]-3-(4,5-difluoro-3-iodo-2-methoxyphenyl)propanoate (Scheme 19) and tert-Butyl (2R)-2-[(tert-butoxycarbonyl)amino]-3-(4,5-difluoro-3-iodo-2-methoxyphenyl)propanoate (Scheme 20) is analogous to that shown in Example 28, below. The racemate was separated by Prep-HPLC [Column: CHIRALCEL OD-H 5 μm, 2×25 mm; Mobile Phase A: Hexanes; Mobile Phase B: IPA (with 0.5% 2M NH₃-MeOH); Flow rate: 20 mL/min; Wavelength: 254/220 nm], affording tert-butyl (2S)-2-[(tert-butoxycarbonyl)amino]-3-(4,5-difluoro-3-iodo-2-methoxyphenyl)propanoate (first eluting isomer, 65.1 mg, 17%) and tert-butyl (2R)-2-[(tert-butoxycarbonyl)amino]-3-(4,5-difluoro-3-iodo-2-methoxyphenyl)propanoate (second eluting isomer, 70.6 mg, 19%) as colorless oils.

For tert-Butyl (2S)-2-[(tert-butoxycarbonyl)amino]-3-(4,5-difluoro-3-iodo-2-methoxyphenyl)propanoate, ¹H NMR (400 MHz, DMSO) δ 7.49-7.35 (m, 1H), *7.26-7.20 (m, 0.85H), *6.90-6.81 (m, 0.15H), 4.15-4.00 (m, 1H), 3.75 (s, 3H), 3.16-3.02 (m, 1H), 2.88-2.76 (m, 1H), 1.58-1.33 (m, 18H). *Partial integration due to presence of rotamers. ¹⁹F NMR (376 MHz, DMSO-d₆) δ −116.89 (d, J=25.5 Hz), 141.11 (d, J=25.9 Hz). LCMS (ES, m/z): 514.15 [M+H]⁺; 99.9% (220 nm).

For tert-Butyl (2R)-2-[(tert-butoxycarbonyl)amino]-3-(4,5-difluoro-3-iodo-2-methoxyphenyl)propanoate, ¹H NMR (400 MHz, DMSO) δ 7.49-7.35 (m, 1H), *7.26-7.20 (m, 0.85H), *6.90-6.81 (m, 0.15H), 4.15-4.00 (m, 1H), 3.75 (s, 3H), 3.16-3.02 (m, 1H), 2.88-2.76 (m, 1H), 1.58-1.33 (m, 18H). *Partial integration due to presence of rotamers. ¹⁹F NMR (376 MHz, DMSO-d₆) δ −116.89 (d, J=25.5 Hz), 141.11 (d, J=25.9 Hz). LCMS (ES, m/z): 514.15 [M+H]⁺; 99.9% (220 nm).

### Example 13: Synthesis of 2-Amino-3-(4,5-difluoro-3-iodo-2-methoxyphenyl)propanoic acid

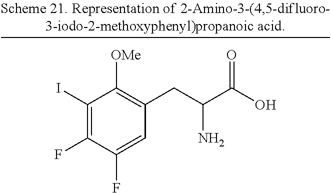 [SMILES: COc1c(CC(N)C(=O)O)cc(F)c(F)c1I]

The preparation of 2-Amino-3-(4,5-difluoro-3-iodo-2-methoxyphenyl)propanoic acid (Scheme 21) is shown schematically in Scheme 22. A mixture of tert-butyl 3-(4,5-difluoro-3-iodo-2-methoxyphenyl)-2-(diphenylmethyleneamino)propanoate (30 mg, 0.05 mmol) in HCl (6 M, 1 mL) was heated at 100° C. for 10 min, whereupon the mixture was cooled to room temperature and purified by reverse flash chromatography column (C18 silica gel, ACN-water, 0-20% with 0.1% FA), affording the title compound (8 mg, 42%) as a white solid.

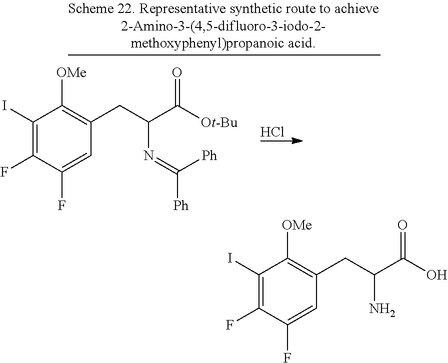 [SMILES: COc1c(CC(N=C(c2ccccc2)c2ccccc2)C(=O)OC(C)(C)C)cc(F)c(F)c1I ; Cl ; COc1c(CC(N)C(=O)O)cc(F)c(F)c1I]

For 2-Amino-3-(4,5-difluoro-3-iodo-2-methoxyphenyl)propanoic acid, ¹H NMR (400 MHz, DMSO-d₆) δ 7.43 (dd, J=11.3, 9.2 Hz, 1H), 3.74 (s, 3H), 3.51-3.42 (m, 1H), 3.26-3.23 (m, 1H), 2.79 (dd, J=14.6, 9.2 Hz, 1H). ¹H NMR (400 MHz, CF₃CO₂D) δ 7.35-7.25 (m, 1H), 4.83 (br s, 1H), 4.07 (s, 3H), 3.79-3.70 (m, 1H), 3.61-3.51 (m, 1H). ¹⁹F NMR (376 MHz, DMSO-d₆) δ −117.31 (d, J=25.9 Hz), −140.82 (d, J=25.9 Hz). LCMS (ES, m/z): 357.90 [M+H]⁺.

### Example 14: Synthesis of tert-Butyl 2-(azetidin-1-yl)-3-(4,5-difluoro-3-iodo-2-methoxyphenyl)propanoate

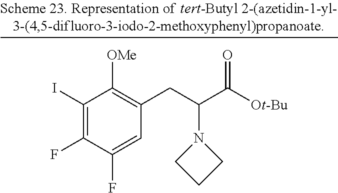 [SMILES: COc1c(CC(C(=O)OC(C)(C)C)N2CCC2)cc(F)c(F)c1I]

The preparation of tert-Butyl 2-(azetidin-1-yl)-3-(4,5-difluoro-3-iodo-2-methoxyphenyl)propanoate (Scheme 23) is shown schematically in Scheme 24. To a mixture of tert-butyl 3-(4,5-difluoro-3-iodo-2-methoxyphenyl)-2-[(diphenylmethylidene)amino]propanoate (2.00 g, 3.47 mmol) in EtOH/H₂O (20 mL, 4:1) was added hydroxylamine hydrochloride (2.39 g, 34.7 mmol). After 16 h, the mixture was partially concentrated under reduced pressure and purified by reverse flash chromatography (C18 silica gel, ACN-water, 5-70% with 0.1% FA) to afford the title compound (800 mg, 55%) as a white solid. LCMS (ES, m/z): 414.03 [M+H]⁺.

A solution of tert-butyl 2-amino-3-(4,5-difluoro-3-iodo-2-methoxyphenyl)propanoate (800 mg, 1.93 mmol), 1,3-dibromopropane (425 mg, 2.13 mmol) and DIEA (498 mg, 3.86 mmol) in ACN (10 mL) was heated at 70° C. After 16 h, the solution was cooled to rt and concentrated under reduced pressure. The crude residue was purified by reverse flash chromatography (C18 silica gel, ACN-water, 0-70% with 10 mM NH₄HCO₃), affording a sample of the title compound (200 mg, 90%). Half (100 mg) of the obtained sample was purified by Prep-HPLC [Column: XBridge Prep OBD C18 5 μm, 30×150 mm; Mobile Phase: ACN-water (with 10 mM NH₄HCO₃), 50-80%], affording the title compound (53 mg, 12%) as an off-white solid.

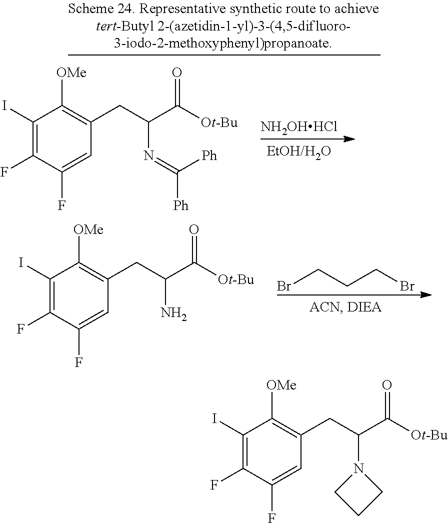

For tert-Butyl 2-(azetidin-1-yl)-3-(4,5-difluoro-3-iodo-2-methoxyphenyl)propanoate, ¹H NMR (400 MHz, DMSO) δ 7.42 (dd, J=11.4, 9.2 Hz, 1H), 3.72 (s, 3H), 3.27-3.13 (m, 5H), 2.83-2.61 (m, 2H), 1.96 (p, J=7.0 Hz, 2H), 1.27 (s, 9H). ¹⁹F NMR (376 MHz, DMSO-d₆) δ −116.99-(−)117.38 (m), −140.98-(−)141.25. LCMS (ES, m/z): 398.00 [M-tBu+H]⁺; 99.2% purity (220 nm).

### Example 15: Synthesis of 2-(Azetidin-1-yl)-3-(4,5-difluoro-3-iodo-2-methoxyphenyl)propanoic acid

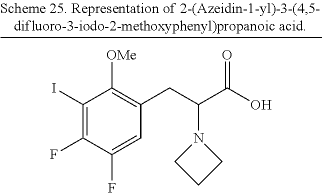 [SMILES: COc1c(CC(C(=O)O)N2CCC2)cc(F)c(F)c1I]

The preparation of 2-(Azetidin-1-yl)-3-(4,5-difluoro-3-iodo-2-methoxyphenyl)propanoic acid (Scheme 25) was undertaken according to analogous methods described herein.

For 2-(Azetidin-1-yl)-3-(4,5-difluoro-3-iodo-2-methoxyphenyl)propanoic acid, ¹H NMR (400 MHz, D₂O) δ 7.12 (t, J=9.7 Hz, 1H), 4.30-3.83 (m, 5H), 3.76 (s, 3H), 3.19-2.96 (m, 2H), 2.57-2.18 (m, 2H). ¹⁹F NMR (376 MHz, D₂O) δ −113.83 (d, J=24.4 Hz), −139.21 (d, J=24.1 Hz). LCMS (ES, m/z): 398.00 [M+H]⁺; 96.1% purity (220 nm).

### Example 16: Synthesis of tert-Butyl (S)-2-((tert-butoxycarbonyl)amino)-3-(2,4-difluoro-5-iodo-3-methoxyphenyl)propanoate and tert-Butyl (R)-2-((tert-butoxycarbonyl)amino)-3-(2,4-difluoro-5-iodo-3-methoxyphenyl)propanoate

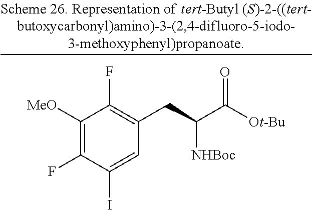 [SMILES: COc1c(F)c(I)cc(C[C@H](NC(=O)OC(C)(C)C)C(=O)OC(C)(C)C)c1F]

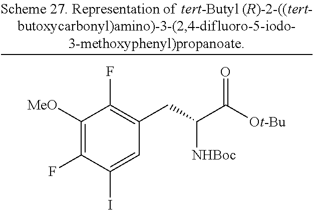 [SMILES: COc1c(F)c(I)cc(C[C@@H](NC(=O)OC(C)(C)C)C(=O)OC(C)(C)C)c1F]

The preparation of tert-Butyl (S)-2-((tert-butoxycarbonyl)amino)-3-(2,4-difluoro-5-iodo-3-methoxyphenyl)propanoate (Scheme 26) and tert-Butyl (R)-2-((tert-butoxycarbonyl)amino)-3-(2,4-difluoro-5-iodo-3-methoxyphenyl)propanoate (Scheme 27) is shown schematically in Scheme 28. To a stirred mixture of 2-amino-4-fluoro-3-methoxybenzoic acid (20.0 g, 108 mmol) in DMF (160 mL) at 0° C. was added NIS (36.5 g, 162 mmol). After 16 h at rt, the mixture was diluted with water (150 mL), the solids were collected by filtration, washed with water (2×20 mL) and dried under vacuum to afford the title compound (30.0 g) as a red solid. LCMS (ES, m/z): 311.95 [M+H]⁺.

To a mixture of 2-amino-4-fluoro-5-iodo-3-methoxybenzoic acid (30.0 g) in THF/MeOH (1:1, 600 mL) at 0° C. was slowly added (diazomethyl)trimethylsilane (2.0 M in THF, 216 mL, 432 mmol). After 16 h at rt, the mixture was concentrated under reduced pressure and the crude residue was purified by silica gel chromatography (EA-Petroleum ether, 20%), affording the title compound (26 g, 74%, two-steps) as a white solid. LCMS (ES, m/z): 325.95 [M+H]⁺.

To a stirred mixture of methyl 2-amino-4-fluoro-5-iodo-3-methoxybenzoate (15.0 g, 46.2 mmol) in HCl (6 M, 120 mL) at 0° C. was added a solution of NaNO₂ (4.78 g, 69.3 mmol) in H₂O (20 mL), dropwise. After 1 h at 0° C., hexafluorophosphoric acid (60% aq, 22.5 g, 92.4 mmol) was added, dropwise. The resulting mixture was held at 0° C. for 0.5 h then filtered. The filter cake was washed with cold water (10 mL) and dried under an IR lamp to afford a diazonium salt (22.5 g). A solution of the salt in HF-pyridine (70%, 920 mL) was circulated through a medium pressure mercury lamp flow reactor (1.3 mL/min) for 1 h at rt, whereupon the mixture was poured into ice-water (2 L) and extracted with EA (3×1.5 L). The combined organic layers were washed with brine (1 L), dried over anhydrous Na₂SO₄ and concentrated under reduced pressure. The crude residue was purified by silica gel chromatography (EA-Petroleum ether, 15%), to afford the title compound (9.60 g, 63%) as a white solid. GCMS (EI, m/z): 328.00 [M].

To a stirred mixture of methyl 2,4-difluoro-5-iodo-3-methoxybenzoate (9.60 g, 29.3 mmol) in THF (90 mL) was added a solution of LiOH·H₂O (12.6 g, 293 mmol) in H₂O (90 mL). After 16 h, the mixture was diluted with water (100 mL), cooled to 0° C., acidified with HCl (2 M) to pH-6 and extracted with EtOAc (3×100 mL). The combined organic layers were washed with brine (100 mL), dried over anhydrous Na₂SO₄ and concentrated under reduced pressure to afford the title compound (9.40 g) as a white solid. LCMS (ES, m/z): 312.90 [M−H]⁻.

To a mixture of 2,4-difluoro-5-iodo-3-methoxybenzoic acid (9.40 g) in THF (100 mL) at 0° C. was added borane-tetrahydrofuran complex (1 M in THF, 293 mL, 293 mmol), dropwise. To the mixture after 16 h at rt was added MeOH (300 mL) followed by heating at 70° C. for 1 h, whereupon the mixture was cooled to rt and concentrated under reduced pressure. The crude residue was purified by silica gel chromatography (EA-Petroleum ether, 35%), affording the title compound (5.50 g, 62%, two-steps) as a colorless oil. GCMS (ES, m/z): 299.90 [M].

To a mixture of (2,4-difluoro-5-iodo-3-methoxyphenyl)methanol (5.50 g, 18.3 mmol) in DCM (200 mL) at 0° C. was added PBr₃ (9.93 g, 36.6 mmol), dropwise. After 4 h at rt, the mixture was concentrated under reduced pressure and the crude residue was purified by silica gel chromatography (EA-Petroleum ether, 25%), affording the title compound (5.80 g, 87%) as a yellow solid. GCMS (ES, m/z): 361.90 [M].

A mixture of 1-(bromomethyl)-2,4-difluoro-5-iodo-3-methoxybenzene (5.80 g, 16.0 mmol), K₂CO₃ (5.54 g, 40.0 mmol), tert-butyl 2-[(diphenylmethylidene)amino]acetate (4.74 g, 16.0 mmol) and tetrabutylammonium bromide (1.03 g, 3.20 mmol) in MeCN (48 mL) was stirred for 16 h, whereupon it was poured into ice-water (100 mL) and extracted with EtOAc (3×100 mL). The combined organic layers were washed with brine (100 mL), dried over anhydrous Na₂SO₄ and concentrated under reduced pressure. The crude residue was purified by silica gel chromatography (EA-Petroleum ether, 20%) to afford the title compound (3.60 g, 39%) as a colorless oil. LCMS (ES, m/z): 578.15 [M+H]⁺.

To a mixture of tert-butyl 3-(2,4-difluoro-5-iodo-3-methoxyphenyl)-2-[(diphenylmethylidene)amino]propanoate (1.30 g, 2.25 mmol) in EtOH (30 mL)/H₂O (8 mL) was added hydroxylamine hydrochloride (2.04 g, 29.3 mmol), whereupon it was heated at 50° C. for 4 h, cooled to rt then concentrated under reduced pressure. The crude residue was purified by reverse flash chromatography (C18 silica gel, ACN-water, 0-80% with 10 mM NH₄HCO₃) to afford the title compound (750 mg, 80%) as a colorless oil. LCMS (ES, m/z): 414.00 [M+H]⁺.

To a mixture of tert-butyl 2-amino-3-(2,4-difluoro-5-iodo-3-methoxyphenyl)propanoate (750 mg, 1.82 mmol) in DCM (5 mL) at 0° C. was added TEA (552 mg, 5.46 mmol) then (Boc)₂O (598 mg, 2.73 mmol). After 2 h, the mixture was poured into water (30 mL) and extracted with DCM (3×30 mL). The combined organic layers were washed with brine (30 mL), dried over anhydrous Na₂SO₄ and concentrated under reduced pressure. The crude residue was purified by silica gel chromatography (EA-Petroleum ether, 15%) to afford tert-butyl-2-((tert-butoxycarbonyl)amino)-3-(2,4-difluoro-5-iodo-3-methoxyphenyl)propanoate (700 mg), which was further purified Prep-SFC [Column: Lux 5 um Cellulose-4 5 μm, 30×150 mm; Mobile Phase A: CO₂, Mobile Phase B: IPA/Hexanes (1:2) 12% (with 1%-2M-NH₃-MeOH); Flow rate: 85 mL/min; Column Temperature: 35° C.; Back Pressure: 100 bar; Wavelength: 220 nm] to afford tert-butyl (S)-2-((tert-butoxycarbonyl)amino)-3-(2,4-difluoro-5-iodo-3-methoxyphenyl)propanoate (first eluting isomer, 250 mg, 27%) and tert-butyl (R)-2-((tert-butoxycarbonyl)amino)-3-(2,4-difluoro-5-iodo-3-methoxyphenyl)propanoate (second eluting isomer, 234 mg, 25%) as white solids.

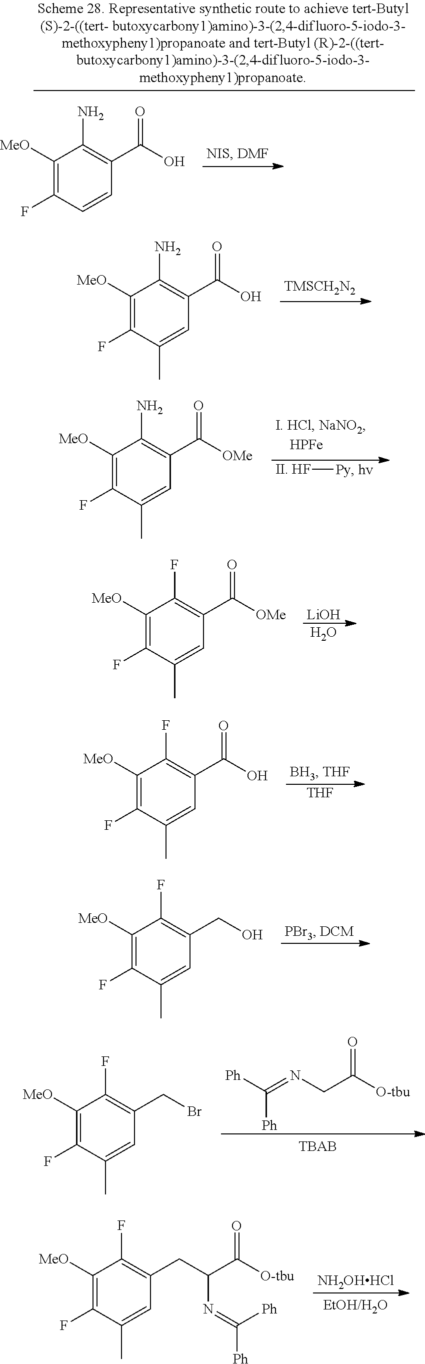

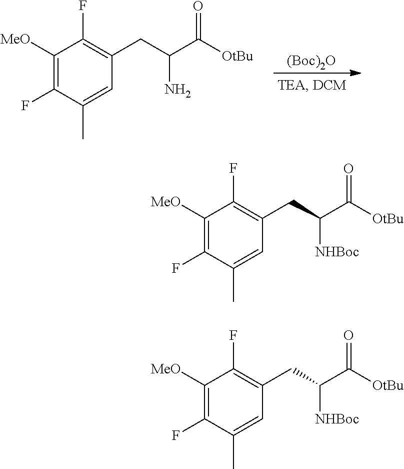

For tert-Butyl (S)-2-((tert-butoxycarbonyl)amino)-3-(2,4-difluoro-5-iodo-3-methoxyphenyl)propanoate, H NMR (400 MHz, DMSO-d₆) δ 7.58-7.43 (m, 1H), *7.21 (d, J=8.2 Hz, 0.8H), *6.91-6.86 (m, 0.1H), *4.10-4.01 (m, 0.85H), *4.00-3.93 (m, 0.25H), 3.90 (s, 3H), *3.00 (dd, J=13.8, 5.9 Hz, 0.82H), *2.97-2.90 (m, 0.15H), 2.84-2.76 (m, 1H), 1.45-1.27 (m, 18H). *Partial integration due to presence of rotamers. ¹⁹F NMR (376 MHz, DMSO-d₆) δ −110.63 (d, J=9.5 Hz), −131.77 (d, J=9.5 Hz). LCMS (ES, m/z): 357.95 [M-tBu-Boc+H]⁺. 98.1% purity (254 nm).

For tert-Butyl (R)-2-((tert-butoxycarbonyl)amino)-3-(2,4-difluoro-5-iodo-3-methoxyphenyl)propanoate, ¹H NMR (400 MHz, DMSO-d₆) δ 7.58-7.43 (m, 1H), *7.21 (d, J=8.2 Hz, 0.8H), *6.91-6.86 (m, 0.1H), *4.10-4.01 (m, 0.85H), *4.00-3.93 (m, 0.25H), 3.90 (s, 3H), *3.00 (dd, J=13.8, 5.9 Hz, 0.82H), *2.97-2.90 (m, 0.15H), 2.84-2.76 (m, 1H), 1.45-1.27 (m, 18H). *Partial integration due to presence of rotamers. ¹⁹F NMR (376 MHz, DMSO-d₆) δ −110.63 (d, J=9.5 Hz), −131.77 (d, J=9.5 Hz). LCMS (ES, m/z): 357.90 [M-tBu-Boc+H]⁺. 99.0% purity (254 nm).

### Example 17: Synthesis of 2-amino-3-(2,4-difluoro-5-iodo-3-methoxyphenyl)propanoic acid

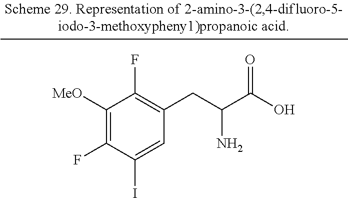 [SMILES: COc1c(F)c(I)cc(CC(N)C(=O)O)c1F]

The preparation of 2-amino-3-(2,4-difluoro-5-iodo-3-methoxyphenyl)propanoic acid (Scheme 29) is undertaken in an analogous manner to that of Example 13.

For 2-amino-3-(2,4-difluoro-5-iodo-3-methoxyphenyl)propanoic acid, ¹H NMR (400 MHz, CF₃CO₂D) δ 7.53 (d, J=6.8 Hz, 1H), 4.77-4.69 (m, 1H), 4.13 (s, 3H), 3.63 (dd, J=15.2, 5.0 Hz, 1H), 3.42 (dd, J=15.2, 7.7 Hz, 1H). ¹⁹F NMR (376 MHz, DMSO-d₆) δ −110.97 (d, J=8.7 Hz), −131.21 (d, J=8.7 Hz). LCMS (ES, m/z): 358.05 [M+H]⁺; 99.5% purity (254 nm).

### Example 18: Synthesis of Methyl 2-amino-3-(2,4-difluoro-5-iodo-3-methoxyphenyl)propanoate

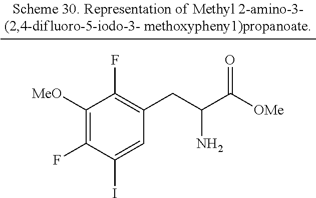 [SMILES: COC(=O)C(N)Cc1cc(I)c(F)c(OC)c1F]

The preparation of Methyl 2-amino-3-(2,4-difluoro-5-iodo-3-methoxyphenyl)propanoate (Scheme 30) is shown schematically in Scheme 31. To a solution of 2-amino-3-(2,4-difluoro-5-iodo-3-methoxyphenyl)propanoic acid (80 mg, 0.22 mmol) in THF/MeOH (1:1, 2 mL) was added dropwise (trimethylsilyl)diazomethane (2 M in THF, 0.44 mL, 0.88 mmol). After 40 min, the mixture was concentrated under reduced pressure and purified by reverse flash chromatography (C18 silica gel, ACN-water, 0-100% with 10 mM NH₄HCO₃) to afford the title compound (15.4 mg, 18%) as a white solid.

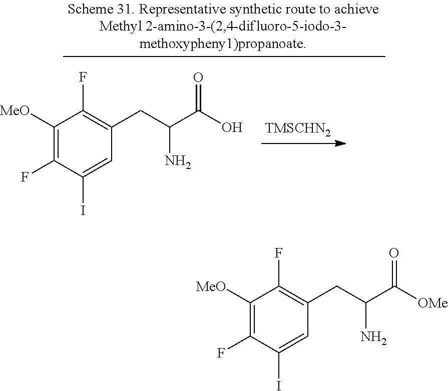 [SMILES: COc1c(F)c(I)cc(CC(N)C(=O)O)c1F ; COC(=O)C(N)Cc1cc(I)c(F)c(OC)c1F]

For Methyl 2-amino-3-(2,4-difluoro-5-iodo-3-methoxyphenyl)propanoate, ¹H NMR (400 MHz, DMSO-d₆) δ 7.49 (t, J=7.2 Hz, 1H), 3.90 (s, 3H), 3.59 (s, 3H), 3.52 (dd, J=8.1, 5.9 Hz, 1H), 2.87 (dd, J=13.7, 5.8 Hz, 1H), 2.71 (dd, J=13.8, 8.1 Hz, 1H). ¹⁹F NMR (376 MHz, DMSO-d₆) δ −110.83 (d, J=9.6 Hz), −131.97 (d, J=8.8 Hz). LCMS (ES, m/z): 371.90 [M+H]⁺; 95.6% purity (220 nm).

### Example 19: Synthesis of Isopropyl 2-amino-3-(2,4-difluoro-5-iodo-3-methoxyphenyl)propanoate

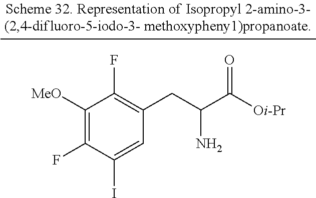

The preparation of Isopropyl 2-amino-3-(2,4-difluoro-5-iodo-3-methoxyphenyl)propanoate (Scheme 32) is shown schematically in Scheme 33. To a mixture of 2-amino-3-(2,4-difluoro-5-iodo-3-methoxyphenyl)propanoic acid (80 mg, 0.22 mmol) in IPA (1 mL) at 0° C. was added SOCl₂ (267 mg, 2.20 mmol), dropwise. The mixture was then heated at 60° C. for 2 h, cooled to room temperature and concentrated under reduced pressure. The crude residue was purified by Prep-HPLC [Column: Xselect CSH Prep C₁₈ OBD 5 μm, 30×150 mm; Mobile Phase: ACN-water, 6-36% (with 0.1% FA); Flow rate: 60 mL/min; Wavelength: 254/220 nm], to afford the title compound (20 mg, 22%) as a white solid.

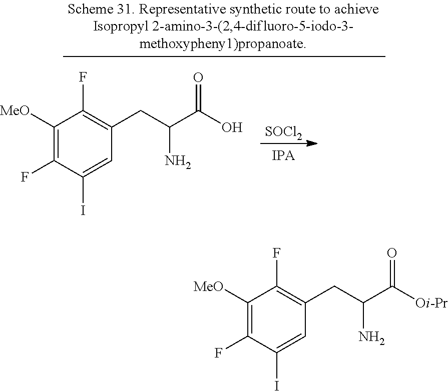

For Isopropyl 2-amino-3-(2,4-difluoro-5-iodo-3-methoxyphenyl)propanoate, ¹H NMR (400 MHz, MeOD) δ 7.40 (t, J=7.0 Hz, 1H), 4.94 (sep, J=6.3 Hz, 1H), 3.97 (s, 3H), 3.65 (t, J=7.1 Hz, 1H), 2.94 (d, J=7.1 Hz, 2H), 1.23 (d, J=6.3 Hz, 3H), 1.15 (d, J=6.3 Hz, 3H). ¹⁹F NMR (376 MHz, MeOD) δ −110.81 (d, J=9.8 Hz), −132.80 (d, J=10.0 Hz). LCMS (ES, m/z): 400.20 [M+H]⁺; 98.5% purity (220 nm).

### Example 20: Synthesis of Isopropyl 2-((tert-butoxycarbonyl)amino)-3-(2,4-difluoro-5-iodo-3-methoxyphenyl)propanoate

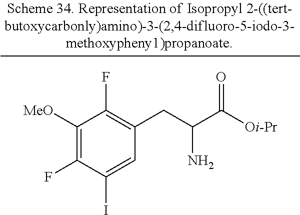

The preparation of the title compound (30 mg) Isopropyl 2-((tert-butoxycarbonyl)amino)-3-(2,4-difluoro-5-iodo-3-methoxyphenyl)propanoate (Scheme 34) is from Example 19 and is undertaken according to analogous methods described herein.

For Isopropyl 2-((tert-butoxycarbonyl)amino)-3-(2,4-difluoro-5-iodo-3-methoxyphenyl)propanoate, ¹H NMR (400 MHz, DMSO-d₆) δ *7.57-7.7.51 (m, 0.15H), *7.47 (t, J=7.1 Hz, 0.8H), *7.28 (d, J=8.1 Hz, 0.8H), *7.00-6.93 (m, 0.13H), 4.87 (sep, J=6.2 Hz, 1H), *4.11 (td, J=9.1, 5.9 Hz, 0.84H), *4.07-3.98 (m, 0.13H), 3.91 (s, 3H), *3.03 (dd, J=13.8, 5.7 Hz, 0.8H), *2.98-2.92 (m, 0.13H), 2.82 (dd, J=13.7, 9.9 Hz, 1H), 1.39-1.26 (m, 9H), 1.17 (d, J=6.2 Hz, 3H), 1.12 (d, J=6.2 Hz, 3H). *Partial integration due to presence of rotamers. ¹⁹F NMR (376 MHz, DMSO-d₆) δ −110.30 (d, J=8.3 Hz, minor rotamer), −110.54 (d, J=9.6 Hz, major rotamer), −131.79 (d, J=9.6 Hz, major rotamer), 131.93 (d, J=8.3 Hz, minor rotamer). LCMS (ES, m/z): 399.95 [M-Boc+H]⁺; 99.2% purity (254 nm).

### Example 21: Synthesis of 2-Amino-3-(2,4-difluoro-5-iodo-3-methoxyphenyl)-2-methylpropanoic acid

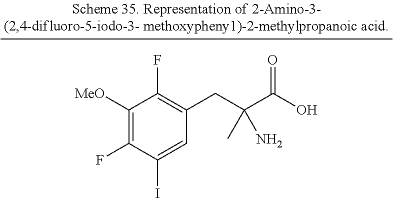 [SMILES: COc1c(F)c(I)cc(CC(C)(N)C(=O)O)c1F]

The preparation of the title compound (22 mg) 2-Amino-3-(2,4-difluoro-5-iodo-3-methoxyphenyl)-2-methylpropanoic acid (Scheme 35) is from the intermediate of Example 16 and is undertaken according to analogous methods provided herein.

For 2-Amino-3-(2,4-difluoro-5-iodo-3-methoxyphenyl)-2-methylpropanoic acid, ¹H NMR (400 MHz, DMSO-d₆) δ 8.32-7.37 (m, 2H), 7.53 (t, J=6.9 Hz, 1H), 3.91 (s, 3H), 2.97 (q, J=14.1 Hz, 2H), 1.30 (s, 3H). ¹⁹F NMR (376 MHz, DMSO-d₆) δ −110.33, −129.01. LCMS (ES, m/z): 371.90 [M+H]⁺; 98.9% purity (220 nm).

### Example 22: Synthesis of tert-Butyl 2-((tert-butoxycarbonyl)amino)-3-(2,4-difluoro-5-iodo-3-methoxyphenyl)-2-methylpropanoate

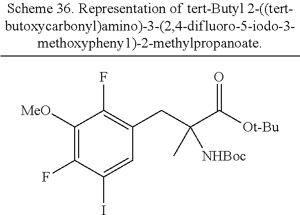 [SMILES: COc1c(F)c(I)cc(CC(C)(NC(=O)OC(C)(C)C)C(=O)OC(C)(C)C)c1F]

The preparation of the title compound (85 mg) tert-Butyl 2-((tert-butoxycarbonyl)amino)-3-(2,4-difluoro-5-iodo-3-methoxyphenyl)-2-methylpropanoate (Scheme 36) is from the intermediate of Example 16 and is undertaken in an analogous manner to methods provided herein.

For tert-Butyl 2-((tert-butoxycarbonyl)amino)-3-(2,4-difluoro-5-iodo-3-methoxyphenyl)-2-methylpropanoate, ¹H NMR (400 MHz, DMSO-d₆) δ 7.22 (t, J=7.0 Hz, 1H), 7.01 (s, 1H), 3.90 (s, 3H), 3.26 (d, J=13.7 Hz, 1H), 2.91 (d, J=13.7 Hz, 1H), 1.44 (s, 9H), 1.40 (s, 9H), 1.10 (s, 3H). ¹⁹F NMR (376 MHz, DMSO-d₆) δ −110.65 (d, J=9.2 Hz), −129.48 (d, J=10.0 Hz). LCMS (ES, m/z): 550.10 [M+Na]⁺; 98.9% purity (220 nm).

### Example 23: Synthesis of tert-Butyl (3-(2,4-difluoro-5-iodo-3-methoxyphenyl)-2-methyl-1-(methylamino)-1-oxopropan-2-yl)carbamate

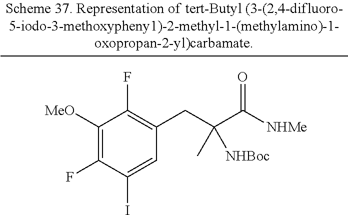 [SMILES: CNC(=O)C(C)(Cc1cc(I)c(F)c(OC)c1F)NC(=O)OC(C)(C)C]

The preparation of the title compound (50 mg) tert-Butyl (3-(2,4-difluoro-5-iodo-3-methoxyphenyl)-2-methyl-1-(methylamino)-1-oxopropan-2-yl)carbamate (Scheme 37) uses an intermediate of Example 21 and is undertaken in an analogous manner to the methods in Example 31, below.

For tert-Butyl (3-(2,4-difluoro-5-iodo-3-methoxyphenyl)-2-methyl-1-(methylamino)-1-oxopropan-2-yl)carbamate, ¹H NMR (400 MHz, DMSO-d₆) δ 7.76-7.65 (m, 1H), 7.21-7.10 (m, 1H), 6.68 (s, 1H), 3.89 (s, 3H), 3.19 (d, J=13.2 Hz, 1H), 3.11 (d, J=13.2 Hz, 1H), 2.57 (d, J=4.4 Hz, 3H), 1.45 (s, 9H), 1.15 (s, 3H). ¹⁹F NMR (376 MHz, DMSO-d₆) δ −110.80 (d, J=8.6 Hz), −129.54 (d, J=8.9 Hz). LCMS (ES, m/z): 485.35 [M+H]⁺; 98.8% purity (220 nm).

### Example 24: Synthesis of 2-Amino-3-(2,4-difluoro-5-iodo-3-methoxyphenyl)-N,2-dimethylpropanamide hydrochloride

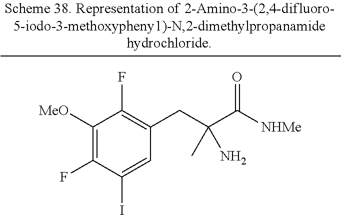 [SMILES: CNC(=O)C(C)(N)Cc1cc(I)c(F)c(OC)c1F]

The preparation of the title compound (20 mg) 2-Amino-3-(2,4-difluoro-5-iodo-3-methoxyphenyl)-N,2-dimethylpropanamide hydrochloride (Scheme 38) is from Example 23 and is undertaken in an analogous manner to the methods described herein.

For 2-Amino-3-(2,4-difluoro-5-iodo-3-methoxyphenyl)-N,2-dimethylpropanamide hydrochloride, ¹H NMR (400 MHz, DMSO-d₆/D₂O) δ 7.39 (t, J=7.2 Hz, 1H), 3.88 (s, 3H), 2.82 (d, J=13.2 Hz, 1H), 2.72 (d, J=13.0 Hz, 1H), 2.55 (s, 3H), 1.12 (s, 3H). ¹⁹F NMR (376 MHz, DMSO-d₆/D₂O) δ −111.19 (d, J=9.4 Hz), −130.04 (d, J=8.9 Hz). LCMS (ES, m/z): 384.95 [M+H]⁺; 99.5% purity (220 nm).

### Example 25: Synthesis of tert-Butyl (1-(cyclopropylamino)-3-(2,4-difluoro-5-iodo-3-methoxyphenyl)-2-methyl-1-oxopropan-2-yl)carbamate

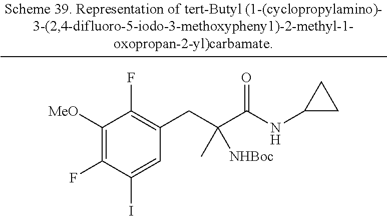 [SMILES: COc1c(F)c(I)cc(CC(C)(NC(=O)OC(C)(C)C)C(=O)NC2CC2)c1F]

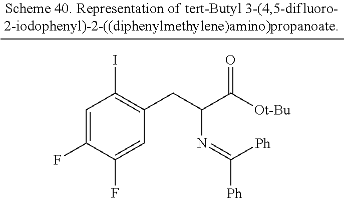 [SMILES: CC(C)(C)OC(=O)C(Cc1cc(F)c(F)cc1I)N=C(c1ccccc1)c1ccccc1]

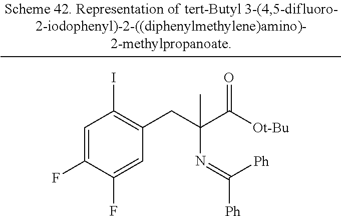 [SMILES: CC(C)(C)OC(=O)C(C)(Cc1cc(F)c(F)cc1I)N=C(c1ccccc1)c1ccccc1]

The preparation of the title compound (50 mg) tert-Butyl (1-(cyclopropylamino)-3-(2,4-difluoro-5-iodo-3-methoxyphenyl)-2-methyl-1-oxopropan-2-yl)carbamate (Scheme 39) is undertaken using a previously described intermediate of Example 21 an in an analogous manner to the methods of Example 31.

For tert-Butyl (1-(cyclopropylamino)-3-(2,4-difluoro-5-iodo-3-methoxyphenyl)-2-methyl-1-oxopropan-2-yl)carbamate, ¹H NMR (400 MHz, DMSO-d₆) δ 7.74 (d, J=4.0 Hz, 1H), 7.20-7.08 (m, 1H), 6.57 (s, 1H), 3.89 (s, 3H), 3.17 (d, J=13.6 Hz, 1H), 3.06 (d, J=13.8 Hz, 1H), 2.63-2.53 (m, 1H), 1.43 (s, 9H), 1.14 (s, 3H), 0.64-0.53 (m, 2H), 0.48-0.36 (m, 2H). ¹⁹F NMR (376 MHz, DMSO-d₆) δ −110.89 (d, J=8.9 Hz), −129.31 (d, J=9.1 Hz). LCMS (ES, m/z): 511.35 [M+H]⁺; 98.5% purity (220 nm).

The Intermediate 01, tert-Butyl 3-(4,5-difluoro-2-iodophenyl)-2-((diphenylmethylene)amino)propanoate (Scheme 40), is produced as shown schematically in Scheme 41. To a solution of 4,5-difluoro-2-iodobenzoic acid (5.00 g, 17.6 mmol) in THF (50 mL) was added borane-tetrahydrofuran complex (1 M in THF, 70 mL, 70.0 mmol), dropwise. After 16 h, MeOH (100 mL) was added and the mixture was heated at 80° C. for 1 h, whereupon it was concentrated under reduced pressure. The crude residue was purified by silica gel chromatography (EA-Petroleum ether, 25%), affording the title compound (3.00 g, 63%) as a white solid. GCMS (ES, m/z): 269.90 [M].

To a mixture of (4,5-difluoro-2-iodophenyl)methanol (3.00 g, 11.1 mmol) in DCM (90 mL) at 0° C. was added PBr₃ (6.00 g, 22.2 mmol), dropwise. After 4 h, the mixture was concentrated under reduced pressure and the crude residue was purified by silica gel chromatography (EA-Petroleum ether, 10%), affording the title compound (2.50 g, 67%) as a clear oil. GCMS (ES, m/z): 331.80, 333.80 [M].

To a mixture of 1-(bromomethyl)-4,5-difluoro-2-iodobenzene (2.50 g, 7.51 mmol) in MeCN (20 mL) was added K₂CO₃ (2.08 g, 15.0 mmol), tert-butyl 2-[(diphenylmethylidene)amino]acetate (2.22 g, 7.51 mmol) and tetrabutylammonium bromide (240 mg, 0.75 mmol). After 16 h, the mixture was poured into ice-water (50 mL) and extracted with EtOAc (3×50 mL). The combined organic layers were washed with brine (50 mL), dried over anhydrous Na₂SO₄ and concentrated under reduced pressure. The crude product was purified by reverse flash chromatography (C18 silica gel, ACN-water, 0-100% with 10 mM NH₄HCO₃) to afford the title compound (1.3 g, 31%) as a yellow oil.

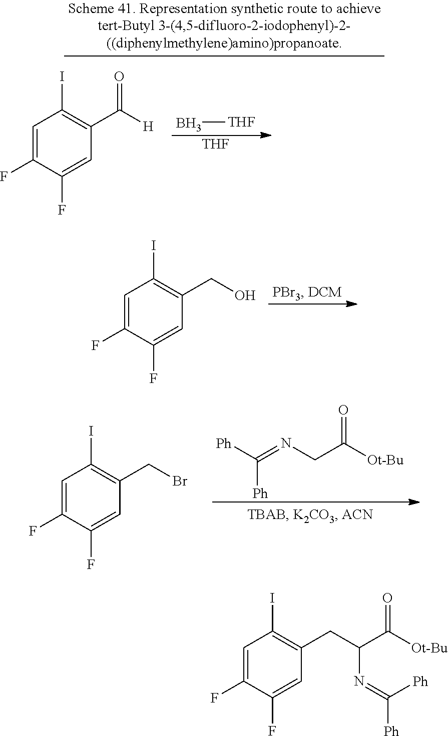

For tert-Butyl 3-(4,5-difluoro-2-iodophenyl)-2-((diphenylmethylene)amino)propanoate, ¹H NMR (400 MHz, DMSO-d₆) δ 7.83 (dd, J=9.9, 8.2 Hz, 1H), 7.56-7.48 (m, 2H), 7.47-7.34 (m, 6H), 7.27 (dd, J=11.6, 8.8 Hz, 1H), 6.63 (br d, J=5.6 Hz, 2H), 4.22 (t, J=6.6 Hz, 1H), 3.22 (d, J=6.8 Hz, 2H), 1.40 (s, 9H). LCMS (ES, m/z): 548.05 [M+H]⁺.

The Intermediate 02, tert-Butyl 3-(4,5-difluoro-2-iodophenyl)-2-((diphenylmethylene)amino)-2-methylpropanoate (Scheme 42), is produced as shown schematically in Scheme 43. To a solution of tert-butyl 3-(4,5-difluoro-2-iodophenyl)-2-[(diphenylmethylidene)amino]propanoate (1.30 g, 2.37 mmol) in THF (10 mL) at 0° C. was added LiHMDS (1.0 M in THF, 4.80 ml, 4.75 mmol), dropwise. After 0.5 h, CH₃I (1.00 g, 7.12 mmol) was added and the mixture was stirred for 2 h at 0° C., whereupon it was poured into water (50 mL) and extracted with EtOAc (3×50 mL). The combined organic layers were washed with brine (30 mL), dried over anhydrous Na₂SO₄ and concentrated under reduced pressure. The crude residue was purified by silica gel chromatography (EA-Petroleum ether, 20%), affording the title compound (1.0 g, 75%) as yellow oil. LCMS (ES, m/z): 562.10 [M+H]⁺.

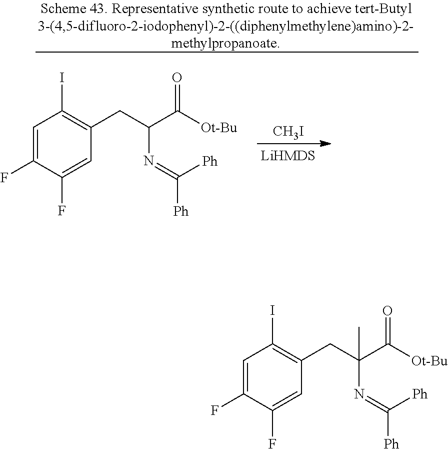 [SMILES: CC(C)(C)OC(=O)C(Cc1cc(F)c(F)cc1I)N=C(c1ccccc1)c1ccccc1 ; "CH3I" ; CC(C)(C)OC(=O)C(C)(Cc1cc(F)c(F)cc1I)N=C(c1ccccc1)c1ccccc1]

### Example 26: Synthesis of 2-amino-3-(4,5-difluoro-2-iodophenyl)-2-methylpropanoic acid

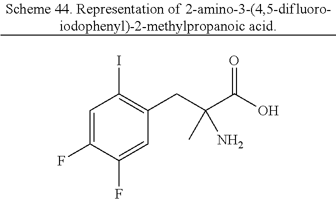 [SMILES: CC(N)(Cc1cc(F)c(F)cc1I)C(=O)O]

The preparation of the title compound (9.4 mg, 77%) 2-amino-3-(4,5-difluoro-2-iodophenyl)-2-methylpropanoic acid (Scheme 44) is undertaken in an analogous manner to methods of Example 13.

For 2-amino-3-(4,5-difluoro-2-iodophenyl)-2-methylpropanoic acid, ¹H NMR (400 MHz, DMSO-d₆) δ 7.95 (dd, J=10.0, 8.5 Hz, 1H), 7.49 (dd, J=12.5, 9.0 Hz, 1H), 3.18 (d, J=14.1 Hz, 1H), 3.05 (d, J=14.2 Hz, 1H), 1.24 (s, 3H). ¹⁹F NMR (376 MHz, DMSO-d₆) δ −138.79, (d, J=22.3 Hz), −139.72 (d, J=21.9 Hz). LCMS (ES, m/z): 341.90 [M+H]⁺; 99.1% purity (220 nm).

### Example 27: Synthesis of 2-amino-3-(4,5-difluoro-2-iodophenyl)propanoic acid

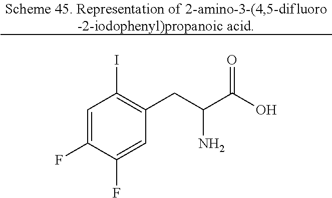 [SMILES: NC(Cc1cc(F)c(F)cc1I)C(=O)O]

The preparation of the title compound (30 mg, 50%) 2-amino-3-(4,5-difluoro-2-iodophenyl)propanoic acid (Scheme 45) is undertaken in an analogous manner to methods of Example 13.

For 2-amino-3-(4,5-difluoro-2-iodophenyl)propanoic acid, ¹H NMR (400 MHz, DMSO-d₆) δ 8.01-7.92 (m, 1H), 7.70-7.20 (m, 2H), 7.45 (dd, J=12.2, 8.2 Hz, 1H), 3.55-3.43 (m, 1H), 3.31-3.23 (m, 1H), 2.82 (dd, J=14.4, 10.0 Hz, 1H). ¹H NMR (400 MHz, CF₃CO₂D) δ 7.86 (dd, J=9.3, 7.7 Hz, 1H), 7.36 (dd, J=10.4, 7.6 Hz, 1H), 4.95 (dd, J=9.6, 5.8 Hz, 1H), 3.88 (dd, J=14.9, 5.7 Hz, 1H), 3.57 (dd, J=14.9, 9.6 Hz, 1H). ¹⁹F NMR (376 MHz, DMSO-d₆) δ −139.13, (d, J=21.8 Hz), −140.12 (d, J=21.8 Hz). LCMS (ES, m/z): 327.95 [M+H]⁺; 99.4% purity (254 nm)

### Example 28: Synthesis of tert-Butyl (2S)-2-[(tert-butoxycarbonyl)amino]-3-(4,5-difluoro-2-iodophenyl)propanoate and tert-Butyl (2R)-2-[(tert-butoxycarbonyl)amino]-3-(4,5-difluoro-2-iodophenyl)propanoate

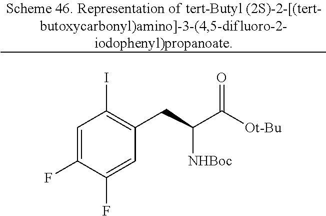 [SMILES: CC(C)(C)OC(=O)N[C@@H](Cc1cc(F)c(F)cc1I)C(=O)OC(C)(C)C]

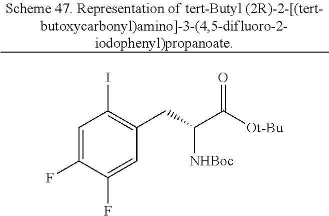 [SMILES: CC(C)(C)OC(=O)N[C@H](Cc1cc(F)c(F)cc1I)C(=O)OC(C)(C)C]

The preparation of tert-Butyl (2S)-2-[(tert-butoxycarbonyl)amino]-3-(4,5-difluoro-2-iodophenyl)propanoate (Scheme 46) and tert-Butyl (2R)-2-[(tert-butoxycarbonyl)amino]-3-(4,5-difluoro-2-iodophenyl)propanoate (Scheme 47) is shown schematically in Scheme 48. A solution of tert-butyl 3-(4,5-difluoro-2-iodophenyl)-2-[(diphenylmethylidene)amino]propanoate (650 mg, 1.19 mmol) and hydroxylamine hydrochloride (825 mg, 11.9 mmol) in EtOH/H₂O (4:1, 5 mL) was heated at 50° C. for 2 h, whereupon the mixture was concentrated under reduced pressure. The crude residue was purified by reverse flash chromatography (C18 silica gel, ACN-water, 10-90% with 0.1% FA), affording the title compound (300 mg, 65%) as a yellow oil. LCMS (ES, m/z): 384.10 [M+H]⁺.

To a solution of tert-butyl 2-amino-3-(4,5-difluoro-2-iodophenyl)propanoate (300 mg, 0.78 mmol) and TEA (236 mg, 2.34 mmol) in DCM (5 mL) was added di-tert-butyl dicarbonate (340 mg, 1.56 mmol). After 1 h, the mixture was concentrated under reduced pressure and the crude residue was purified by silica gel chromatography (THF-Petroleum ether, 15%) to afford tert-butyl 2-[(tert-butoxycarbonyl)amino]-3-(4,5-difluoro-2-iodophenyl)propanoate (300 mg, 79%) as a white solid.

LCMS (ES, m/z): 506.05 [M+Na]⁺.

The racemic sample was further separated by Prep-HPLC [Column: CHIRALPAK AD-H 5 μm, 2×25 cm; Mobile Phase A: Hexanes; Mobile Phase B: 10% IPA (with 0.5% 2M NH₃-MeOH); Flow rate: 20 mL/min; Wavelength: 220/254 nm] to afford tert-butyl (2S)-2-[(tert-butoxycarbonyl)amino]-3-(4,5-difluoro-2-iodophenyl)propanoate (first eluting isomer, 86.3 mg, 28%) and tert-butyl (2R)-2-[(tert-butoxycarbonyl)amino]-3-(4,5-difluoro-2-iodophenyl)propanoate (second eluting isomer, 86.2 mg, 28%) as white solids.

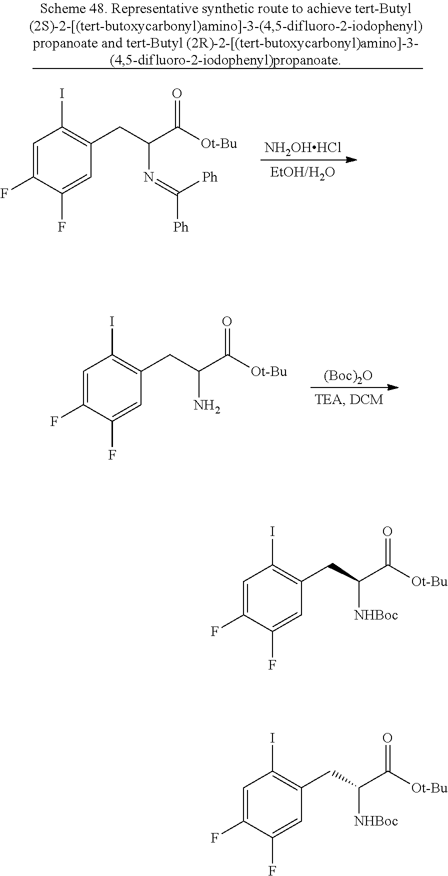

For tert-Butyl (2S)-2-[(tert-butoxycarbonyl)amino]-3-(4,5-difluoro-2-iodophenyl)propanoate, ¹H NMR (400 MHz, DMSO-d₆) δ 8.03-7.92 (m, 1H), 7.50-7.37 (m, 1H), *7.27 (d, J=8.5 Hz, 0.81H), *6.90 (d, J=9.2 Hz, 0.15H), 4.16-4.07 (m, 1H), 3.12-3.01 (m, 1H), 2.89 (dd, J=13.8, 10.5 Hz, 1H), 1.39 (s, 9H), 1.37-1.23 (m, 9H). *Partial integration due to presence of rotamers. ¹H NMR (400 MHz, 56° C., DMSO-d₆) δ 7.87 (dd, J=10.0, 8.8 Hz, 1H), 7.37 (dd, J=11.6, 8.8 Hz, 1H), 6.90 (br s, 1H), 4.22-4.12 (m, 1H), 3.09 (dd, J=14.0, 5.2 Hz, 1H), 2.92 (dd, J=13.6, 10.9 Hz, 1H), 1.40 (s, 9H), 1.32 (s, 9H). LCMS (ES, m/z): 506.10 [M+Na]⁺; 99.5% purity (220 nm)

For tert-Butyl (2R)-2-[(tert-butoxycarbonyl)amino]-3-(4,5-difluoro-2-iodophenyl)propanoate, ¹H NMR (400 MHz, DMSO-d₆) δ 8.03-7.92 (m, 1H), 7.50-7.37 (m, 1H), *7.27 (d, J=8.5 Hz, 0.81H), *6.90 (d, J=9.2 Hz, 0.15H), 4.16-4.07 (m, 1H), 3.12-3.01 (m, 1H), 2.89 (dd, J=13.8, 10.5 Hz, 1H), 1.39 (s, 9H), 1.37-1.23 (m, 9H). *Partial integration due to presence of rotamers. ¹H NMR (400 MHz, 56° C., DMSO-d₆) δ 7.87 (dd, J=10.0, 8.8 Hz, 1H), 7.37 (dd, J=11.6, 8.8 Hz, 1H), 6.90 (br s, 1H), 4.22-4.12 (m, 1H), 3.09 (dd, J=14.0, 5.2 Hz, 1H), 2.92 (dd, J=13.6, 10.9 Hz, 1H), 1.40 (s, 9H), 1.32 (s, 9H). LCMS (ES, m/z): 506.10 [M+Na]⁺; 99.7% purity (220 nm)

### Example 29: Synthesis of tert-Butyl 2-((tert-butoxycarbonyl)amino)-3-(4,5-difluoro-2-iodophenyl)-2-methylpropanoate

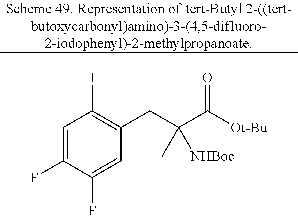 [SMILES: CC(C)(C)OC(=O)NC(C)(Cc1cc(F)c(F)cc1I)C(=O)OC(C)(C)C]

The preparation of the title compound (41.4 mg, 6%) tert-Butyl 2-((tert-butoxycarbonyl)amino)-3-(4,5-difluoro-2-iodophenyl)-2-methylpropanoate (Scheme 49) is undertaken from an intermediate (Scheme 42) in an analogous manner to the method of Example 28.

For tert-Butyl 2-((tert-butoxycarbonyl)amino)-3-(4,5-difluoro-2-iodophenyl)-2-methylpropanoate, ¹H NMR (400 MHz, DMSO-d₆) δ 7.98 (t, J=9.2 Hz, 1H), 7.19 (s, 1H), 7.03 (t, J=10.3 Hz, 1H), 3.39 (d, J=14.0 Hz, 1H), 3.20 (d, J=14.0 Hz, 1H), 1.44 (s, 9H), 1.41 (s, 9H), 1.13 (s, 3H). ¹⁹F NMR (376 MHz, DMSO-d₆) δ −139.39, −139.45. LCMS (ES, m/z): 341.95 [M-tBu-Boc+H]⁺; 97.6% purity (220 nm).

### Example 30: Synthesis of tert-Butyl 2-[(tert-butoxycarbonyl)(methyl)amino]-3-(4,5-difluoro-2-iodophenyl)-2-methylpropanoate

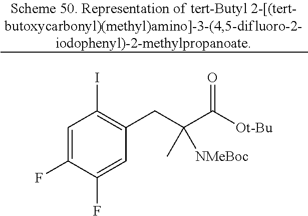

The preparation of tert-Butyl 2-[(tert-butoxycarbonyl)(methyl)amino]-3-(4,5-difluoro-2-iodophenyl)-2-methylpropanoate (Scheme 50) is shown schematically in Scheme 51. To a mixture of tert-butyl 2-[(tert-butoxycarbonyl)amino]-3-(4,5-difluoro-2-iodophenyl)-2-methylpropanoate (100 mg, 0.20 mmol) in DMF (3 mL) at 0° C. was added NaH (60% in mineral oil, 16 mg, 0.40 mmol), portion-wise. After 30 min at 0° C., Mel (85 mg, 0.60 mmol) was added and it was held at 0° C. for an additional 2 h, whereupon the mixture was poured into water (20 mL) then extracted with EtOAc (3×20 mL). The combined organic layers were washed with brine (20 mL), dried over anhydrous Na₂SO₄ and concentrated under reduced pressure. The crude residue was purified by reverse flash chromatography (C18 silica gel, ACN-water, 30-100% with 0.1% FA), affording the title compound (26.7 mg, 25%) as yellow oil.

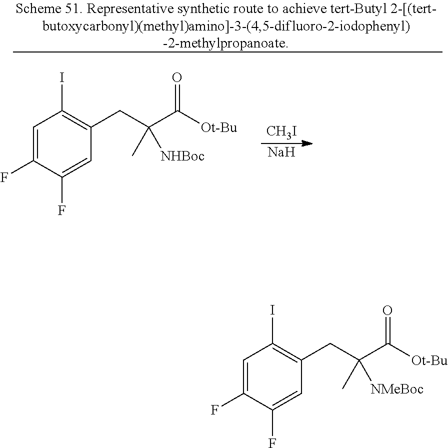

For tert-Butyl 2-[(tert-butoxycarbonyl)(methyl)amino]-3-(4,5-difluoro-2-iodophenyl)-2-methylpropanoate, ¹H NMR (400 MHz, CDCl₃) δ 7.63 (t, J=8.8 Hz, 1H), 6.94 (dd, J=11.5, 8.6 Hz, 1H), 3.76-3.62 (m, 1H), 3.44-3.26 (m, 1H), 2.75-2.49 (m, 3H), 1.52 (s, 9H), 1.47 (s, 9H), 1.36 (s, 3H). ¹⁹F NMR (377 MHz, CDCl₃) δ −138.13, −138.35. LCMS (ES, m/z): 355.90 [M-tBu-Boc+H]⁺; 95.1% purity (220 nm).

### Example 31: Synthesis of tert-Butyl (3-(4,5-difluoro-2-iodophenyl)-2-methyl-1-(methylamino)-1-oxopropan-2-yl)carbamate

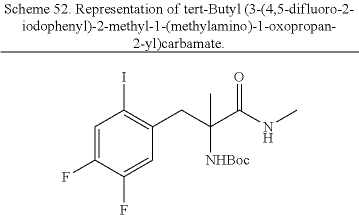 [SMILES: CNC(=O)C(C)(Cc1cc(F)c(F)cc1I)NC(=O)OC(C)(C)C]

The preparation of tert-Butyl (3-(4,5-difluoro-2-iodophenyl)-2-methyl-1-(methylamino)-1-oxopropan-2-yl)carbamate (Scheme 52) is shown schematically in Scheme 53. A mixture of 2-amino-3-(4,5-difluoro-2-iodophenyl)-2-methylpropanoic acid (150 mg, 0.44 mmol), (Boc)₂O (192 mg, 0.88 mmol) and NaHCO₃ (110 mg, 1.32 mmol) in DCM (3 mL) was stirred at 30° C. for 1 h, then concentrated under reduced pressure and purified by silica gel chromatography (EA-petroleum ether, 25%), to afford the title compound (150 mg, 77%) as white solid. LCMS (ES, m/z): 440.10 [M−H]⁻.

To a solution of 2-[(tert-butoxycarbonyl)amino]-3-(4,5-difluoro-2-iodophenyl)-2-methylpropanoic acid (60 mg, 0.14 mmol), methylamine hydrochloride (18 mg, 0.27 mmol) and HATU (80 mg, 0.20 mmol) in DMF (2 mL) was added DIEA (54 mg, 0.41 mmol). After 1 h, the mixture was directly purified by reversed-phase flash chromatography (C18 silica gel, ACN-water, 10-50% with 0.1% FA) to afford the title compound (21.2 mg, 34%) as a white solid.

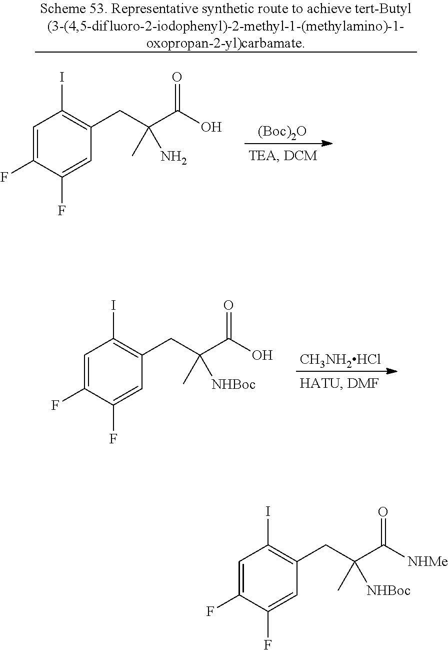

For tert-Butyl (3-(4,5-difluoro-2-iodophenyl)-2-methyl-1-(methylamino)-1-oxopropan-2-yl)carbamate, ¹H NMR (400 MHz, DMSO-d₆) δ 8.01-7.93 (m, 1H), 7.72-7.62 (m, 1H), 7.04-6.87 (m, 2H), 3.32-3.25 (m, 2H), 2.58 (d, J=4.5 Hz, 3H), 1.42 (s, 9H), 1.16 (s, 3H). ¹⁹F NMR (376 MHz, DMSO-d₆) δ −139.66-(−)139.85 (m). LCMS (ES, m/z): 476.95 [M+Na]⁺; 99.5% purity (220 nm).

### Example 32: Synthesis of Methyl 2-((tert-butoxycarbonyl)amino)-3-(4,5-difluoro-2-iodophenyl)-2-methylpropanoate

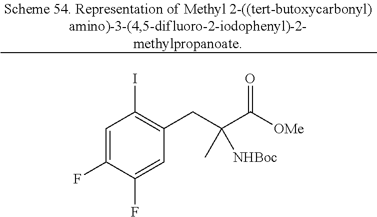 [SMILES: COC(=O)C(C)(Cc1cc(F)c(F)cc1I)NC(=O)OC(C)(C)C]

The preparation of Methyl 2-((tert-butoxycarbonyl)amino)-3-(4,5-difluoro-2-iodophenyl)-2-methylpropanoate (Scheme 54) is shown schematically in Scheme 55. To a solution of 2-[(tert-butoxycarbonyl)amino]-3-(4,5-difluoro-2-iodophenyl)-2-methylpropanoic acid (80 mg, 0.18 mmol) in THF/MeOH (4:1, 2.5 mL) was added (trimethylsilyl)diazomethane (2M in hexanes, 0.36 mL, 0.72 mmol). After 2 h, the mixture was concentrated under reduced pressure and purified by prep-HPLC [Column: Xselect CSH Prep Fluoro-Phenyl 5 μm 30×150 mm; Mobile Phase: ACN-water, 41-71% (with 0.1% FA); Flow rate: 60 mL/min; Wavelength: 254/220 nm] to afford the title compound (31.9 mg, 38%) as a white solid.

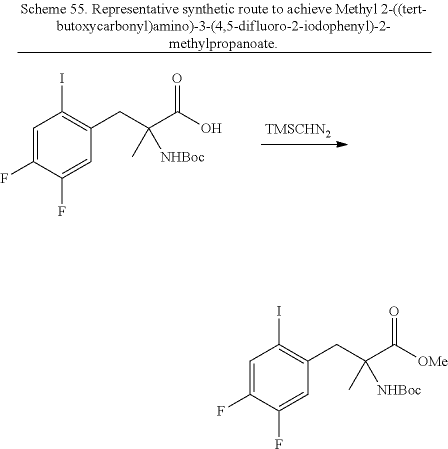 [SMILES: CC(C)(C)OC(=O)NC(C)(Cc1cc(F)c(F)cc1I)C(=O)O ; COC(=O)C(C)(Cc1cc(F)c(F)cc1I)NC(=O)OC(C)(C)C]

For Methyl 2-((tert-butoxycarbonyl)amino)-3-(4,5-difluoro-2-iodophenyl)-2-methylpropanoate, ¹H NMR (400 MHz, DMSO) δ 8.03-7.95 (m, 1H), 7.34 (br s, 1H), 7.12-6.99 (m, 1H), 3.64 (s, 3H), 3.41 (br d, J=14.4 Hz, 1H), 3.23 (d, J=14.0 Hz, 1H), 1.42 (s, 9H), 1.18 (s, 3H). ¹⁹F NMR (376 MHz, DMSO-d₆) δ −138.68-(−)139.59 (m). LCMS (ES, m/z): 355.85 [M-Boc+H]⁺; 99.5% purity (220 nm).

### Example 33: Synthesis of tert-Butyl (S)-2-((tert-butoxycarbonyl)amino)-3-(4-(4-chlorophenoxy)-5-fluoro-2-iodophenyl)propanoate and tert-Butyl (R)-2-((tert-butoxycarbonyl)amino)-3-(4-(4-chlorophenoxy)-5-fluoro-2-iodophenyl)propanoate

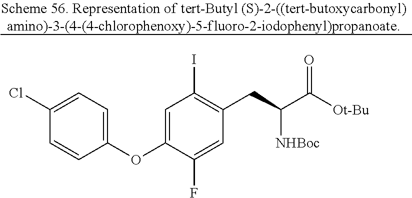 [SMILES: CC(C)(C)OC(=O)N[C@@H](Cc1cc(F)c(Oc2ccc(Cl)cc2)cc1I)C(=O)OC(C)(C)C]

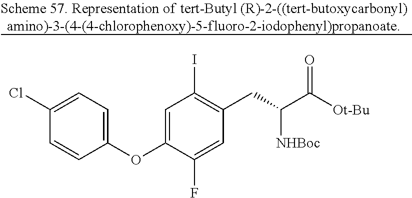 [SMILES: CC(C)(C)OC(=O)N[C@H](Cc1cc(F)c(Oc2ccc(Cl)cc2)cc1I)C(=O)OC(C)(C)C]

The preparation of tert-Butyl (S)-2-((tert-butoxycarbonyl)amino)-3-(4-(4-chlorophenoxy)-5-fluoro-2-iodophenyl)propanoate (Scheme 56) and tert-Butyl (R)-2-((tert-butoxycarbonyl)amino)-3-(4-(4-chlorophenoxy)-5-fluoro-2-iodophenyl)propanoate (Scheme 57) is shown schematically in Scheme 58. To a solution of 4,5-difluoro-2-iodobenzoic acid (10.0 g, 35.2 mmol) in tert-butyl acetate (100 mL) at 0° C. was added a solution of trifluoromethanesulfonimide (198 mg, 0.70 mmol) in DCM (9 mL), dropwise. After 16 h at rt, the mixture was poured into ice-water (200 mL) and extracted with DCM (3×200 mL). The combined organic layers were washed with brine (200 mL), dried over anhydrous Na₂SO₄ and concentrated under reduced pressure. The crude residue was purified by silica gel chromatography (EA-petroleum ether, 20%) to afford the title compound (6.50 g, 54%) as a clear oil. GCMS (EI, m/z): 340.00 [M].

A mixture of tert-butyl 4,5-difluoro-2-iodobenzoate (6.50 g, 19.1 mmol), 4-chlorophenol (2.46 g, 19.1 mmol) and Cs₂CO₃ (12.5 g, 38.2 mmol) in DMF (120 mL) was heated at 80° C. for 16 h, whereupon it was cooled to rt, poured into ice-water (200 mL) and extracted with EtOAc (3×200 mL). The combined organic layers were washed with brine (200 mL), dried over anhydrous Na₂SO₄ and concentrated under reduced pressure. The crude residue was purified by silica gel chromatography (EA-petroleum ether, 20%) to afford the title compound (5.30 g, 61%) as a white solid. ¹H NMR (400 MHz, DMSO-d₆) δ 7.73 (dd, J=11.3, 1.0 Hz, 1H), 7.61 (dd, J=7.9, 1.1 Hz, 1H), 7.51-7.45 (m, 2H), 7.18-7.12 (m, 2H), 1.57 (s, 9H).

To a solution of tert-butyl 4-(4-chlorophenoxy)-5-fluoro-2-iodobenzoate (5.30 g, 11.8 mmol) in DCM (100 mL) was added TFA (30 mL). After 4 h, the mixture was concentrated under reduced pressure and the crude residue was purified by silica gel chromatography (EA-petroleum ether, 50%) to afford the title compound (3.40 g, 73%) as a colorless oil. LCMS (ES, m/z): 391.10 [M−H]⁻.

To a solution of 4-(4-chlorophenoxy)-5-fluoro-2-iodobenzoic acid (3.40 g, 8.67 mmol) in THF (30 mL) at 0° C. was added borane-tetrahydrofuran complex (1.0 M in THF, 87.0 mL, 86.7 mmol), dropwise. After 16 h at rt, the mixture was quenched with MeOH (100 mL), heated at 70° C. for 1 h, then cooled to rt and concentrated under reduced pressure. The crude residue was purified by silica gel chromatography (EA-petroleum ether, 50%) to afford the title compound (2.50 g, 76%) as a colorless oil. ¹H NMR (400 MHz, DMSO-d₆) δ 7.64 (d, J=8.0 Hz, 1H), 7.47-7.38 (m, 3H), 7.06-6.99 (m, 2H), 5.65 (t, J=5.5 Hz, 1H), 4.38 (d, J=5.4 Hz, 2H).

To a solution of [4-(4-chlorophenoxy)-5-fluoro-2-iodophenyl]methanol (2.50 g, 6.61 mmol) in DCM (80 mL) at 0° C. was added PBr₃ (3.58 g, 13.2 mmol), dropwise. After 4 h at rt, the mixture was concentrated under reduced pressure and purified by silica gel chromatography (EA-petroleum ether, 25%) to afford the title compound (2.70 g, 92%) as a yellow solid.

To a solution of 1-(bromomethyl)-4-(4-chlorophenoxy)-5-fluoro-2-iodobenzene (2.70 g, 6.14 mmol) in MeCN (20 mL) was added K₂CO₃ (2.54 g, 18.4 mmol), tert-butyl 2-[(diphenylmethylidene)amino]acetate (1.82 g, 6.14 mmol) and tetrabutylammonium bromide (200 mg, 0.62 mmol). After 16 h, the mixture was poured into ice-water (50 mL) and extracted with EtOAc (3×50 mL). The combined organic layers were washed with brine (50 mL), dried over anhydrous Na₂SO₄ and concentrated under reduced pressure. The crude residue was purified by silica gel chromatography (EA-petroleum ether, 20%) to afford the title compound (2.00 g, 49%) as a colorless oil. LCMS (ES, m/z): 656.05 [M+H]⁺.

To a solution of tert-butyl 3-[4-(4-chlorophenoxy)-5-fluoro-2-iodophenyl]-2-[(diphenylmethylidene)amino]propanoate (2.00 g, 3.05 mmol) in EtOH (32 mL) was added a solution of hydroxylamine hydrochloride (2.76 g, 39.7 mmol) in H₂O (8 mL). The mixture was heated at 50° C. for 4 h, then cooled to rt followed by addition of NaOH (1 M) until pH-8 was achieved. The resulting mixture was concentrated under reduced pressure and the crude residue was purified by reverse flash chromatography (C18 silica gel, ACN-water, 0-60% with 10 mM NH₄HCO₃) to afford the title compound (1.15 g, 76%) as a white solid. LCMS (ES, m/z): 492.10 [M+H]⁺.

To a solution of tert-butyl 2-amino-3-[4-(4-chlorophenoxy)-5-fluoro-2-iodophenyl]propanoate (400 mg, 0.82 mmol) in DCM (2 mL) at 0° C. was added TEA (248 mg, 2.45 mmol) followed by di-tert-butyl dicarbonate (267 mg, 1.23 mmol). After 2 h at rt, the mixture was poured into ice-water (20 mL) and extracted with DCM (3×20 mL). The combined organic layers were washed with brine (20 mL), dried over anhydrous Na₂SO₄ and concentrated under reduced pressure. The crude residue was purified by silica gel chromatography (EA-petroleum ether, 15%) to afford tert-butyl 2-((tert-butoxycarbonyl)amino)-3-(4-(4-chlorophenoxy)-5-fluoro-2-iodophenyl)propanoate (330 mg). The racemic material was further separated by Prep-SFC [Column: Chiral ART Cellulose-SA 5 μm 2×25 cm; Mobile Phase A: Hexanes; Mobile Phase B: EtOH/DCM (1:1), 5%; Flow rate: 20 mL/min; Wavelength: 254/220 nm] to afford tert-butyl (S)-2-((tert-butoxycarbonyl)amino)-3-(4-(4-chlorophenoxy)-5-fluoro-2-iodophenyl)propanoate (first eluting isomer, 100 mg, 20%) and tert-butyl (R)-2-((tert-butoxycarbonyl)amino)-3-(4-(4-chlorophenoxy)-5-fluoro-2-iodophenyl)propanoate (second eluting isomer, 90.2 mg, 18%) as white solids.

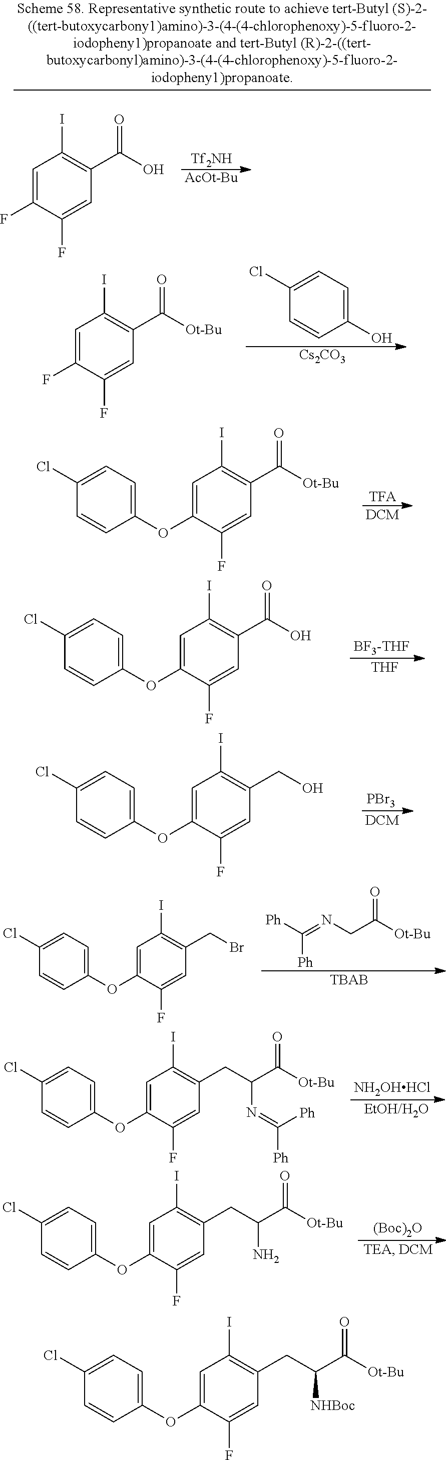

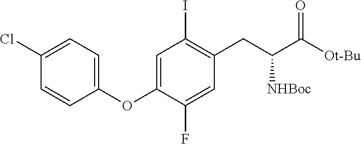 [SMILES: CC(C)(C)OC(=O)N[C@H](Cc1cc(F)c(Oc2ccc(Cl)cc2)cc1I)C(=O)OC(C)(C)C]

For tert-Butyl (S)-2-((tert-butoxycarbonyl)amino)-3-(4-(4-chlorophenoxy)-5-fluoro-2-iodophenyl)propanoate, ¹H NMR (400 MHz, DMSO-d₆) δ 7.64 (br d, J=8.2 Hz, 1H), 7.48-7.43 (m, 2H), 7.40 (d, J=12.1 Hz, 1H) 7.31 (d, J=8.5 Hz, 1H), 7.05-6.92 (m, 2H), 4.18-4.09 (m, 1H), 3.14-3.02 (m, 1H), 2.92 (dd, J=13.6, 10.6 Hz, 1H), 1.41 (s, 9H), 1.37-1.26 (m, 9H). ¹⁹F NMR (376 MHz, DMSO-d₆) δ −132.14. LCMS (ES, m/z): 435.95 [M-tBu-Boc+H]⁺; 99.8% purity (220 nm).

For tert-Butyl (R)-2-((tert-butoxycarbonyl)amino)-3-(4-(4-chlorophenoxy)-5-fluoro-2-iodophenyl)propanoate, ¹H NMR (400 MHz, DMSO-d₆) δ 7.64 (br d, J=8.2 Hz, 1H), 7.48-7.43 (m, 2H), 7.40 (d, J=12.1 Hz, 1H) 7.31 (d, J=8.5 Hz, 1H), 7.05-6.92 (m, 2H), 4.18-4.09 (m, 1H), 3.14-3.02 (m, 1H), 2.92 (dd, J=13.6, 10.6 Hz, 1H), 1.41 (s, 9H), 1.37-1.26 (m, 9H). ¹⁹F NMR (376 MHz, DMSO-d₆) δ −132.14. LCMS (ES, m/z): 435.95 [M-tBu-Boc+H]⁺; 99.6% purity (254 nm).

### Example 34: Synthesis of tert-Butyl 2-((tert-butoxycarbonyl)amino)-3-(4-(4-chlorophenoxy)-5-fluoro-2-iodophenyl)-2-methylpropanoate

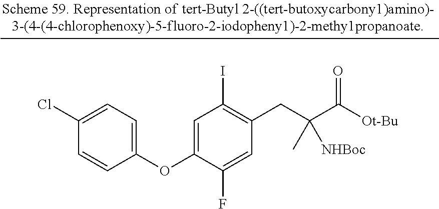 [SMILES: CC(C)(C)OC(=O)NC(C)(Cc1cc(F)c(Oc2ccc(Cl)cc2)cc1I)C(=O)OC(C)(C)C]

The preparation of the title compound (60 mg) tert-Butyl 2-((tert-butoxycarbonyl)amino)-3-(4-(4-chlorophenoxy)-5-fluoro-2-iodophenyl)-2-methylpropanoate (Scheme 59) from an intermediate of Example 33 is undertaken in an analogous manner to intermediate in Scheme 43 and of Example 26 and Scheme 48 of Example 28.

For tert-Butyl 2-((tert-butoxycarbonyl)amino)-3-(4-(4-chlorophenoxy)-5-fluoro-2-iodophenyl)-2-methylpropanoate, ¹H NMR (400 MHz, DMSO-d₆) δ 7.63 (br d, J=8.4 Hz, 1H), 7.49-7.42 (m, 2H), 7.22 (s, 1H), 7.09-6.99 (m, 3H), 3.41 (d, J=14.0 Hz, 1H), 3.22 (d, J=14.1 Hz, 1H), 1.48-1.37 (m, 18H), 1.16 (s, 3H). ¹⁹F NMR (376 MHz, DMSO-d₆) δ −132.17. LCMS (ES, m/z): 450.00 [M-tBu-Boc+H]⁺; 96.7% purity (254 nm).

### Example 35: Synthesis of tert-Butyl (R)-2-((tert-butoxycarbonyl)amino)-3-(4-(4-chlorophenoxy)-5-fluoro-2-iodophenyl)-2-methylpropanoate and tert-Butyl (S)-2-((tert-butoxycarbonyl)amino)-3-(4-(4-chlorophenoxy)-5-fluoro-2-iodophenyl)-2-methylpropanoate

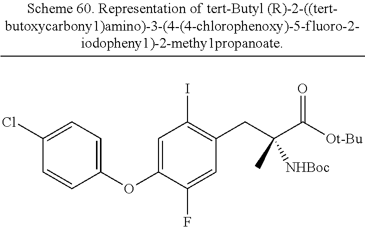 [SMILES: CC(C)(C)OC(=O)N[C@](C)(Cc1cc(F)c(Oc2ccc(Cl)cc2)cc1I)C(=O)OC(C)(C)C]

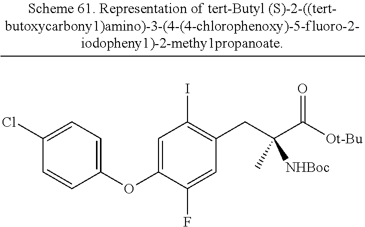 [SMILES: CC(C)(C)OC(=O)N[C@@](C)(Cc1cc(F)c(Oc2ccc(Cl)cc2)cc1I)C(=O)OC(C)(C)C]

The preparation of tert-Butyl (R)-2-((tert-butoxycarbonyl)amino)-3-(4-(4-chlorophenoxy)-5-fluoro-2-iodophenyl)-2-methylpropanoate (Scheme 60) and tert-Butyl (S)-2-((tert-butoxycarbonyl)amino)-3-(4-(4-chlorophenoxy)-5-fluoro-2-iodophenyl)-2-methylpropanoate (Scheme 61) was undertaken by further separating the racemate of Example 34. Separation was by Prep-HPLC (Column: CHIRALPAK IE 5 m, 2×25 cm; Mobile Phase: MtBE-Hexanes, 20%; Flow rate: 20 mL/min; Wavelength: 254/220 nm) to afford tert-butyl (R)-2-((tert-butoxycarbonyl)amino)-3-(4-(4-chlorophenoxy)-5-fluoro-2-iodophenyl)-2-methylpropanoate (first eluting isomer, 15.2 mg) and tert-butyl (S)-2-((tert-butoxycarbonyl)amino)-3-(4-(4-chlorophenoxy)-5-fluoro-2-iodophenyl)-2-methylpropanoate (second eluting isomer, 11.6 mg) as white solids.

For tert-Butyl (R)-2-((tert-butoxycarbonyl)amino)-3-(4-(4-chlorophenoxy)-5-fluoro-2-iodophenyl)-2-methylpropanoate, ¹H NMR (400 MHz, DMSO-d₆) δ 7.63 (d, J=8.4 Hz, 1H), 7.49-7.42 (m, 2H), 7.22 (s, 1H), 7.09-6.99 (m, 3H), 3.41 (d, J=14.0 Hz, 1H), 3.22 (d, J=13.8 Hz, 1H), 1.48-1.37 (m, 18H), 1.16 (s, 3H). ¹⁹F NMR (376 MHz, DMSO-d₆) δ −132.17. LCMS (ES, m/z): 450.00 [M-tBu-Boc+H]⁺; 99.0% purity (254 nm).

For tert-Butyl (S)-2-((tert-butoxycarbonyl)amino)-3-(4-(4-chlorophenoxy)-5-fluoro-2-iodophenyl)-2-methylpropanoate, ¹H NMR (400 MHz, DMSO-d₆) δ 7.63 (d, J=8.4 Hz, 1H), 7.49-7.42 (m, 2H), 7.22 (s, 1H), 7.09-6.99 (m, 3H), 3.41 (d, J=13.9 Hz, 1H), 3.22 (d, J=13.8 Hz, 1H), 1.48-1.37 (m, 18H), 1.16 (s, 3H). ¹⁹F NMR (376 MHz, DMSO-d₆) δ −132.17. LCMS (ES, m/z): 449.95 [M-tBu-Boc+H]⁺; 98.0% purity (254 nm).

### Example 36: Synthesis of tert-Butyl 2-((tert-butoxycarbonyl)(methyl)amino)-3-(4-(4-chlorophenoxy)-5-fluoro-2-iodophenyl)-2-methylpropanoate

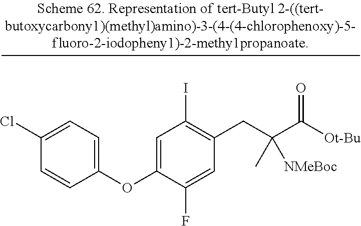

The preparation of the title compound (50 mg) tert-Butyl 2-((tert-butoxycarbonyl)(methyl)amino)-3-(4-(4-chlorophenoxy)-5-fluoro-2-iodophenyl)-2-methylpropanoate (Scheme 62) is undertaken from a previously described intermediate and according to analogous methods of Example 30.

For tert-Butyl 2-((tert-butoxycarbonyl)(methyl)amino)-3-(4-(4-chlorophenoxy)-5-fluoro-2-iodophenyl)-2-methylpropanoate, ¹H NMR (400 MHz, DMSO-d₆) δ 7.65 (d, J=8.4 Hz, 1H), 7.50-7.42 (m, 2H), 7.12-7.03 (m, 2H), 6.97 (br d, J=11.5 Hz, 1H), 3.56 (d, J=14.2 Hz, 1H), 3.28-3.17 (m, 1H), 2.55 (s, 3H), 1.46 (s, 9H), 1.42 (s, 9H), 1.33 (s, 3H). ¹⁹F NMR (376 MHz, DMSO-d₆) δ −131.91. LCMS (ES, m/z): 464.00 [M-tBu-Boc+H]⁺; 99.6% purity (254 nm).

### Example 37: Synthesis of tert-Butyl (3-(4-(4-chlorophenoxy)-5-fluoro-2-iodophenyl)-2-methyl-1-(methylamino)-1-oxopropan-2-yl)carbamate

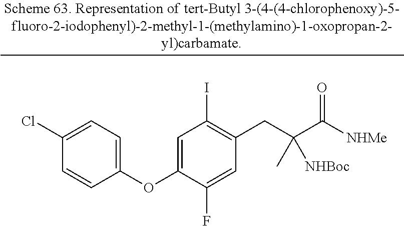 [SMILES: CNC(=O)C(C)(Cc1cc(F)c(Oc2ccc(Cl)cc2)cc1I)NC(=O)OC(C)(C)C]

The preparation of tert-Butyl (3-(4-(4-chlorophenoxy)-5-fluoro-2-iodophenyl)-2-methyl-1-(methylamino)-1-oxopropan-2-yl)carbamate (Scheme 63) is undertaken according to the schematic of Scheme 64. To a solution of tert-butyl 3-(4-(4-chlorophenoxy)-5-fluoro-2-iodophenyl)-2-((diphenylmethylene)amino)-2-methylpropanoate (600 mg, 0.90 mmol) in DCM (10 mL) was added TFA (10 mL), dropwise. After 16 h, the mixture was concentrated under reduced pressure and the crude residue was purified by reverse flash chromatography (C18 silica gel, ACN-water, 0-80% with 10 mM NH₄HCO₃) to afford the title compound (350 mg, 86%) as a white solid. ¹H NMR (400 MHz, DMSO) δ 7.65 (d, J=8.4 Hz, 1H), 7.59 (br s, 2H), 7.49 (d, J=12.5 Hz, 1H), 7.47-7.42 (m, 2H), 7.07-7.00 (m, 2H), 3.23 (d, J=14.4 Hz, 1H), 3.08 (d, J=14.4 Hz, 1H), 1.28 (s, 3H). ¹⁹F NMR (376 MHz, DMSO) δ −130.98. LCMS (ES, m/z): 449.90 [M+H]⁺.

To a mixture of 2-amino-3-(4-(4-chlorophenoxy)-5-fluoro-2-iodophenyl)-2-methylpropanoic acid (150 mg, 0.33 mmol) in DMF (1.5 mL) was added DIEA (216 mg, 1.67 mmol), HATU (190 mg, 0.50 mmol) and methylamine hydrochloride (112 mg, 1.67 mmol). After 2 h, the crude product was directly purified by reverse flash chromatography (C18 silica gel, ACN-water, 0-100% with 10 mM NH₄HCO₃) to afford the title compound (80 mg, 52%) as a white solid. LCMS (ES, m/z): 462.95 [M+H]⁺.

To a mixture of 2-amino-3-(4-(4-chlorophenoxy)-5-fluoro-2-iodophenyl)-N,2-dimethylpropanamide (80 mg, 0.17 mmol) in MeOH (2 mL) was added a solution of K₂CO₃ (72 mg, 0.52 mmol) in H₂O (1 mL) followed by di-tert-butyl dicarbonate (56 mg, 0.26 mmol). After 4 h, the crude product was directly purified by reverse flash chromatography (C18 silica gel, ACN-water, 0-100% with 10 mM NH₄HCO₃) to afford the title compound (50.3 mg, 51%) as a white solid.

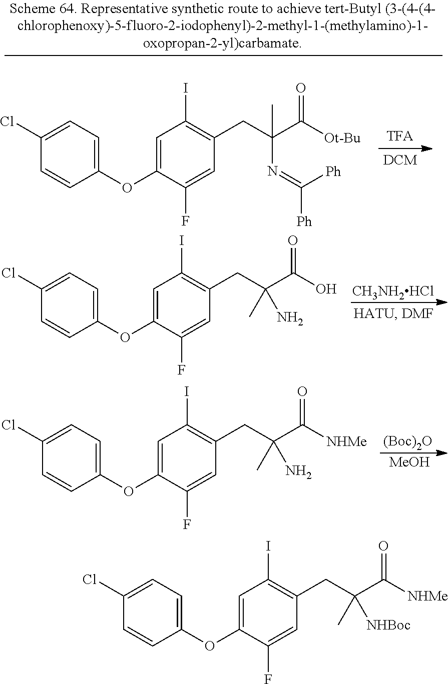

For tert-Butyl (3-(4-(4-chlorophenoxy)-5-fluoro-2-iodophenyl)-2-methyl-1-(methylamino)-1-oxopropan-2-yl)carbamate, ¹H NMR (400 MHz, DMSO-d₆) δ 7.71-7.64 (m, 1H), 7.63 (d, J=8.4 Hz, 1H), 7.48-7.42 (m, 2H), 7.08-7.02 (m, 2H), 7.00-6.92 (m, 2H), 3.44-3.28 (m, 2H), 2.59 (d, J=4.4 Hz, 3H), 1.42 (s, 9H), 1.18 (s, 3H). ¹⁹F NMR (376 MHz, DMSO-d₆) δ −132.31. LCMS (ES, m/z): 484.95 [M-Boc+Na]⁺; 97.5% purity (220 nm).

### Example 38: Synthesis of Methyl 2-((tert-butoxycarbonyl)amino)-3-(4-(4-chlorophenoxy)-5-fluoro-2-iodophenyl)-2-methylpropanoate

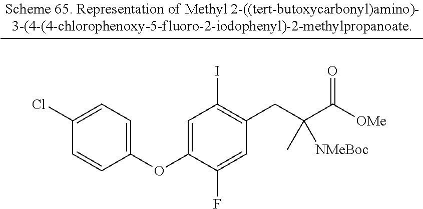

The preparation of the title compound (50.7 mg) Methyl 2-((tert-butoxycarbonyl)amino)-3-(4-(4-chlorophenoxy)-5-fluoro-2-iodophenyl)-2-methylpropanoate (Scheme 65) is according to analogous methods described herein.

For Methyl 2-((tert-butoxycarbonyl)amino)-3-(4-(4-chlorophenoxy)-5-fluoro-2-iodophenyl)-2-methylpropanoate, ¹H NMR (400 MHz, DMSO-d₆) δ 7.64 (d, J=8.4 Hz, 1H), 7.49-7.42 (m, 2H), 7.36 (br s, 1H), 7.10-7.01 (m, 3H), 3.64 (s, 3H), 3.43 (d, J=14.1 Hz, 1H), 3.25 (d, J=13.9 Hz, 1H), 1.42 (s, 9H), 1.20 (s, 3H). ¹⁹F NMR (376 MHz, DMSO-d₆) δ −132.12. LCMS (ES, m/z): 463.95 [M-Boc+H]⁺; 97.5% purity (254 nm).

### Example 39: Synthesis of Methyl 2-((tert-butoxycarbonyl)amino)-3-(2,4-difluoro-5-iodo-3-methoxyphenyl)propanoate

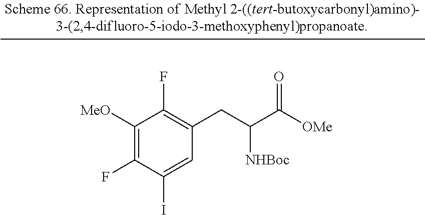 [SMILES: COC(=O)C(Cc1cc(I)c(F)c(OC)c1F)NC(=O)OC(C)(C)C]

The preparation of Methyl 2-((tert-butoxycarbonyl)amino)-3-(2,4-difluoro-5-iodo-3-methoxyphenyl)propanoate (Scheme 66) is according to analogous methods described herein.

For Methyl 2-((tert-butoxycarbonyl)amino)-3-(2,4-difluoro-5-iodo-3-methoxyphenyl)propanoate, ¹H NMR (400 MHz, DMSO-d₆) δ *7.56-7.48 (m, 0.2H), *7.47 (t, J=7.1 Hz, 0.8H), *7.30 (d, J=8.6 Hz, 0.8H), *7.01-6.94 (m, 0.2H), *4.26-4.15 (m, 0.8H), *4.14-4.03 (m, 0.2H), 3.90 (s, 3H), 3.63 (s, 3H), 3.13-2.97 (m, 1H), 2.85-2.73 (m, 1H), 1.37-1.14 (m, 9H). *Partial integration due to presence of rotamers. ¹⁹F NMR (376 MHz, DMSO-d₆) δ −110.34 (d, J=10.0 Hz, minor rotamer), −110.50 (d, J=9.5 Hz, major rotamer), −131.94 (d, J=9.6 Hz, major rotamer), −132.14 (d, J=8.2 Hz, minor rotamer). LCMS (ES, m/z): 372.00 [M-Boc+H]⁺; 95.0% purity (254 nm).

### Example 40: Synthesis of Benzyl 2-((tert-butoxycarbonyl)amino)-3-(2,4-difluoro-5-iodo-3-methoxyphenyl)propanoate

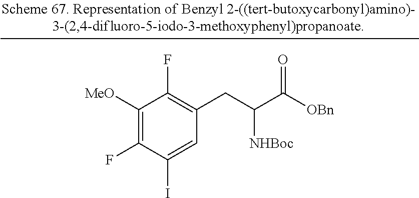 [SMILES: COc1c(F)c(I)cc(CC(NC(=O)OC(C)(C)C)C(=O)OCc2ccccc2)c1F]

The preparation of the title compound (20.4 mg) Benzyl 2-((tert-butoxycarbonyl)amino)-3-(2,4-difluoro-5-iodo-3-methoxyphenyl)propanoate (Scheme 67) is according to analogous methods described herein.

For Benzyl 2-((tert-butoxycarbonyl)amino)-3-(2,4-difluoro-5-iodo-3-methoxyphenyl)propanoate, ¹H NMR (400 MHz, DMSO-d₆) δ *7.58-7.51 (m, 0.1H), 7.48 (t, J=7.1 Hz, 0.9H), 7.44-7.28 (m, 5H), 5.11 (s, 2H), *4.25 (td, J=9.6, 4.3 Hz, 0.9H), *4.20-4.11 (m, 0.1H), 3.87 (s, 3H), *3.09 (dd, J=13.7, 5.1 Hz, 0.9H), *3.04-2.99 (m, 0.1H), 2.92-2.80 (m, 1H), 1.40-1.17 (m, 9H). *Partial integration due to presence of rotamers. ¹⁹F NMR (376 MHz, DMSO-d₆) δ −110.26 (d, J=9.4 Hz, minor rotamer), −110.43 (d, J=9.2 Hz, major rotamer), −131.86 (d, J=9.5 Hz, major rotamer), −132.03 (d, J=9.2 Hz, minor rotamer). LCMS (ES, m/z): 448.00 [M-Boc+H]⁺; 99.2% purity (254 nm).

### Example 41: Synthesis of tert-Butyl 2-((tert-butoxycarbonyl)(methyl)amino)-3-(2,4-difluoro-5-iodo-3-methoxyphenyl)-2-methylpropanoate

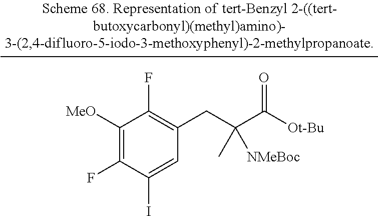

The preparation of the title compound (50 mg) tert-Butyl 2-((tert-butoxycarbonyl)(methyl)amino)-3-(2,4-difluoro-5-iodo-3-methoxyphenyl)-2-methylpropanoate (Scheme 68) is according to analogous methods described herein.

For tert-Butyl 2-((tert-butoxycarbonyl)(methyl)amino)-3-(2,4-difluoro-5-iodo-3-methoxyphenyl)-2-methylpropanoate, ¹H NMR (400 MHz, DMSO-d₆) δ 7.36-7.19 (m, 1H), 3.90 (s, 3H), 3.47 (d, J=13.6 Hz, 1H), 2.85 (d, J=12.4 Hz, 1H), 2.42 (s, 3H), 1.46 (s, 9H), 1.39 (s, 9H), 1.26 (s, 3H). ¹⁹F NMR (376 MHz, DMSO-d₆) δ −110.42, −129.91. LCMS (ES, m/z): 386.00 [M-tBu-Boc+H]⁺; 99.7% purity (254 nm).

### Example 42: Synthesis of 3-(2,4-Difluoro-5-iodo-3-methoxyphenyl)-2-methyl-2-(methylamino)propanoic acid

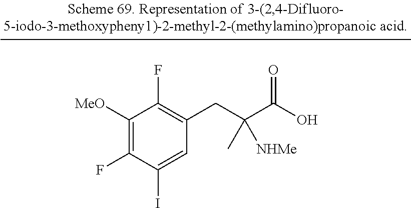 [SMILES: CNC(C)(Cc1cc(I)c(F)c(OC)c1F)C(=O)O]

The preparation of the title compound (22 mg) 3-(2,4-Difluoro-5-iodo-3-methoxyphenyl)-2-methyl-2-(methylamino)propanoic acid (Scheme 69) is according to analogous methods described herein.

For 3-(2,4-Difluoro-5-iodo-3-methoxyphenyl)-2-methyl-2-(methylamino)propanoic acid, ¹H NMR (400 MHz, DMSO-d₆) δ 7.93 (br s, 1H), 7.53 (t, J=7.2 Hz, 1H), 3.89 (s, 3H), 2.95-2.85 (m, 2H), 2.37 (s, 3H), 1.26-1.18 (m, 3H). ¹⁹F NMR (376 MHz, DMSO-d₆) δ −110.66 (d, J=8.9 Hz), −129.57 (d, J=9.6 Hz). LCMS (ES, m/z): 386.00 [M+H]⁺; 98.7% purity (220 nm).

### Example 43: Synthesis of tert-Butyl (3-(2,4-difluoro-5-iodo-3-methoxyphenyl)-1-oxo-1-(pyrrolidin-1-yl)propan-2-yl)carbamate

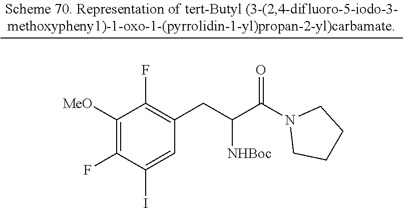 [SMILES: COc1c(F)c(I)cc(CC(NC(=O)OC(C)(C)C)C(=O)N2CCCC2)c1F]

The preparation of the title compound (50 mg) tert-Butyl (3-(2,4-difluoro-5-iodo-3-methoxyphenyl)-1-oxo-1-(pyrrolidin-1-yl)propan-2-yl)carbamate (Scheme 70) is according to analogous methods described herein.

For tert-Butyl (3-(2,4-difluoro-5-iodo-3-methoxyphenyl)-1-oxo-1-(pyrrolidin-1-yl)propan-2-yl)carbamate, ¹H NMR (400 MHz, DMSO-d₆) δ *7.57-7.50 (m, 0.16H), *7.48 (t, J=7.2 Hz, 0.85H), *6.99 (d, J=8.8 Hz, 0.8H), *6.66 (d, J=7.3 Hz, 0.2H), *4.40 (td, J=9.4, 4.7 Hz, 0.8H), *4.34-4.23 (m, 0.2H), 3.89 (s, 3H), 3.51-3.40 (m, 1H), 3.37-3.32 (m, 1H), 3.30-3.20 (m, 2H), *2.92 (dd, J=13.7, 4.3 Hz, 0.84H), 2.88-2.82 (m, 0.14H), 2.76-2.65 (m, 1H), 1.93-1.81 (m, 2H), 1.80-1.68 (m, 2H), 1.35-1.15 (m, 9H). *Partial integration due to presence of rotamers. ¹⁹F NMR (376 MHz, DMSO-d₆) δ −110.53 (d, J=9.0 Hz, minor rotamer), −110.78 (d, J=9.2 Hz, major rotamer), −131.78 (d, J=9.2 Hz, major rotamer), −132.11 (d, J=8.8 Hz, minor rotamer). LCMS (ES, m/z): 411.05 [M-Boc+H]⁺.

### Example 44: Synthesis of 2-Amino-3-(2,4-difluoro-5-iodo-3-methoxyphenyl)-1-(pyrrolidin-1-yl)propan-1-one

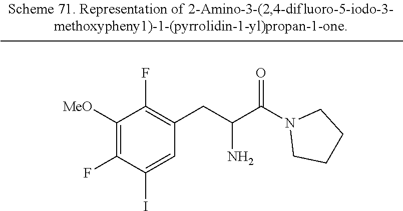 [SMILES: COc1c(F)c(I)cc(CC(N)C(=O)N2CCCC2)c1F]

The preparation of the title compound (22.5 mg) 2-Amino-3-(2,4-difluoro-5-iodo-3-methoxyphenyl)-1-(pyrrolidin-1-yl)propan-1-one (Scheme 71) is according to analogous methods described herein.

For 2-Amino-3-(2,4-difluoro-5-iodo-3-methoxyphenyl)-1-(pyrrolidin-1-yl)propan-1-one, ¹H NMR (400 MHz, DMSO-d₆) δ 7.47 (t, J=7.2 Hz, 1H), 3.90 (s, 3H), 3.60 (dd, J=8.2, 5.7 Hz, 1H), 3.52 (dt, J=10.0, 6.3 Hz, 1H), 3.32-3.12 (m, 3H), 2.72 (dd, J=13.7, 5.4 Hz, 1H), 2.60 (dd, J=13.2, 8.4 Hz, 1H), 1.99 (br s, 2H), 1.91-1.64 (m, 4H). ¹⁹F NMR (376 MHz, DMSO-d₆) δ −111.13 (d, J=8.6 Hz), −132.04 (d, J=8.6 Hz). LCMS (ES, m/z): 411.05 [M+H]⁺; 99.6% purity (254 nm).

### Example 45: Synthesis of tert-Butyl (S)-2-((tert-butoxycarbonyl)amino)-3-(4,5-difluoro-2-(4,4,5,5-tetramethyl-1,3,2-dioxaborolan-2-yl)phenyl)propanoate

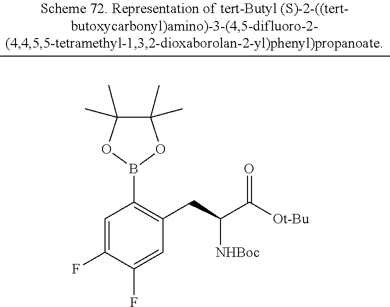 [SMILES: CC(C)(C)OC(=O)N[C@@H](Cc1cc(F)c(F)cc1B1OC(C)(C)C(C)(C)O1)C(=O)OC(C)(C)C]

The preparation of tert-Butyl (S)-2-((tert-butoxycarbonyl)amino)-3-(4,5-difluoro-2-(4,4,5,5-tetramethyl-1,3,2-dioxaborolan-2-yl)phenyl)propanoate (Scheme 72) is undertaken with the addition of 4,4,5,5-tetramethyl-1,3,2-dioxaborolane (139 mg, 1.09 mmol), TEA (221 mg, 2.19 mmol), P(o-Tol)₃ (44 mg, 0.15 mmol) and Pd(OAc)₂ (16 mg, 0.07 mmol) to a solution of tert-butyl (2S)-2-[(tert-butoxycarbonyl)amino]-3-(4,5-difluoro-2-iodophenyl)propanoate (350 mg, 0.73 mmol) (Scheme 46) under a N₂ atmosphere. After heating the mixture to 77° C. for 1 h, it was cooled to rt and concentrated under reduced pressure. The crude residue was purified by reversed phase flash chromatography (C18 silica gel, ACN-water, 50-90% with 0.1% FA) to afford the title compound (120 mg, 34%) as a white solid. The process is shown schematically in Scheme 73.

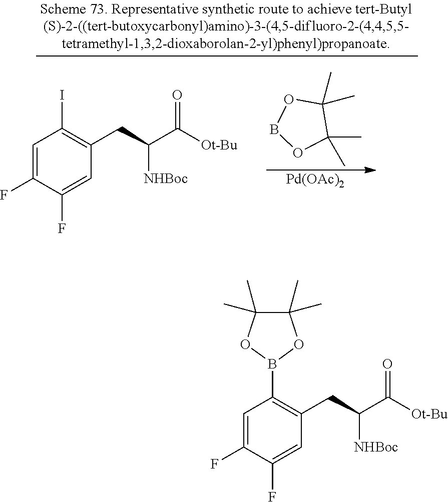

For tert-Butyl (S)-2-((tert-butoxycarbonyl)amino)-3-(4,5-difluoro-2-(4,4,5,5-tetramethyl-1,3,2-dioxaborolan-2-yl)phenyl)propanoate, ¹H NMR (400 MHz, DMSO-d₆) δ 7.56-7.44 (m, 1H), 7.31 (dd, J=12.2, 7.7 Hz, 1H), *7.00 (d, J=8.8 Hz, 0.8H), *6.71 (d, J=8.3 Hz, 0.2H), 4.09 (ddd, J=10.4, 8.8, 5.3 Hz, 1H), 3.39 (dd, J=13.2, 5.3 Hz, 1H), 2.89-2.76 (m, 1H), 1.44-1.10 (m, 30H). *Partial integration due to presence of rotamers. ¹⁹F NMR (376 MHz, DMSO-d₆) δ −135.68 (d, J=22.7 Hz, minor rotamer), −135.82 (d, J=22.4 Hz, major rotamer), −143.14 (dd, J=22.4 Hz, minor rotamer), −143.27 (dd, J=22.6 Hz, major rotamer). LCMS (ES, m/z): 482.15 [M−H]⁻; 99.2% purity (220 nm).

### Example 46: Synthesis of tert-Butyl (R)-2-((tert-butoxycarbonyl)amino)-3-(4,5-difluoro-2-(4,4,5,5-tetramethyl-1,3,2-dioxaborolan-2-yl)phenyl)propanoate

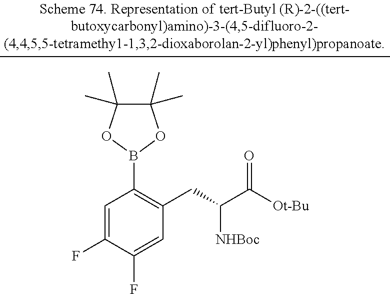 [SMILES: CC(C)(C)OC(=O)N[C@H](Cc1cc(F)c(F)cc1B1OC(C)(C)C(C)(C)O1)C(=O)OC(C)(C)C]

The preparation of the title compound (100 mg) tert-Butyl (R)-2-((tert-butoxycarbonyl)amino)-3-(4,5-difluoro-2-(4,4,5,5-tetramethyl-1,3,2-dioxaborolan-2-yl)phenyl)propanoate (Scheme 74) is undertaken according to analogous conditions described in Example 45 using Example 28.

For tert-Butyl (R)-2-((tert-butoxycarbonyl)amino)-3-(4,5-difluoro-2-(4,4,5,5-tetramethyl-1,3,2-dioxaborolan-2-yl)phenyl)propanoate, ¹H NMR (400 MHz, DMSO-d₆) δ 7.56-7.44 (m, 1H), 7.31 (dd, J=12.2, 7.7 Hz, 1H), *7.00 (d, J=8.8 Hz, 0.8H), *6.71 (d, J=8.3 Hz, 0.2H), 4.09 (ddd, J=10.4, 8.8, 5.3 Hz, 1H), 3.39 (dd, J=13.2, 5.3 Hz, 1H), 2.89-2.76 (m, 1H), 1.44-1.10 (m, 30H). *Partial integration due to presence of rotamers. ¹⁹F NMR (376 MHz, DMSO-d₆) δ −135.68 (d, J=22.6 Hz, minor rotamer), −135.82 (d, J=22.4 Hz, major rotamer), −143.14 (dd, J=22.7 Hz, minor rotamer), −143.26 (dd, J=22.4 Hz, major rotamer). LCMS (ES, m/z): 482.15 [M−H]⁻; 99.3% purity (220 nm).

### Example 47: Synthesis of (S)-(2-(3-(tert-Butoxy)-2-((tert-butoxycarbonyl)amino)-3-oxopropyl)-4,5-difluorophenyl)boronic acid

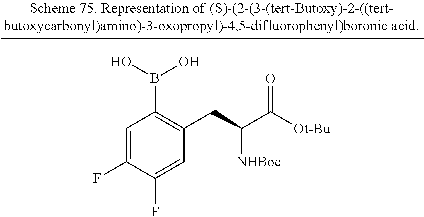 [SMILES: CC(C)(C)OC(=O)N[C@@H](Cc1cc(F)c(F)cc1B(O)O)C(=O)OC(C)(C)C]

The preparation of (S)-(2-(3-(tert-Butoxy)-2-((tert-butoxycarbonyl)amino)-3-oxopropyl)-4,5-difluorophenyl)boronic acid (Scheme 75) is as follows: A mixture of tert-butyl (2S)-2-[(tert-butoxycarbonyl)amino]-3-[4,5-difluoro-2-(4,4,5,5-tetramethyl-1,3,2-dioxaborolan-2-yl)phenyl]propanoate (90 mg, 0.19 mmol), NaIO₄ (121 mg, 0.56 mmol) and NH₄OAc (43 mg, 0.56 mmol) in THF/H₂O (5:1, 1.8 mL) was stirred at 30° C. for 16 h, whereupon it was concentrated under reduced pressure. The crude residue was purified by reverse phase flash chromatography (C18 silica gel, ACN-water, 10-50% with 0.1% FA) to afford a sample of the title compound (70 mg, 94%) as a white solid. 25 mg of the obtained solid was further purified by Prep-HPLC (Column: XSELECT CSH Prep OBD C18 5 m, 30×150 mm; Mobile phase: ACN-water, 40-70% (with 0.1% FA); Flow rate: 60 mL/min; Wavelength: 254/220 nm), affording the title compound (6.0 mg, 24%) as a white solid.

For (S)-(2-(3-(tert-Butoxy)-2-((tert-butoxycarbonyl)amino)-3-oxopropyl)-4,5-difluorophenyl)boronic acid, ¹H NMR (400 MHz, DMSO-d₆) δ 8.47-8.22 (m, 2H), *7.56 (dd, J=10.7, 8.9 Hz, 0.2H), *7.52-7.42 (m, 0.8H), 7.34-7.17 (m, 1H), *7.09 (d, J=8.0 Hz, 0.8H), *6.74 (d, J=8.5 Hz, 0.2H), *4.25-4.10 (m, 0.2H), *4.09-3.95 (m, 0.8H), 3.29 (dd, J=13.5, 5.0 Hz, 1H), 2.90 (dd, J=13.2, 10.3 Hz, 1H), 1.55-1.16 (m, 18H). *Partial integration due to presence of rotamers. ¹⁹F NMR (376 MHz, DMSO-d₆) δ −138.78 (d, J=22.4 Hz, minor rotamer), −139.03 (d, J=22.4 Hz, major rotamer), −143.89 (d, J=22.9 Hz, minor rotamer), −144.07 (d, J=22.6 Hz, major rotamer). LCMS (ES, m/z): 400.05 [M−H]⁻; 97.4% purity (220 nm).

### Example 48: Synthesis of (R)-(2-(3-(tert-Butoxy)-2-((tert-butoxycarbonyl)amino)-3-oxopropyl)-4,5-difluorophenyl)boronic acid

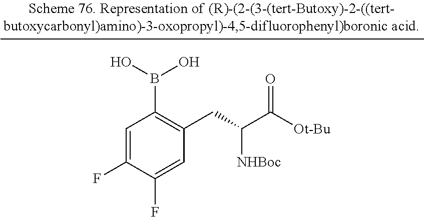 [SMILES: CC(C)(C)OC(=O)N[C@H](Cc1cc(F)c(F)cc1B(O)O)C(=O)OC(C)(C)C]

The preparation of the title compound (7 mg) (R)-(2-(3-(tert-Butoxy)-2-((tert-butoxycarbonyl)amino)-3-oxopropyl)-4,5-difluorophenyl)boronic acid (Scheme 76) is undertaken according to analogous conditions described for Example 47 and using Example 46.

For (R)-(2-(3-(tert-Butoxy)-2-((tert-butoxycarbonyl)amino)-3-oxopropyl)-4,5-difluorophenyl)boronic acid, ¹H NMR (400 MHz, DMSO-d₆) δ 8.47-8.22 (m, 2H), *7.56 (dd, J=10.4, 9.0 Hz, 0.2H), *7.52-7.42 (m, 0.8H), 7.34-7.18 (m, 1H), *7.09 (d, J=8.0 Hz, 0.8H), *6.74 (d, J=8.1 Hz, 0.2H), *4.24-4.09 (m, 0.2H), *4.08-3.95 (m, 0.8H), 3.29 (dd, J=13.6, 5.3 Hz, 1H), 2.90 (dd, J=13.2, 10.3 Hz, 1H), 1.45-1.17 (m, 18H). *Partial integration due to presence of rotamers. ¹⁹F NMR (376 MHz, DMSO-d₆) δ −138.78 (d, J=22.6 Hz, minor rotamer), −139.03 (d, J=22.7 Hz, major rotamer), −143.89 (d, J=23.5 Hz, minor rotamer), −144.07 (d, J=22.4 Hz, major rotamer). LCMS (ES, m/z): 400.05 [M−H]⁻; 96.1% purity (220 nm).

### Example 49: Synthesis of (S)-2-Amino-3-(2-borono-4,5-difluorophenyl)propanoic acid

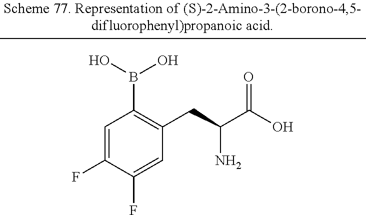 [SMILES: N[C@@H](Cc1cc(F)c(F)cc1B(O)O)C(=O)O]

The preparation of (S)-2-Amino-3-(2-borono-4,5-difluorophenyl)propanoic acid (Scheme 77) is undertaken as follows: To a solution of 2-[(2S)-3-(tert-butoxy)-2-[(tert-butoxycarbonyl)amino]-3-oxopropyl]-4,5-difluorophenylboronic acid (40 mg, 0.10 mmol) in DCM (1 mL) was added TFA (0.5 mL). After 2 h, the mixture was concentrated under reduced pressure and purified by reverse phase flash chromatography (C18 silica gel, ACN-water, 0-20% with 0.1% FA), affording the title compound (9.0 mg, 37%) as a white solid.

For (S)-2-Amino-3-(2-borono-4,5-difluorophenyl)propanoic acid, ¹H NMR (400 MHz, DMSO-d₆/D₂O) δ 7.35 (dd, J=11.1, 9.3 Hz, 1H), 7.09 (dd, J=12.0, 7.4 Hz, 1H), 4.01-3.91 (m, 1H), 3.18 (dd, J=18.0, 5.0 Hz, 1H), 2.90 (d, J=18.0 Hz, 1H). ¹⁹F NMR (376 MHz, DMSO-d₆/D₂O) δ −141.97 (d, J=21.8 Hz), −144.34 (d, J=21.8 Hz). LCMS (ES, m/z): 246.00 [M+H]⁺; 99.9% purity (220 nm).

### Example 50: Synthesis of (R)-2-Amino-3-(2-borono-4,5-difluorophenyl)propanoic acid

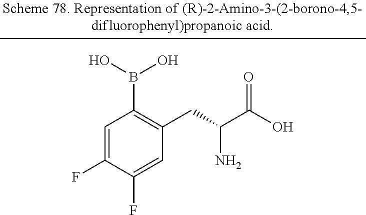 [SMILES: N[C@H](Cc1cc(F)c(F)cc1B(O)O)C(=O)O]

The preparation of (R)-2-Amino-3-(2-borono-4,5-difluorophenyl)propanoic acid (Scheme 50) is undertaken according to analogous conditions described in Example 49 using Example 48.

For (R)-2-Amino-3-(2-borono-4,5-difluorophenyl)propanoic acid, ¹H NMR (400 MHz, DMSO-d₆/D₂O) δ 7.36 (dd, J=11.2, 9.3 Hz, 1H), 7.09 (dd, J=12.0, 7.5 Hz, 1H), 4.01-3.91 (m, 1H), 3.18 (dd, J=17.9, 5.1 Hz, 1H), 2.90 (d, J=18.0 Hz, 1H). ¹⁹F NMR (376 MHz, DMSO-d₆/D₂O) δ −141.99 (d, J=21.4 Hz), −144.35 (d, J=21.5 Hz). LCMS (ES, m/z): 246.00 [M+H]⁺; 99.6% purity (220 nm).

### Examples 51, 52, 53, and 54: Synthesis of: tert-Butyl (S)-3-(3-cyclobutoxy-2,4-difluoro-5-iodophenyl)-2-((diphenylmethylene)amino)propanoate (Example 51)

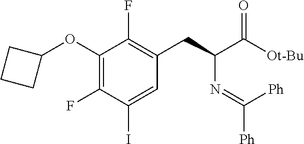 [SMILES: CC(C)(C)OC(=O)[C@H](Cc1cc(I)c(F)c(OC2CCC2)c1F)N=C(c1ccccc1)c1ccccc1]

### tert-Butyl (R)-3-(3-cyclobutoxy-2,4-difluoro-5-iodophenyl)-2-((diphenylmethylene)amino)propanoate (Example 52)

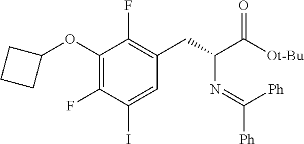 [SMILES: CC(C)(C)OC(=O)[C@@H](Cc1cc(I)c(F)c(OC2CCC2)c1F)N=C(c1ccccc1)c1ccccc1]

### tert-Butyl (S)-3-(3-cyclobutoxy-2,5-difluoro-4-iodophenyl)-2-((diphenylmethylene)amino)propanoate (Example 53)

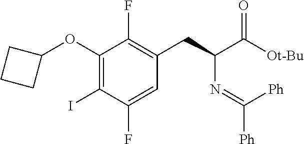 [SMILES: CC(C)(C)OC(=O)[C@H](Cc1cc(F)c(I)c(OC2CCC2)c1F)N=C(c1ccccc1)c1ccccc1]

### tert-Butyl (R)-3-(3-cyclobutoxy-2,5-difluoro-4-iodophenyl)-2-((diphenylmethylene)amino)propanoate (Example 54)

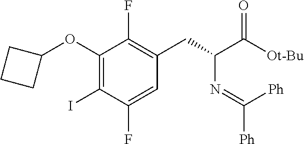 [SMILES: CC(C)(C)OC(=O)[C@@H](Cc1cc(F)c(I)c(OC2CCC2)c1F)N=C(c1ccccc1)c1ccccc1]

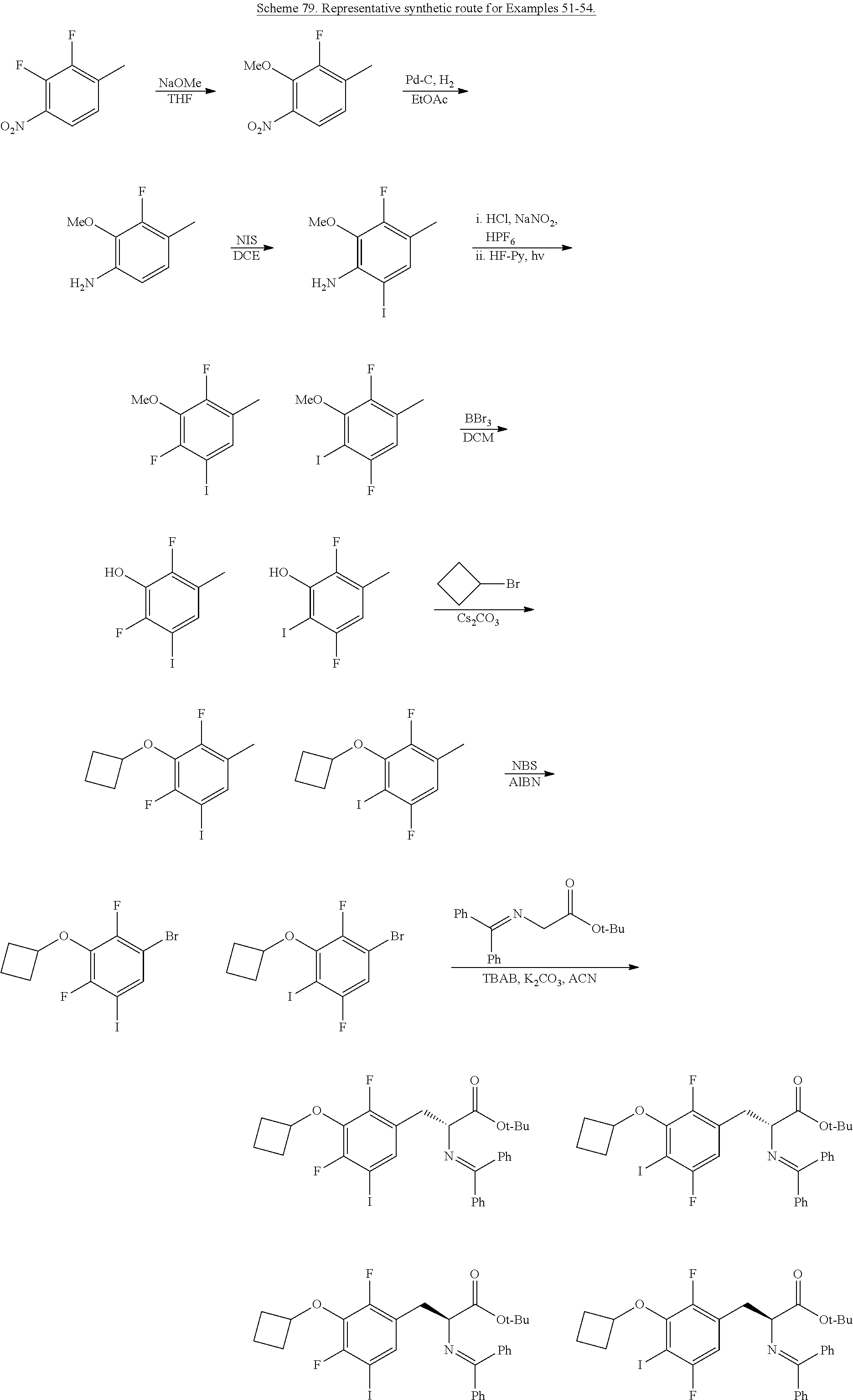

### Step 1: 2-fluoro-3-methoxy-1-methyl-4-nitrobenzene

To a stirred mixture of 2,3-difluoro-1-methyl-4-nitrobenzene (45.0 g, 260 mmol) in THF (800 mL) was added NaOMe (30% in MeOH, 46.8 g, 260 mmol), dropwise. After stirring for 2 h, the mixture was diluted with ice-water (2 L), partially concentrated under reduced pressure and extracted with EtOAc (2×1.5 L). The combined organic layers were washed with brine (2 L), dried over anhydrous Na₂SO₄ and concentrated under reduced pressure. The crude residue was purified by silica gel column chromatography (EA-petroleum ether, 9%) to afford the title compound (40 g, 83%) as a yellow oil. GCMS (EI, m/z): 185.10 [M].

### Step 2: 3-fluoro-2-methoxy-4-methylaniline

To a solution of 2-fluoro-3-methoxy-1-methyl-4-nitrobenzene (26.0 g, 140 mmol) in EtOAc (500 mL) was added Pd—C (10% on carbon, 26 g). The mixture was evacuated and backfilled with (3×), then pressurized with H₂ (˜2 atm) and stirred at 30° C. After 16 h, the system was vented, flushed with N₂ and the mixture was filtered through a celite pad, washing with EtOAc. The filtrate was concentrated under reduced pressure and the crude residue was purified by silica gel chromatography (EA-petroleum ether, 9%), affording the title compound (18 g, 82%) as a red oil. LCMS (ES, m/z): 156.20 [M+H]⁺.

### Step 3: 3-fluoro-6-iodo-2-methoxy-4-methylaniline

To a solution of N-iodosuccinimide (17.4 g, 77.3 mmol) and thianthrene (1.39 g, 6.44 mmol) in DCE (400 mL) was added TfOH (0.97 g, 6.44 mmol) followed by 3-fluoro-2-methoxy-4-methylaniline (10.0 g, 64.4 mmol). After 2 h, the mixture was concentrated under reduced pressure and the crude product was purified by reversed-phase flash chromatography (C18 silica gel, ACN-water, 10-100% with 0.1% FA), affording the title compound (6.7 g, 37%) as a yellow oil. LCMS (ES, m/z): 282.20 [M+H]⁺

### Step 4: 2,4-difluoro-1-iodo-3-methoxy-5-methylbenzene and 1,4-difluoro-2-iodo-3-methoxy-5-methylbenzene

To a mixture of 3-fluoro-6-iodo-2-methoxy-4-methylaniline (6.7 g, 23.8 mmol) in HCl (6 M, 135 mL) at 0° C. was added a solution of NaNO₂ (2.47 g, 35.7 mmol) in water (5 mL), dropwise. The mixture was stirred for. After 1 h at 0° C., hexafluorophosphoric acid (60% in H₂O, 11.6 g, 47.7 mmol) was added to the mixture, dropwise. After an additional 30 min at 0° C., the mixture was filtered, the filter cake was washed with cold water (10 mL) then dried under an IR lamp to afford a diazonium salt (10 g). The diazonium salt was dissolved in hydrogen fluoride-pyridine (70%, 400 mL) and the resulting solution was circulated through a medium pressure mercury lamp flow reactor (1.3 mL/min) for 1 h at rt, whereupon the mixture was poured into ice-water (1.5 L) and extracted with EA (2×1 L). The combined organic layers were washed with brine (1.5 L), dried over anhydrous Na₂SO₄ and concentrated under reduced pressure. The crude product was purified by silica gel chromatography (THF-petroleum ether, 9%) to afford a 1.2:1 mixture 2,4-difluoro-1-iodo-3-methoxy-5-methylbenzene to 1,4-difluoro-2-iodo-3-methoxy-5-methylbenzene (5 g, 73%) as a light-yellow oil. GCMS (EI, m/z): 284.10 [M].

### Step 5: 2,6-difluoro-3-iodo-5-methylphenol and 2,5-difluoro-6-iodo-3-methylphenol

To a solution of 2,4-difluoro-1-iodo-3-methoxy-5-methylbenzene and 1,4-difluoro-2-iodo-3-methoxy-5-methylbenzene (1.00 g, 3.52 mmol) in DCM (15 mL) at 0° C. was added PBr₃ (7.00 mL, 7.04 mmol). After 16 h at rt, the mixture was diluted with ice-water (100 mL) and extracted with EtOAc (2×100 mL). The combined organic layers were washed with brine (100 mL), dried over anhydrous Na₂SO₄ and concentrated under reduced pressure. The crude residue was purified by silica gel chromatography (EA-petroleum ether, 9%) to afford a mixture of the title compounds (750 mg, 79%) as a light brown oil. LCMS (ES, m/z): 269.30 [M−H]⁻.

### Step 6: 3-cyclobutoxy-2,4-difluoro-1-iodo-5-methylbenzene and 3-cyclobutoxy-1,4-difluoro-2-iodo-5-methylbenzene

A mixture of 2,6-difluoro-3-iodo-5-methylphenol and 2,5-difluoro-6-iodo-3-methylphenol (750 mg, 2.78 mmol), bromocyclobutane (558 mg, 4.17 mmol) and Cs₂CO₃ (1.81 g, 5.56 mmol) in DMF (20 mL) was heated at 60° C. After 16 h, the mixture was cool to rt, diluted with water (150 mL) and extracted with EtOAc (2×120 mL). The combined organic layers were washed with brine (3×100 mL), dried over anhydrous Na₂SO₄ and concentrated under reduced pressure. The residue was purified by silica gel chromatography (EA-petroleum ether, 9%) to afford a mixture of the title compounds (600 mg, 67%) as a yellow oil. GCMS (ES, m/z): 323.90 [M].

### Step 7: 1-(bromomethyl)-3-cyclobutoxy-2,4-difluoro-5-iodobenzene and 1-(bromomethyl)-3-cyclobutoxy-2,5-difluoro-4-iodobenzene

A mixture of 3-cyclobutoxy-2,4-difluoro-1-iodo-5-methylbenzene and 3-cyclobutoxy-1,4-difluoro-2-iodo-5-methylbenzene (600 mg, 1.85 mmol), NBS (494 mg, 2.78 mmol) and AIBN (30 mg, 0.19 mmol) in DCE (4 mL) was heated at 70° C. After 16 h, the mixture was cooled to rt, concentrated under reduced pressure and purified by silica gel chromatography (EA-petroleum ether, 9%) to afford a mixture of the title compounds (300 mg, 40%) as a colorless oil. GCMS (ELI, m/z): 401.90, 403.9 [M].

### Step 7: (S)-tert-butyl 3-(3-cyclobutoxy-2,4-difluoro-5-iodophenyl)-2-(diphenylmethyleneamino)propanoate, (R)-tert-butyl 3-(3-cyclobutoxy-2,4-difluoro-5-iodophenyl)-2-(diphenylmethyleneamino)propanoate, (S)-tert-butyl 3-(3-cyclobutoxy-2,5-difluoro-4-iodophenyl)-2-(diphenylmethyleneamino)propanoate and (R)-tert-butyl 3-(3-cyclobutoxy-2,5-difluoro-4-iodophenyl)-2-(diphenylmethyleneamino)propanoate

A mixture of 1-(bromomethyl)-3-cyclobutoxy-2,4-difluoro-5-iodobenzene and 1-(bromomethyl)-3-cyclobutoxy-2,5-difluoro-4-iodobenzene (300 mg, 0.75 mmol), tetrabutylammonium bromide (48 mg, 0.15 mmol), K₂CO₃ (309 mg, 2.24 mmol) and tert-butyl 2-(diphenylmethyleneamino)acetate (221 mg, 0.75 mmol) in MeCN (6 mL) was stirred for 16 h at rt, whereupon it was filtered directly purified by reversed-phase flash chromatography (C18 silica gel, ACN-water, 10-100% with 0.1% FA), affording a mixture of products. The mixture was further purified by achiral-SFC (Column: greensep naphthyl 5 um, 30×250 mm; Mobile phase A: CO₂, mobile phase B: 24% MeOH (with 0.3%-7M-NH₃-MeOH); Flow rate: 75 mL/min; Column temperature: 35° C.; Back pressure: 100 bar; Wavelength: 220 nm) to afford a mixture of tert-butyl 3-(3-cyclobutoxy-2,4-difluoro-5-iodophenyl)-2-(diphenylmethyleneamino)propanoate and tert-butyl 3-(3-cyclobutoxy-2,5-difluoro-4-iodophenyl)-2-(diphenylmethyleneamino)propanoate (180 mg). The mixture was further purified by Prep-SFC [Column: (R, R)-WHELK-O1 5 μm, 3×25 cm; Mobile Phase A: CO₂, Mobile Phase B: 30% IPA (with 1% 2M NH₃ in MeOH); Flow rate: 100 mL/min; Column Temperature: 35° C.; Back Pressure: 100 bar; WaveLength: 220 nm] to afford a mixture of (S)-tert-butyl 3-(3-cyclobutoxy-2,4-difluoro-5-iodophenyl)-2-(diphenylmethyleneamino)propanoate and (S)-tert-butyl 3-(3-cyclobutoxy-2,5-difluoro-4-iodophenyl)-2-(diphenylmethyleneamino)propanoate (first eluting sample, 80 mg), then (R)-tert-butyl 3-(3-cyclobutoxy-2,5-difluoro-4-iodophenyl)-2-(diphenylmethyleneamino)propanoate (second eluting sample, 10 mg, 2%) followed by (R)-tert-butyl 3-(3-cyclobutoxy-2,4-difluoro-5-iodophenyl)-2-(diphenylmethyleneamino)propanoate (third eluting sample, 60 mg, 13%) as colorless oils. The mixture of (S)-tert-butyl 3-(3-cyclobutoxy-2,4-difluoro-5-iodophenyl)-2-(diphenylmethyleneamino)propanoate and (S)-tert-butyl 3-(3-cyclobutoxy-2,5-difluoro-4-iodophenyl)-2-(diphenylmethyleneamino)propanoate was further purified by Prep-chiral HPLC (Column: CHIRALPAK AD-H 5 m, 2×25 cm; Mobile Phase A: Hexanes; Mobile Phase B: IPA; Flow rate: 20 mL/min; Wavelength: 254/221 nm) to afford (S)-tert-butyl 3-(3-cyclobutoxy-2,5-difluoro-4-iodophenyl)-2-(diphenylmethyleneamino)propanoate (first eluting isomer, 10 mg, 2%) and (S)-tert-butyl 3-(3-cyclobutoxy-2,4-difluoro-5-iodophenyl)-2-(diphenylmethyleneamino)propanoate (60 mg, 13%) as colorless oils.

### tert-butyl (S)-3-(3-cyclobutoxy-2,4-difluoro-5-iodophenyl)-2-((diphenylmethylene)amino)propanoate

¹H NMR (400 MHz, DMSO-d₆) δ 7.51-7.35 (m, 8H), 7.32 (t, J=7.1 Hz, 1H), 6.69 (d, J=6.0 Hz, 2H), 4.41 (p, J=7.4 Hz, 1H), 4.00 (dd, J=8.5, 5.2 Hz, 1H), 3.13-2.99 (m, 2H), 2.15-2.05 (m, 1H), 2.04-1.89 (m, 3H), 1.63-1.52 (m, 1H), 1.38 (s, 9H), 1.37-1.28 (m, 1H).

¹⁹F NMR (376 MHz, DMSO-d₆) δ −109.93 (d, J=8.6 Hz), −129.99 (d, J=9.3 Hz).

LCMS (ES, m/z): 618.05 [M+H]⁺.

### tert-butyl (R)-3-(3-cyclobutoxy-2,4-difluoro-5-iodophenyl)-2-((diphenylmethylene)amino)propanoate

¹H NMR (400 MHz, DMSO-d₆) δ 7.53-7.35 (m, 8H), 7.32 (t, J=7.1 Hz, 1H), 6.69 (d, J=5.6 Hz, 2H), 4.41 (p, J=7.3 Hz, 1H), 4.00 (dd, J=8.1, 5.2 Hz, 1H), 3.13-2.99 (m, 2H), 2.16-2.05 (m, 1H), 2.05-1.89 (m, 3H), 1.64-1.52 (m, 1H), 1.38 (s, 9H), 1.37-1.27 (m, 1H).

¹⁹F NMR (376 MHz, DMSO-d₆) δ −109.93 (d, J=8.8 Hz), −129.99 (d, J=9.1 Hz).

LCMS (ES, m/z): 618.05 [M+H]⁺; 97.2% purity (254 nm).

### (S)-tert-butyl 3-(3-cyclobutoxy-2,5-difluoro-4-iodophenyl)-2-(diphenylmethyleneamino)propanoate

¹H NMR (400 MHz, DMSO-d₆) δ 7.53-7.35 (m, 8H), 6.83 (dd, J=8.2, 5.3 Hz, 1H), 6.68 (d, J=6.6 Hz, 2H), 4.39 (p, J=7.3 Hz, 1H), 4.05 (dd, J=8.0, 5.4 Hz, 1H), 3.17-3.03 (m, 2H), 2.22-2.03 (m, 3H), 2.02-1.90 (m, 1H), 1.68-1.57 (m, 1H), 1.38 (s, 9H), 1.37-1.27 (m, 1H).

¹⁹F NMR (376 MHz, DMSO-d₆) δ −98.74 (d, J=14.5 Hz), −135.35 (d, J=14.4 Hz).

LCMS (ES, m/z): 618.00 [M+H]⁺.

### (R)-tert-butyl 3-(3-cyclobutoxy-2,5-difluoro-4-iodophenyl)-2-(diphenylmethyleneamino)propanoate

¹H NMR (400 MHz, DMSO-d₆) δ 7.53-7.35 (m, 8H), 6.83 (dd, J=8.1, 5.3 Hz, 1H), 6.68 (d, J=6.6 Hz, 2H), 4.39 (p, J=7.3 Hz, 1H), 4.05 (dd, J=7.9, 5.4 Hz, 1H), 3.16-3.03 (m, 2H), 2.23-2.02 (m, 3H), 2.02-1.90 (m, 1H), 1.69-1.57 (m, 1H), 1.38 (s, 9H), 1.37-1.27 (m, 1H).

¹⁹F NMR (376 MHz, DMSO-d₆) δ −98.74 (d, J=14.9 Hz), −135.35 (d, J=14.2 Hz).

LCMS (ES, m/z): 618.10 [M+H]⁺.

### Example 55: General Methods and Materials for Photoredox [¹⁸F]Fluorination

Photocatalyst (Mes-Acr-Ph⁺ClO₄⁻), anhydrous MeCN, anhydrous [¹⁸F]TBAF, and 20% TBAB MeCN solution, t-BuOH, dichloroethane were obtained according to the reported literature. Radio-HPLC was carried out with a Shimadzu LC-20AT Prominence equipped with a UV detector followed by a γ-detector.

HPLC condition 1-1 (for isolation and analysis of [¹⁸F]F-amino acid derivatives before hydrolysis): Column: Phenomenex, Kinetex® EVO C18 5 μm, 100 Å, 250×4.6 mm; Solvent A: 0.1% TFA water; Solvent B: 0.1% TFA ACN; Grad/isocrat: 0 to 2 min: 20% to 20% solvent B, 2 to 22 min: 20% to 95% solvent B, 22 to 30 min: 95% to 95% solvent B, 30 to 30.1 min: 95% to 20% solvent B, 30.1 to 35 min: isocratic elution at 20% solvent B. Flow rate: 1 mL/min, column temperature: 19 to 21° C.

HPLC condition 1-2 (for isolation and analysis of [¹⁸F]F-amino acid derivatives before hydrolysis): Column: Phenomenex, Kinetex® EVO C18 5 μm, 100 Å, 250×4.6 mm. Solvent A: 0.1% TFA water; Solvent B: 0.1% TFA MeCN; Grad/isocrat: 0 to 2 min: 5% to 5% solvent B, 2 to 22 min: 5% to 95% solvent B, 22 to 30 min: 95% to 95% solvent B, 30 to 30.1 min: 95% to 5% solvent B, 30.1 to 35 min: isocratic elution at 5% solvent B. Flow rate: 1 mL/min, column temperature: 19 to 21° C.

HPLC condition 2 (for isolation and analysis of [¹⁸F]F-amino acids): Column: Phenomenex, Kinetex® EVO C18 5 μm 100 Å, 250×4.6 mm; Solvent A: 0.1% TFA water; Solvent B: 0.1% TFA MeCN; Grad/isocrat: 0 to 12 min: 15% solvent B, 12 to 22 min: 15% to 95% solvent B, 22 to 28 min: 95% solvent B, 28 to 30 min: 95% to 15% solvent B, 30 to 35 min: isocratic elution at 15% solvent B. Flow rate: 1 mL/min, column temperature: 19 to 21° C.

HPLC condition 3 (for isolation and analysis of [¹⁸F]F-amino acids): Column: Phenomenex, Kinetex® EVO C18 5 μm 100 Å, 250×4.6 mm; Solvent A: 0.1% TFA water; Solvent B: 0.1% TFA MeCN; Grad/isocrat: 0 to 8 min: 15% solvent B, 8 to 22 min: 15% to 35% solvent B, 22 to 28 min: 95% solvent B, 28 to 30 min: 95% to 15% solvent B, 30 to 35 min: isocratic elution at 15% solvent B. Flow rate: 1 mL/min, column temperature: 19 to 21° C.

HPLC condition 4 (for isolation and analysis of [¹⁸F]F-amino acids): Column: Phenomenex, Kinetex® EVO C18 5 μm 100 Å, 250×4.6 mm; Solvent A: 0.1% TFA water; Solvent B: 0.1% TFA MeCN; Grad/isocrat: 0 to 13 min: 10% solvent B, 13 to 22 min: 15% to 45% solvent B, 22 to 28 min: 95% solvent B, 28 to 30 min: 95% to 10% solvent B, 30 to 35 min: isocratic elution at 10% solvent B. Flow rate: 1 mL/min, column temperature: 19 to 21° C.

Procedure A1 for Organic Photoredox ¹⁹F/¹⁸F Exchange:

[¹⁸F]Fluoride was produced via the ¹⁸O(p,n)¹⁸F reaction in a GE PETTrace cyclotron. The aqueous (H₂¹⁸O) solution of [¹⁸F]fluoride was directly trapped on a pre-activated QMA cartridge before elution into the reactor vessel (5 mL V-vial) with an aqueous MeCN solution of the photocatalyst (Mes-Acr-Ph⁺ClO₄⁻, 3.5 mg). This solution was azeotropically dried under nitrogen and then cooled down for later usage before being measured by the dose calibrator (typically 3.0 to 7.0 GBq).

The precursor (0.03 mmol) was added in an Eppendorf tube (2 mL) and dissolved with a mixed solvent to form a solution, which contains anhydrous MeCN (100 μL in total), t-BuOH (400 μL) and dichloromethane (DCE, 350 μL). The resulting solution (˜850 μL) was added to the V-vial containing the [¹⁸F]fluoride source and photocatalyst. Then the open V-vial was illuminated top-down using a 450 nm laser (450 nm, 3.5 W after fiber coupling) with a nitrogen (N₂) balloon sparge in an ice bath for 30 min. The resulting reaction solution was diluted with MeCN (0.5 mL) and passed through an aluminum cartridge (preconditioned with 10 mL DI water) to remove the unconverted ¹⁸F-fluoride. Then the reaction vial and cartridge were rinsed with additional MeCN (0.4 mL). The total elution was then measured by the dose calibrator for radio-chemical yield analysis purposes. An aliquot of the elution was added into an Eppendorf tube containing a MeCN/EtOH solution (50/50 μL), followed by an acetic acid aqueous solution (5%, 700 μL) before the whole solution was injected into a C18-reversed-phase HPLC for radio-HPLC analysis (by using HPLC condition 1).

### Procedure A2 for Deprotection and Radio-Product Isolation:

All the rest of the elution obtained from the aluminum cartridge above was then transferred into another 5 mL V-vial before being capped with an aluminum crimp seal with PTFE/white silicone septa and then the solvent was removed under 95° C. with a nitrogen stream. HCl (6N, 300 L) was then added to the V-vial, and the mixture was heated under 110° C. for 10 min. After cooling down, a saturated NaHCO₃ solution (500 μL) was slowly added to the cooled and vented V-vial. The resulting solution was purified on HPLC (by using HPLC condition 2) to yield the final ¹⁸F-labeled amino acid product (3.7 MBq to 37.0 MBq) with a high radiochemical purity (RCP, 98.0%). The radiochemical yields (RCYs) of all ¹⁸F-labeled molecules were calculated based on the HPLC-isolated products. The ¹⁸F-radiolabeled products were confirmed by HPLC co-injection with the ¹⁹F standard. Quality control (QC) was run separately to ensure the purity of the isolated radiolabeled products.

In vivo formulation: a certain volume of NaOH (1N) was added into the vial containing the final product HPLC solution (containing 0.1% TFA) according to the volume of the solution to ensure the pH was close to 9. Then the solution was rotary evaporated till less than 100 μL solution remained in the vial. Phosphate-buffered saline (PBS, lx) and sodium ascorbate (10 mM) were added in a volume ratio of 3/1 to the vial to form an injectable solution for the mice imaging study (150 L/mouse).

### Procedure B1 for Organic Photoredox Deoxyradiofluorination:

[¹⁸F]Fluoride was produced via the ¹⁸O(p,n)¹⁸F reaction in a GE PETTrace cyclotron. The aqueous (H₂¹⁸O) solution of [¹⁸F]fluoride was directly trapped on a pre-activated QMA cartridge before elution into the reactor vessel (5 mL V-vial) with an aqueous solution of tetrabutylammonium bicarbonate (TBAB). This solution was azeotropically dried under nitrogen and then redissolved with anhydrous MeCN.

The photocatalyst (1.5 mg), precursor (0.03 mmol), anhydrous TBAHCO₃ solution (25 μL in MeCN) and [¹⁸F]TBAF solution in MeCN (typically 0.37 to 3.7 GBq, typically less than 40 μL) were added in a 5 mL V-vial and dissolved with a mixed solvent to form a solution, which contains anhydrous MeCN (100 μL in total), t-BuOH (400 μL) and dichloromethane (350 μL). The total amount of [¹⁸F]TBAF activity in the reaction was measured by the dose calibrator. The resulting solution (˜850 μL) was illuminated top-down using a 450 nm laser (450 nm, 3.5 W after fiber coupling) with an oxygen (O₂) balloon sparge in an ice bath for 30 min. The resulting reaction solution was diluted with MeCN (0.5 mL) and passed through an aluminum cartridge (preconditioned with 10 mL DI water) to remove the unconverted ¹⁸F-fluoride. Then the reaction vial and cartridge were rinsed with additional MeCN (0.4 mL). The total elution was then measured by the dose calibrator for radio-chemical yield analysis purposes. An aliquot of the elution was added into an Eppendorf tube containing a MeCN/EtOH solution (50/50 μL), followed by an acetic acid aqueous solution (5%, 700 μL) before the whole solution was injected into a C18-reversed-phase HPLC for radio-HPLC analysis (by using HPLC condition 1).

### Procedure B2 for Deprotection and Radio-Product Isolation:

All the rest of the elution obtained from the aluminum cartridge above was then transferred into another 5 mL V-vial before being capped with an aluminum crimp seal with PTFE/white silicone septa and then the solvent was removed under 95° C. with a nitrogen stream. HCl (250 μL , 6N) was then added to the V-vial, and the mixture was heated at 95° C. for 15 min. After cooling down, a saturated NaHCO₃ solution (400 μL) was slowly added to the cooled and vented V-vial. The resulting solution was purified on HPLC (by using HPLC condition 3 or 4) to yield the final ¹⁸F-labeled amino acid product (3.7 MBq to 14.0 MBq) with a high radiochemical purity (RCP, 98.0%). The RCYs of all ¹⁸F-labeled molecules were calculated based on the HPLC-isolated products. The ¹⁸F-radiolabeled products were confirmed by HPLC co-injection with the ¹⁹F standard. Quality control (QC) was run separately to ensure the purity of the isolated radiolabeled products.

In vivo formulation: a certain volume of NaOH (1N) was added into the vial containing the final product HPLC solution (containing 0.1% TFA) according to the volume of the solution to ensure the pH was close to 7. Then the solution was rotary evaporated till less than 100 μL solution remained in the vial. Phosphate-buffered saline (PBS, lx) and sodium ascorbate (10 mM) were added in a volume ratio of 3/1 to the vial to form an injectable solution for the mice imaging study (150 μL/mouse).

### In Vivo Studies (Tumor Xenografts in Murine Model)

All animal studies were carried out according to the protocols approved by the Institutional Animal Care and Use Committee at the University of North Carolina at Chapel Hill. Female Nude mice (5-6 weeks old) were obtained from the Division of Comparative Medicine at University of North Carolina at Chapel Hill. Mice were housed in Tecniplast GM500 ventilated cages in a temperature-controlled room with 12 h light:12 h dark cycles, with food and water ad libitum.

To induce human breast cancer MCF-7 xenografts, 17-beta-estradiol pellets (7.5 mg/pellet, 60-day release, Cat #: SE-121) were surgically implanted subcutaneously behind the neck. Approximately 7 days later, after the suture was removed, approximately 4 million cells, resuspended in PBS and mixed with Matrigel (BD Bioscience) at a 1:1 (v:v) ratio, were inoculated subcutaneously in the left mammary fat pad. Tumor growth was monitored using a caliper, and the tumor volume was calculated using the following formula: Volume=Length*width*width*0.5. Animals were used for PET imaging or efficacy studies when the tumor size ranges between 150-1000 mm³.

For efficacy studies, animals were excluded if, before potential dosing with radionuclide-containing molecules, tumor volume (as measured above) deviated from the range of 250 mm³+/−125 mm³.

18—F studies: Each treated mouse was given quantities of [18]F containing molecule, indicated in figure labels and formulated for in vivo studies as described, via tail vein injection. When collected, PET images were acquired at the indicated times with the PET/CT imaging system SuperArgus 4R (Sedecal Inc.). List-mode data were collected and reconstructed using the algorithm described before. Regions of interest were drawn using AMIDE software (Amide's Medical Image Data Examiner). Organ uptake was expressed as mean±SD of the percentage injected dose per gram (% ID/g) and corrected for radioactive decay.

I-124 studies: NaI (10 mg/kg) was intravenously dosed (via tail vein) at 24 h and 3 h before the injection of radiolabeled molecules. Each treated mouse was given quantities of [18]F containing molecule indicated in figure labels and formulated for in vivo studies as described, via tail vein injection. When collected, PET images were acquired at the indicated times with the PET/CT imaging system SuperArgus 4R (Sedecal Inc.). List-mode data were collected and reconstructed using the algorithm described before. Regions of interest were drawn using AMIDE software (Amide's Medical Image Data Examiner). Organ uptake was expressed as mean±SD of the percentage injected dose per gram (% ID/g) and corrected for radioactive decay.

I-131 studies: NaI (10 mg/kg) was intravenously dosed (via tail vein) at 24 h and 3 h before injection of the radiolabeled molecules. Each treated mouse was given approximately ˜550 uCi of I-131 containing molecule in the first dose, and ˜1050 uCi of I-131 containing molecule 6 days later in a second dose, formulated for in vivo studies as described, via tail vein injection. Tumor volume was measured by the caliper method described above on the day of dosing and at 3 day intervals post-dosing.

For all studies, the following euthanasia criteria were used: Tumor size >2000 mm³, body weight decrease >10% for three consecutive measurements, BCS≤2.

As is readily appreciated by those skilled in the art, a method of radiolabeling a substrate with one radionuclide can be applicable to radiolabeling a substrate with another radionuclide. For example, a method of radiolabeling with [18]F, [124]I, or [131]I as described herein can be applicable to radiolabeling with., e.g., [123]I, [125]I, [211]At, [211]At, [76]Br, [77]Br, and [82]Br.

### Example 56: Labeling and imaging of (R)-2-((Diphenylmethylene)amino)-3-(3-fluoro-5-(fluoro-¹⁸F)-2-iodo-6-methoxyphenyl)propanoic acid and (R)-2-((Diphenylmethylene)amino)-3-(3-fluoro-5-(fluoro-¹⁸F)-6-iodo-2-methoxyphenyl)propanoic acid

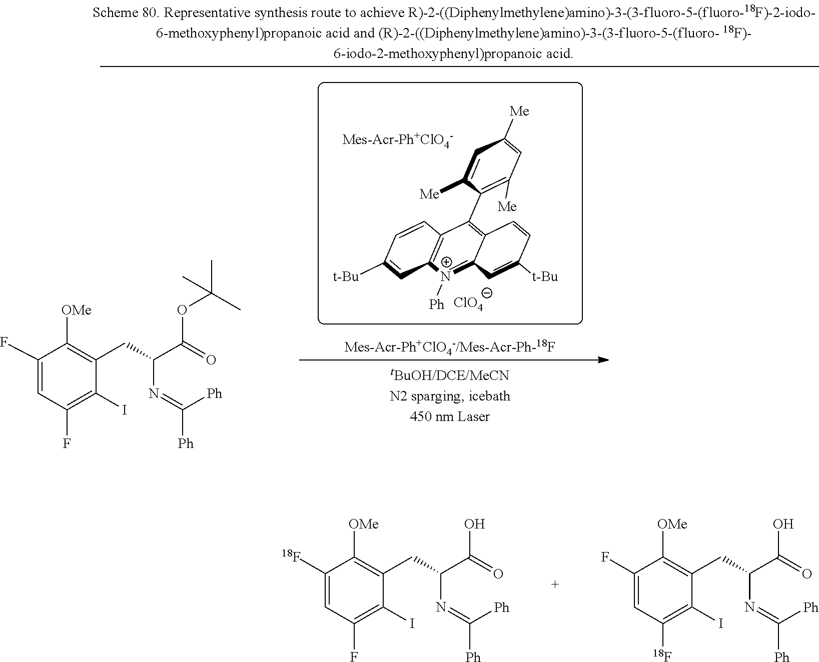

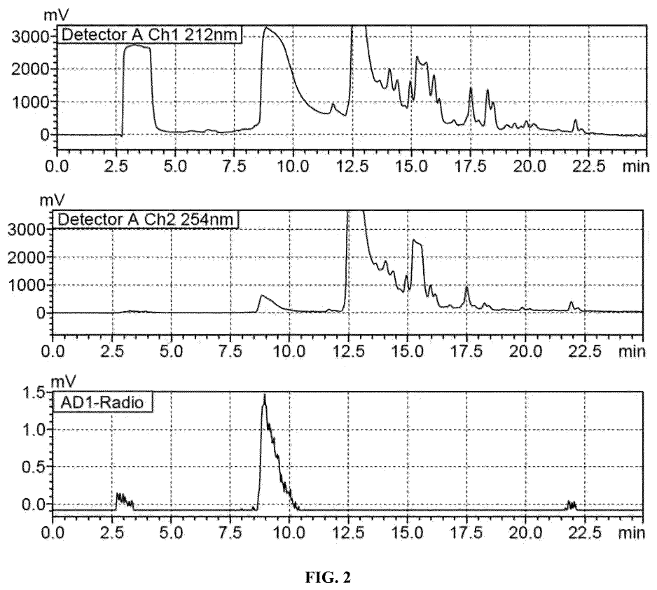

tert-Butyl (R)-3-(3,5-difluoro-2-iodo-6-methoxyphenyl)-2-((diphenylmethylene)amino)propanoate (17.3 mg, 0.03 mmol) was subjected to Procedure A1, resulting in a mixture of the title compounds (combined RCY of 12.4%, decay corrected). RadioHPLC analysis by using HPLC condition 1-1 is shown in FIG. 2.

### Example 57: Labeling and imaging of (R)-2-Amino-3-(3-fluoro-5-(fluoro-¹⁸F)-2-iodo-6-methoxyphenyl)propanoic acid and (R)-2-Amino-3-(3-fluoro-5-(fluoro-¹⁸F)-6-iodo-2-methoxyphenyl)propanoic acid

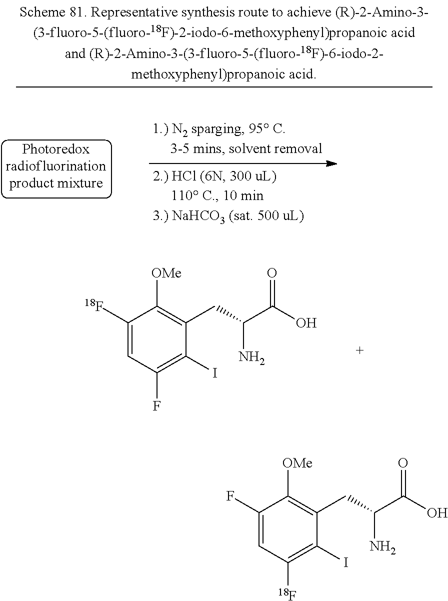 [SMILES: COc1c([18F])cc(F)c(I)c1C[C@@H](N)C(=O)O ; COc1c(F)cc([18F])c(I)c1C[C@@H](N)C(=O)O]

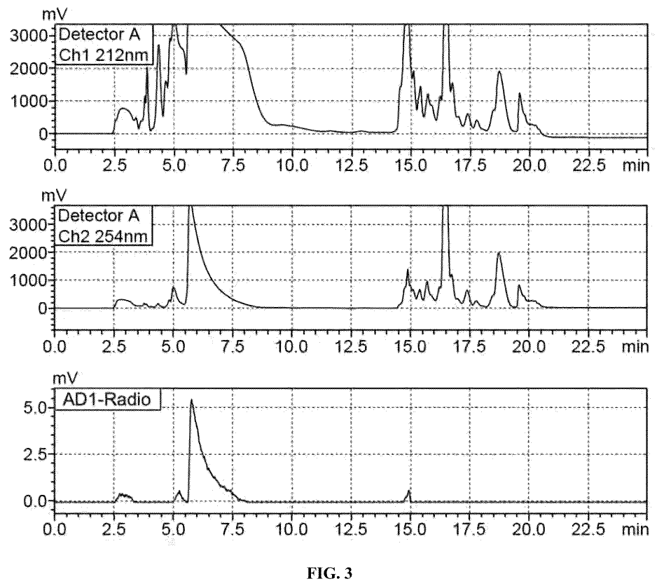

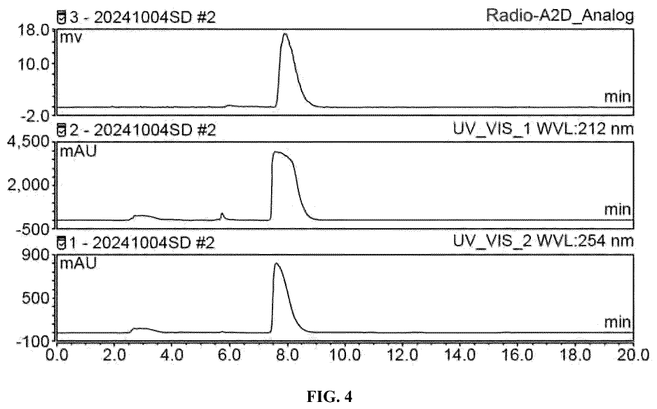

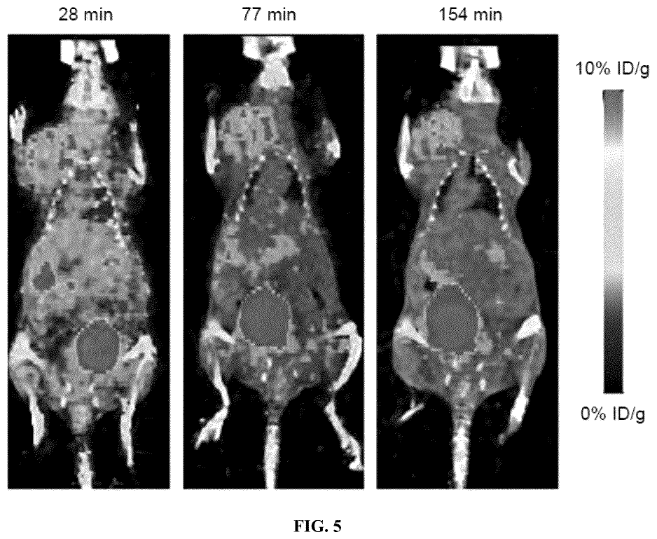

The title compounds (RCY of 1.2%, 2-steps) were produced from Example 5 by following Procedure A2. RadioHPLC isolation by using HPLC condition 2 is shown in FIG. 3. Quality Control, HPLC condition 2 is shown in FIG. 4. F-18 imaging, biodistribution for (R)-2-amino-3-(3-fluoro-5-(fluoro-¹⁸F)-2-iodo-6-methoxyphenyl)propanoic acid and (R)-2-amino-3-(3-fluoro-5-(fluoro-¹⁸F)-6-iodo-2-methoxyphenyl)propanoic acid is shown in FIG. 5.

The non-radiolabeled versions of the title compounds (cold standard compound) are made in a manner similar to those described in Example 13.

### Example 58: Labeling and imaging of (S)-2-((Diphenylmethylene)amino)-3-(3-fluoro-5-(fluoro-¹⁸F)-2-iodo-6-methoxyphenyl)propanoic acid and (S)-2-((Diphenylmethylene)amino)-3-(3-fluoro-5-(fluoro-¹⁸F)-6-iodo-2-methoxyphenyl)propanoic acid

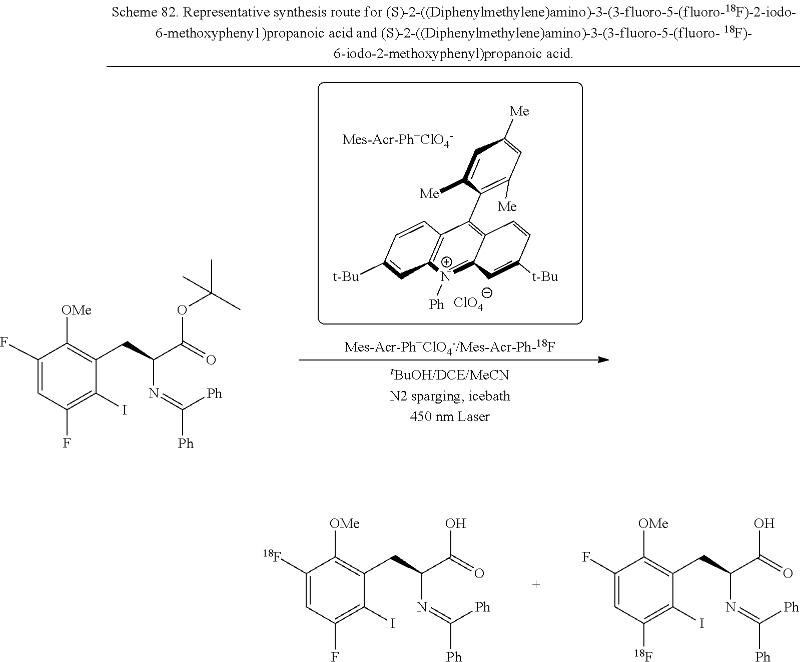

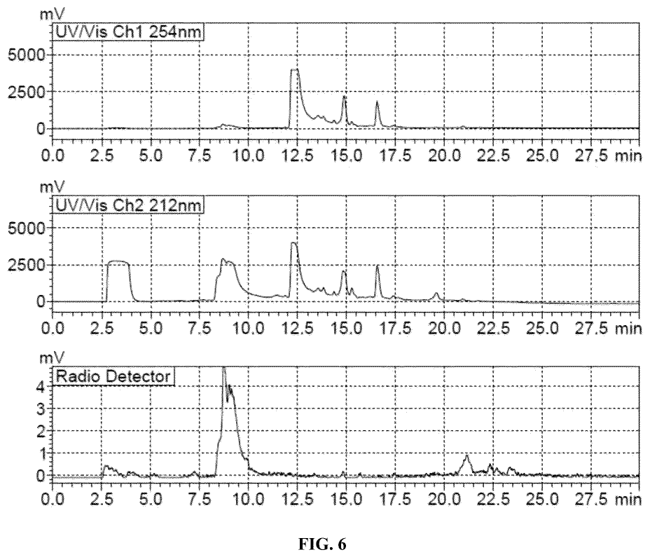

tert-Butyl (S)-3-(3,5-difluoro-2-iodo-6-methoxyphenyl)-2-((diphenylmethylene)amino)propanoate (17.3 mg, 0.03 mmol) was subjected to Procedure A1, resulting in a mixture of the title compounds (RCY of 11.7%, decay corrected). RadioHPLC analysis by using HPLC condition 1-1 is shown in FIG. 6.

### Example 59: Labeling and imaging of (S)-2-Amino-3-(3-fluoro-5-(fluoro-¹⁸F)-2-iodo-6-methoxyphenyl)propanoic acid and (S)-2-Amino-3-(3-fluoro-5-(fluoro-¹⁸F)-6-iodo-2-methoxyphenyl)propanoic acid

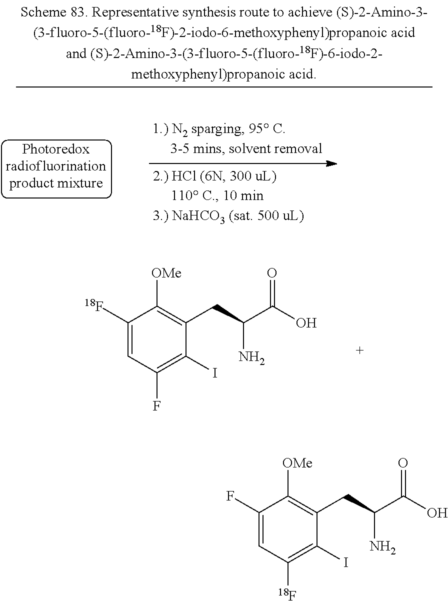 [SMILES: COc1c([18F])cc(F)c(I)c1C[C@H](N)C(=O)O ; COc1c(F)cc([18F])c(I)c1C[C@H](N)C(=O)O]

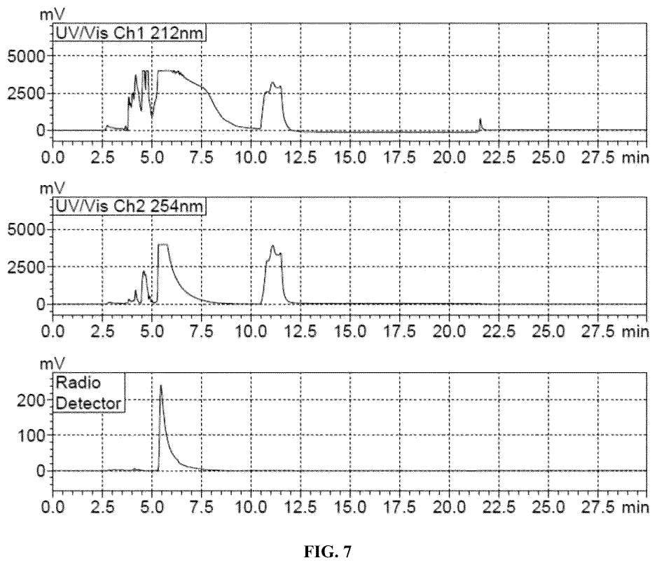

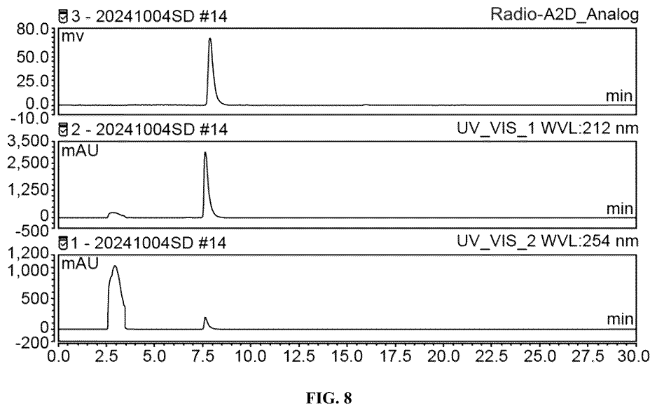

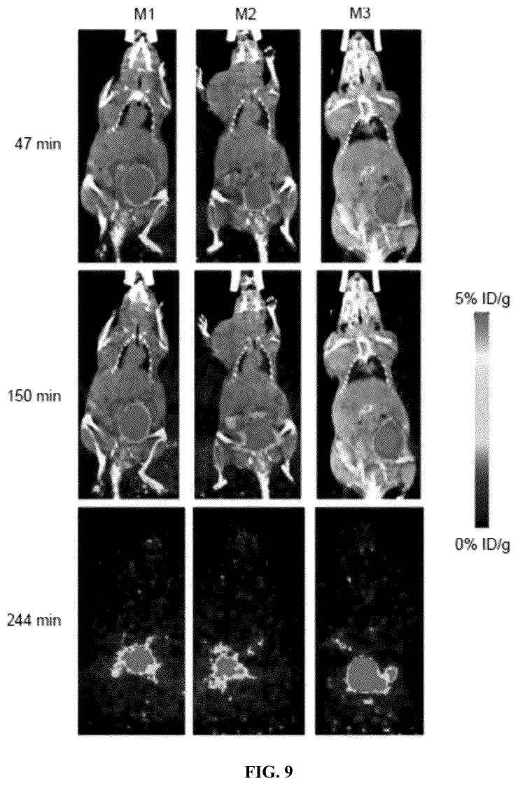

The title compounds (RCY of 6.0%, 2-steps) were produced from Example 5 by following Procedure A2. RadioHPLC isolation by using HPLC condition 2 is shown in FIG. 7. Quality Control, HPLC condition 2 is shown in FIG. 8. F-18 imaging, biodistribution for (S)-2-amino-3-(3-fluoro-5-(fluoro-¹⁸F)-2-iodo-6-methoxyphenyl)propanoic acid and (S)-2-amino-3-(3-fluoro-5-(fluoro-¹⁸F)-6-iodo-2-methoxyphenyl)propanoic acid is shown in FIG. 9.

The non-radiolabeled versions of the title compounds (cold standard compound) are made in a manner similar to those described in Example 13.

### Example 60: Labeling and imaging of (S)-2-((Diphenylmethylene)amino)-3-(2-fluoro-5-(fluoro-¹⁸F)-3-iodo-6-methoxyphenyl)propanoic acid

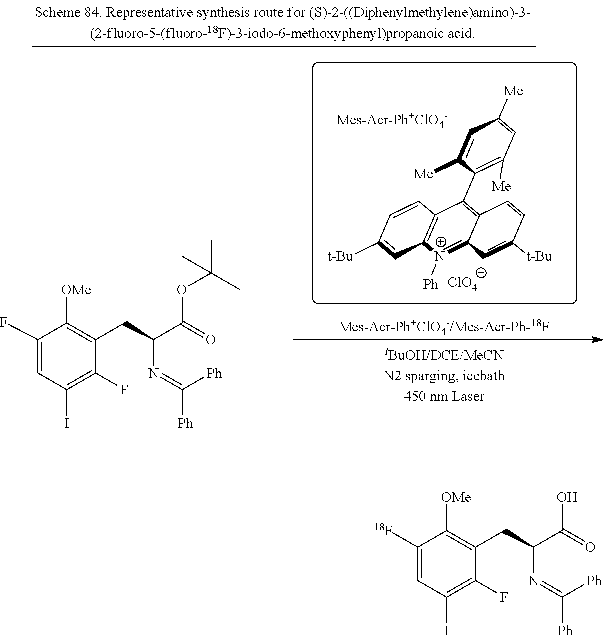

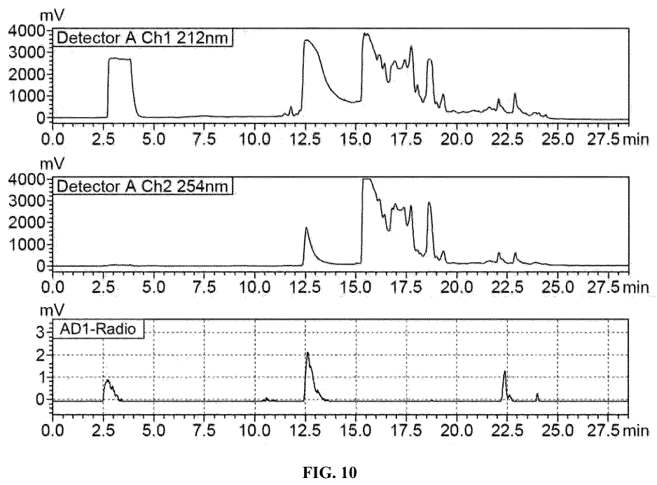

The title compound (RCY of 5.5%, decay corrected) was produced from tert-butyl (S)-3-(2,5-difluoro-3-iodo-6-methoxyphenyl)-2-((diphenylmethylene)amino)propanoate (17.3 mg, 0.03 mmol) by following Procedure A1. RadioHPLC analysis by using HPLC condition 1-2 is shown in FIG. 10.

### Example 61: Labeling and imaging of (R)-2-((Diphenylmethylene)amino)-3-(2-fluoro-5-(fluoro-¹⁸F)-3-iodo-6-methoxyphenyl)propanoic acid

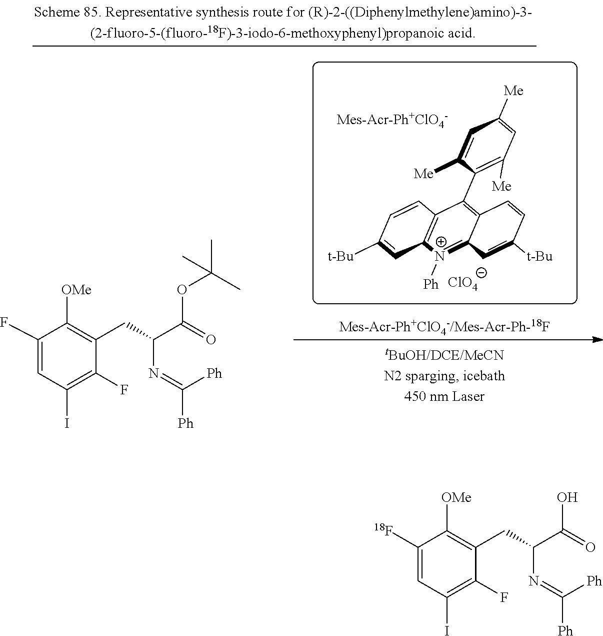

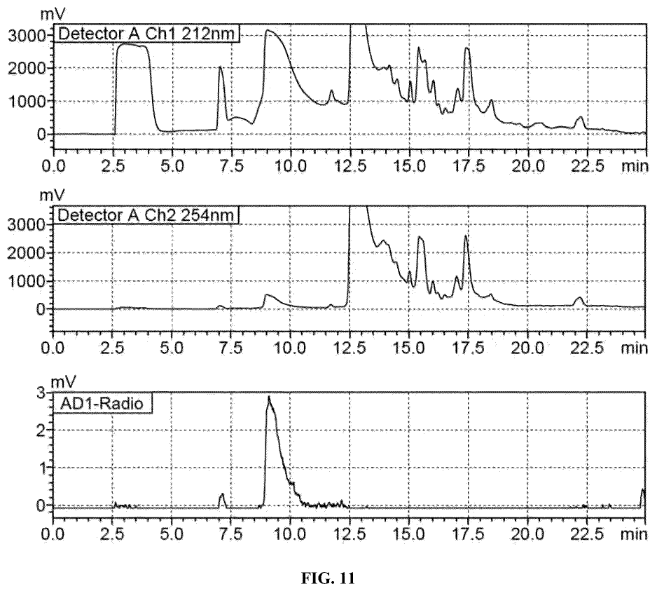

The title compound (RCY of 3.4%, decay corrected) was produced from tert-butyl (R)-3-(2,5-difluoro-3-iodo-6-methoxyphenyl)-2-((diphenylmethylene)amino)propanoate (17.3 mg, 0.03 mmol) by following Procedure A1. RadioHPLC analysis by using HPLC condition 1-1 is shown in FIG. 11.

### Example 62: Labeling and imaging of (R)-2-Amino-3-(2-fluoro-5-(fluoro-¹⁸F)-3-iodo-6-methoxyphenyl)propanoic acid

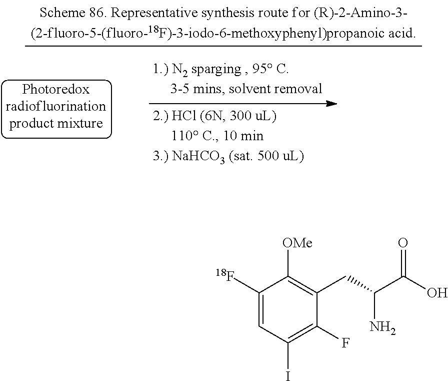 [SMILES: COc1c([18F])cc(I)c(F)c1C[C@@H](N)C(=O)O]

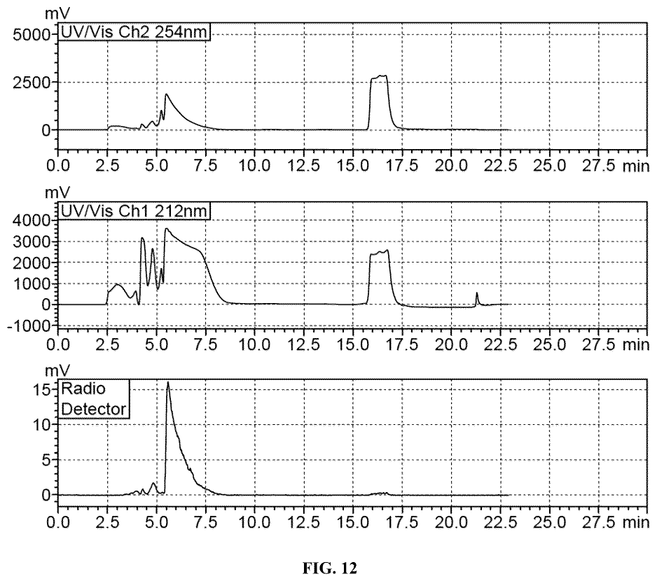

The title compound (RCY of 1.1%, 2-steps) was produced by subjecting example 7 (above) to Procedure A2. RadioHPLC isolation by using HPLC condition 2 as shown in FIG. 12.

### Example 63: Labeling and imaging of (S)-2-amino-3-(5-fluoro-4-(fluoro-¹⁸F)-2-iodophenyl)propanoic acid

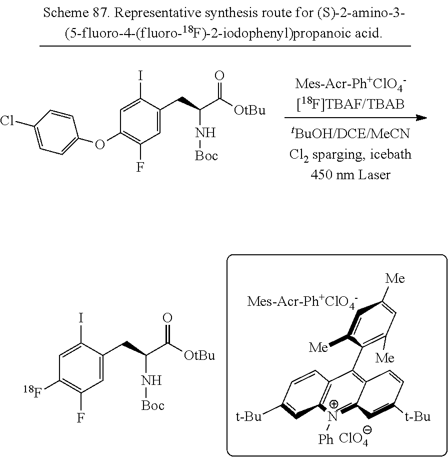

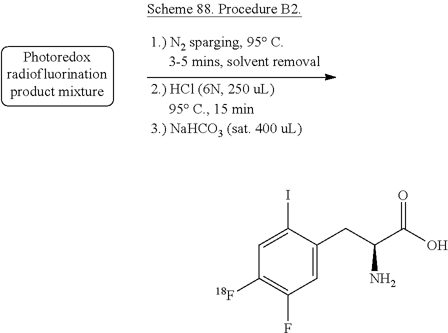 [SMILES: N[C@@H](Cc1cc(F)c([18F])cc1I)C(=O)O]

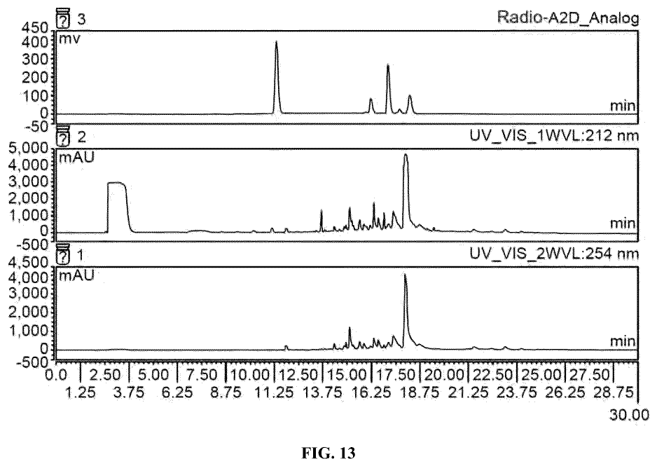

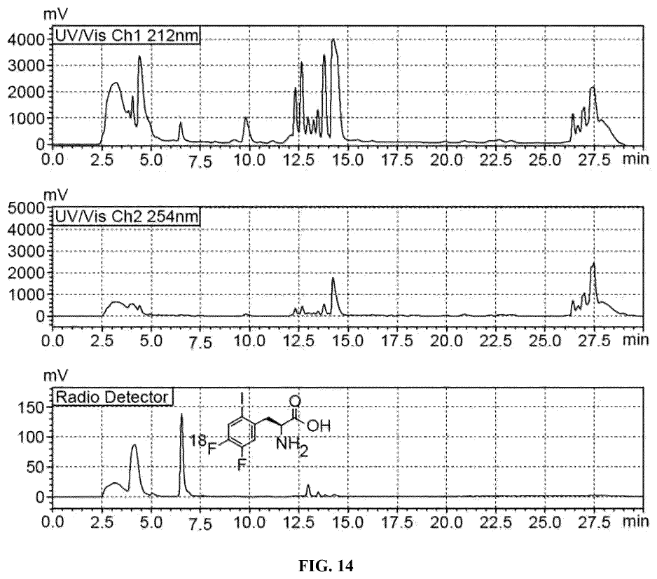

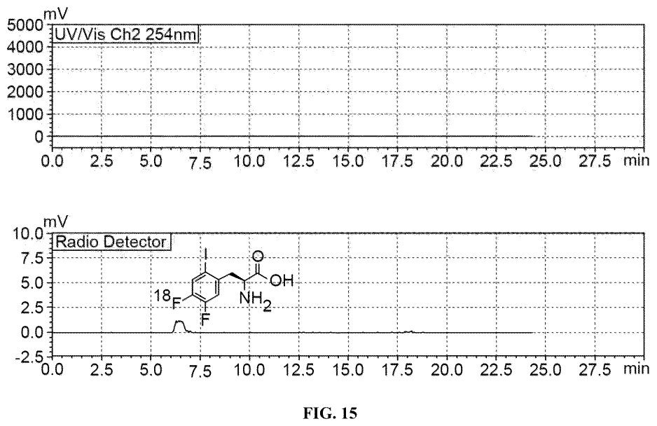

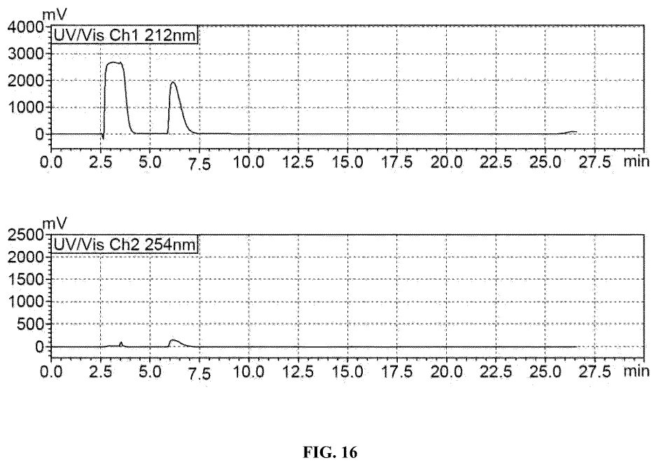

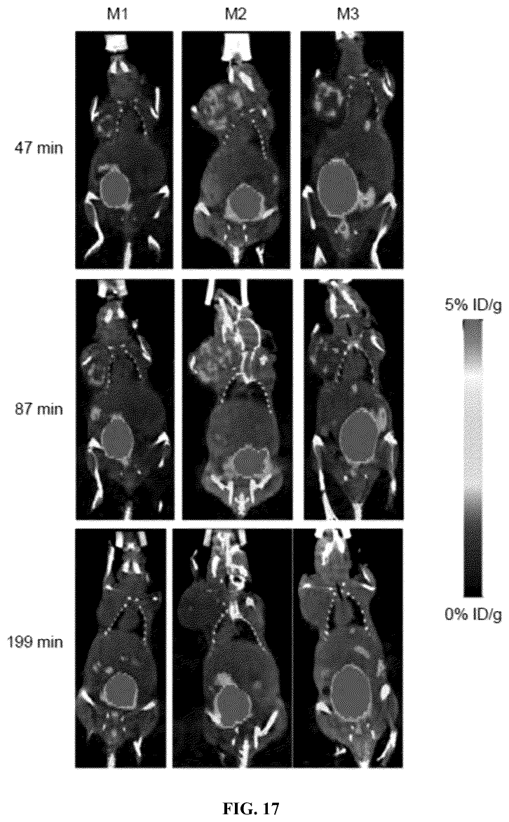

The title compound (RCY of 4.04%, 2-steps) was produced from subjecting tert-butyl (S)-2-((tert-butoxycarbonyl)amino)-3-(4-(4-chlorophenoxy)-5-fluoro-2-iodophenyl)propanoate (11.8 mg, 0.02 mmol) to Procedures B1 and B2, sequentially. Procedure B1 is shown in Scheme 87. Under procedure B1, radioHPLC was used to analyze the radiolabeling and desired radio product, as shown in FIG. 13. Procedure B2 is shown in Scheme 88. RadioHPLC isolation after B2: HPLC condition 3 is shown in FIG. 14. Quality Control: HPLC Condition 3 is shown in FIG. 15. ¹⁹F-standard is shown in FIG. 16. F-18 imaging, biodistribution for (S)-2-amino-3-(5-fluoro-4-(fluoro-¹⁸F)-2-iodophenyl)propanoic acid is shown in FIG. 17.

### Example 64: Labeling and imaging of (R)-2-amino-3-(5-fluoro-4-(fluoro-¹⁸F)-2-iodophenyl)propanoic acid

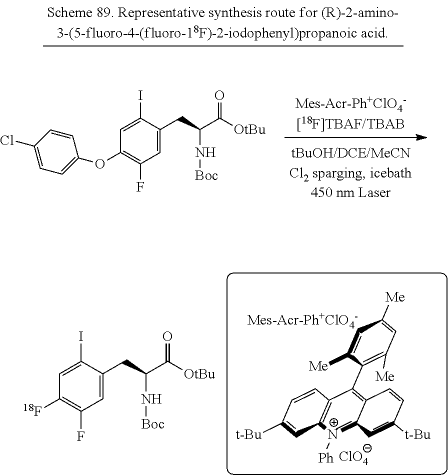

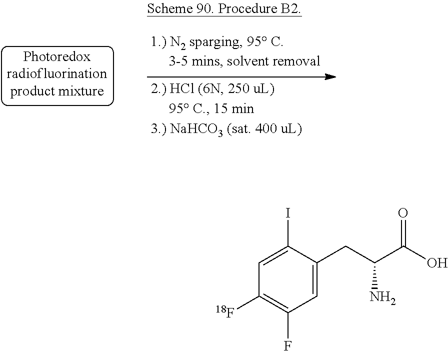 [SMILES: N[C@H](Cc1cc(F)c([18F])cc1I)C(=O)O]

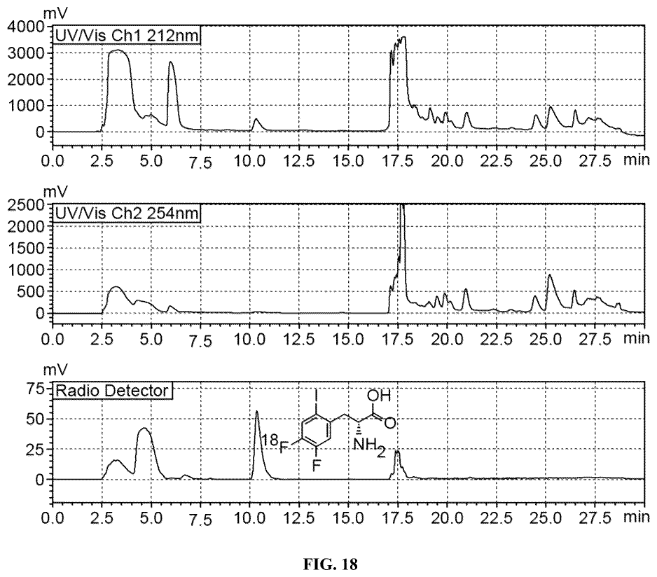

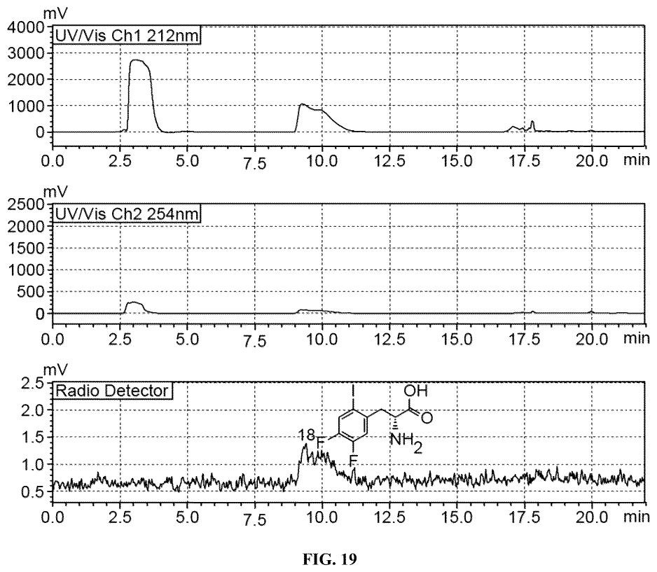

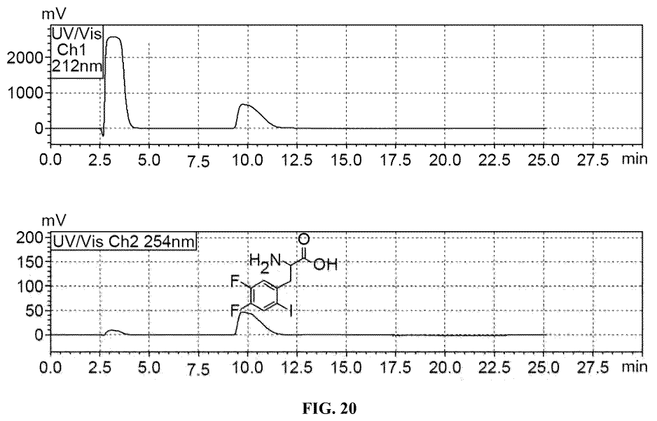

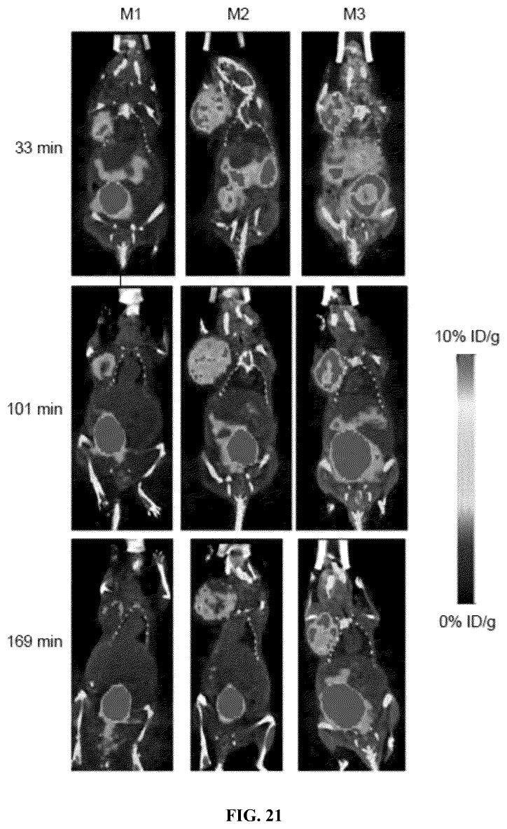

The title compound (RCY of 4.75%, 2-steps) was produced from subjecting tert-butyl (R)-2-((tert-butoxycarbonyl)amino)-3-(4-(4-chlorophenoxy)-5-fluoro-2-iodophenyl)propanoate (11.8 mg, 0.02 mmol) to Procedures B1 and B2, sequentially. Procedure B1 is shown in Scheme 89. Procedure B2 is shown in Scheme 90. RadioHPLC isolation: HPLC condition 4 is shown in FIG. 18. Co-injection analysis of the radio product by spiking with the ¹⁹F-standard: HPLC condition 4 is shown in FIG. 19. ¹⁹F-standard: HPLC condition 4 is shown in FIG. 20. F-18 imaging, biodistribution for (R)-2-amino-3-(5-fluoro-4-(fluoro-¹⁸F)-2-iodophenyl)propanoic acid is shown in FIG. 21.

### Example 65: General Methods and Materials for I-124/I-131 Radiolabeling

2,5-dihydroxybenzoic acid (>99.0%), Tin(II) sulfate (SnSO₄, >99.0%), Copper(II) sulfate pentahydrate (CuSO₄·5H₂O, >99.0%), citric acid (>99.0%) and glacial acetic acid were used as is from vendors. The Mili-Q water was used after degassed under vacuum and ultrasonication. [¹²⁴I]NaI (3 mCi) in NaOH (0.1 M) solution was purchased from 3D Imaging (Little Rock, AR). [¹³¹I]NaI solution (30 mCi) was purchased from Cardinal Health. The ¹²⁷I-amino acid precursors were prepared from the Boc and tert-butyl protected ¹²⁷I-amino acid derivatives using hydrochloric acid (6N).

HPLC condition 5 (for isolation and analysis of I-amino acids): Column: Phenomenex, Kinetex® EVO C18, 5 μm 100 Å, 250×4.6 mm LC Column. Solvent A: 0.1% TFA water; Solvent B: 0.1% TFA MeCN; Grad/isocrat: 0 to 11 min: 5% solvent B, 11 to 22 min: 5% to 95% solvent B, 22 to 28 min: 95% solvent B, 28 to 30 min: 95% to 5% solvent B, 30 to 35 min: isocratic elution at 5% solvent B. Flow rate: 1 mL/min, column temperature: 19 to 21° C.

HPLC condition 6 (for isolation and analysis of I-amino acids): Column: Phenomenex, Kinetex® EVO C18, 5 μm, 100 Å, 250×4.6 mm LC Column. Solvent A: 0.1% TFA water; Solvent B: 0.1% TFA MeCN; Grad/isocrat: 0 to 2 min: 5% solvent B, 2 to 22 min: 5% to 95% solvent B, 22 to 30 min: 95% to 95% solvent B, 30 to 30.1 min: 95% to 5% solvent B, 30.1 to 35 min: isocratic elution at 5% solvent B. Flow rate: 1 mL/min, column temperature: 19 to 21° C.

### Procedure C for the Copper-Mediated Halogen/¹²⁴I Exchange Reaction was Carried Out as Follows:

A stock of acidic reducing solution was prepared by dissolving 2,5-dihydroxybenzoic acid (25 mg, gentisic acid), citric acid (35 mg), glacial acetic acid (35 μL), and SnSO₄ (1 mg) in degassed (vacuum-sonication method) Mili-Q water (2.5 mL). A stock solution of CuSO₄ was prepared by dissolving CuSO₄·5H₂O (32.5 mg) in degassed Mili-Q water (10 mL). A 5 mL quartz tube was charged with non-radioactive 2-Iodo-4,5-difluoro phenylalanine (0.5 mg), water (50 μL), the stock reducing solution (455 μL) and the stock solution of CuSO₄ (30 μL). The vessel was placed under an atmosphere of nitrogen by gently blowing nitrogen into the solution for 10 mins. Then 124I-NaI solution (272 μCi to 1.665 mCi) was added into the solution by petite gun, sealed with a PTFE-lined screw cap, and heated at 140° C. for 30-35 minutes in an aluminum heating block. After cooling to rt, the reaction mixture was diluted to approximately 300 μL with acetic acid (5% aq) and purified using HPLC condition 5 or 6.

In vivo formulation: a certain volume of NaOH (1N) was added into the vial containing the final product HPLC solution (containing 0.1% TFA) according to the volume of the solution to ensure the pH was close to 9. Then the solution was rotary evaporated till less than 100 μL solution remained in the vial. Phosphate-buffered saline (PBS, lx) and sodium ascorbate (10 mM) were added in a volume ratio of 3/1 to the vial to form an injectable solution for the mice imaging study (150 L/mouse). Aliquots of the samples were analyzed by analytical HPLC using HPLC condition 6 at 0-hour (QC), 7-hour (stability), and 24-hour (stability) post reformulation.

### Procedure D for the Copper-Mediated Halogen/¹³¹I Exchange Reaction was Carried Out as Follows:

A stock of acidic reducing solution was prepared by dissolving 2,5-dihydroxybenzoic acid (gentisic acid, 25 mg), citric acid (35 mg), glacial acetic acid (35 μL), and SnSO₄ (1 mg) in degassed (vacuum-sonication method) Mili-Q water (2.5 mL). A stock solution of CuSO₄ was prepared by dissolving CuSO₄·5H₂O (32.5 mg) in degassed Mili-Q water (10 mL). A 5 mL quartz tube was charged with non-radioactive 2-Iodo-4,5-difluoro phenylalanine (0.5 mg), NaOH (0.1 M, 80 μL), stock reducing solution (455 μL) and a stock solution of CuSO₄ (30 μL). The vessel was placed under an atmosphere of nitrogen by gently blowing nitrogen into the solution for 10 mins. Then ¹³¹I-NaI solution (126 μCi to 3.26 mCi) was added into the solution by using a petite gun, sealed with a PTFE-lined screw cap, and heated at 140° C. for 30-35 minutes in an aluminum heating block. After cooling down to room temperature, the reaction mixture was diluted to approximately 300 μL with acetic acid (5% aq) and purified using HPLC condition 5.

In vivo formulation: a certain volume of NaOH (1N) was added into the vial containing the final product HPLC solution (containing 0.1% TFA) according to the volume of the solution to ensure the pH was close to 9. Then the solution was rotary evaporated till less than 100 μL solution remained in the vial. Phosphate-buffered saline (PBS, lx) and sodium ascorbate (10 mM) were added in a volume ratio of 3/1 to the vial to form an injectable solution for the mice efficacy study (150 to 180 L/mouse). An aliquot of sample was analyzed by analytical HPLC using HPLC condition 5 or 6.

### Example 66: Imaging of (S)-2-amino-3-(4,5-difluoro-2-(iodo-¹²⁴I)phenyl)propanoic acid

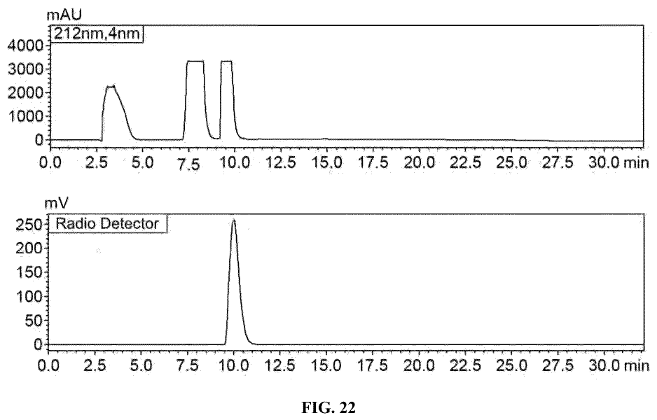

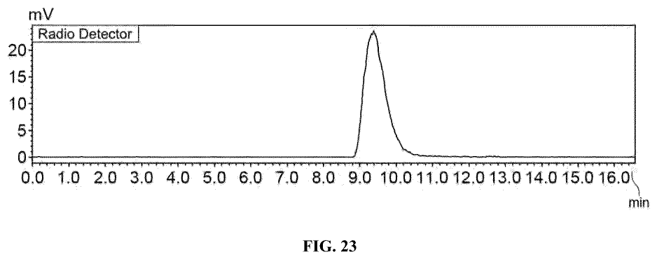

The title compound (97% isolated RCY) was produced from (S)-2-amino-3-(4,5-difluoro-2-iodophenyl)propanoic acid with procedure C (10 mins) using 272 μCi of [¹²⁴I]NaI and purified by HPLC condition 6 (FIG. 22). Quality control by HPLC condition 6 is shown in FIG. 23.

### Example 67: Imaging of (R)-2-amino-3-(4,5-difluoro-2-(iodo-¹²⁴I)phenyl)propanoic acid

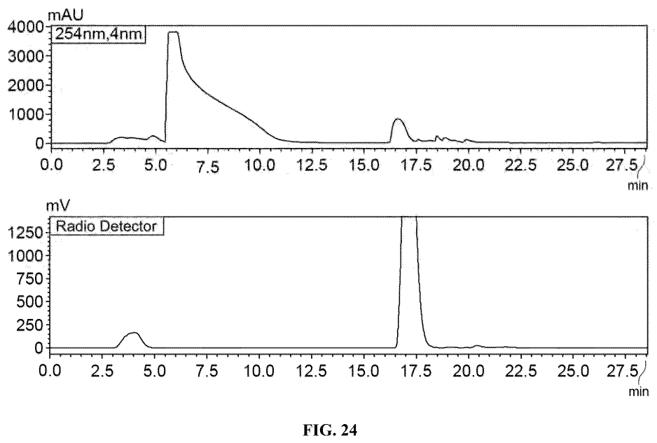

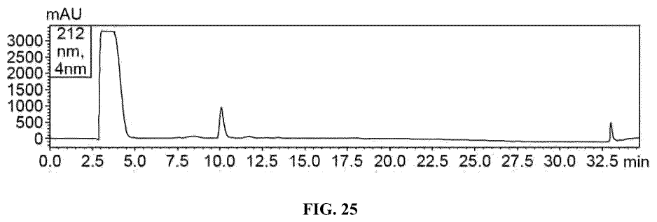

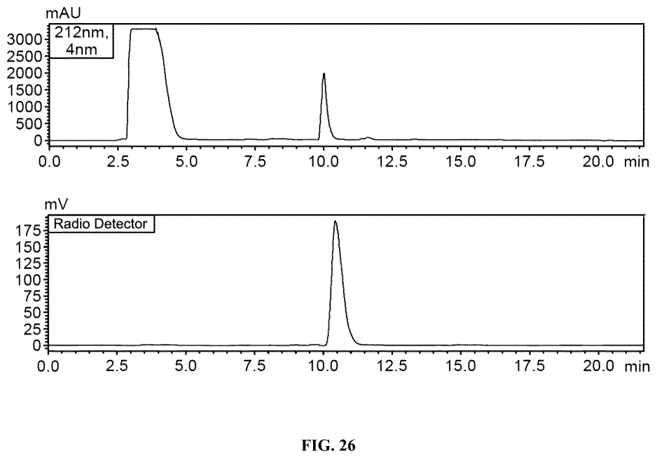

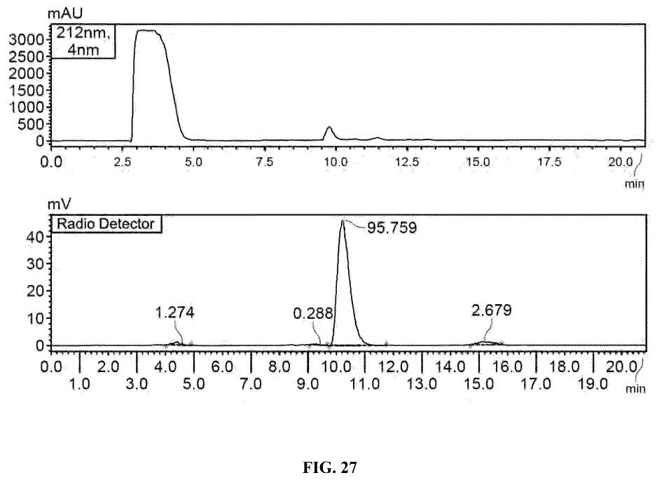

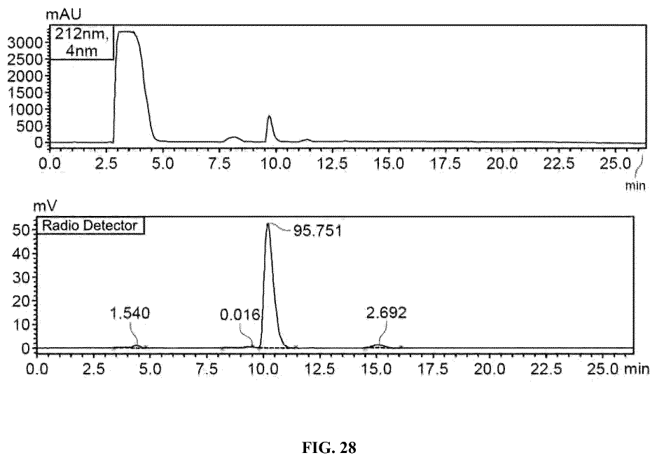

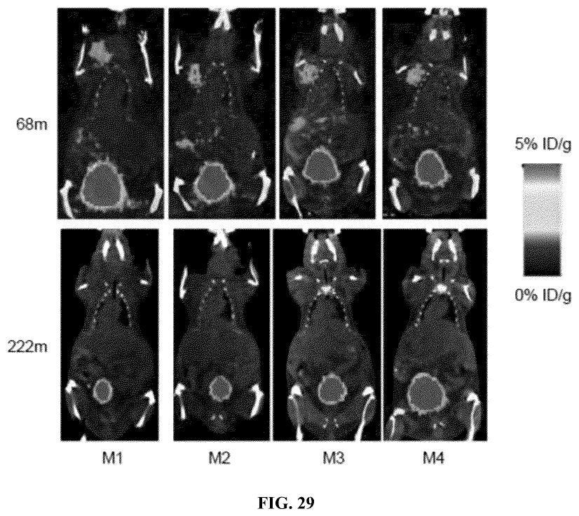

The title compound (99% isolated RCY) was prepared from (R)-2-amino-3-(4,5-difluoro-2-iodophenyl)propanoic acid with procedure C (17 mins) using 1.665 mCi [¹²⁴I]NaI and purified by HPLC condition 5 (FIG. 24). ¹²⁷I-standard is shown in FIG. 25. Quality control by HPLC condition 6 is shown in FIG. 26. In-vitro stability test: 7-hour post in vivo formulation is shown in FIG. 27. In-vitro stability test: 24-hour post in vivo formulation is shown in FIG. 28. I-124 imaging, biodistribution for (R)-2-amino-3-(4,5-difluoro-2-(iodo-¹²⁴I)phenyl)propanoic acid is shown in FIG. 29.

### Example 68: I-131 labeling and efficacy for (R)-2-amino-3-(4,5-difluoro-2-(iodo-¹³¹I)phenyl)propanoic acid

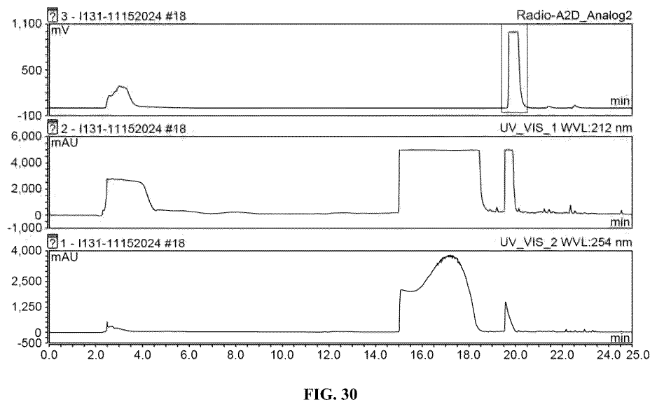

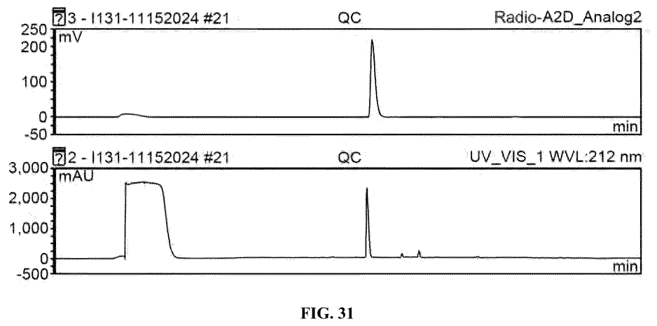

The title compound (36.6±8.9% isolated RCY, n=3) was prepared from (R)-2-amino-3-(4,5-difluoro-2-iodophenyl)propanoic acid with procedure D (17 mins average) using 2.75 to 3.0 mCi [¹³¹I]NaI and purified by HPLC condition 5 (FIG. 30). Quality Control: HPLC condition 6 is shown in FIG. 31.

The present Example demonstrates that the title compound inhibited tumor growth in MCF-7 xenografts, shortly after injection of the title compound. Those skilled in the art would appreciate that analogs of the title compound that are labeled with other radionuclides (e.g., At-211) are also expected to provide therapeutic effects in cancer, as detailed further herein.

### Example 69: Synthesis of benzyl-2-(benzyl(tert-butoxycarbonyl)amino)-3-(2,4-difluoro-5-iodo-3-methoxyphenyl)propanoate

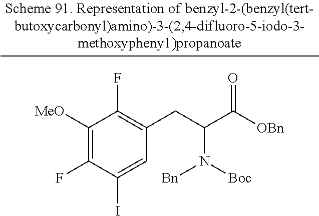 [SMILES: COc1c(F)c(I)cc(CC(C(=O)OCc2ccccc2)N(Cc2ccccc2)C(=O)OC(C)(C)C)c1F]

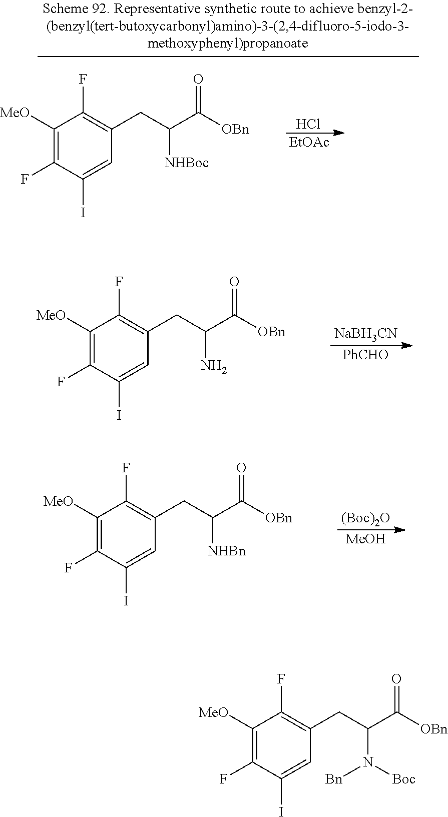

### Step 1: benzyl 2-amino-3-(2,4-difluoro-5-iodo-3-methoxyphenyl)propanoate

To a mixture of benzyl 2-((tert-butoxycarbonyl)amino)-3-(2,4-difluoro-5-iodo-3-methoxyphenyl)propanoate (300 mg, 0.55 mmol) in EtOAc (3 mL) was added hydrogen chloride (4 M in EtOAc, 6 mL). After 2 h, the mixture was concentrated under reduced pressure, dissolved in NaHCO₃ (satd, aq, 15 mL) and extracted with EtOAc (3×15 mL). The combined organic layers were dried over anhydrous Na₂SO₄ and concentrated under reduced pressure to afford benzyl 2-amino-3-(2,4-difluoro-5-iodo-3-methoxyphenyl)propanoate (200 mg, 80%) as a colorless oil. LCMS (ES, m/z): 448.00 [M+H]⁺.

### Step 2: benzyl 2-(benzylamino)-3-(2,4-difluoro-5-iodo-3-methoxyphenyl)propanoate

To a mixture of benzyl 2-amino-3-(2,4-difluoro-5-iodo-3-methoxyphenyl)propanoate (200 mg, 0.45 mmol) and benzaldehyde (52 mg, 0.49 mmol) in MeOH (5 mL) was added AcOH (one drop). After 1 h, the solution was cooled to 10° C. followed by addition of NaBH₃CN (56 mg, 0.90 mmol). After 1 h at rt, the mixture was directly purified by reverse flash chromatography (C18 silica gel, ACN-water, 0-100% with 10 mM NH₄HCO₃), affording benzyl 2-(benzylamino)-3-(2,4-difluoro-5-iodo-3-methoxyphenyl)propanoate (200 mg, 82%) as a colorless oil. LCMS (ES, m/z): 538.15 [M+H]⁺.

### Step 3: benzyl 2-(benzyl(tert-butoxycarbonyl)amino)-3-(2,4-difluoro-5-iodo-3-methoxyphenyl)propanoate

To a solution of benzyl 2-(benzylamino)-3-(2,4-difluoro-5-iodo-3-methoxyphenyl)propanoate (40 mg, 0.08 mmol) in MeOH (1 mL) at 0° C. was added NaHCO₃ (31 mg, 0.37 mmol) and (Boc)₂O (24 mg, 0.12 mmol). After 4 h, the mixture was poured into ice-water (10 mL) and extracted with EtOAc (3×10 mL). The combined organic layers were washed with brine (10 mL), dried over anhydrous Na₂SO₄ and concentrated under reduced pressure. The crude residue was purified by Prep-TLC (EA-hexanes, 15%) to afford the title compound (10.0 mg, 74%) as a white solid. ¹H NMR (400 MHz, DMSO-d₆) δ 7.43-7.24 (m, 6H), 7.17-7.06 (m, 3H), 7.06-6.98 (m, 2H), 5.09 (dt, J=34.0, 12.7 Hz, 2H), 4.49-4.32 (m, 1H), 4.28-4.04 (m, 2H), 3.81 (s, 3H), 3.28-3.10 (m, 2H), 1.34 (s, 9H). ¹⁹F NMR (376 MHz, DMSO-d₆) δ −110.72 (t, J=8.4 Hz), −132.19 (d, J=9.3 Hz), −132.31 (d, J=9.2 Hz). LCMS (ES, m/z): 538.20 [M-Boc+H]⁺; 99.8% purity (220 nm).

### Example 70: Synthesis of tert-butyl-(S)-2-((tert-butoxycarbonyl)amino)-3-(3-cyclobutoxy-2,4-difluoro-5-iodophenyl)propanoate

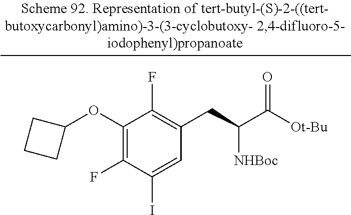 [SMILES: CC(C)(C)OC(=O)N[C@@H](Cc1cc(I)c(F)c(OC2CCC2)c1F)C(=O)OC(C)(C)C]

The title compound (16 mg) was prepared from (2S)-3-(3-cyclobutoxy-2,4-difluoro-5-iodophenyl)-2-[(diphenylmethylidene)amino]propanoate according to methods described herein. ¹H NMR (400 MHz, DMSO-d₆) δ *7.58-7.48 (m, 0.16H), *7.46 (t, J=7.1 Hz, 0.84H), *7.19 (d, J=8.3 Hz, 0.82H), *6.88 (br d, J=7.6 Hz, 0.14H), 4.62 (p, J=7.5 Hz, 1H), *4.12-4.01 (m, 0.85H), *4.01-3.92 (m, 0.17H), *2.99 (dd, J=13.7, 6.0 Hz, 0.87H), *2.95-2.89 (m, 0.12H), 2.78 (dd, J=13.7, 9.5 Hz, 1H), 2.29-2.18 (m, 2H), 2.15-2.02 (m, 2H), 1.75-1.63 (m, 1H), 1.53-1.41 (m, 1H), 1.35 (s, 9H), 1.33 (s, 9H). *Partial integration due to presence of rotamers. ¹⁹F NMR (376 MHz, DMSO-d₆) δ −109.58 (d, J=8.6 Hz, minor rotamer), −109.78 (d, J=8.9 Hz, major rotamer), −130.78 (d, J=9.1 Hz, major rotamer), −130.91 (d, J=8.1 Hz, minor rotamer). LCMS (ES, m/z): 442.00 [M-2tBu+H]⁺; 397.95 [M-Boc-tBu+H]⁺; 99.7% purity (220 nm).

### Example 72: (S)-2-amino-3-(3-cyclobutoxy-2,4-difluoro-5-iodophenyl)propanoic acid

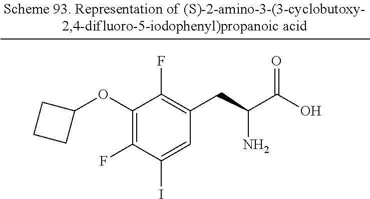 [SMILES: N[C@@H](Cc1cc(I)c(F)c(OC2CCC2)c1F)C(=O)O]

The title compound (2.0 mg) was prepared from tert-butyl (S)-2-((tert-butoxycarbonyl)amino)-3-(3-cyclobutoxy-2,4-difluoro-5-iodophenyl)propanoate according to methods described herein. ¹H NMR (400 MHz, DMSO-d₆/D₂O) δ 7.49 (t, J=7.1 Hz, 1H), 4.61 (p, J=7.3 Hz, 1H), 3.41-3.29 (m, 1H), 3.19 (dd, J=14.6, 4.2 Hz, 1H), 2.74 (dd, J=14.4, 9.2 Hz, 1H), 2.30-2.18 (m, 2H), 2.09 (br p, J=9.8 Hz, 2H), 1.75-1.63 (m, 1H), 1.53-1.39 (m, 1H). ¹⁹F NMR (376 MHz, DMSO-d₆/D₂O) δ −109.95 (d, J=8.3 Hz), −130.10 (d, J=8.3 Hz). LCMS (ES, m/z) 397.90 [M+H]⁺; 98.5% purity (220 nm).

### Example 73: Synthesis of tert-butyl-(R)-2-((tert-butoxycarbonyl)amino)-3-(3-cyclobutoxy-2,4-difluoro-5-iodophenyl)propanoate

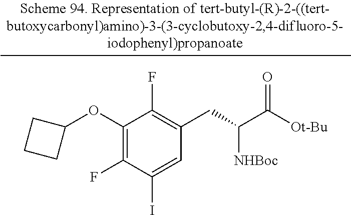 [SMILES: CC(C)(C)OC(=O)N[C@H](Cc1cc(I)c(F)c(OC2CCC2)c1F)C(=O)OC(C)(C)C]

The title compound (10 mg) was prepared from (2R)-3-(3-cyclobutoxy-2,4-difluoro-5-iodophenyl)-2-[(diphenylmethylidene)amino]propanoate according to methods described. ¹H NMR (400 MHz, DMSO-d₆) δ *7.57-7.48 (m, 0.15H), *7.46 (t, J=7.1 Hz, 0.8H), *7.19 (d, J=8.3 Hz, 0.8H), δ *6.88 (br d, J=6.6 Hz, 0.13H), 4.62 (p, J=7.5 Hz, 1H), *4.10-4.01 (m, 0.82H), *4.01-3.91 (m, 0.15H), *2.99 (dd, J=13.7, 6.0 Hz, 0.84H), *2.95-2.89 (m, 0.13H), 2.78 (dd, J=13.2, 9.4 Hz, 1H), 2.30-2.18 (m, 2H), 2.15-2.01 (m, 2H), 1.75-1.63 (m, 1H), 1.54-1.41 (m, 1H), 1.35 (s, 9H), 1.33 (s, 9H). *Partial integration due to presence of rotamers. ¹⁹F NMR (376 MHz, DMSO-d₆) δ −109.58 (d, J=9.7 Hz, minor rotamer), −109.78 (d, J=9.4 Hz, major rotamer), −130.78 (d, J=9.2 Hz, major rotamer), −130.91 (d, J=8.8 Hz, minor rotamer). LCMS (ES, m/z): 442.00 [M-2tBu+H]⁺; 397.95 [M-Boc-tBu+H]⁺; 99.7% purity (220 nm).

### Example 74: Synthesis of (R)-2-amino-3-(3-cyclobutoxy-2,4-difluoro-5-iodophenyl)propanoic acid

### Scheme 95. Representation of R)-2-amino-3-(3-cyclobutoxy-2,4-difluoro-5-iodophenyl)propanoic acid

The title compound (3.5 mg) was prepared from tert-butyl (R)-2-((tert-butoxycarbonyl)amino)-3-(3-cyclobutoxy-2,4-difluoro-5-iodophenyl)propanoate according to methods described herein. ¹H NMR (400 MHz, DMSO-d₆/D₂O) δ 7.48 (t, J=7.1 Hz, 1H), 4.60 (p, J=7.4 Hz, 1H), 3.47-3.33 (m, 1H), 3.18 (dd, J=14.2, 4.1 Hz, 1H), 2.76 (dd, J=14.4, 9.1 Hz, 1H), 2.29-2.17 (m, 2H), 2.08 (br p, J=9.8 Hz, 2H), 1.75-1.62 (m, 1H), 1.53-1.38 (m, 1H). ¹⁹F NMR (376 MHz, DMSO-d₆/D₂O) δ −110.13 (d, J=9.2 Hz), −130.31 (d, J=9.2 Hz). LCMS (ES, m/z) 397.90 [M+H]⁺; 99.3% purity (220 nm).

### Examples 75 and 76: Synthesis of tert-Butyl-(R)-2-((tert-butoxycarbonyl)amino)-3-(4,5-difluoro-2-iodophenyl)-2-methylpropanoate and tert-Butyl-(S)-2-((tert-butoxycarbonyl)amino)-3-(4,5-difluoro-2-iodophenyl)-2-methylpropanoate

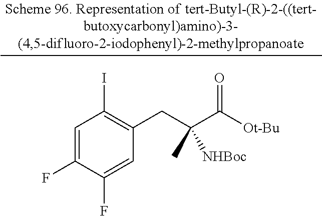 [SMILES: CC(C)(C)OC(=O)N[C@](C)(Cc1cc(F)c(F)cc1I)C(=O)OC(C)(C)C]

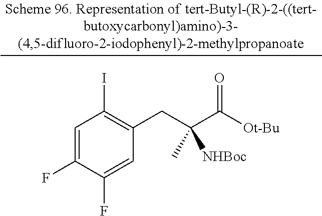 [SMILES: CC(C)(C)OC(=O)N[C@@](C)(Cc1cc(F)c(F)cc1I)C(=O)OC(C)(C)C]

Racemic tert-butyl 2-((tert-butoxycarbonyl)amino)-3-(4,5-difluoro-2-iodophenyl)-2-methylpropanoate was resolved by Prep-HPLC [Column: (R, R)-WHELK-O1-Kromasil 5 μm, 2.12×25 cm; Mobile Phase: MTBE-Hexanes, 5%; Flow rate: 20 mL/min; Wavelength: 254/220 nm] to afford tert-butyl (R)-2-((tert-butoxycarbonyl)amino)-3-(4,5-difluoro-2-iodophenyl)-2-methylpropanoate (first eluting isomer, 220 mg, 23%) and tert-butyl (S)-2-((tert-butoxycarbonyl)amino)-3-(4,5-difluoro-2-iodophenyl)-2-methylpropanoate (second eluting isomer, 240 mg, 25%) as white solids.

### tert-butyl-(R)-2-((tert-butoxycarbonyl)amino)-3-(4,5-difluoro-2-iodophenyl)-2-methylpropanoate

¹H NMR (400 MHz, DMSO-d₆) δ 7.98 (t, J=9.3 Hz, 1H), 7.19 (s, 1H), 7.02 (t, J=10.3 Hz, 1H), 3.38 (d, J=14.0 Hz, 1H), 3.19 (d, J=13.9 Hz, 1H), 1.44 (s, 9H), 1.40 (s, 9H), 1.12 (s, 3H). ¹⁹F NMR (376 MHz, DMSO-d₆) δ −139.46. LCMS (ES, m/z): 341.95 [M-tBu-Boc+H]⁺; 99.8% purity (254 nm).

### tert-butyl-(S)-2-((tert-butoxycarbonyl)amino)-3-(4,5-difluoro-2-iodophenyl)-2-methylpropanoate

¹H NMR (400 MHz, DMSO-d₆) δ 7.98 (t, J=9.3 Hz, 1H), 7.19 (s, 1H), 7.02 (t, J=10.3 Hz, 1H), 3.38 (d, J=14.0 Hz, 1H), 3.19 (d, J=14.0 Hz, 1H), 1.44 (s, 9H), 1.40 (s, 9H), 1.12 (s, 3H). ¹⁹F NMR (376 MHz, DMSO-d₆) δ −139.46. LCMS (ES, m/z): 341.90 [M-tBu-Boc+H]⁺; 99.9% purity (254 nm).

### Example 77: Synthesis of (R)-2-amino-3-(2-borono-4,5-difluorophenyl)-2-methylpropanoic acid

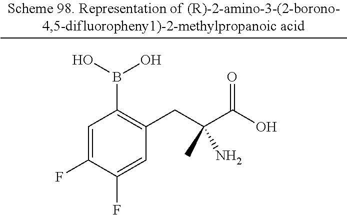 [SMILES: C[C@@](N)(Cc1cc(F)c(F)cc1B(O)O)C(=O)O]

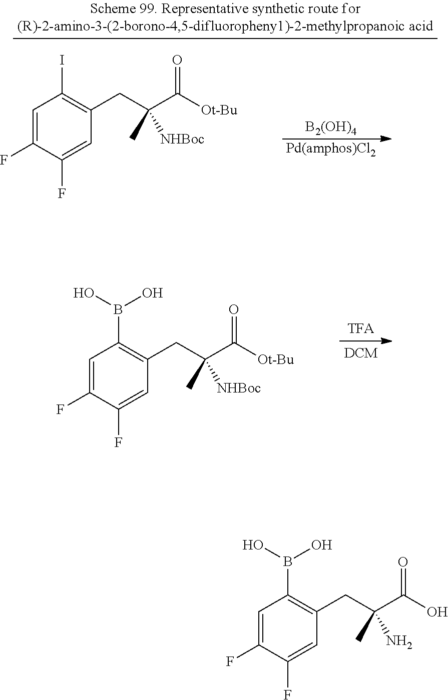 [SMILES: CC(C)(C)OC(=O)N[C@](C)(Cc1cc(F)c(F)cc1I)C(=O)OC(C)(C)C ; "B2(OH)4" ; "Pd(amphos)Cl2" ; CC(C)(C)OC(=O)N[C@](C)(Cc1cc(F)c(F)cc1B(O)O)C(=O)OC(C)(C)C ; C[C@@](N)(Cc1cc(F)c(F)cc1B(O)O)C(=O)O]

### Step 1: (R)-(2-(3-(tert-butoxy)-2-((tert-butoxycarbonyl)amino)-2-methyl-3-oxopropyl)-4,5-difluorophenyl)boronic acid

To a mixture of tert-butyl (R)-2-((tert-butoxycarbonyl)amino)-3-(4,5-difluoro-2-iodophenyl)-2-methylpropanoate (180 mg, 0.36 mmol) in 2-MeTHF/MeOH (4:1, 2 mL) was added DIEA (93 mg, 0.72 mmol), Pd(amphos)Cl₂ (26 mg, 0.04 mmol) and B₂(OH)₄ (39 mg, 0.43 mmol). The mixture was heated at 50° C. for 6 h, cooled to rt then concentrated under reduced pressure. The crude residue was purified by reverse flash chromatography (C18 silica gel, ACN-water, 0-80% with 0.1% FA) to afford (R)-(2-(3-(tert-butoxy)-2-((tert-butoxycarbonyl)amino)-2-methyl-3-oxopropyl)-4,5-difluorophenyl)boronic acid (90 mg, 60%) as a white solid. LCMS (ES, m/z): 414.20 [M−H]⁻.

### Step 2: (R)-2-amino-3-(2-borono-4,5-difluorophenyl)-2-methylpropanoic acid

To a mixture of (R)-(2-(3-(tert-butoxy)-2-((tert-butoxycarbonyl)amino)-2-methyl-3-oxopropyl)-4,5-difluorophenyl)boronic acid (80 mg, 0.19 mmol) in DCM (1.5 mL) was added TFA (1.5 mL). After 4 h, the mixture was concentrated under reduced pressure. The crude residue was purified by reverse flash chromatography (C18 silica gel, ACN-water, 0-30% with 0.1% FA), affording the title compound (20.2 mg, 40%) as a white solid. ¹H NMR (400 MHz, DMSO-d₆) δ 7.38 (dd, J=11.1, 9.3 Hz, 1H), 7.07 (dd, J=12.0, 7.5 Hz, 1H), 2.99 (d, J=17.8 Hz, 1H), 2.86 (d, J=17.9 Hz, 1H), 1.43 (s, 3H). ¹⁹F NMR (376 MHz, DMSO-d₆) δ −142.05 (d, J=21.9 Hz), −144.38 (dd, J=21.9 Hz). LCMS (ES, m/z): 260.00 [M+H]⁺; 98.1% purity (254 nm).

### Example 78: Synthesis of (S)-2-amino-3-(2-borono-4,5-difluorophenyl)-2-methylpropanoic acid

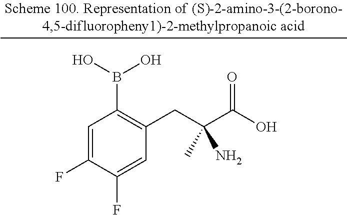 [SMILES: C[C@](N)(Cc1cc(F)c(F)cc1B(O)O)C(=O)O]

The title compound (15 mg) was prepared according to analogous methods described for Example 77 using tert-butyl (S)-2-((tert-butoxycarbonyl)amino)-3-(4,5-difluoro-2-iodophenyl)-2-methylpropanoate. LCMS (ES, m/z): 260.00 [M+H]⁺. ¹H NMR (400 MHz, DMSO-d₆) δ 7.42-7.33 (m, 1H), 7.07 (dd, J=12.0, 7.5 Hz, 1H), 2.99 (d, J=17.8 Hz, 1H), 2.86 (d, J=17.9 Hz, 1H), 1.43 (s, 3H). ¹⁹F NMR (376 MHz, DMSO-d₆) δ −142.01-(−)142.11 (m), −144.34-(−)144.43 (m). LCMS (ES, m/z): 260.00 [M+H]⁺; 98.9% purity (220 nm).

### Example 79: General Methods and Materials for ²¹¹At Radiolabeling

1,10-Phenanthroline (>99.0%), copper (II) acetate (>99.0%), sodium acetate (>99.0%), and methanol (HPLC grade) were used as purchased from the vendors. Mili-Q water was used directly after being collected from a Mili-Q system. [²¹¹At]NaAt in NaCl solution (2.5 mCi at the time of arrival) was obtained from the DOE.

HPLC condition 1 (for isolation of ²¹¹At-labeled product): Column: Phenomenex, Kinetex® EVO C18, 5 μm 100 Å, 250×4.6 mm; Mobile Phase A: water (with 0.1% TFA); Mobile Phase B: ACN (with 0.1% TFA); 0-22 min: 10-20% B; 22-30 min: 95% B; 30-35 min: 10% B; Flow rate: 1 mL/min; Column temperature: 19-21° C.

HPLC condition 2 (for isolation of ²¹¹At-labeled product): Column: Phenomenex, Kinetex® 10 m Synergi 80 Å, 250×10.00 mm; Mobile Phase A: water (with 0.1% TFA); Mobile Phase B: ACN (with 0.1% TFA); 0-22 min: 15-75% B; 22 to 30 min: 95% B; 30 to 35 min: 15% B. Flow rate: 3 mL/min; Column temperature: 19-21° C.

HPLC condition 3 (for analysis of ²¹¹At-labeled product): Column: Phenomenex, Kinetex® EVO C18, 5 μm 100 Å, 250×4.6 mm; Mobile Phase A: water (with 0.1% TFA); Mobile Phase B: ACN (with 0.1% TFA); 0-22 min: 5-95% B; 22-30 min: 95% B; 30 to 35 min: 5% B. Flow rate: 1 mL/min; Column temperature: 19-21° C.

### Procedure a for Boronic Acid/²¹¹At Exchange Reaction:

Stock solution A was prepared by dissolving 1,10-Phenanthroline (9 mg) and copper (II) acetate (4.6 mg) in MeOH (200 μL). Stock solution B was prepared by dissolving sodium acetate (205 mg) in Mili-Q water (10 mL). A borosilicate glass vial (5 mL) was charged with (R)-2-amino-3-(2-borono-4,5-difluorophenyl)propanoic acid (Example 50 above, 0.5 mg), solution A (30 l) and MeOH (30 μL). To the resulting solution was added solution B (70 μL), followed by a solution of [²¹¹At]NaAt (in aq NaCl, 480 μL, 1.86 mCi). The vial was then sealed (screw cap, PTFE liner) and heated at 100° C. After 60 min, the mixture was cooled to rt and diluted with a solution of Mili-Q water (5% acetic acid in 1.7 mL) to ˜300 μL. The resultant solution was applied to a C18 cartridge (Sep-Pak C18 Plus Short Cartridge, 360 mg Sorbent, pre-activated by using 10 mL EtOH and 10 mL water), followed by a Mili-Q water wash (2 mL, discarded). The ²¹¹At-labeled product was eluted with ACN/water (1 mL, 1:1) followed by ACN (500 μL). The eluent (˜1.5 mL) was partially concentrated (˜600 μL) under reduced pressure prior to addition of acetic acid (5% aq, 200 μL) and water (100 μL). The resulting mixture (˜900 μL) was purified by HPLC condition 1. After concentration of the eluent under reduced pressure, the isolated product was further purified by HPLC condition 2. Concentration of the eluent under reduced pressure afforded the desired [²¹¹At]-labeled substrate.

In vivo formulation: A certain volume of NaOH (1 N) was added into the vial containing the final product HPLC solution (containing 0.1% TFA) according to the volume of the solution to ensure the pH was ˜7. The resulting solution was then concentrated under reduced pressure (to remove organic solvents) followed by addition of sodium ascorbate in PBS (50 mM ascorbate in PBS 1×, pH=8) to form an injectable solution for the mice therapeutic study.

### In Vivo Studies (Tumor Xenografts in Murine Model)

All animal studies were carried out according to the protocols approved by the Institutional Animal Care and Use Committee at the University of North Carolina at Chapel Hill. Female Nude mice (5-6 weeks old) were obtained from the Division of Comparative Medicine at University of North Carolina at Chapel Hill. Mice were housed in Tecniplast M500 ventilated cages in a temperature-controlled room with 12 h light:12 h dark cycles, with food and water ad libitum.

To induce human breast cancer MCF-7 xenografts, 17-beta-estradiol pellets (7.5 mg/pellet, 60-day release, Cat #: SE-121) were surgically implanted subcutaneously behind the neck. Approximately 7 days later, after the suture was removed, approximately 4 million cells, resuspended in PBS and mixed with Matrigel (BD Bioscience) at a 1:1 (v:v) ratio, were inoculated subcutaneously in the left mammary fat pad. Tumor growth was monitored using a caliper, and the tumor volume was calculated using the following formula: Volume=Length×width×width×0.5. Animals were used for efficacy studies when the tumor sizes were approximately 100 mm³±40 mm³.

At-211 studies: NaI (10 mg/kg) was intravenously dosed (via tail vein) at 24 h before injection of the radiolabeled molecules. Each treated mouse was given approximately-54 uCi of the ²¹¹At-containing molecule via tail vein injection. Tumor volume was measured by the caliper method described above on the day of dosing and at 3-day intervals post-dosing.

For all studies, the following euthanasia criteria were used: Tumor size >2000 mm³, body weight decrease >10% for three consecutive measurements, BCS≥2.

### Example 80: Synthesis of (R)-2-amino-3-(2-(astato-²¹¹At)-4,5-difluorophenyl)propanoic acid

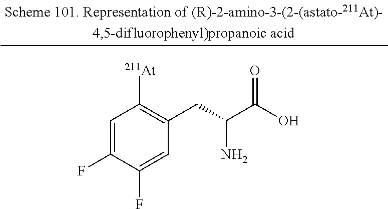

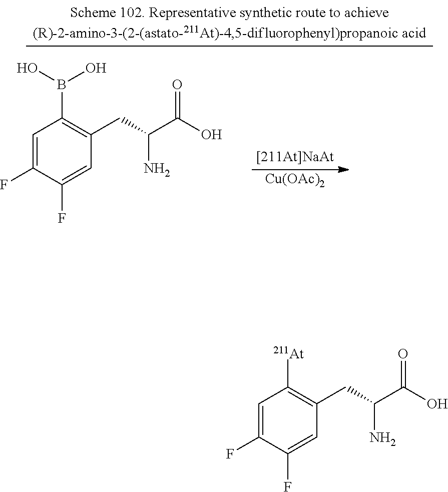

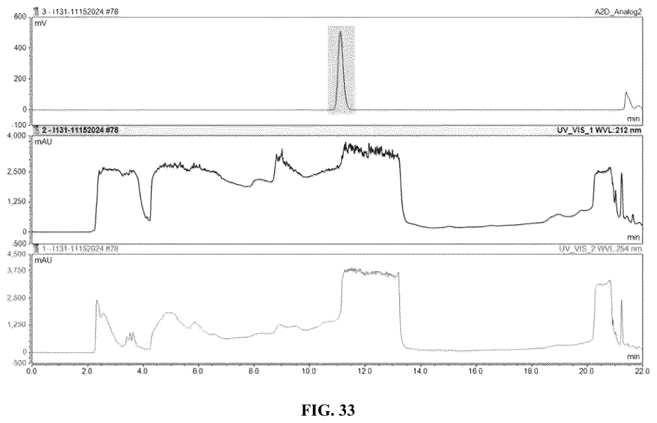

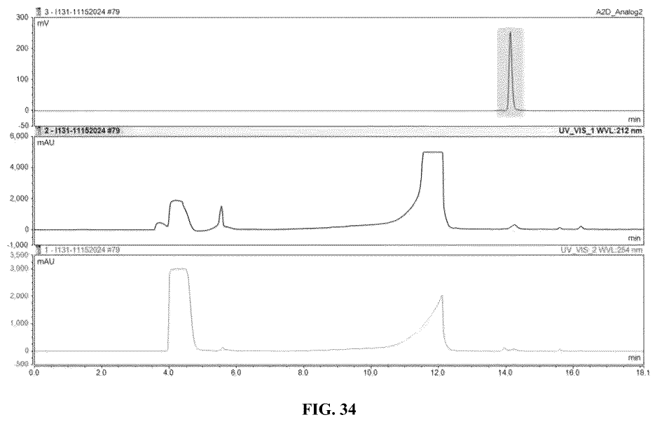

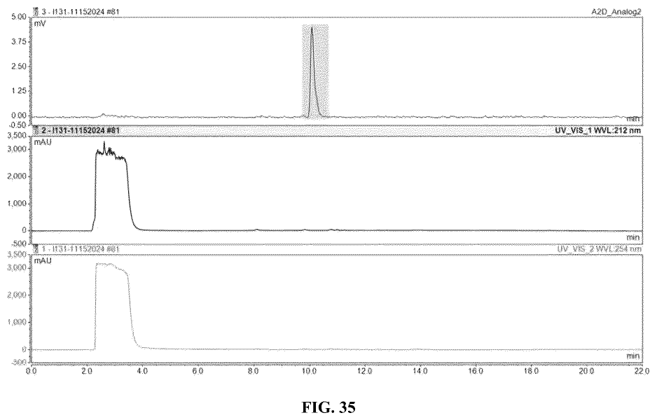

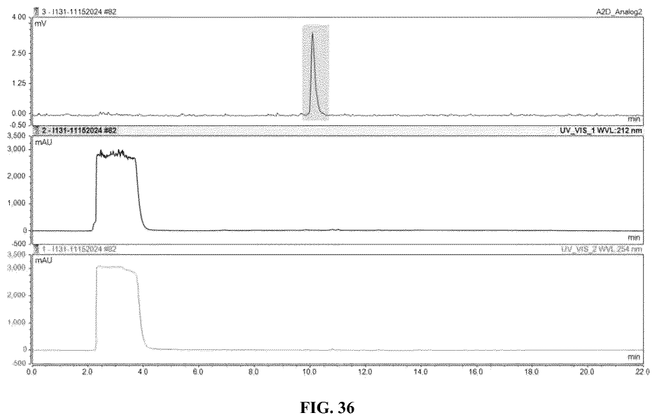

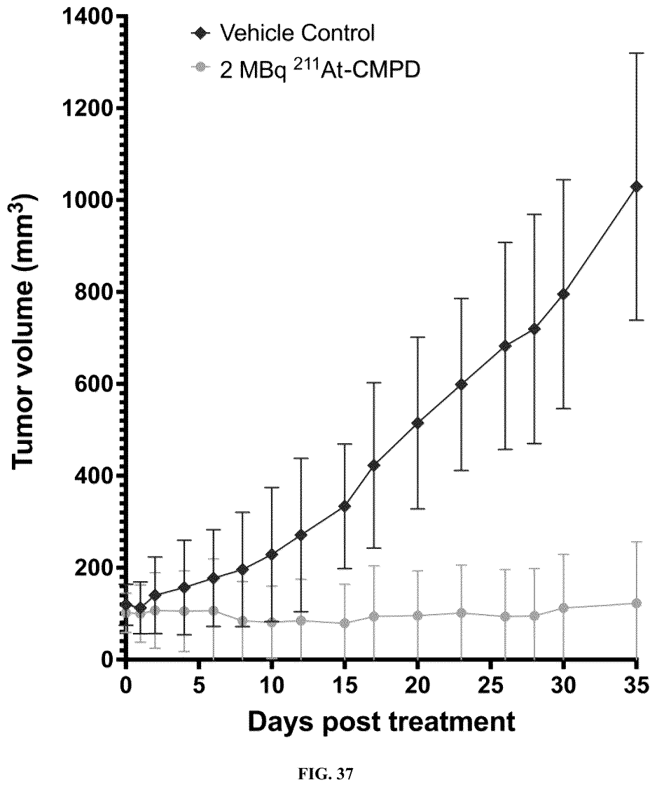

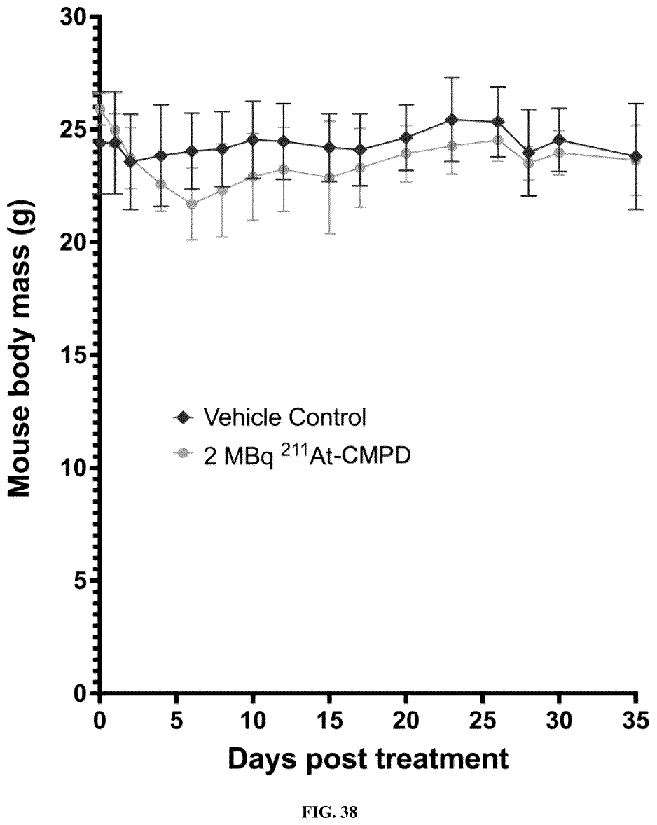

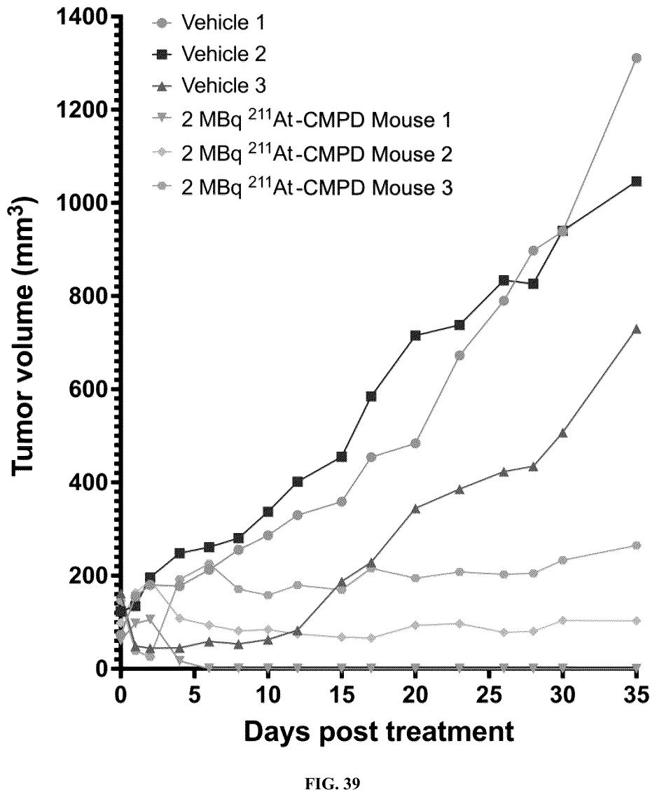

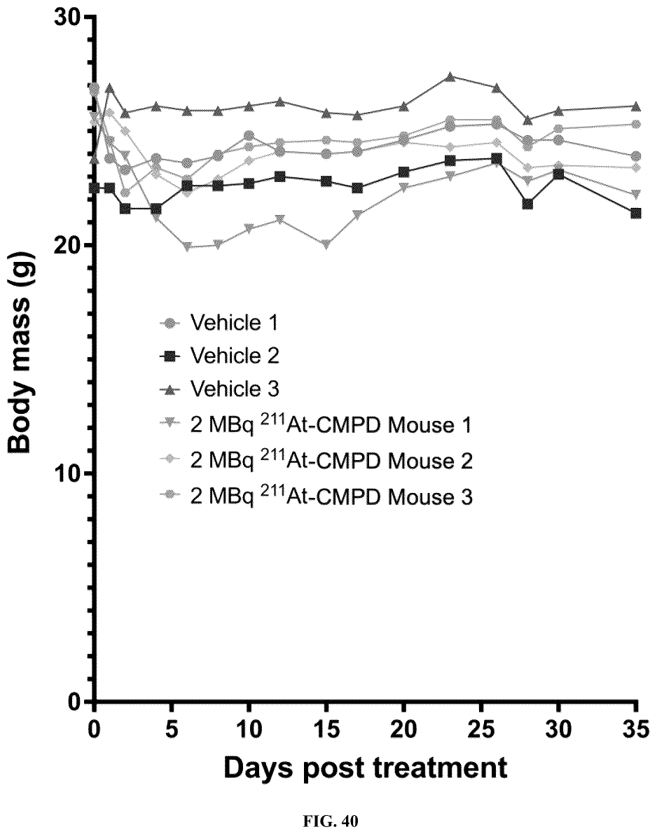

The title compound [550 μCi, RCY=31.6% (non-decay corrected); RCP>98.0%] was prepared from (R)-2-amino-3-(2-borono-4,5-difluorophenyl)propanoic acid (Example 50) described in herein by following procedure A and purified by HPLC condition 1 (FIG. 33) then HPLC condition 2 (FIG. 34). Generation of the ²¹¹At-radiolabeled product was supported by comparison to the cold ¹²⁷I standard (Example 67) under the same HPLC conditions (not shown). Quality control by HPLC condition 3 is shown in FIG. 35. Stability after 2 h of incubation at rt in the in vivo formulation solution was assessed using HPLC condition 3 (FIG. 36). Averaged tumor volumes for the At-211 efficacy study in MCF-7 tumorized mice are shown in FIG. 37. Averaged body weights for the At-211 efficacy study in MCF-7 tumorized mice are shown in FIG. 38. Individual tumor volumes for the At-211 efficacy study in MCF-7 tumorized mice are shown in FIG. 39. Individual body weights for the At-211 efficacy study in MCF-7 tumorized mice are shown in FIG. 40.

Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

That which is claimed is:

1. A compound of Formula (I): 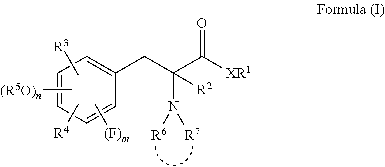
wherein:
X is —O—, —NH—, —N[(C₃-C₆) cycloalkyl]-, or —N[(C₁-C₆) alkyl]-;
R¹ and R² are independently selected from the group consisting of —H, -PG, —CH₂Ar, —(C₁-C₄) alkyl, and —(C₃-C₇) cycloalkyl;
or X and R¹ together form a (C₃-C₅) heterocycle;
R³ is a radioisotope R* selected from the group consisting of [210]At and [211]At, wherein R³ is in the 2-position on the phenyl ring;
R⁴ is —F, or radioisotope [18]F;
R⁵ is -PG, —(C₁-C₆) alkyl, or —(C₃-C₆) cycloalkyl;
R⁶ and R⁷ are independently selected from the group consisting of —H, -PG, —CH₂Ar, —(C₁-C₄) alkyl, and —(C₃-C₆) cycloalkyl;
or R⁶ and R⁷ together form a (C₃-C₅) heterocycle;
PG, in each instance, is a protecting group;
m is 1, 2, or 3; and
n is 0, 1, or 2;
or any stereoisomer and/or pharmaceutically acceptable salt thereof.

2. The compound of claim 1, wherein n is 1.

3. The compound of claim 1, wherein m is 1.

4. The compound of claim 1, wherein R⁴ is —F.

5. The compound of claim 1, wherein R⁴ is the radioisotope [18]F.

6. The compound of claim 1, wherein R³ is [211]At.

7. The compound of claim 1, wherein R⁵ is —CH₃.

8. The compound of claim 1, wherein R⁵ is cyclopropyl, cyclobutyl, cyclopentyl, or cyclohexyl.

9. The compound of claim 1, wherein X is —O— or —NH—.

10. The compound of claim 1 having the formula:

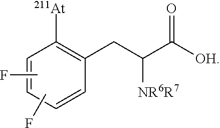

11. The compound of claim 1 having the formula:

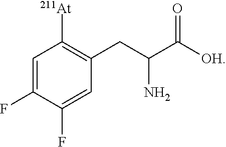

12. A compound of Formula (I): 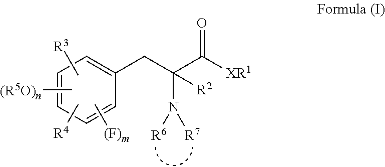
wherein:
X is —O—, —NH—, —N[(C₃-C₆) cycloalkyl]-, or —N[(C₁-C₆) alkyl]-;
R¹ and R² are independently selected from the group consisting of —H, -PG, —CH₂Ar, —(C₁-C₄) alkyl, and —(C₃-C₇) cycloalkyl;
or X and R¹ together form a (C₃-C₅) heterocycle;
R³ is a radioisotope R* selected from the group consisting of [210]At and [211]At;
R⁴ is —H, —F, or radioisotope [18]F;
R⁵ is -PG, —(C₁-C₆) alkyl, or —(C₃-C₆) cycloalkyl;
R⁶ and R⁷ are independently selected from the group consisting of —H, -PG, —CH₂Ar, —(C₁-C₄) alkyl, and —(C₃-C₆) cycloalkyl;
or R⁶ and R⁷ together form a (C₃-C₅) heterocycle;
PG, in each instance, is a protecting group;
m is 0, 1, 2, or 3; and
n is 1 or 2;
or any stereoisomer and/or pharmaceutically acceptable salt thereof.

13. The compound of claim 12, wherein R⁵ is —CH₃.

14. The compound of claim 13, wherein R⁵ is cyclopropyl, cyclobutyl, cyclopentyl, or cyclohexyl.

15. The compound of claim 12, wherein R⁴ is —F, or the radioisotope [18]F.

16. The compound of claim 12, wherein m is 1, 2, or 3.

17. The compound of claim 14, wherein m is 1, 2, or 3.

18. The compound of claim 12, wherein X is —O— or —NH—.

19. The compounds of claim 12, wherein R³ is in the 2-position on the phenyl ring.

20. The compound of claim 1, wherein the compound is a PET imaging agent, a SPECT imaging agent, or a radiolabeled-based therapeutic agent.

21. A pharmaceutical formulation comprising a compound of claim 1 and at least one pharmaceutically acceptable carrier.
